(19) United States
(12) Patent Application Publication
(10) Pub. No.: US 2023/0147959 A1
(43) Pub. Date: May 11, 2023

(54) COMPOUND FOR ORGANIC OPTOELECTRONIC DEVICE, COMPOSITION FOR ORGANIC OPTOELECTRONIC DEVICE, ORGANIC OPTOELECTRONIC DEVICE AND DISPLAY DEVICE

(71) Applicant: SAMSUNG SDI CO., LTD., Yongin-si (KR)

(72) Inventors: Jiyun KWON, Suwon-si (KR); Dong Min KANG, Suwon-si (KR); Kipo JANG, Suwon-si (KR); Junmo PARK, Suwon-si (KR); Hansol SEO, Suwon-si (KR); Chanwoo KIM, Suwon-si (KR); Sangil LEE, Suwon-si (KR); Sung-Hyun JUNG, Suwon-si (KR); Ho Kuk JUNG, Suwon-si (KR)

(21) Appl. No.: 17/978,417

(22) Filed: Nov. 1, 2022

(30) Foreign Application Priority Data
Nov. 5, 2021 (KR) 10-2021-0151651
Oct. 31, 2022 (KR) 10-2022-0142668

(51) Int. Cl. H01L 51/00; C07D 405/04; C07D 211/54; C07D 405/14; C07D 409/14; C07D 405/10; C07D 495/04; C07D 409/04; C07D 493/04; C07D 403/04; C07D 403/10; C07F 7/08; C07D 401/04; C07D 413/04; C07D 417/04; H01L 51/50

(52) U.S. Cl. CPC H01L 51/0067; H01L 51/0073; C07D 405/04; H01L 51/0052; H01L 51/0064; C07D 211/54; C07D 405/14; H01L 51/0054; C07D 409/14; H01L 51/0074; H01L 51/0072; C07D 405/10; H01L 51/0071; C07D 495/04; C07D 409/04; C07D 493/04; C07D 403/04; C07D 403/10; C07F 7/0812; H01L 51/0094; C07D 401/04; C07D 413/04; C07D 417/04; H01L 51/5024; C07B 2200/13

(57) ABSTRACT

A compound for an organic optoelectronic device, a composition for an organic optoelectronic device including the same, an organic optoelectronic device, and a display device, the compound being represented by Chemical Formula 1:

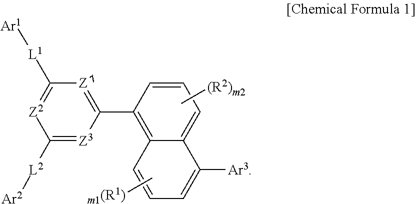

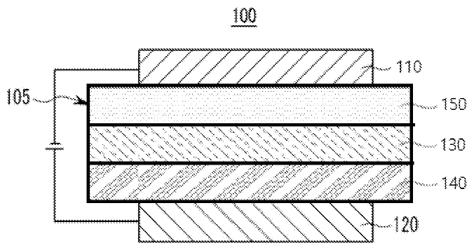

### CROSS-REFERENCE TO RELATED APPLICATION

This application claims priority to and the benefit of Korean Patent Application No. 10-2021-0151651 filed in the Korean Intellectual Property Office on Nov. 5, 2021, and Korean Patent Application No. 10-2022-0142668 filed in the Korean Intellectual Property Office on Oct. 31, 2022, the entire contents of which are incorporated herein by reference.

### BACKGROUND

### 1. Field

Embodiments relate to a compound for an organic optoelectronic device, a composition for an organic optoelectronic device, an organic optoelectronic device, and a display device.

### 2. Description of the Related Art

An organic optoelectronic device (e.g., organic optoelectronic diode) is a device capable of converting electrical energy and optical energy to each other.

Organic optoelectronic devices may be largely divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device may include an organic photoelectric device, an organic light emitting diode, an organic solar cell, and an organic photo conductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode is greatly influenced by an organic material between electrodes.

### SUMMARY

The embodiments may be realized by providing a compound for an organic optoelectronic device, the compound being represented by Chemical Formula 1:

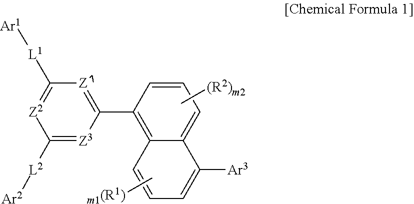

wherein, in Chemical Formula 1, Z¹ to Z³ are each independently N or CR^{a}, provided that at least two of Z¹ to Z³ are N, L¹ and L² are each independently a single bond, a substituted or unsubstituted C6 to C30 arylene group, or a substituted or unsubstituted C2 to C30 heterocyclic group, Ar¹ and Ar² are each independently a substituted or unsubstituted C6 to C30 aryl group or a substituted or unsubstituted C2 to C30 heterocyclic group, AO is a substituted or unsubstituted C6 to C30 aryl group, a substituted or unsubstituted pyridyl group, a substituted or unsubstituted pyrimidinyl group, a substituted or unsubstituted triazinyl group, a substituted or unsubstituted dibenzofuranyl group, a substituted or unsubstituted dibenzothiophenyl group, a substituted or unsubstituted benzonaphthofuranyl group, or a substituted or unsubstituted benzonaphthothiophenyl group, R^{a}, R¹, and R² are each independently hydrogen, deuterium, a substituted or unsubstituted C1 to C20 alkyl group, or a substituted or unsubstituted C6 to C20 aryl group, m1 and m2 are each independently an integer of 1 to 3, and when m2 is 2 or 3, each R² is separately present, or adjacent R²s are bonded with each other to form a ring.

The embodiments may be realized by providing a composition for an organic optoelectronic device, the composition including a first compound; and a second compound, wherein the first compound is the compound according to an embodiment, and the second compound is represented by Chemical Formula 2,

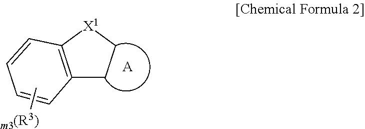

in Chemical Formula 2, X¹ is O, S, NR^{b}, CR^{c}R^{d}, or SiR^{e}R^{f}, R^{b}, R^{c}, R^{d}, R^{e}, R^{f}, and R³ are each independently hydrogen, deuterium, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, m3 is an integer of 1 to 4, and ring A is a ring of Group II,

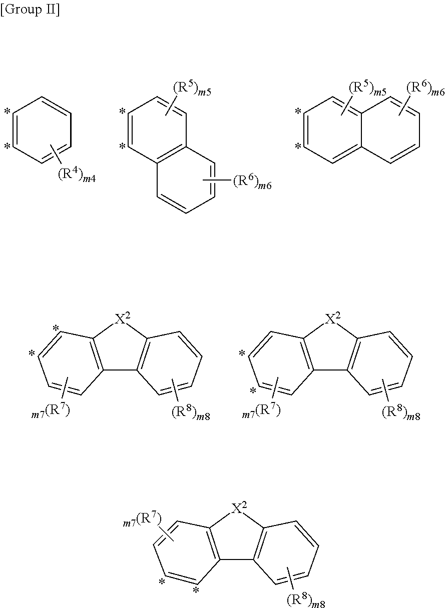

in Group II, * is a linking point, X² is O, S, NR^{g}, CR^{h}Rⁱ, or SiR^{j}R^{k}, R^{g}, R^{h}, Rⁱ, R^{k}, and R⁴ to R⁸ are each independently hydrogen, deuterium, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, m4, m6, and m8 are each independently an integer of 1 to 4, m5 and m7 are each independently 1 or 2, and at least one of R³ to R⁸ is a group represented by Chemical Formula a,

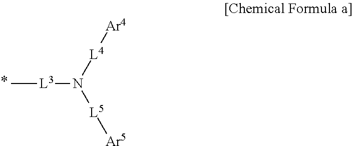

in Chemical Formula a, L³ to L⁵ are each independently a single bond or a substituted or unsubstituted C6 to C30 arylene group, Ar⁴ and Ar⁵ are each independently a substituted or unsubstituted amine group, a substituted or unsubstituted C6 to C30 aryl group, or a substituted or unsubstituted C2 to C30 heterocyclic group, and * is a linking point.

The embodiments may be realized by providing an organic optoelectronic device including an anode and a cathode facing each other, and at least one organic layer between the anode and the cathode, wherein the at least one organic layer includes a light emitting layer, and the light emitting layer includes the compound according to an embodiment.

The embodiments may be realized by providing an organic optoelectronic device including an anode and a cathode facing each other, and at least one organic layer between the anode and the cathode, wherein the at least one organic layer includes a light emitting layer, and the light emitting layer includes the composition according to an embodiment.

The embodiments may be realized by providing a display device including the organic optoelectronic device according to an embodiment.

### BRIEF DESCRIPTION OF THE DRAWING

Features will be apparent to those of skill in the art by describing in detail exemplary embodiments with reference to the attached drawing in which:

the FIGURE is a cross-sectional view illustrating an organic light emitting diode according to an embodiment.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. It will also be understood that when a layer or element is referred to as being “on” another layer or element, it can be directly on the other layer or element, or intervening layers may also be present. In addition, it will also be understood that when a layer is referred to as being “between” two layers, it can be the only layer between the two layers, or one or more intervening layers may also be present. Like reference numerals refer to like elements throughout. As used herein, the term “or” is not an exclusive term, e.g., “A or B” would include A, B, or A and B.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example, “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In addition, in specific examples, “substituted” refers to replacement of at least one hydrogen of a substituent or a compound by deuterium, a C1 to C20 alkyl group, a C6 to C30 aryl group, or a cyano group. In addition, in specific examples, “substituted” refers to replacement of at least one hydrogen of a substituent or a compound by deuterium, a C1 to C5 alkyl group, a C6 to C18 aryl group, or a cyano group. In addition, in specific examples, “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

As used herein, “unsubstituted” refers to non-replacement of a hydrogen atom by another substituent and remaining of the hydrogen atom.

As used herein, “hydrogen substitution (—H)” may include deuterium substitution (-D) or “tritium substitution (-T).

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and may include a group in which all elements of the hydrocarbon aromatic moiety have p-orbitals which form conjugation, for example a phenyl group, a naphthyl group, and the like, a group in which two or more hydrocarbon aromatic moieties may be linked by a sigma bond, for example a biphenyl group, a terphenyl group, a quarterphenyl group, and the like, and a group in which two or more hydrocarbon aromatic moieties are fused directly or indirectly to provide a non-aromatic fused ring, for example, a fluorenyl group, and the like.

The aryl group may include a monocyclic, polycyclic or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” refers to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzothiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, a substituted or unsubstituted dibenzothiophenyl group, a substituted or unsubstituted benzonaphthofuranyl group, a substituted or unsubstituted benzonaphthothiophenyl group, a substituted or unsubstituted dibenzosilolyl group, or a combination thereof, but is not limited thereto.

In the present specification, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a compound for an organic optoelectronic device according to an embodiment is described.

The compound for an organic optoelectronic device according to an embodiment may be represented by, e.g., Chemical Formula 1.

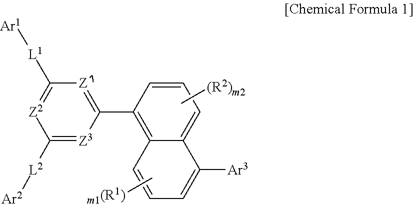

In Chemical Formula 1, Z¹ to Z³ may each independently be, e.g., N or CR^{a}. In an implementation, at least two of Z¹ to Z³ may be N.

L¹ and L² may each independently be or include, e.g., a single bond, a substituted or unsubstituted C6 to C30 arylene group, or a substituted or unsubstituted C2 to C30 heterocyclic group.

Ar¹ and Ar² may each independently be or include, e.g., a substituted or unsubstituted C6 to C30 aryl group or a substituted or unsubstituted C2 to C30 heterocyclic group.

Ar³ may be or may include, e.g., a substituted or unsubstituted C6 to C30 aryl group, a substituted or unsubstituted pyridyl group, a substituted or unsubstituted pyrimidinyl group, a substituted or unsubstituted triazinyl group, a substituted or unsubstituted dibenzofuranyl group, a substituted or unsubstituted dibenzothiophenyl group, a substituted or unsubstituted benzonaphthofuranyl group, or a substituted or unsubstituted benzonaphthothiophenyl group.

R^{a}, R¹, and R² may each independently be or include, e.g., hydrogen, deuterium, a substituted or unsubstituted C1 to C20 alkyl group, or a substituted or unsubstituted C6 to C20 aryl group.

m1 and m2 may each independently be, e.g., an integer of 1 to 3.

In an implementation, when m2 is 2 or 3, each R² may be separately or independently present, or adjacent R²s may be bonded or fused with each other to form a ring.

The compound represented by Chemical Formula 1 may have a structure in which a nitrogen-containing 6-membered ring and AO are substituted at or bonded on 1st and 5th positions of a naphthalene moiety, respectively.

By being substituted at the 1st and 5th positions of the naphthalene moiety, a bond angle may be maximized and stability between the host-host and the host-dopant may be improved by giving a sterically distorted effect, thereby inducing a reduction in a full width at half maximum and improving the efficiency and life-span of the device.

In an implementation, Ar³ may be, e.g., a substituted or unsubstituted C6 to C30 aryl group, a substituted or unsubstituted dibenzofuranyl group, a substituted or unsubstituted dibenzothiophenyl group, a substituted or unsubstituted benzonaphthofuranyl group, a substituted or unsubstituted benzonaphthothiophenyl group, a substituted or unsubstituted pyridyl group, a substituted or unsubstituted pyrimidinyl group, or a substituted or unsubstituted triazinyl group. In an implementation, when Ar³ is a C6 to C30 aryl group substituted with a cyano group, the cyano group may have a strong electron withdrawing property and acts as an excellent electron acceptor, overall LUMO region is expanded as in the case where Ar³ is a substituted or unsubstituted pyridyl group, a substituted or unsubstituted pyrimidinyl group, or a substituted or unsubstituted triazinyl group. It may effectively separate LUMO/HOMO according to the structure, and efficiency and life-span improvement effects may be expected.

In an implementation, the cyano group may have little structural change in the non-emission-inactivated state and the excited state, and PL efficiency may be improved.

In Chemical Formula 1, when m1 is 2 or more, each R¹ may be the same or different from each other.

In Chemical Formula 1, when m2 is 2 or more, each R² may be the same or different from each other.

Chemical Formula 1 is represented by Chemical Formula 1-1 or Chemical Formula 1-2.

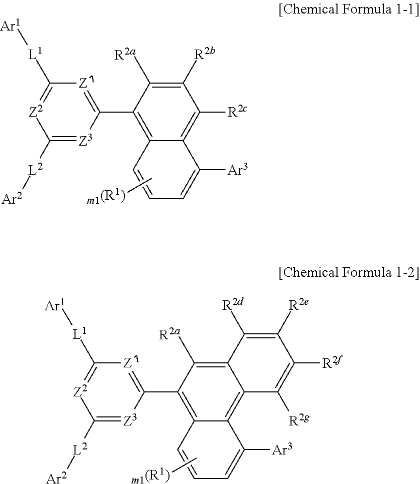

wherein, in Chemical Formula 1-1 and Chemical Formula 1-2,

Z¹ to Z³, L¹, L², Ar¹ to Ar², R¹, and m1 are the same as defined above, and

R^{2a}, R^{2b}, R^{2c}, R^{2d}, R^{2e}, R^{2f} and R^{2g} are each independently hydrogen, deuterium, a substituted or unsubstituted C1 to C20 alkyl group, or a substituted or unsubstituted C6 to C20 aryl group.

In an implementation, Ar¹ and Ar² may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted chrysenyl group, a substituted or unsubstituted benzophenanthrenyl group, a substituted or unsubstituted triphenylene group, a substituted or unsubstituted fluorenyl group, a substituted or unsubstituted dibenzosilolyl group, a substituted or unsubstituted dibenzofuranyl group, a substituted or unsubstituted dibenzothiophenyl group, a substituted or unsubstituted carbazolyl group, a substituted or unsubstituted benzonaphthofuranyl group, a substituted or unsubstituted benzonaphthothiophenyl group, a substituted or unsubstituted dinaphthofuranyl group, a substituted or unsubstituted dinaphthothiophenyl group, a substituted or unsubstituted fused dibenzofuranyl group, a substituted or unsubstituted fused dibenzothiophenyl group, a substituted or unsubstituted benzocarbazolyl group, a substituted or unsubstituted phenoxazinyl group, a substituted or unsubstituted thiophenoxazinyl group, a substituted or unsubstituted benzophenoxazinyl group, a substituted or unsubstituted benzothiophenoxazinyl group, a substituted or unsubstituted 10-phenyl-10H-spiro[acridine-9,9′-fluorenyl group], a substituted or unsubstituted 10H-spiro[acridine-9,9′-fluorenyl group], or a substituted or unsubstituted spiro[fluorene-9,9′-xanthenyl group].

In an implementation, Ar¹ and Ar² may each independently be, e.g., a substituted or phenyl group, a substituted or unsubstituted biphenyl group, a substituted or unsubstituted terphenyl group, a substituted or unsubstituted naphthyl group, a substituted or unsubstituted triphenylene group, a substituted or unsubstituted fluorenyl group, a substituted or unsubstituted dibenzosilolyl group, a substituted or unsubstituted dibenzofuranyl group, a substituted or unsubstituted dibenzothiophenyl group, or a substituted or unsubstituted carbazolyl group.

In an implementation, Ar¹ and Ar² may each independently be, e.g., a substituted or phenyl group, a substituted or unsubstituted biphenyl group, a substituted or unsubstituted naphthyl group, a substituted or unsubstituted dibenzosilolyl group, a substituted or unsubstituted dibenzofuranyl group, or a substituted or unsubstituted dibenzothiophenyl group.

In an implementation, Ar³ may be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted fluorenyl group, a substituted or unsubstituted dibenzofuranyl group, a substituted or unsubstituted dibenzothiophenyl group, a substituted or unsubstituted benzonaphthofuranyl group, a substituted or unsubstituted benzonaphthothiophenyl group, a substituted or unsubstituted pyridyl group, a substituted or unsubstituted pyrimidinyl group, or a substituted or unsubstituted triazinyl group.

In an implementation, Ar³ may be, e.g., a substituted or unsubstituted phenyl group, a substituted or unsubstituted biphenyl group, a substituted or unsubstituted naphthyl group, a substituted or unsubstituted anthracenyl group, a substituted or unsubstituted phenanthrenyl group, a substituted or unsubstituted fluorenyl group, a substituted or unsubstituted dibenzofuranyl group, or a substituted or unsubstituted benzonaphthofuranyl group.

In an implementation, Ar³ may be, e.g., a substituted or unsubstituted phenyl group, a substituted or unsubstituted naphthyl group, a substituted or unsubstituted anthracenyl group, a substituted or unsubstituted phenanthrenyl group, or a substituted or unsubstituted dibenzofuranyl group.

In an implementation, when AO is substituted, the substituent may be, e.g., a cyano group.

In an implementation, L¹ and L² may each independently be, e.g., a single bond, a substituted or unsubstituted phenylene group, a substituted or unsubstituted biphenylene group, a substituted or unsubstituted naphthylene group, a substituted or unsubstituted phenanthrenylene group, a substituted or unsubstituted dibenzofuranylene group, or a substituted or unsubstituted dibenzothiophenylene group.

In an implementation, L¹ and L² may each be a single bond.

In an implementation, moieties *-L¹-Ar¹ and *-L²-Ar² may each independently be a moiety of Group I.

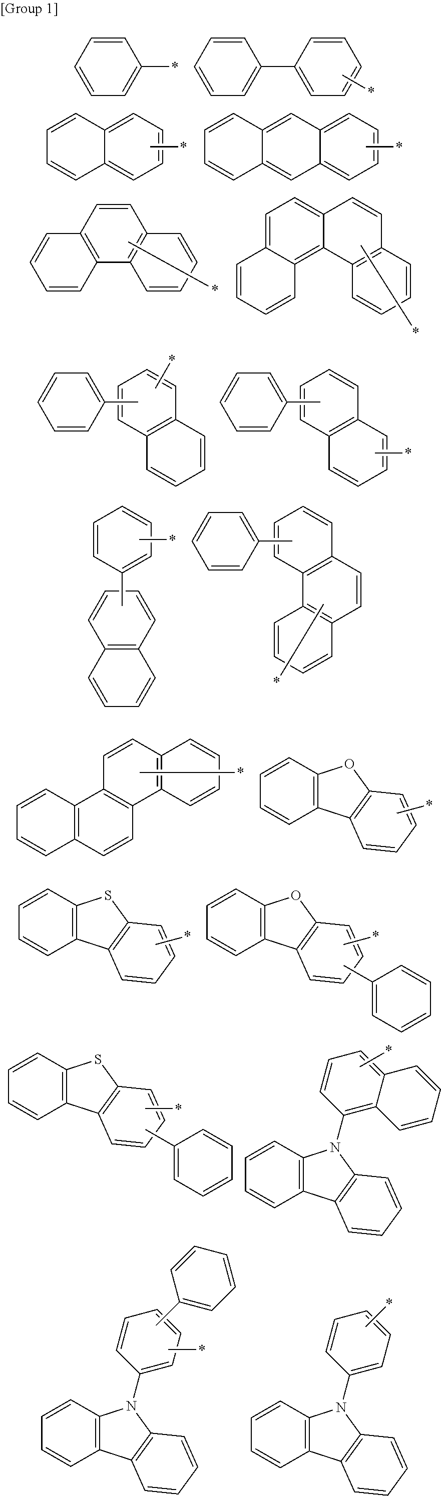

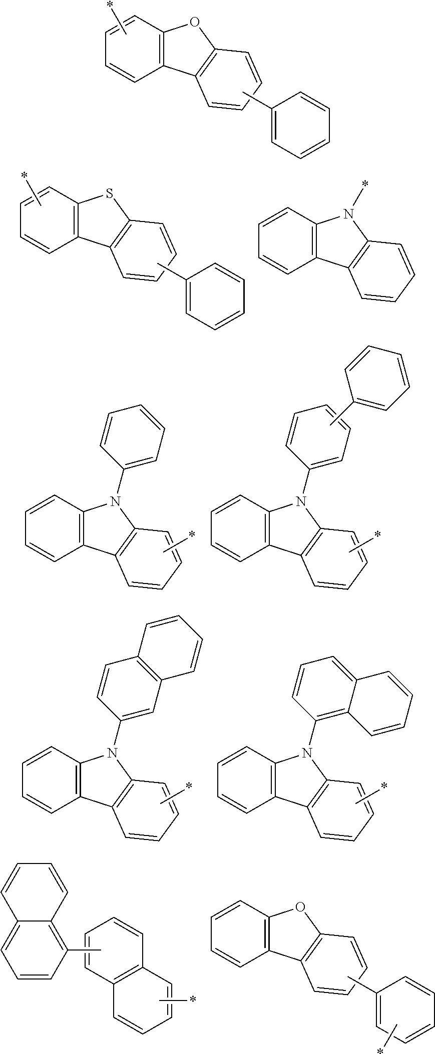

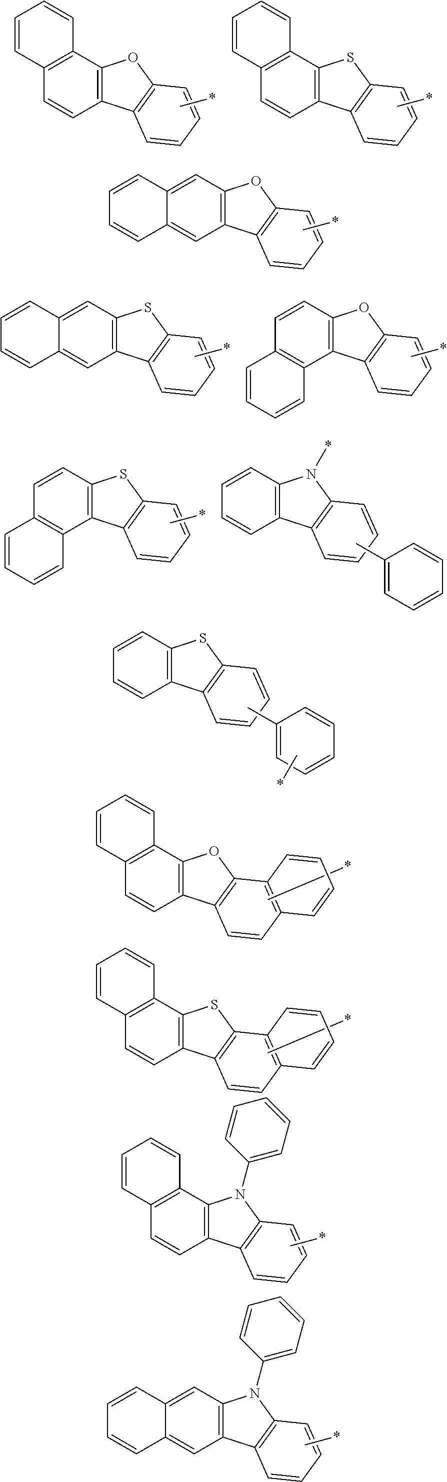

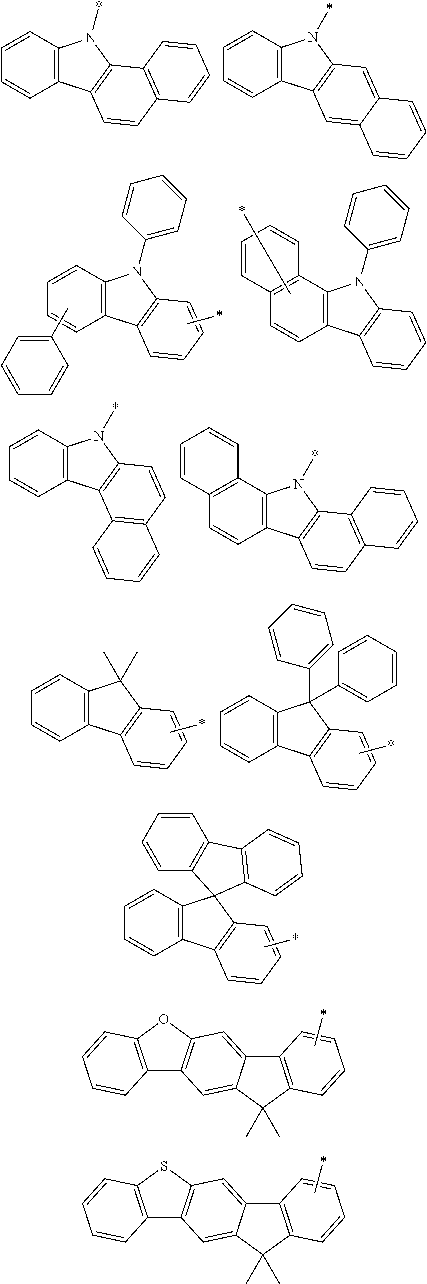

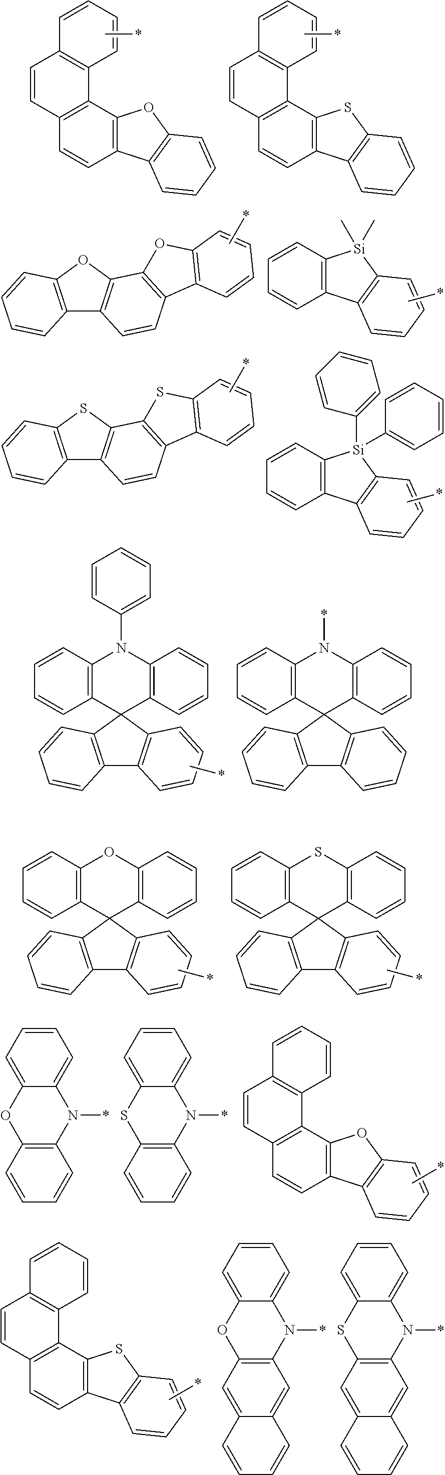

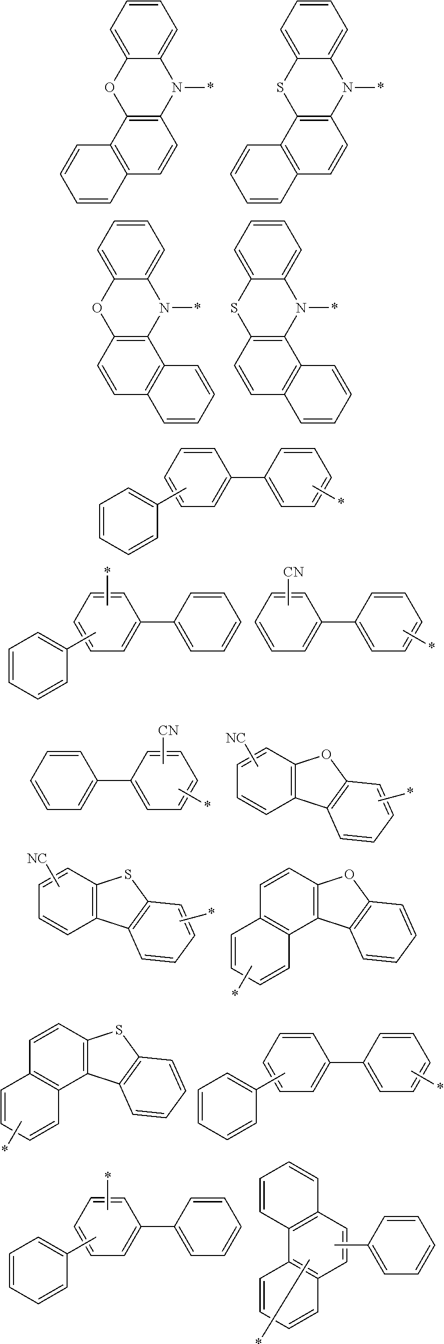

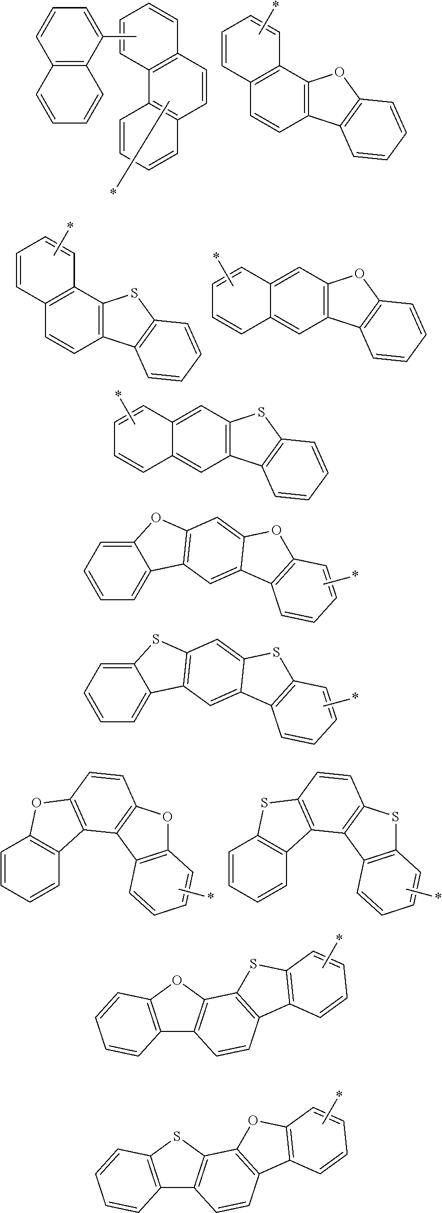

In Group I, * is a linking point.

The moieties of Group I may be unsubstituted (e.g., as illustrated), or may be substituted with at least one substituent.

In an implementation, the substituent may be, e.g., deuterium, a C1 to C10 alkyl group, or a C6 to C12 aryl group.

In an implementation, R^{a}, R¹, and R² may each independently be, e.g., hydrogen, deuterium, a substituted or unsubstituted C1 to C10 alkyl group, or a substituted or unsubstituted C6 to C12 aryl group.

In an implementation, Ar¹ and Ar² may each independently be, e.g., a substituted or unsubstituted phenyl group, a substituted or unsubstituted dibenzofuranyl group or a substituted or unsubstituted dibenzothiophenyl group, Ar³ may be, e.g., a substituted or unsubstituted phenyl group, a substituted or unsubstituted naphthyl group, a substituted or unsubstituted phenanthrenyl group, a substituted or unsubstituted dibenzofuranyl group, or a substituted or unsubstituted dibenzothiophenyl group, and L¹ and L² may each be, e.g., a single bond.

In an implementation, R¹ and R² may each independently be, e.g., hydrogen, a substituted or unsubstituted methyl group, or a substituted or unsubstituted phenyl group, and m1 and m2 may each be, e.g., 1 or 2. In an implementation, m1 and m2 may each be 1.

In an implementation, the compound for the organic optoelectronic device represented by Chemical Formula 1 may be, e.g., a compound of Group 1.

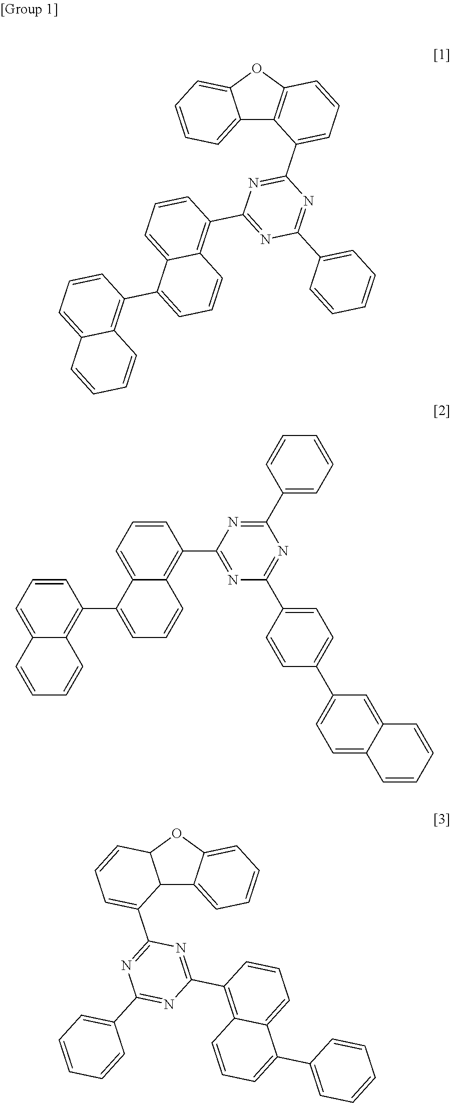 [SMILES: c1ccc(-c2nc(-c3cccc4c(-c5cccc6ccccc56)cccc34)nc(-c3cccc4oc5ccccc5c34)n2)cc1 ; c1ccc(-c2nc(-c3ccc(-c4ccc5ccccc5c4)cc3)nc(-c3cccc4c(-c5cccc6ccccc56)cccc34)n2)cc1 ; C1=CC2Oc3ccccc3C2C(c2nc(-c3ccccc3)nc(-c3cccc4c(-c5ccccc5)cccc34)n2)=C1]

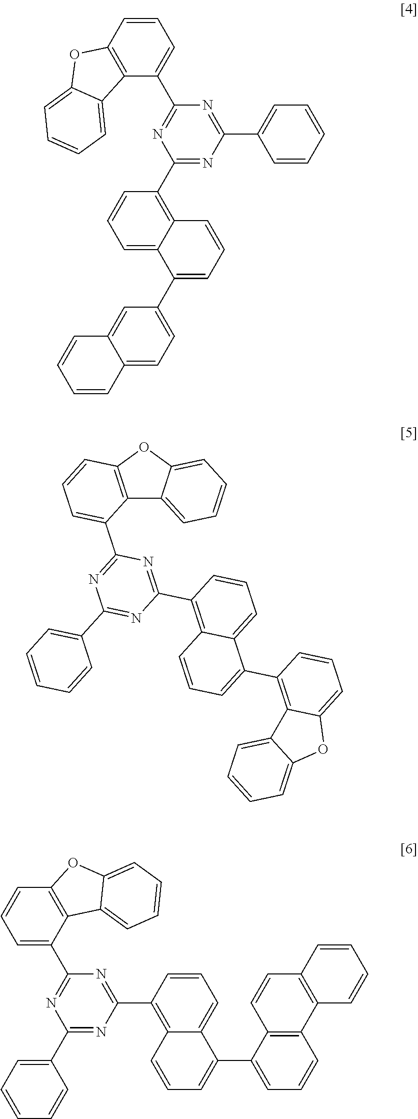 [SMILES: c1ccc(-c2nc(-c3cccc4c(-c5ccc6ccccc6c5)cccc34)nc(-c3cccc4oc5ccccc5c34)n2)cc1 ; c1ccc(-c2nc(-c3cccc4c(-c5cccc6oc7ccccc7c56)cccc34)nc(-c3cccc4oc5ccccc5c34)n2)cc1 ; c1ccc(-c2nc(-c3cccc4c(-c5cccc6c5ccc5ccccc56)cccc34)nc(-c3cccc4oc5ccccc5c34)n2)cc1]

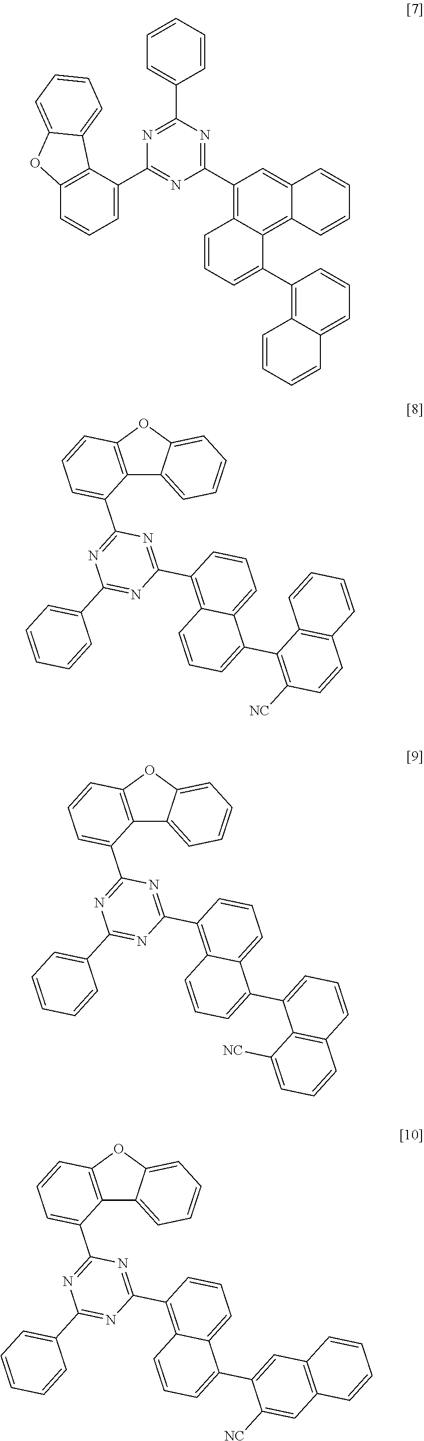 [SMILES: c1ccc(-c2nc(-c3cc4ccccc4c4c(-c5cccc6ccccc56)cccc34)nc(-c3cccc4oc5ccccc5c34)n2)cc1 ; N#Cc1ccc2ccccc2c1-c1cccc2c(-c3nc(-c4ccccc4)nc(-c4cccc5oc6ccccc6c45)n3)cccc12 ; N#Cc1cccc2cccc(-c3cccc4c(-c5nc(-c6ccccc6)nc(-c6cccc7oc8ccccc8c67)n5)cccc34)c12 ; N#Cc1cc2ccccc2cc1-c1cccc2c(-c3nc(-c4ccccc4)nc(-c4cccc5oc6ccccc6c45)n3)cccc12]

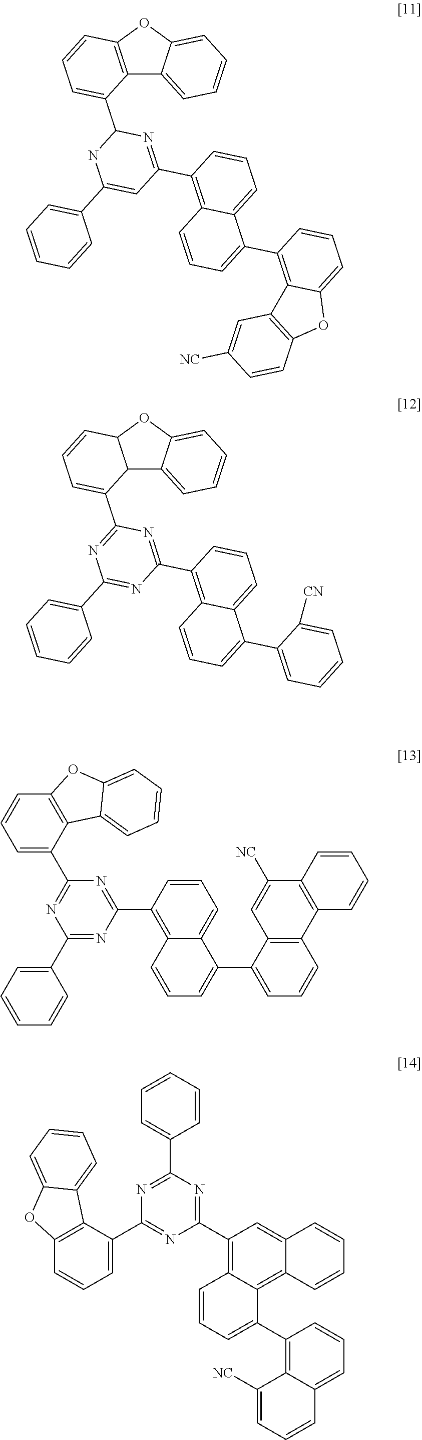

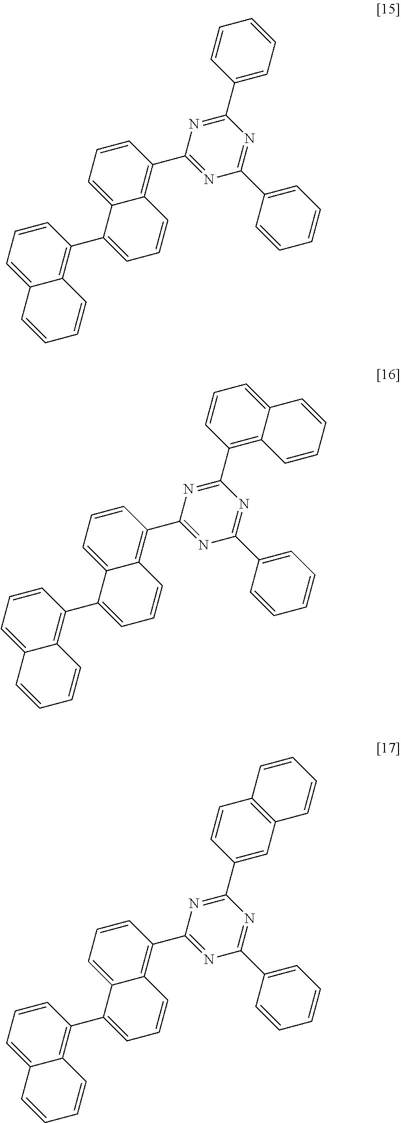 [SMILES: c1ccc(-c2nc(-c3ccccc3)nc(-c3cccc4c(-c5cccc6ccccc56)cccc34)n2)cc1 ; c1ccc(-c2nc(-c3cccc4ccccc34)nc(-c3cccc4c(-c5cccc6ccccc56)cccc34)n2)cc1 ; c1ccc(-c2nc(-c3ccc4ccccc4c3)nc(-c3cccc4c(-c5cccc6ccccc56)cccc34)n2)cc1]

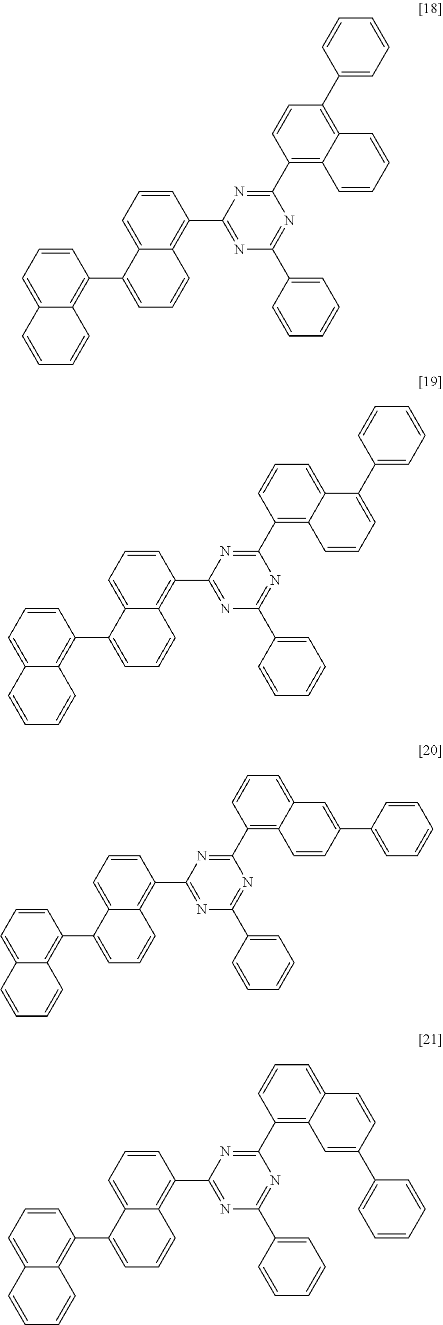 [SMILES: c1ccc(-c2nc(-c3ccc(-c4ccccc4)c4ccccc34)nc(-c3cccc4c(-c5cccc6ccccc56)cccc34)n2)cc1 ; c1ccc(-c2nc(-c3cccc4c(-c5ccccc5)cccc34)nc(-c3cccc4c(-c5cccc6ccccc56)cccc34)n2)cc1 ; c1ccc(-c2ccc3c(-c4nc(-c5ccccc5)nc(-c5cccc6c(-c7cccc8ccccc78)cccc56)n4)cccc3c2)cc1 ; c1ccc(-c2ccc3cccc(-c4nc(-c5ccccc5)nc(-c5cccc6c(-c7cccc8ccccc78)cccc56)n4)c3c2)cc1]

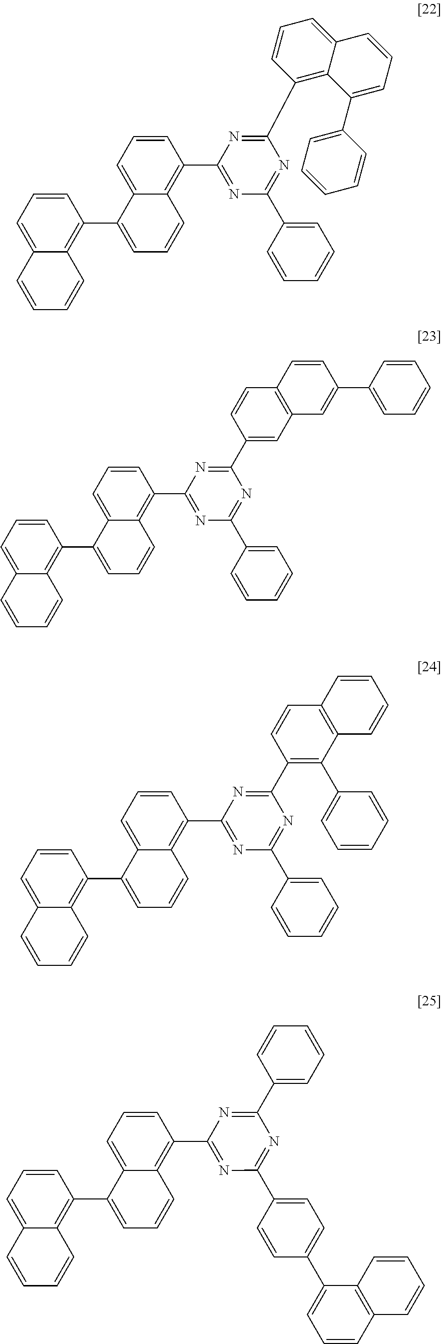 [SMILES: c1ccc(-c2nc(-c3cccc4c(-c5cccc6ccccc56)cccc34)nc(-c3cccc4cccc(-c5ccccc5)c34)n2)cc1 ; c1ccc(-c2ccc3ccc(-c4nc(-c5ccccc5)nc(-c5cccc6c(-c7cccc8ccccc78)cccc56)n4)cc3c2)cc1 ; c1ccc(-c2nc(-c3ccc4ccccc4c3-c3ccccc3)nc(-c3cccc4c(-c5cccc6ccccc56)cccc34)n2)cc1 ; c1ccc(-c2nc(-c3ccc(-c4cccc5ccccc45)cc3)nc(-c3cccc4c(-c5cccc6ccccc56)cccc34)n2)cc1]

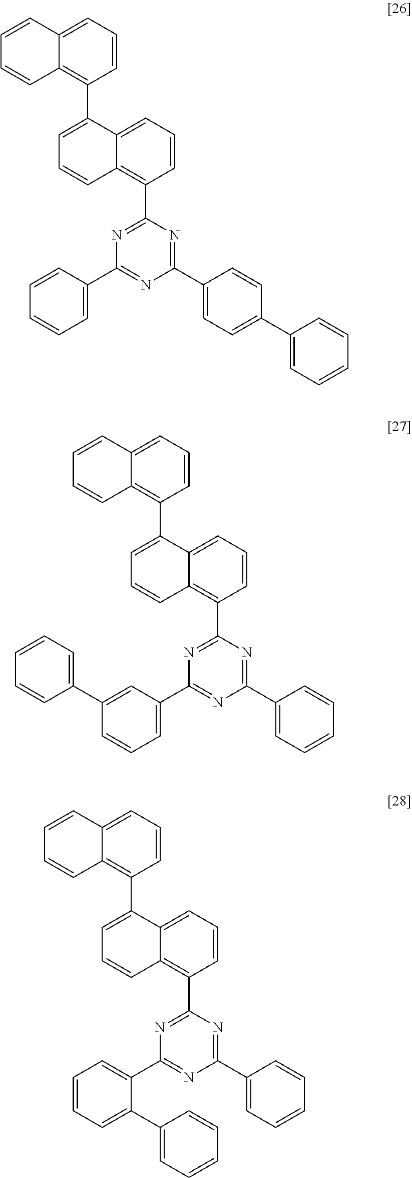 [SMILES: c1ccc(-c2ccc(-c3nc(-c4ccccc4)nc(-c4cccc5c(-c6cccc7ccccc67)cccc45)n3)cc2)cc1 ; c1ccc(-c2cccc(-c3nc(-c4ccccc4)nc(-c4cccc5c(-c6cccc7ccccc67)cccc45)n3)c2)cc1 ; c1ccc(-c2nc(-c3ccccc3-c3ccccc3)nc(-c3cccc4c(-c5cccc6ccccc56)cccc34)n2)cc1]

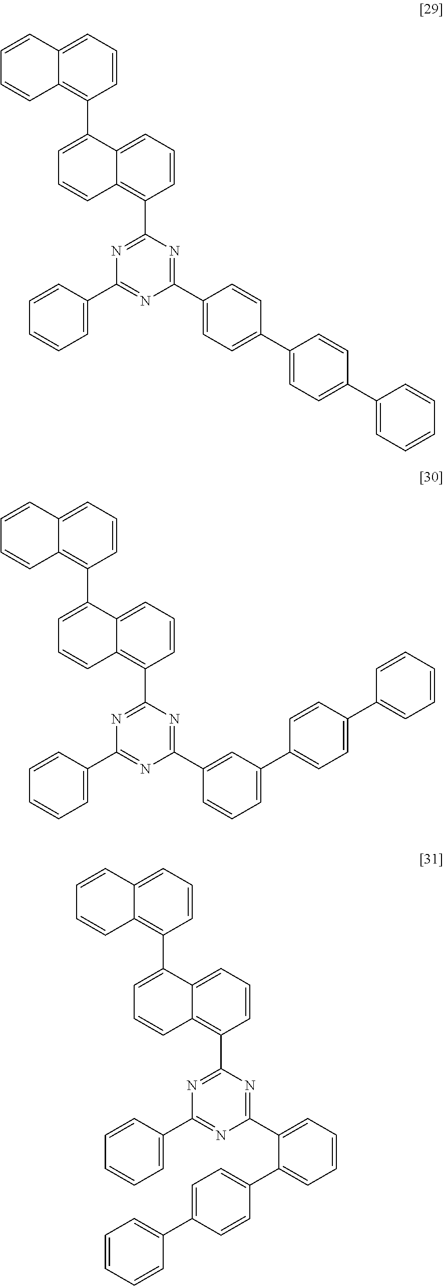 [SMILES: c1ccc(-c2ccc(-c3ccc(-c4nc(-c5ccccc5)nc(-c5cccc6c(-c7cccc8ccccc78)cccc56)n4)cc3)cc2)cc1 ; c1ccc(-c2ccc(-c3cccc(-c4nc(-c5ccccc5)nc(-c5cccc6c(-c7cccc8ccccc78)cccc56)n4)c3)cc2)cc1 ; c1ccc(-c2ccc(-c3ccccc3-c3nc(-c4ccccc4)nc(-c4cccc5c(-c6cccc7ccccc67)cccc45)n3)cc2)cc1]

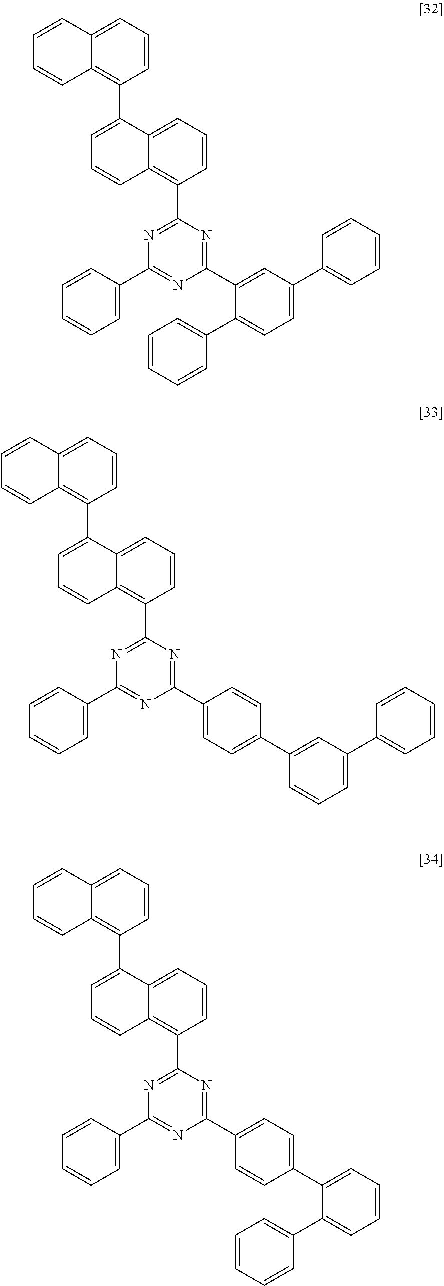 [SMILES: c1ccc(-c2ccc(-c3ccccc3)c(-c3nc(-c4ccccc4)nc(-c4cccc5c(-c6cccc7ccccc67)cccc45)n3)c2)cc1 ; c1ccc(-c2cccc(-c3ccc(-c4nc(-c5ccccc5)nc(-c5cccc6c(-c7cccc8ccccc78)cccc56)n4)cc3)c2)cc1 ; c1ccc(-c2nc(-c3ccc(-c4ccccc4-c4ccccc4)cc3)nc(-c3cccc4c(-c5cccc6ccccc56)cccc34)n2)cc1]

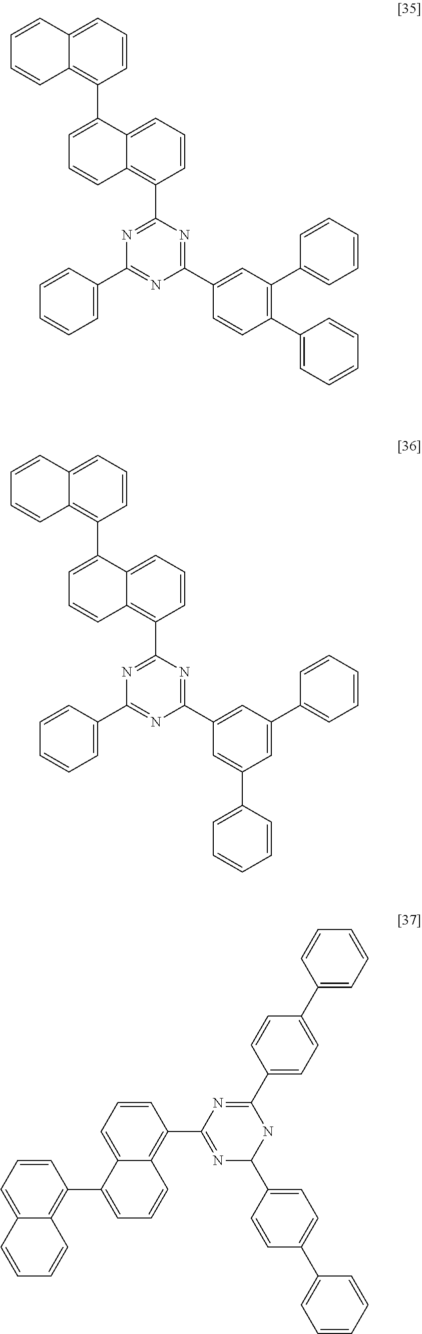

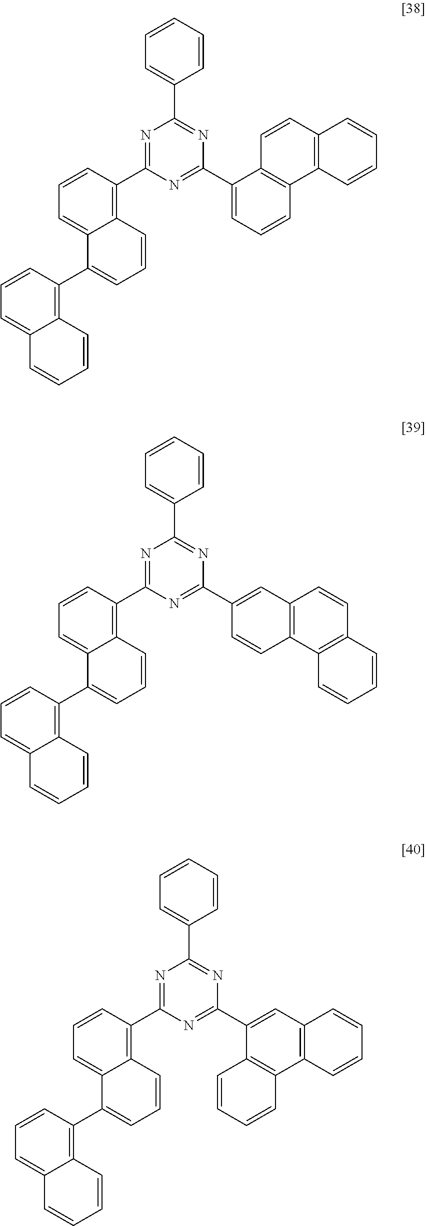 [SMILES: c1ccc(-c2nc(-c3cccc4c(-c5cccc6ccccc56)cccc34)nc(-c3cccc4c3ccc3ccccc34)n2)cc1 ; c1ccc(-c2nc(-c3ccc4c(ccc5ccccc54)c3)nc(-c3cccc4c(-c5cccc6ccccc56)cccc34)n2)cc1 ; c1ccc(-c2nc(-c3cccc4c(-c5cccc6ccccc56)cccc34)nc(-c3cc4ccccc4c4ccccc34)n2)cc1]

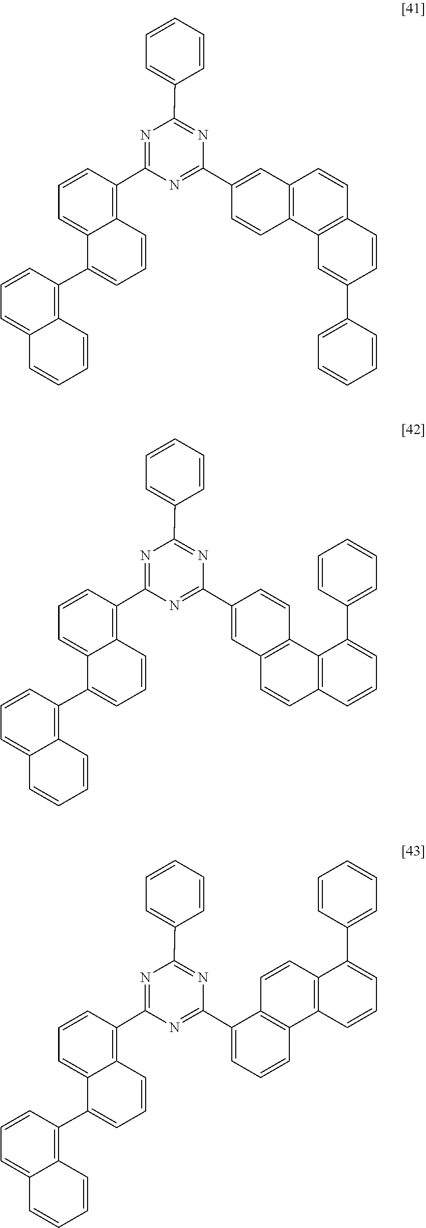 [SMILES: c1ccc(-c2ccc3ccc4cc(-c5nc(-c6ccccc6)nc(-c6cccc7c(-c8cccc9ccccc89)cccc67)n5)ccc4c3c2)cc1 ; c1ccc(-c2nc(-c3ccc4c(ccc5cccc(-c6ccccc6)c54)c3)nc(-c3cccc4c(-c5cccc6ccccc56)cccc34)n2)cc1 ; c1ccc(-c2nc(-c3cccc4c(-c5cccc6ccccc56)cccc34)nc(-c3cccc4c3ccc3c(-c5ccccc5)cccc34)n2)cc1]

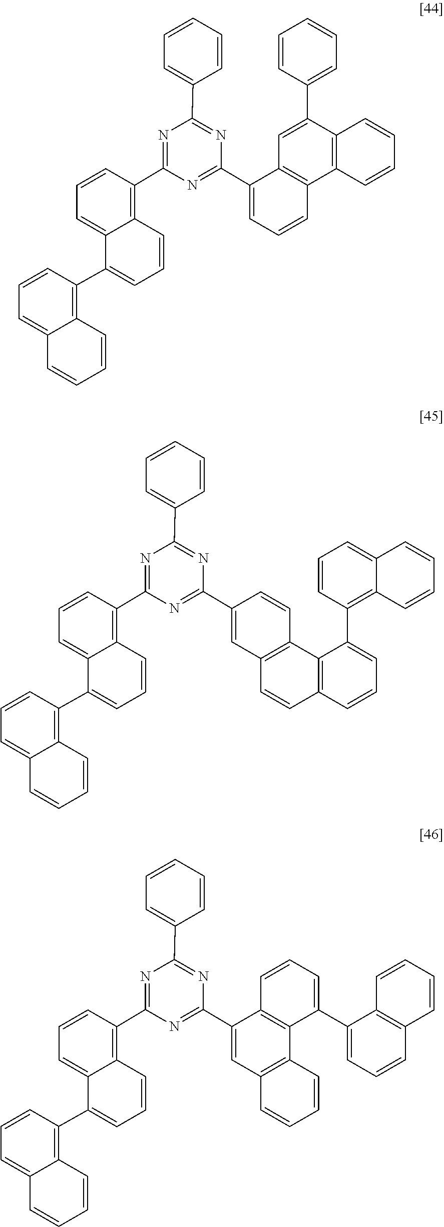 [SMILES: c1ccc(-c2nc(-c3cccc4c(-c5cccc6ccccc56)cccc34)nc(-c3cccc4c3cc(-c3ccccc3)c3ccccc34)n2)cc1 ; c1ccc(-c2nc(-c3ccc4c(ccc5cccc(-c6cccc7ccccc67)c54)c3)nc(-c3cccc4c(-c5cccc6ccccc56)cccc34)n2)cc1 ; c1ccc(-c2nc(-c3cccc4c(-c5cccc6ccccc56)cccc34)nc(-c3cc4ccccc4c4c(-c5cccc6ccccc56)cccc34)n2)cc1]

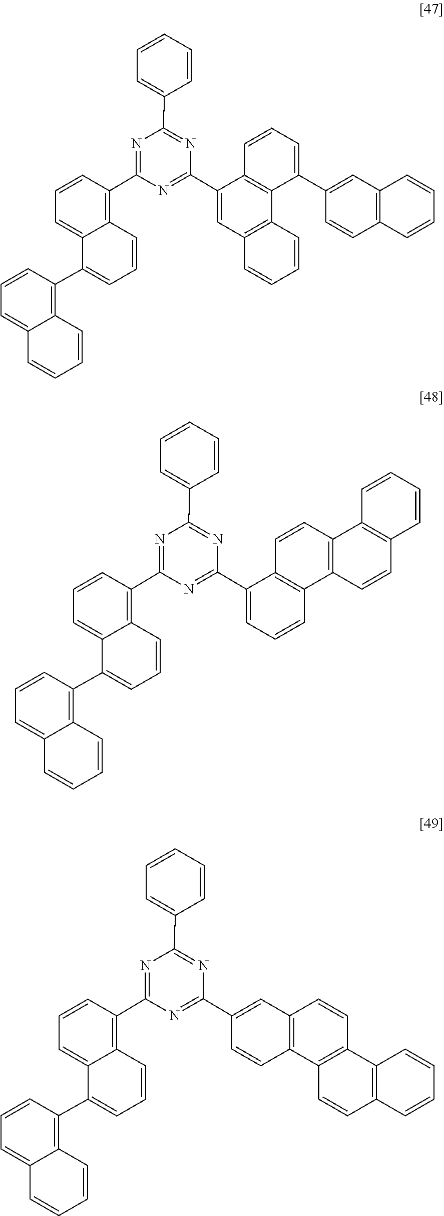 [SMILES: c1ccc(-c2nc(-c3cccc4c(-c5cccc6ccccc56)cccc34)nc(-c3cc4ccccc4c4c(-c5ccc6ccccc6c5)cccc34)n2)cc1 ; c1ccc(-c2nc(-c3cccc4c(-c5cccc6ccccc56)cccc34)nc(-c3cccc4c3ccc3c5ccccc5ccc43)n2)cc1 ; c1ccc(-c2nc(-c3ccc4c(ccc5c6ccccc6ccc45)c3)nc(-c3cccc4c(-c5cccc6ccccc56)cccc34)n2)cc1]

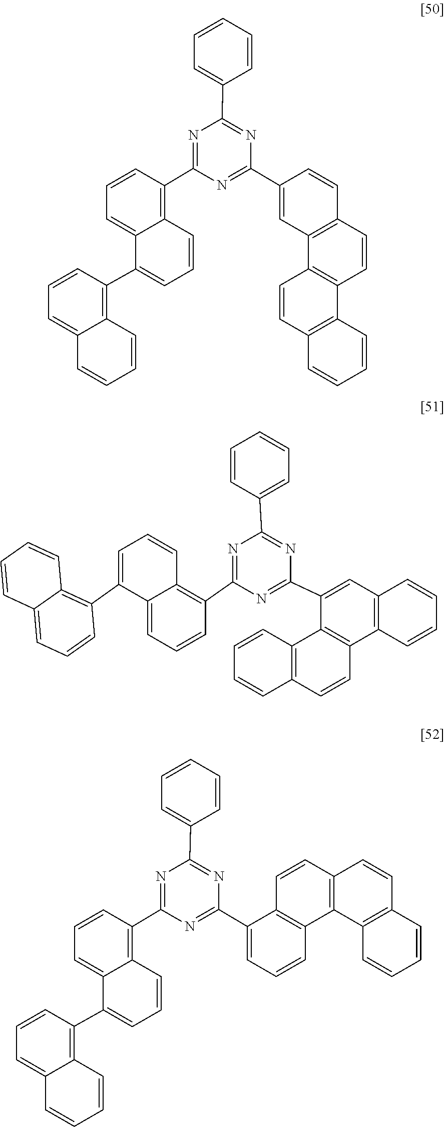 [SMILES: c1ccc(-c2nc(-c3ccc4ccc5c6ccccc6ccc5c4c3)nc(-c3cccc4c(-c5cccc6ccccc56)cccc34)n2)cc1 ; c1ccc(-c2nc(-c3cccc4c(-c5cccc6ccccc56)cccc34)nc(-c3cc4ccccc4c4ccc5ccccc5c34)n2)cc1 ; c1ccc(-c2nc(-c3cccc4c(-c5cccc6ccccc56)cccc34)nc(-c3cccc4c3ccc3ccc5ccccc5c34)n2)cc1]

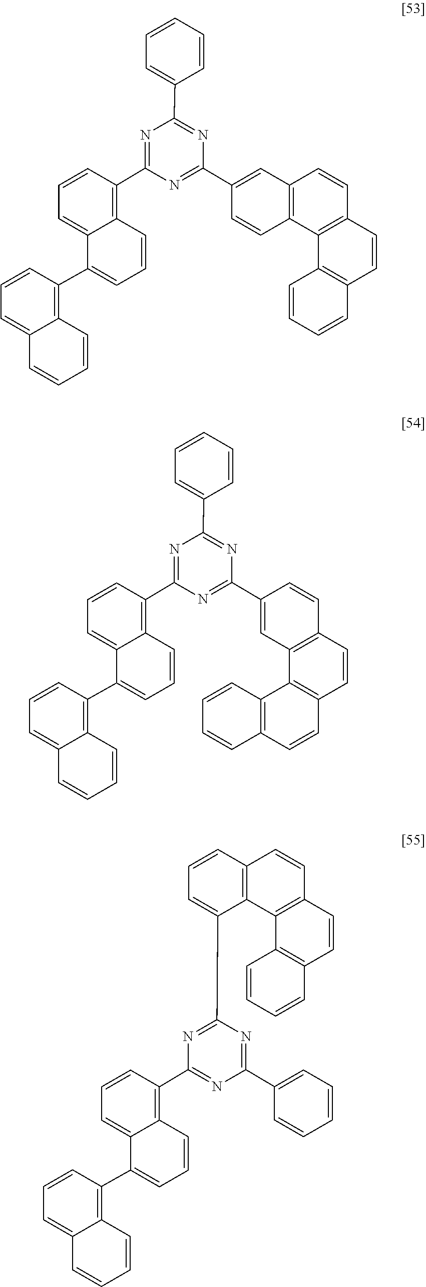 [SMILES: c1ccc(-c2nc(-c3ccc4c(ccc5ccc6ccccc6c54)c3)nc(-c3cccc4c(-c5cccc6ccccc56)cccc34)n2)cc1 ; c1ccc(-c2nc(-c3ccc4ccc5ccc6ccccc6c5c4c3)nc(-c3cccc4c(-c5cccc6ccccc56)cccc34)n2)cc1 ; c1ccc(-c2nc(-c3cccc4c(-c5cccc6ccccc56)cccc34)nc(-c3cccc4ccc5ccc6ccccc6c5c34)n2)cc1]

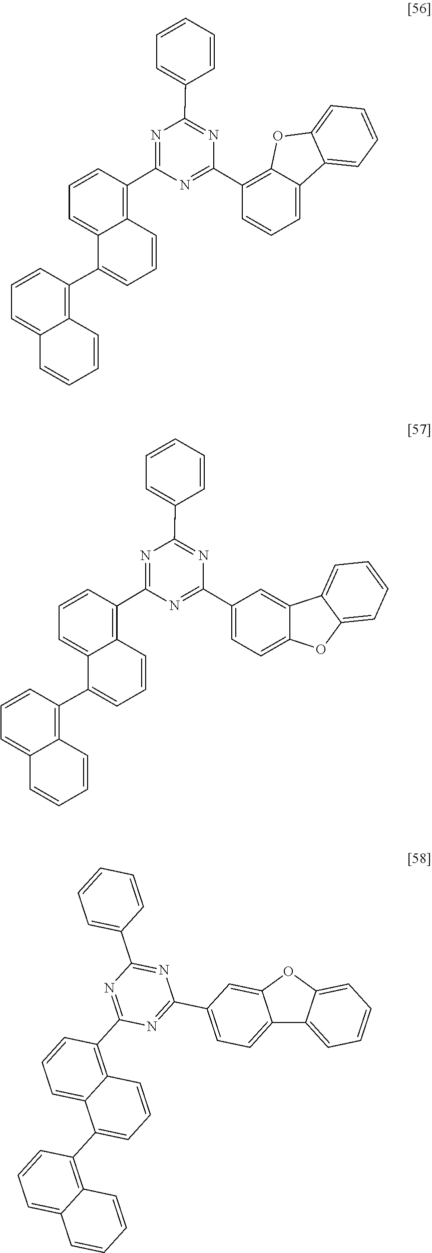 [SMILES: c1ccc(-c2nc(-c3cccc4c(-c5cccc6ccccc56)cccc34)nc(-c3cccc4c3oc3ccccc34)n2)cc1 ; c1ccc(-c2nc(-c3ccc4oc5ccccc5c4c3)nc(-c3cccc4c(-c5cccc6ccccc56)cccc34)n2)cc1 ; c1ccc(-c2nc(-c3ccc4c(c3)oc3ccccc34)nc(-c3cccc4c(-c5cccc6ccccc56)cccc34)n2)cc1]

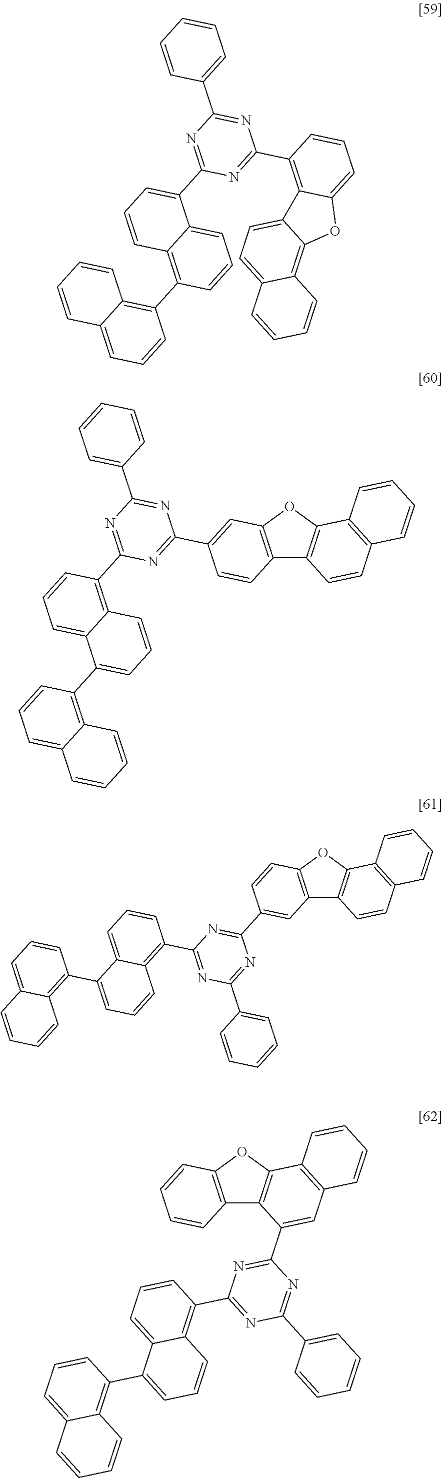 [SMILES: c1ccc(-c2nc(-c3cccc4c(-c5cccc6ccccc56)cccc34)nc(-c3cccc4oc5c6ccccc6ccc5c34)n2)cc1 ; c1ccc(-c2nc(-c3ccc4c(c3)oc3c5ccccc5ccc43)nc(-c3cccc4c(-c5cccc6ccccc56)cccc34)n2)cc1 ; c1ccc(-c2nc(-c3ccc4oc5c6ccccc6ccc5c4c3)nc(-c3cccc4c(-c5cccc6ccccc56)cccc34)n2)cc1 ; c1ccc(-c2nc(-c3cccc4c(-c5cccc6ccccc56)cccc34)nc(-c3cc4ccccc4c4oc5ccccc5c34)n2)cc1]

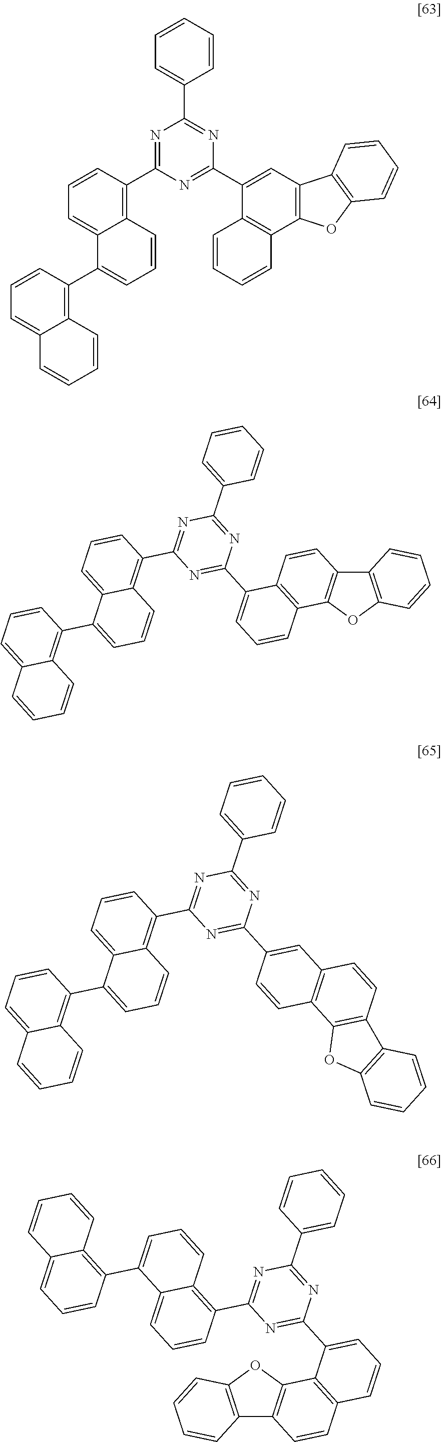 [SMILES: c1ccc(-c2nc(-c3cccc4c(-c5cccc6ccccc56)cccc34)nc(-c3cc4c5ccccc5oc4c4ccccc34)n2)cc1 ; c1ccc(-c2nc(-c3cccc4c(-c5cccc6ccccc56)cccc34)nc(-c3cccc4c3ccc3c5ccccc5oc43)n2)cc1 ; c1ccc(-c2nc(-c3ccc4c(ccc5c6ccccc6oc45)c3)nc(-c3cccc4c(-c5cccc6ccccc56)cccc34)n2)cc1 ; c1ccc(-c2nc(-c3cccc4c(-c5cccc6ccccc56)cccc34)nc(-c3cccc4ccc5c6ccccc6oc5c34)n2)cc1]

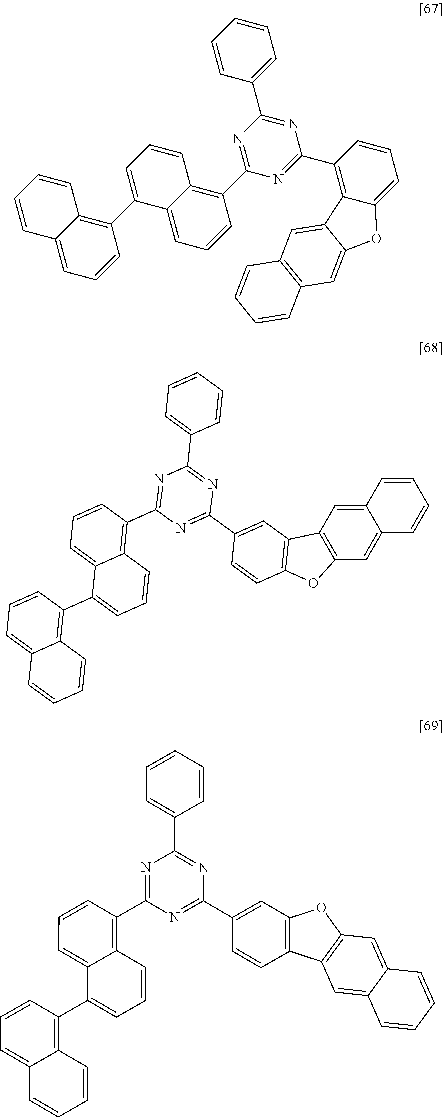 [SMILES: c1ccc(-c2nc(-c3cccc4c(-c5cccc6ccccc56)cccc34)nc(-c3cccc4oc5cc6ccccc6cc5c34)n2)cc1 ; c1ccc(-c2nc(-c3ccc4oc5cc6ccccc6cc5c4c3)nc(-c3cccc4c(-c5cccc6ccccc56)cccc34)n2)cc1 ; c1ccc(-c2nc(-c3ccc4c(c3)oc3cc5ccccc5cc34)nc(-c3cccc4c(-c5cccc6ccccc56)cccc34)n2)cc1]

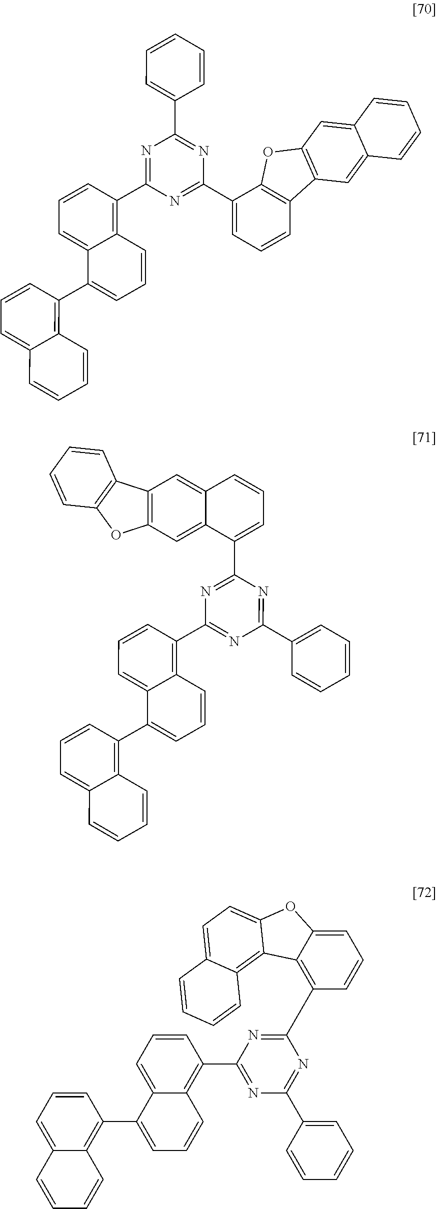 [SMILES: c1ccc(-c2nc(-c3cccc4c(-c5cccc6ccccc56)cccc34)nc(-c3cccc4c3oc3cc5ccccc5cc34)n2)cc1 ; c1ccc(-c2nc(-c3cccc4cc5c(cc34)oc3ccccc35)nc(-c3cccc4c(-c5cccc6ccccc56)cccc34)n2)cc1 ; c1ccc(-c2nc(-c3cccc4c(-c5cccc6ccccc56)cccc34)nc(-c3cccc4oc5ccc6ccccc6c5c34)n2)cc1]

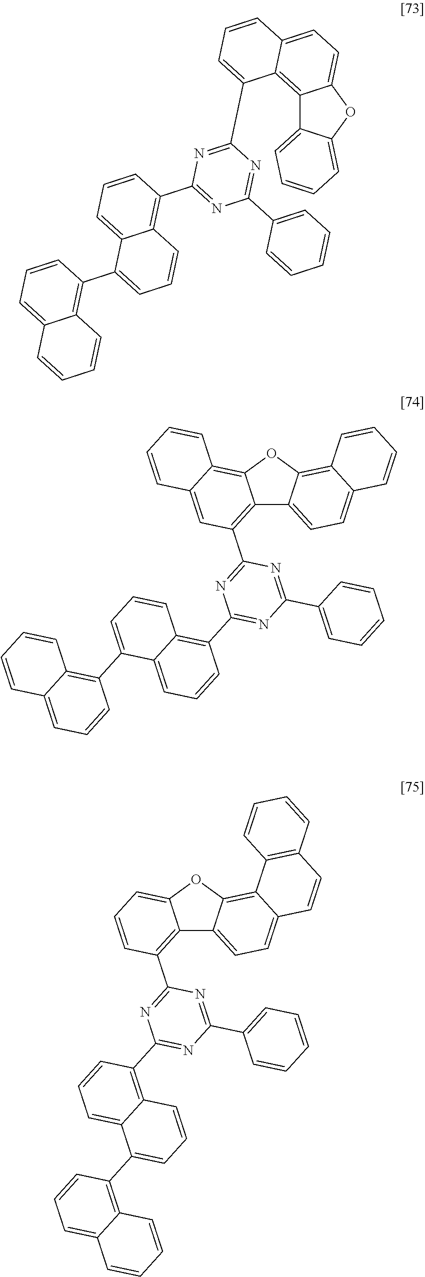 [SMILES: c1ccc(-c2nc(-c3cccc4c(-c5cccc6ccccc56)cccc34)nc(-c3cccc4ccc5oc6ccccc6c5c34)n2)cc1 ; c1ccc(-c2nc(-c3cccc4c(-c5cccc6ccccc56)cccc34)nc(-c3cc4ccccc4c4oc5c6ccccc6ccc5c34)n2)cc1 ; c1ccc(-c2nc(-c3cccc4c(-c5cccc6ccccc56)cccc34)nc(-c3cccc4oc5c(ccc6ccc7ccccc7c65)c34)n2)cc1]

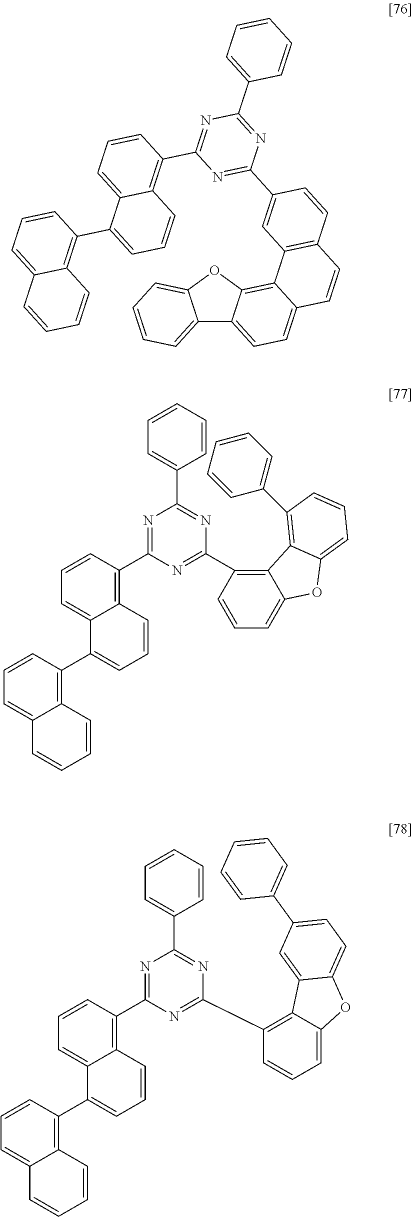 [SMILES: c1ccc(-c2nc(-c3ccc4ccc5ccc6c7ccccc7oc6c5c4c3)nc(-c3cccc4c(-c5cccc6ccccc56)cccc34)n2)cc1 ; c1ccc(-c2nc(-c3cccc4c(-c5cccc6ccccc56)cccc34)nc(-c3cccc4oc5cccc(-c6ccccc6)c5c34)n2)cc1 ; c1ccc(-c2ccc3oc4cccc(-c5nc(-c6ccccc6)nc(-c6cccc7c(-c8cccc9ccccc89)cccc67)n5)c4c3c2)cc1]

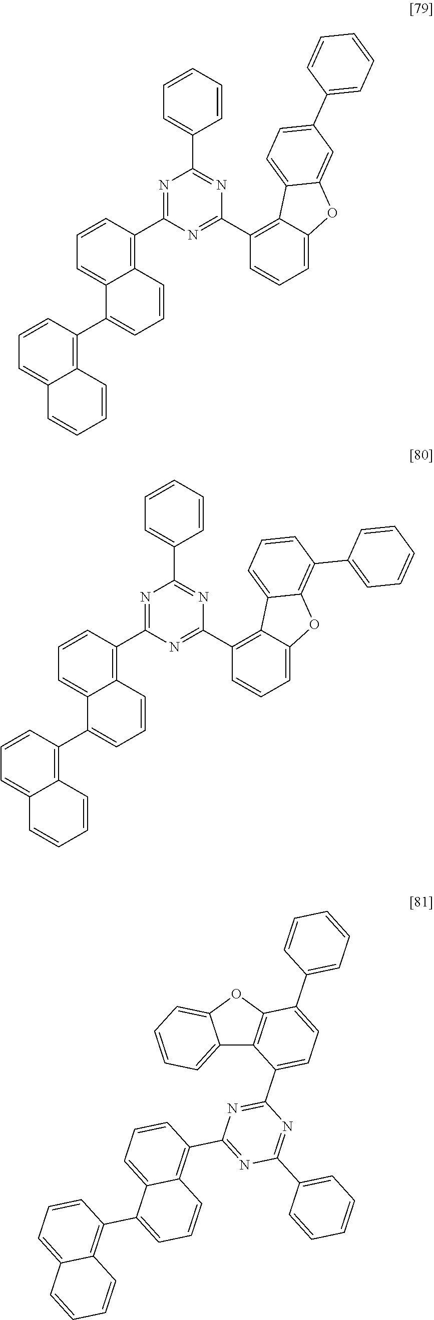 [SMILES: c1ccc(-c2ccc3c(c2)oc2cccc(-c4nc(-c5ccccc5)nc(-c5cccc6c(-c7cccc8ccccc78)cccc56)n4)c23)cc1 ; c1ccc(-c2nc(-c3cccc4c(-c5cccc6ccccc56)cccc34)nc(-c3cccc4oc5c(-c6ccccc6)cccc5c34)n2)cc1 ; c1ccc(-c2nc(-c3cccc4c(-c5cccc6ccccc56)cccc34)nc(-c3ccc(-c4ccccc4)c4oc5ccccc5c34)n2)cc1]

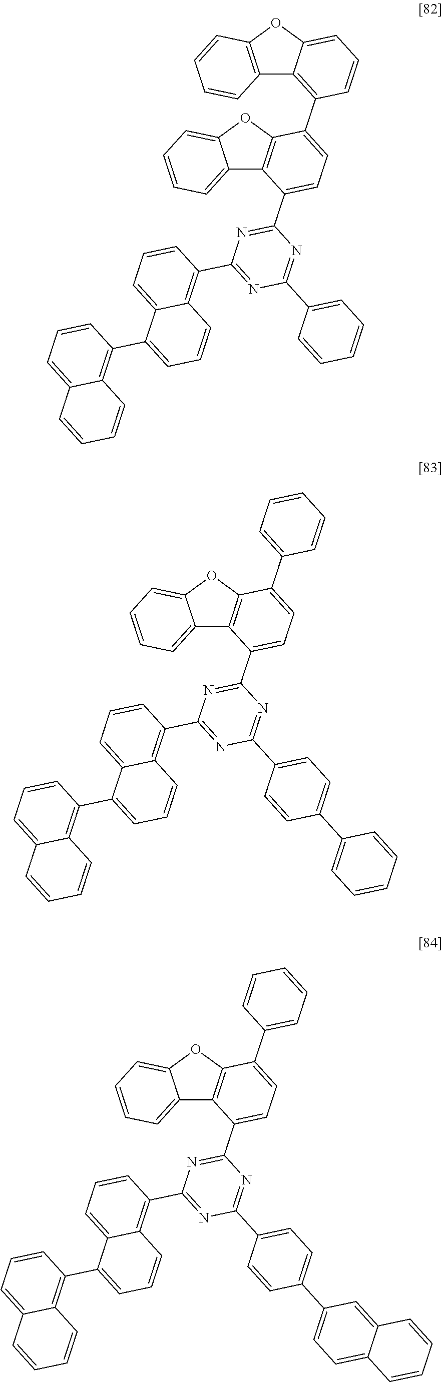 [SMILES: c1ccc(-c2nc(-c3cccc4c(-c5cccc6ccccc56)cccc34)nc(-c3ccc(-c4cccc5oc6ccccc6c45)c4oc5ccccc5c34)n2)cc1 ; c1ccc(-c2ccc(-c3nc(-c4cccc5c(-c6cccc7ccccc67)cccc45)nc(-c4ccc(-c5ccccc5)c5oc6ccccc6c45)n3)cc2)cc1 ; c1ccc(-c2ccc(-c3nc(-c4ccc(-c5ccc6ccccc6c5)cc4)nc(-c4cccc5c(-c6cccc7ccccc67)cccc45)n3)c3c2oc2ccccc23)cc1]

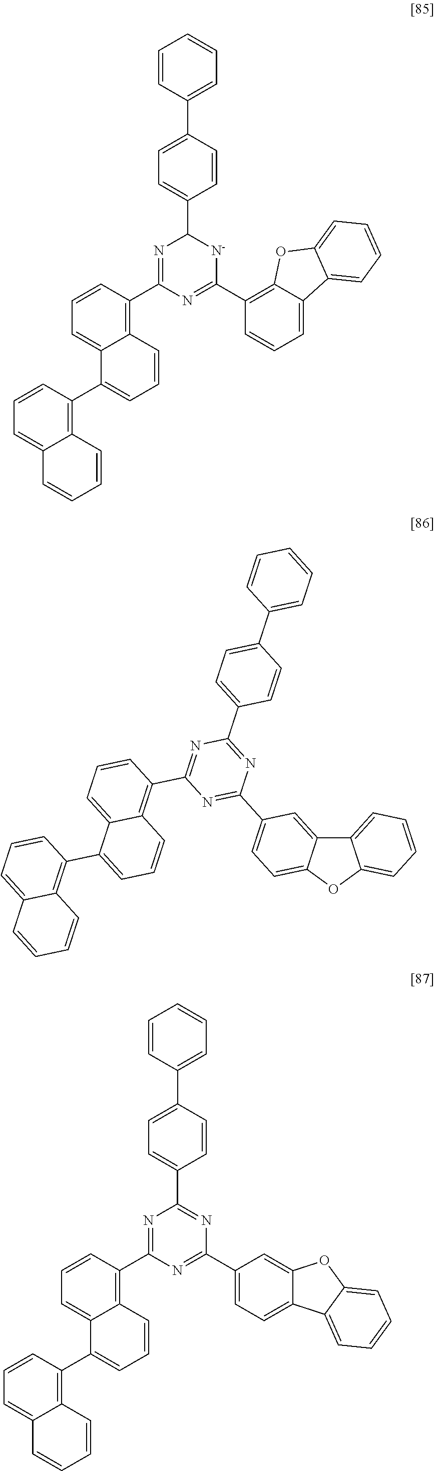 [SMILES: c1ccc(-c2ccc(C3N=C(c4cccc5c(-c6cccc7ccccc67)cccc45)N=C(c4cccc5c4oc4ccccc45)[N-]3)cc2)cc1 ; c1ccc(-c2ccc(-c3nc(-c4ccc5oc6ccccc6c5c4)nc(-c4cccc5c(-c6cccc7ccccc67)cccc45)n3)cc2)cc1 ; c1ccc(-c2ccc(-c3nc(-c4ccc5c(c4)oc4ccccc45)nc(-c4cccc5c(-c6cccc7ccccc67)cccc45)n3)cc2)cc1]

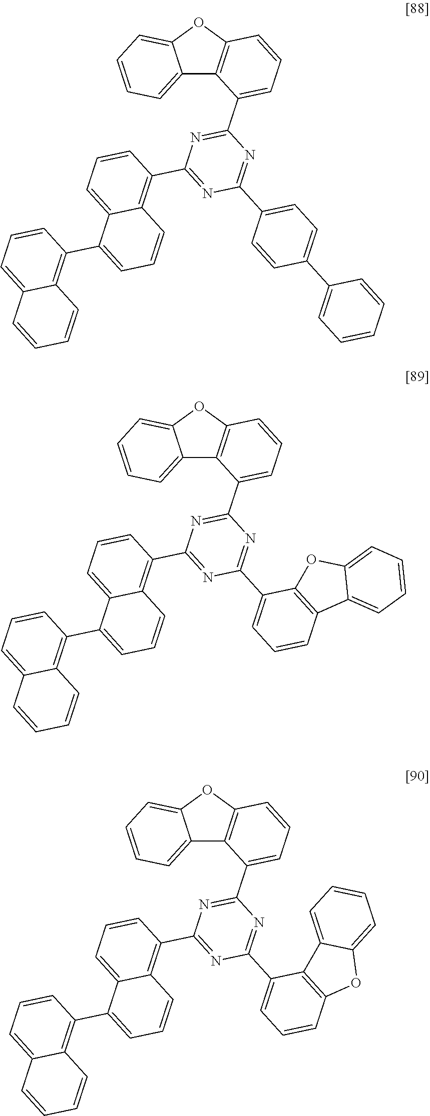 [SMILES: c1ccc(-c2ccc(-c3nc(-c4cccc5c(-c6cccc7ccccc67)cccc45)nc(-c4cccc5oc6ccccc6c45)n3)cc2)cc1 ; c1ccc2c(-c3cccc4c(-c5nc(-c6cccc7c6oc6ccccc67)nc(-c6cccc7oc8ccccc8c67)n5)cccc34)cccc2c1 ; c1ccc2c(-c3cccc4c(-c5nc(-c6cccc7oc8ccccc8c67)nc(-c6cccc7oc8ccccc8c67)n5)cccc34)cccc2c1]

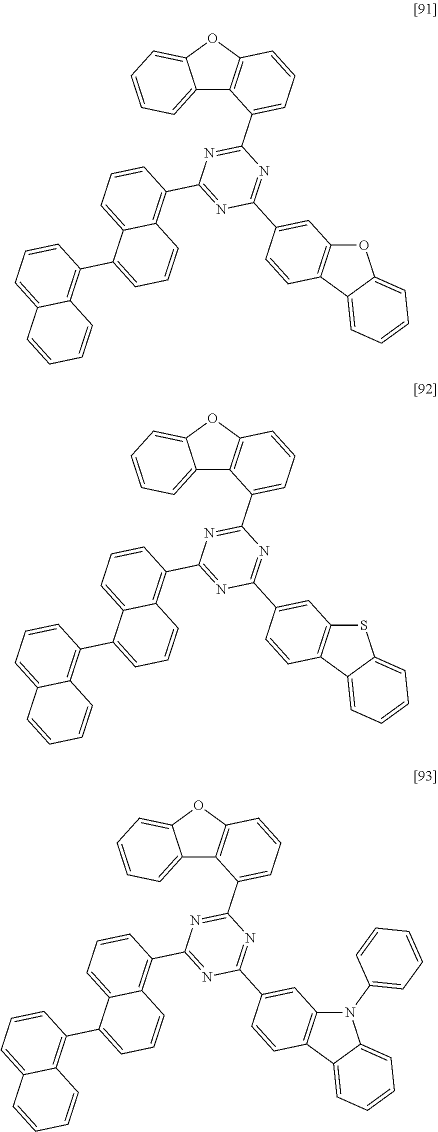 [SMILES: c1ccc2c(-c3cccc4c(-c5nc(-c6ccc7c(c6)oc6ccccc67)nc(-c6cccc7oc8ccccc8c67)n5)cccc34)cccc2c1 ; c1ccc2c(-c3cccc4c(-c5nc(-c6ccc7c(c6)sc6ccccc67)nc(-c6cccc7oc8ccccc8c67)n5)cccc34)cccc2c1 ; c1ccc(-n2c3ccccc3c3ccc(-c4nc(-c5cccc6c(-c7cccc8ccccc78)cccc56)nc(-c5cccc6oc7ccccc7c56)n4)cc32)cc1]

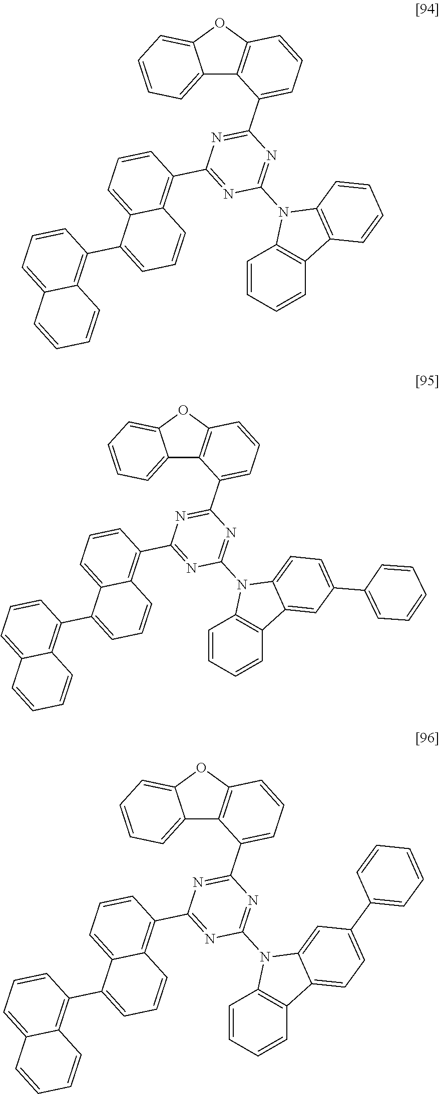 [SMILES: c1ccc2c(-c3cccc4c(-c5nc(-c6cccc7oc8ccccc8c67)nc(-n6c7ccccc7c7ccccc76)n5)cccc34)cccc2c1 ; c1ccc(-c2ccc3c(c2)c2ccccc2n3-c2nc(-c3cccc4c(-c5cccc6ccccc56)cccc34)nc(-c3cccc4oc5ccccc5c34)n2)cc1 ; c1ccc(-c2ccc3c4ccccc4n(-c4nc(-c5cccc6c(-c7cccc8ccccc78)cccc56)nc(-c5cccc6oc7ccccc7c56)n4)c3c2)cc1]

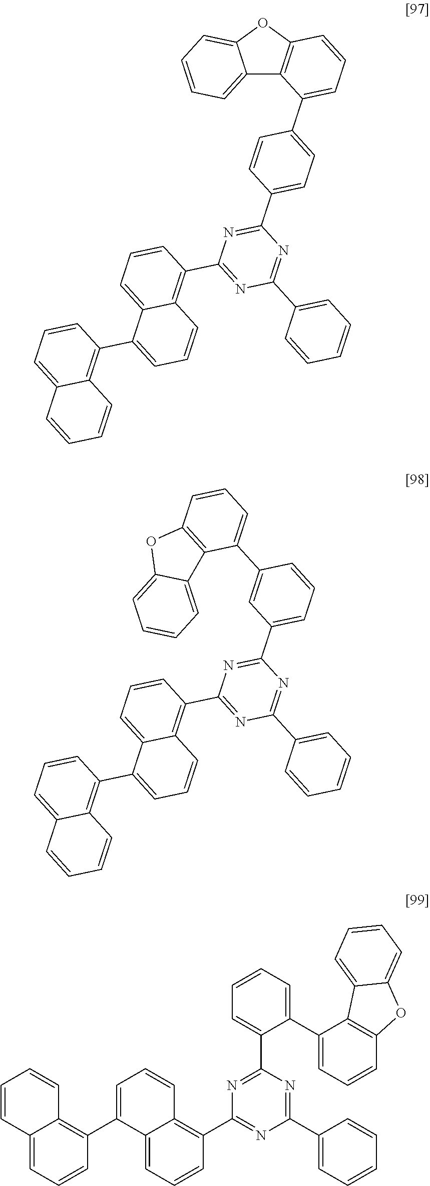 [SMILES: c1ccc(-c2nc(-c3ccc(-c4cccc5oc6ccccc6c45)cc3)nc(-c3cccc4c(-c5cccc6ccccc56)cccc34)n2)cc1 ; c1ccc(-c2nc(-c3cccc(-c4cccc5oc6ccccc6c45)c3)nc(-c3cccc4c(-c5cccc6ccccc56)cccc34)n2)cc1 ; c1ccc(-c2nc(-c3ccccc3-c3cccc4oc5ccccc5c34)nc(-c3cccc4c(-c5cccc6ccccc56)cccc34)n2)cc1]

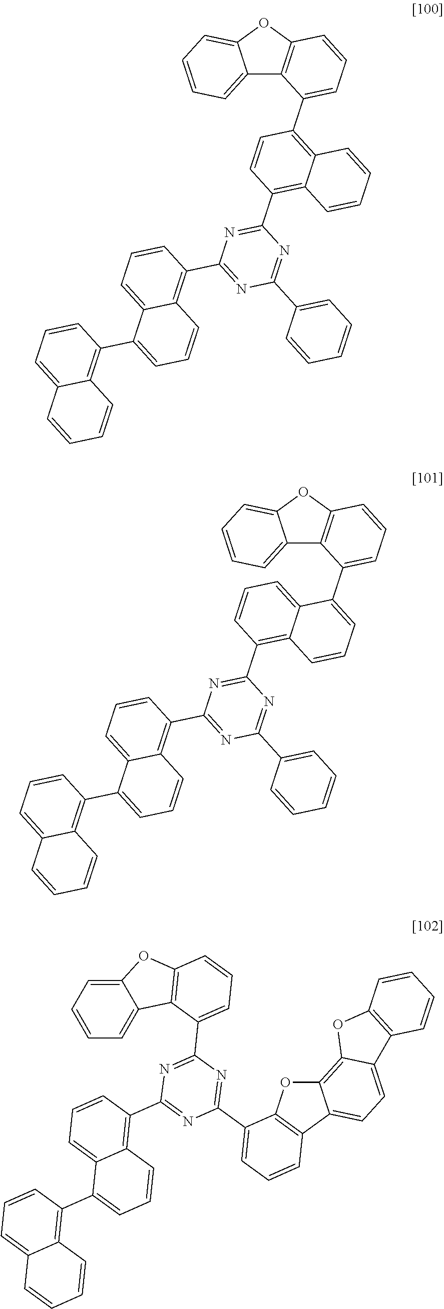 [SMILES: c1ccc(-c2nc(-c3cccc4c(-c5cccc6ccccc56)cccc34)nc(-c3ccc(-c4cccc5oc6ccccc6c45)c4ccccc34)n2)cc1 ; c1ccc(-c2nc(-c3cccc4c(-c5cccc6ccccc56)cccc34)nc(-c3cccc4c(-c5cccc6oc7ccccc7c56)cccc34)n2)cc1 ; c1ccc2c(-c3cccc4c(-c5nc(-c6cccc7c6oc6c7ccc7c8ccccc8oc76)nc(-c6cccc7oc8ccccc8c67)n5)cccc34)cccc2c1]

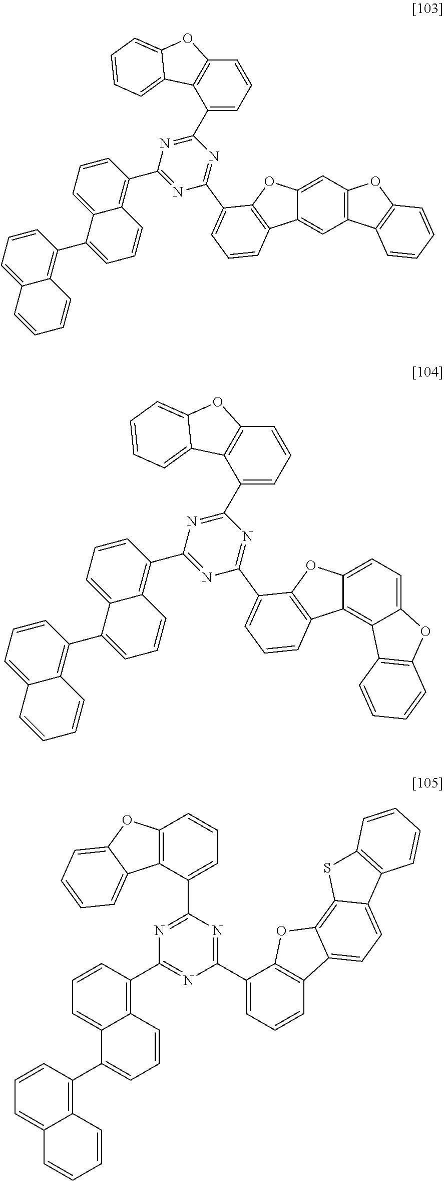 [SMILES: c1ccc2c(-c3cccc4c(-c5nc(-c6cccc7c6oc6cc8oc9ccccc9c8cc67)nc(-c6cccc7oc8ccccc8c67)n5)cccc34)cccc2c1 ; c1ccc2c(-c3cccc4c(-c5nc(-c6cccc7c6oc6ccc8oc9ccccc9c8c67)nc(-c6cccc7oc8ccccc8c67)n5)cccc34)cccc2c1 ; c1ccc2c(-c3cccc4c(-c5nc(-c6cccc7c6oc6c7ccc7c8ccccc8sc76)nc(-c6cccc7oc8ccccc8c67)n5)cccc34)cccc2c1]

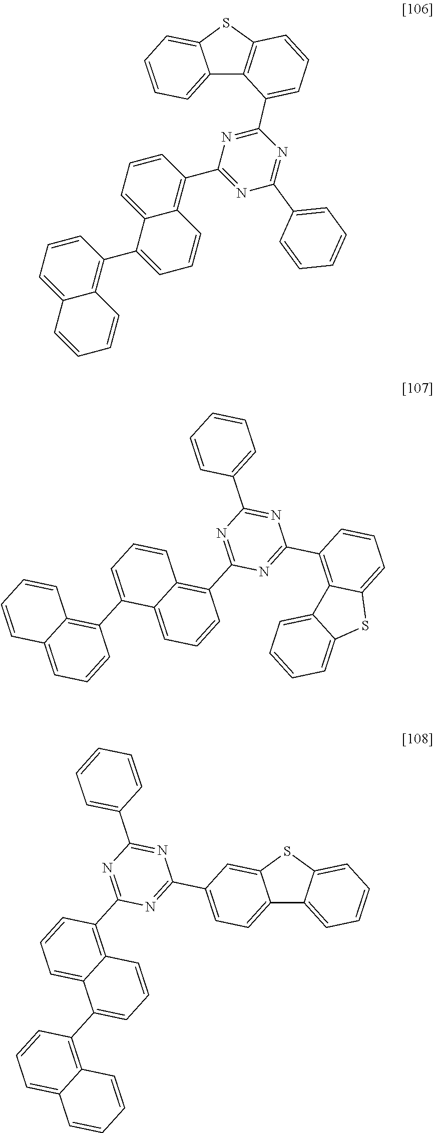 [SMILES: c1ccc(-c2nc(-c3cccc4c(-c5cccc6ccccc56)cccc34)nc(-c3cccc4sc5ccccc5c34)n2)cc1 ; c1ccc(-c2nc(-c3cccc4c(-c5cccc6ccccc56)cccc34)nc(-c3cccc4sc5ccccc5c34)n2)cc1 ; c1ccc(-c2nc(-c3ccc4c(c3)sc3ccccc34)nc(-c3cccc4c(-c5cccc6ccccc56)cccc34)n2)cc1]

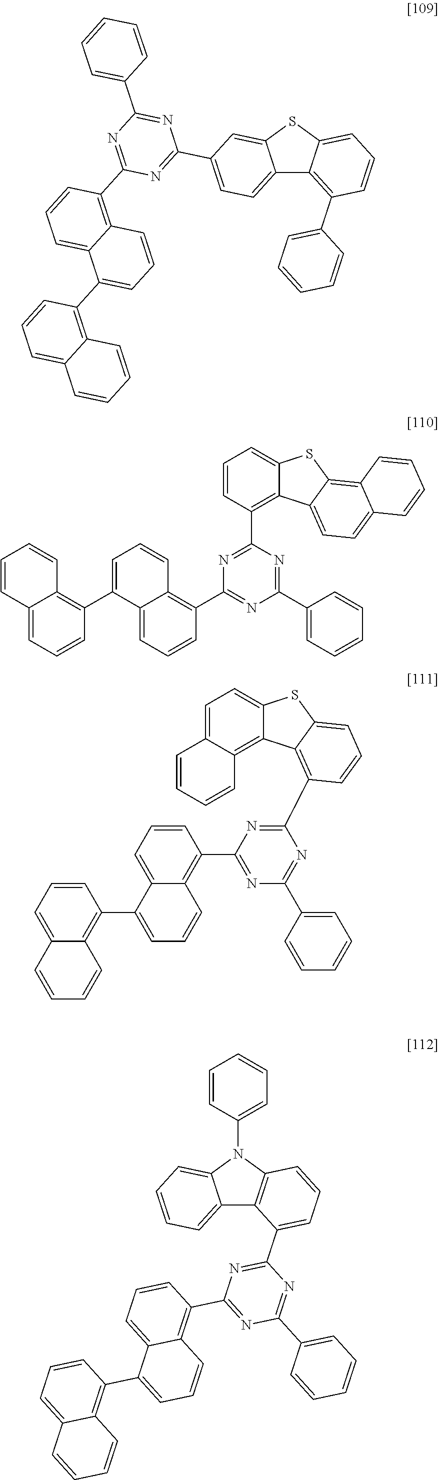 [SMILES: c1ccc(-c2nc(-c3ccc4c(c3)sc3cccc(-c5ccccc5)c34)nc(-c3cccc4c(-c5cccc6ccccc56)cccc34)n2)cc1 ; c1ccc(-c2nc(-c3cccc4c(-c5cccc6ccccc56)cccc34)nc(-c3cccc4sc5c6ccccc6ccc5c34)n2)cc1 ; c1ccc(-c2nc(-c3cccc4c(-c5cccc6ccccc56)cccc34)nc(-c3cccc4sc5ccc6ccccc6c5c34)n2)cc1 ; c1ccc(-c2nc(-c3cccc4c(-c5cccc6ccccc56)cccc34)nc(-c3cccc4c3c3ccccc3n4-c3ccccc3)n2)cc1]

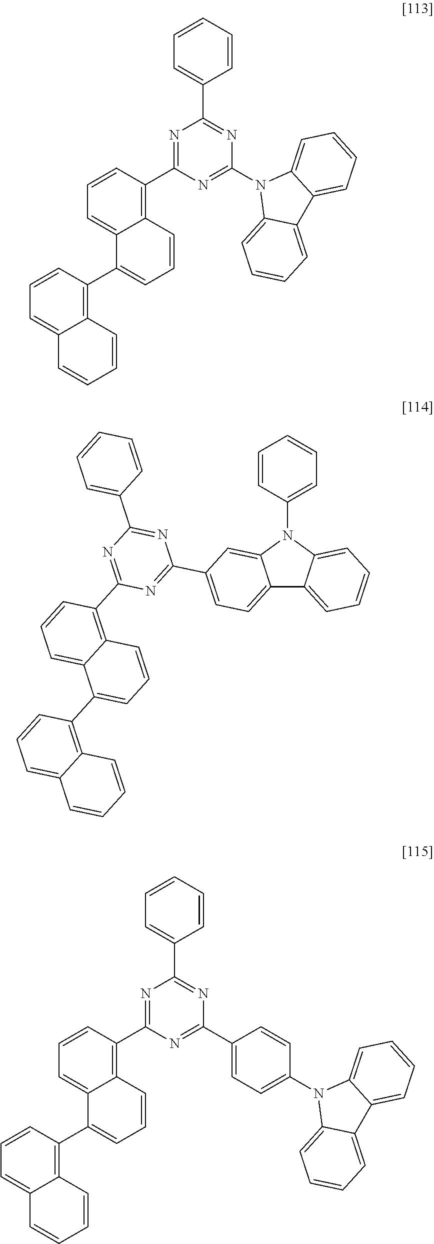 [SMILES: c1ccc(-c2nc(-c3cccc4c(-c5cccc6ccccc56)cccc34)nc(-n3c4ccccc4c4ccccc43)n2)cc1 ; c1ccc(-c2nc(-c3ccc4c5ccccc5n(-c5ccccc5)c4c3)nc(-c3cccc4c(-c5cccc6ccccc56)cccc34)n2)cc1 ; c1ccc(-c2nc(-c3ccc(-n4c5ccccc5c5ccccc54)cc3)nc(-c3cccc4c(-c5cccc6ccccc56)cccc34)n2)cc1]

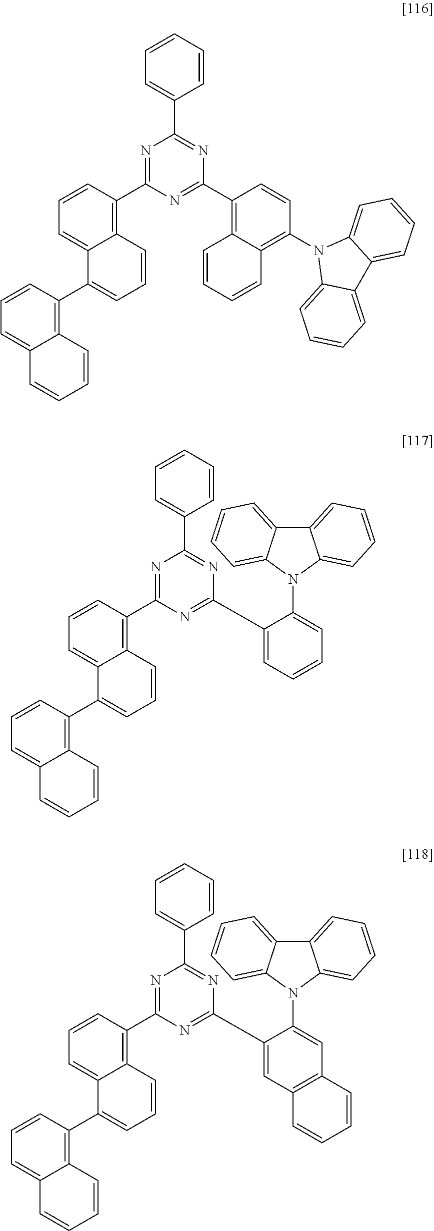 [SMILES: c1ccc(-c2nc(-c3cccc4c(-c5cccc6ccccc56)cccc34)nc(-c3ccc(-n4c5ccccc5c5ccccc54)c4ccccc34)n2)cc1 ; c1ccc(-c2nc(-c3ccccc3-n3c4ccccc4c4ccccc43)nc(-c3cccc4c(-c5cccc6ccccc56)cccc34)n2)cc1 ; c1ccc(-c2nc(-c3cc4ccccc4cc3-n3c4ccccc4c4ccccc43)nc(-c3cccc4c(-c5cccc6ccccc56)cccc34)n2)cc1]

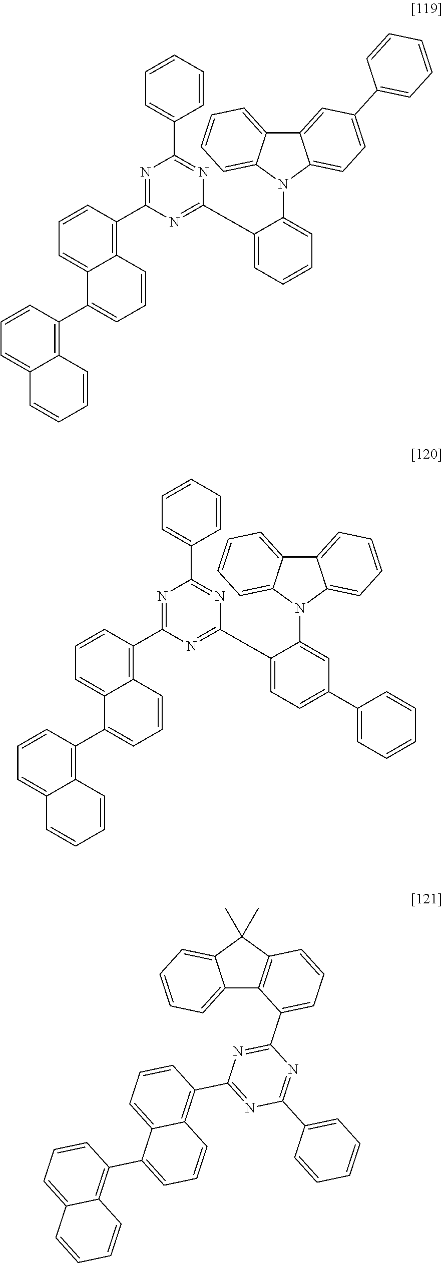 [SMILES: c1ccc(-c2ccc3c(c2)c2ccccc2n3-c2ccccc2-c2nc(-c3ccccc3)nc(-c3cccc4c(-c5cccc6ccccc56)cccc34)n2)cc1 ; c1ccc(-c2ccc(-c3nc(-c4ccccc4)nc(-c4cccc5c(-c6cccc7ccccc67)cccc45)n3)c(-n3c4ccccc4c4ccccc43)c2)cc1 ; CC1(C)c2ccccc2-c2c(-c3nc(-c4ccccc4)nc(-c4cccc5c(-c6cccc7ccccc67)cccc45)n3)cccc21]

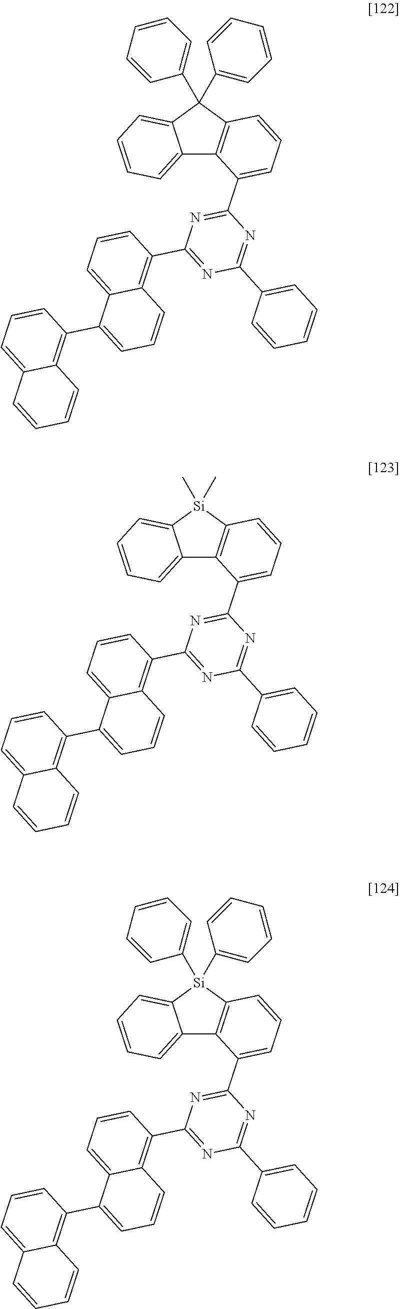 [SMILES: c1ccc(-c2nc(-c3cccc4c3-c3ccccc3C4(c3ccccc3)c3ccccc3)nc(-c3cccc4c(-c5cccc6ccccc56)cccc34)n2)cc1 ; C[Si]1(C)c2ccccc2-c2c(-c3nc(-c4ccccc4)nc(-c4cccc5c(-c6cccc7ccccc67)cccc45)n3)cccc21 ; c1ccc(-c2nc(-c3cccc4c3-c3ccccc3[Si]4(c3ccccc3)c3ccccc3)nc(-c3cccc4c(-c5cccc6ccccc56)cccc34)n2)cc1]

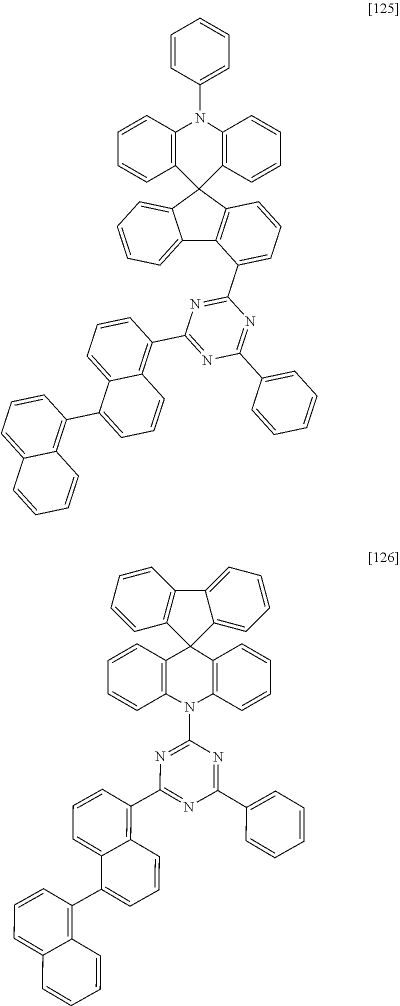 [SMILES: c1ccc(-c2nc(-c3cccc4c3-c3ccccc3C43c4ccccc4N(c4ccccc4)c4ccccc43)nc(-c3cccc4c(-c5cccc6ccccc56)cccc34)n2)cc1 ; c1ccc(-c2nc(-c3cccc4c(-c5cccc6ccccc56)cccc34)nc(N3c4ccccc4C4(c5ccccc5-c5ccccc54)c4ccccc43)n2)cc1]

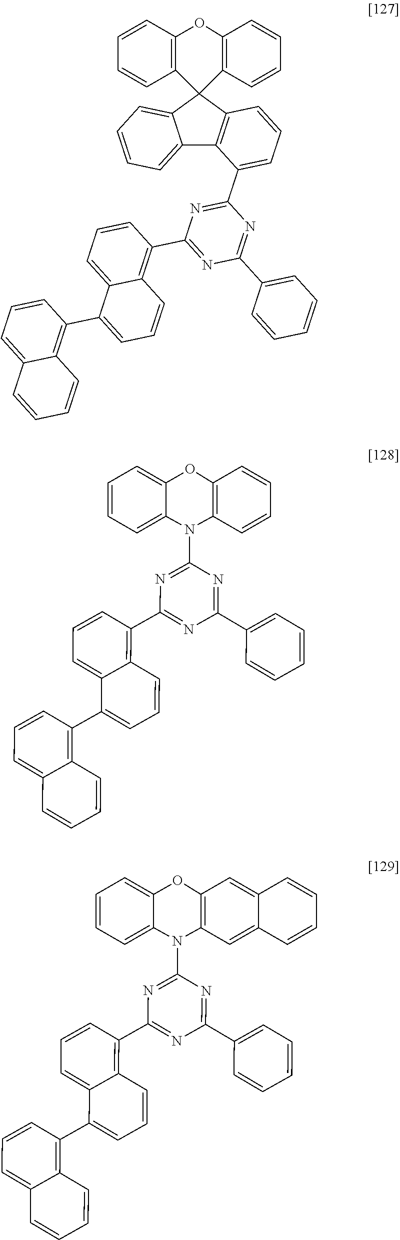 [SMILES: c1ccc(-c2nc(-c3cccc4c3-c3ccccc3C43c4ccccc4Oc4ccccc43)nc(-c3cccc4c(-c5cccc6ccccc56)cccc34)n2)cc1 ; c1ccc(-c2nc(-c3cccc4c(-c5cccc6ccccc56)cccc34)nc(N3c4ccccc4Oc4ccccc43)n2)cc1 ; c1ccc(-c2nc(-c3cccc4c(-c5cccc6ccccc56)cccc34)nc(N3c4ccccc4Oc4cc5ccccc5cc43)n2)cc1]

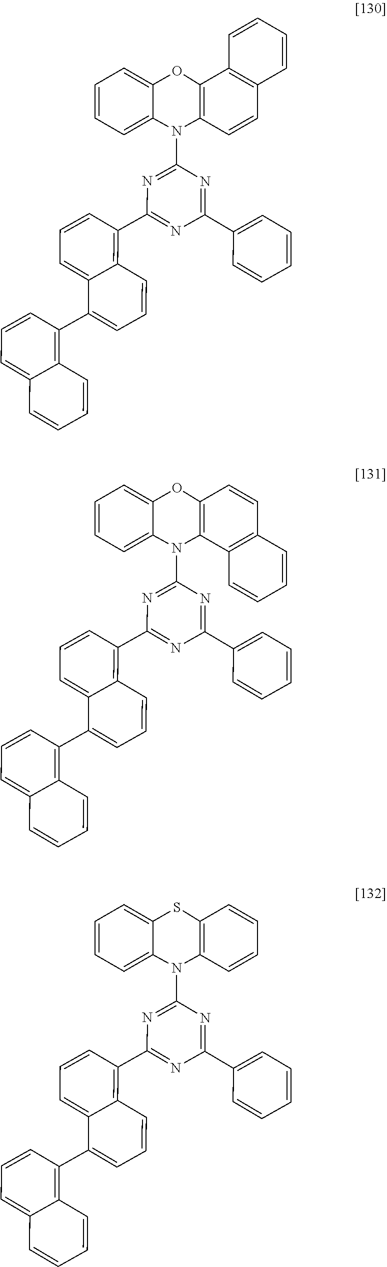 [SMILES: c1ccc(-c2nc(-c3cccc4c(-c5cccc6ccccc56)cccc34)nc(N3c4ccccc4Oc4c3ccc3ccccc43)n2)cc1 ; c1ccc(-c2nc(-c3cccc4c(-c5cccc6ccccc56)cccc34)nc(N3c4ccccc4Oc4ccc5ccccc5c43)n2)cc1 ; c1ccc(-c2nc(-c3cccc4c(-c5cccc6ccccc56)cccc34)nc(N3c4ccccc4Sc4ccccc43)n2)cc1]

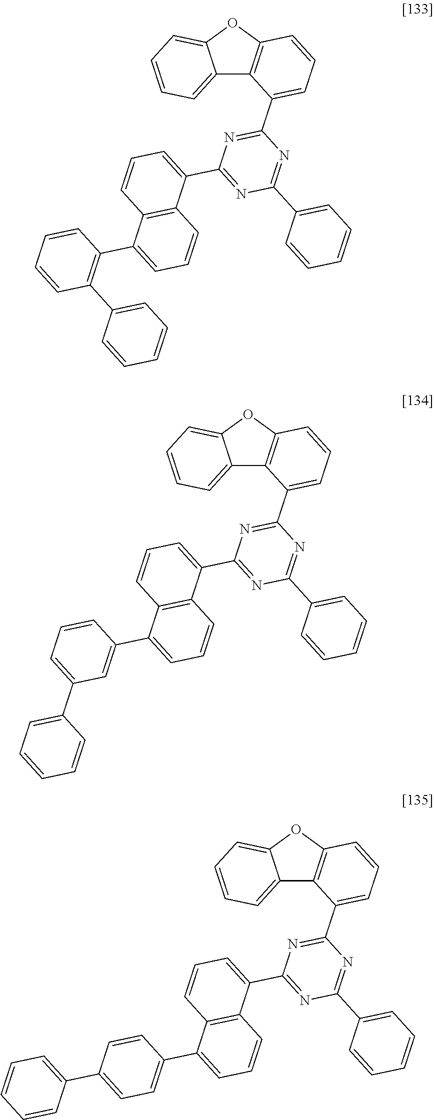 [SMILES: c1ccc(-c2nc(-c3cccc4c(-c5ccccc5-c5ccccc5)cccc34)nc(-c3cccc4oc5ccccc5c34)n2)cc1 ; c1ccc(-c2cccc(-c3cccc4c(-c5nc(-c6ccccc6)nc(-c6cccc7oc8ccccc8c67)n5)cccc34)c2)cc1 ; c1ccc(-c2ccc(-c3cccc4c(-c5nc(-c6ccccc6)nc(-c6cccc7oc8ccccc8c67)n5)cccc34)cc2)cc1]

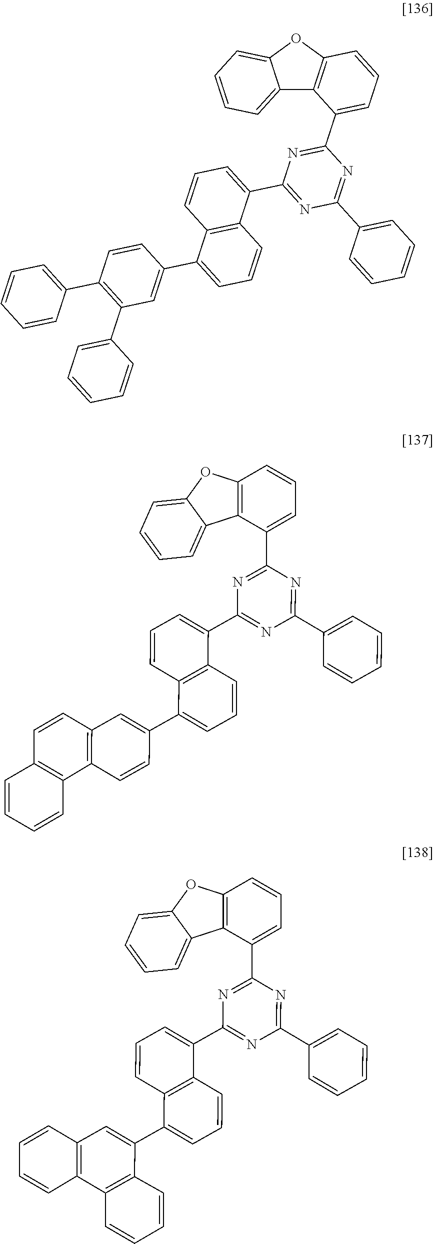 [SMILES: c1ccc(-c2nc(-c3cccc4c(-c5ccc(-c6ccccc6)c(-c6ccccc6)c5)cccc34)nc(-c3cccc4oc5ccccc5c34)n2)cc1 ; c1ccc(-c2nc(-c3cccc4c(-c5ccc6c(ccc7ccccc76)c5)cccc34)nc(-c3cccc4oc5ccccc5c34)n2)cc1 ; c1ccc(-c2nc(-c3cccc4c(-c5cc6ccccc6c6ccccc56)cccc34)nc(-c3cccc4oc5ccccc5c34)n2)cc1]

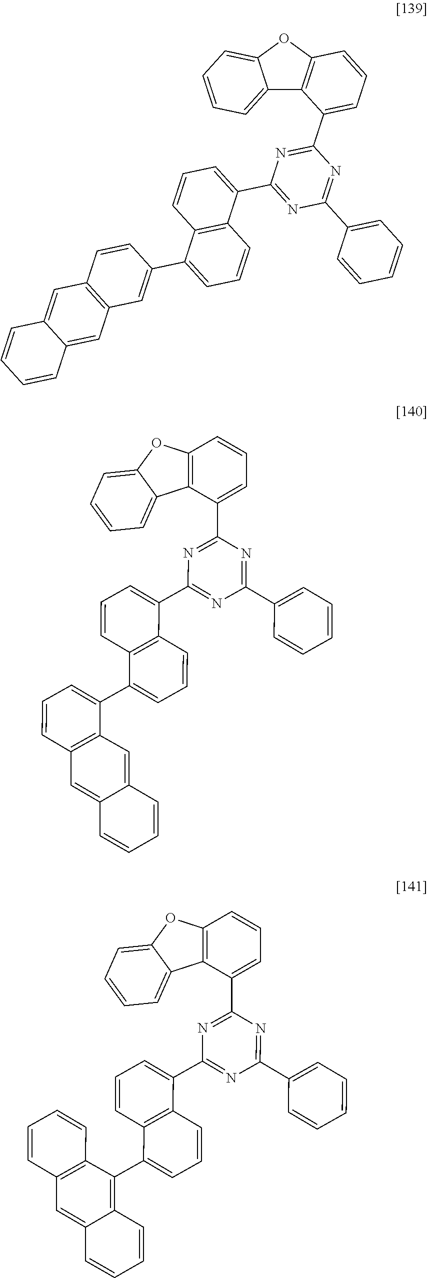 [SMILES: c1ccc(-c2nc(-c3cccc4c(-c5ccc6cc7ccccc7cc6c5)cccc34)nc(-c3cccc4oc5ccccc5c34)n2)cc1 ; c1ccc(-c2nc(-c3cccc4c(-c5cccc6cc7ccccc7cc56)cccc34)nc(-c3cccc4oc5ccccc5c34)n2)cc1 ; c1ccc(-c2nc(-c3cccc4c(-c5c6ccccc6cc6ccccc56)cccc34)nc(-c3cccc4oc5ccccc5c34)n2)cc1]

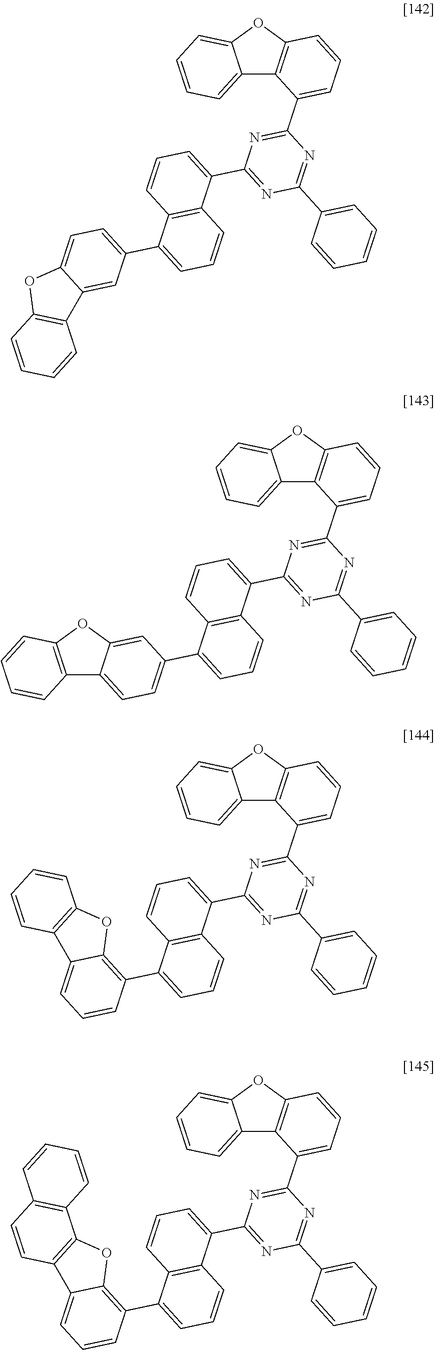 [SMILES: c1ccc(-c2nc(-c3cccc4c(-c5ccc6oc7ccccc7c6c5)cccc34)nc(-c3cccc4oc5ccccc5c34)n2)cc1 ; c1ccc(-c2nc(-c3cccc4c(-c5ccc6c(c5)oc5ccccc56)cccc34)nc(-c3cccc4oc5ccccc5c34)n2)cc1 ; c1ccc(-c2nc(-c3cccc4c(-c5cccc6c5oc5ccccc56)cccc34)nc(-c3cccc4oc5ccccc5c34)n2)cc1 ; c1ccc(-c2nc(-c3cccc4c(-c5cccc6c5oc5c7ccccc7ccc65)cccc34)nc(-c3cccc4oc5ccccc5c34)n2)cc1]

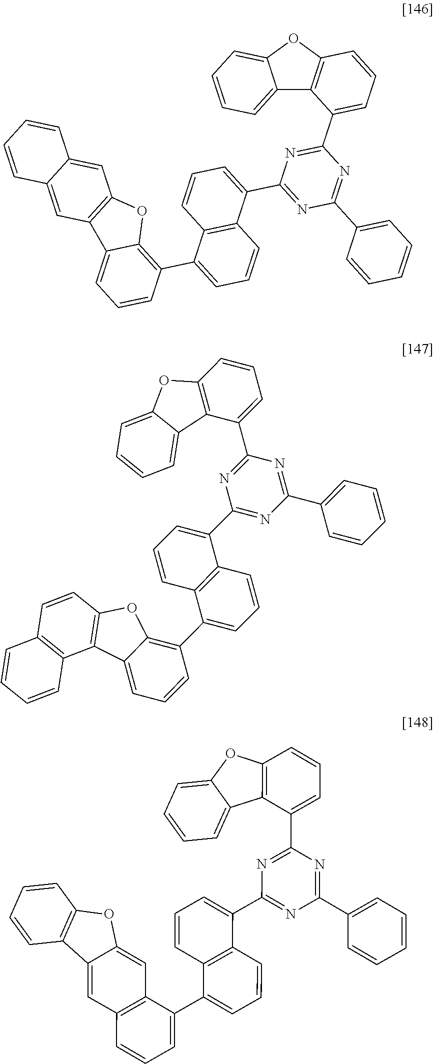 [SMILES: c1ccc(-c2nc(-c3cccc4c(-c5cccc6c5oc5cc7ccccc7cc56)cccc34)nc(-c3cccc4oc5ccccc5c34)n2)cc1 ; c1ccc(-c2nc(-c3cccc4c(-c5cccc6c5oc5ccc7ccccc7c56)cccc34)nc(-c3cccc4oc5ccccc5c34)n2)cc1 ; c1ccc(-c2nc(-c3cccc4c(-c5cccc6cc7c(cc56)oc5ccccc57)cccc34)nc(-c3cccc4oc5ccccc5c34)n2)cc1]

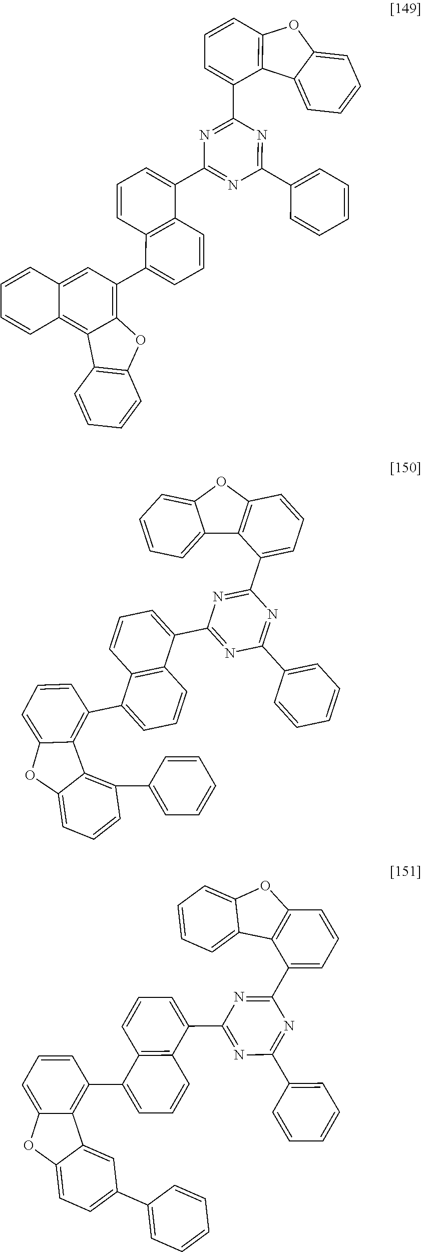 [SMILES: c1ccc(-c2nc(-c3cccc4c(-c5cc6ccccc6c6c5oc5ccccc56)cccc34)nc(-c3cccc4oc5ccccc5c34)n2)cc1 ; c1ccc(-c2nc(-c3cccc4c(-c5cccc6oc7cccc(-c8ccccc8)c7c56)cccc34)nc(-c3cccc4oc5ccccc5c34)n2)cc1 ; c1ccc(-c2ccc3oc4cccc(-c5cccc6c(-c7nc(-c8ccccc8)nc(-c8cccc9oc%10ccccc%10c89)n7)cccc56)c4c3c2)cc1]

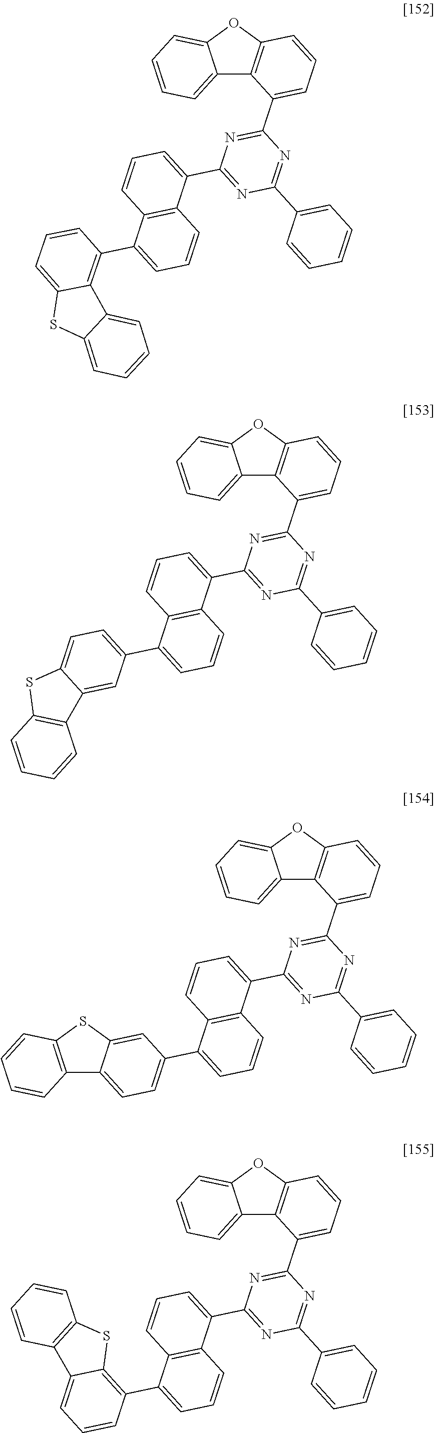 [SMILES: c1ccc(-c2nc(-c3cccc4c(-c5cccc6sc7ccccc7c56)cccc34)nc(-c3cccc4oc5ccccc5c34)n2)cc1 ; c1ccc(-c2nc(-c3cccc4c(-c5ccc6sc7ccccc7c6c5)cccc34)nc(-c3cccc4oc5ccccc5c34)n2)cc1 ; c1ccc(-c2nc(-c3cccc4c(-c5ccc6c(c5)sc5ccccc56)cccc34)nc(-c3cccc4oc5ccccc5c34)n2)cc1 ; c1ccc(-c2nc(-c3cccc4c(-c5cccc6c5sc5ccccc56)cccc34)nc(-c3cccc4oc5ccccc5c34)n2)cc1]

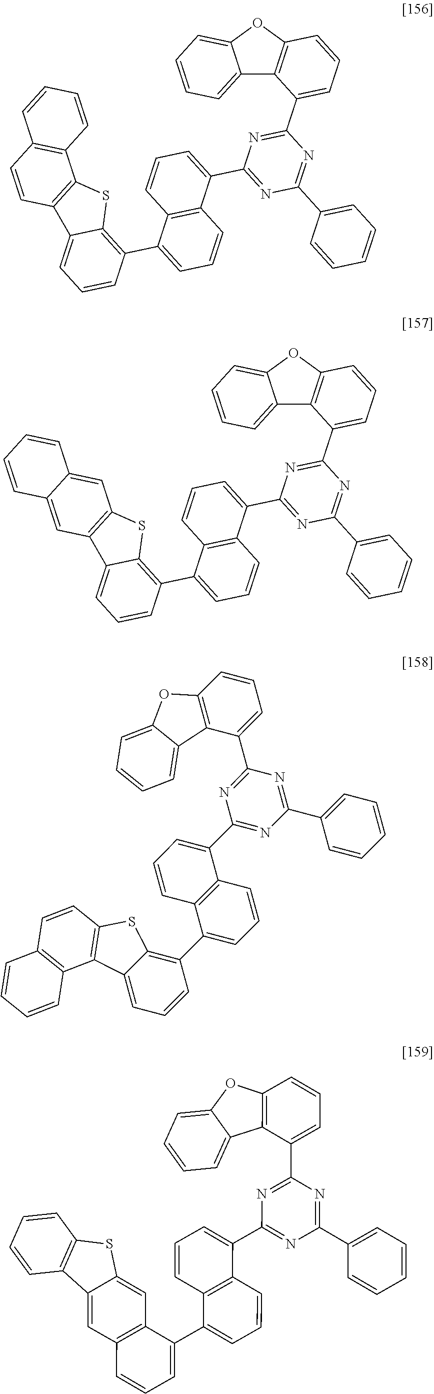 [SMILES: c1ccc(-c2nc(-c3cccc4c(-c5cccc6c5sc5c7ccccc7ccc65)cccc34)nc(-c3cccc4oc5ccccc5c34)n2)cc1 ; c1ccc(-c2nc(-c3cccc4c(-c5cccc6c5sc5cc7ccccc7cc56)cccc34)nc(-c3cccc4oc5ccccc5c34)n2)cc1 ; c1ccc(-c2nc(-c3cccc4c(-c5cccc6c5sc5ccc7ccccc7c56)cccc34)nc(-c3cccc4oc5ccccc5c34)n2)cc1 ; c1ccc(-c2nc(-c3cccc4c(-c5cccc6cc7c(cc56)sc5ccccc57)cccc34)nc(-c3cccc4oc5ccccc5c34)n2)cc1]

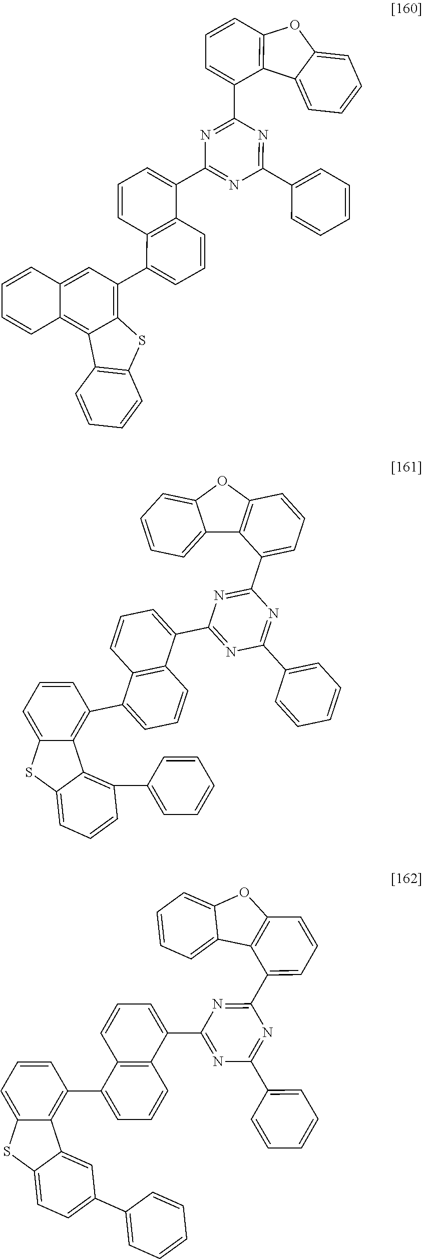 [SMILES: c1ccc(-c2nc(-c3cccc4c(-c5cc6ccccc6c6c5sc5ccccc56)cccc34)nc(-c3cccc4oc5ccccc5c34)n2)cc1 ; c1ccc(-c2nc(-c3cccc4c(-c5cccc6sc7cccc(-c8ccccc8)c7c56)cccc34)nc(-c3cccc4oc5ccccc5c34)n2)cc1 ; c1ccc(-c2ccc3sc4cccc(-c5cccc6c(-c7nc(-c8ccccc8)nc(-c8cccc9oc%10ccccc%10c89)n7)cccc56)c4c3c2)cc1]

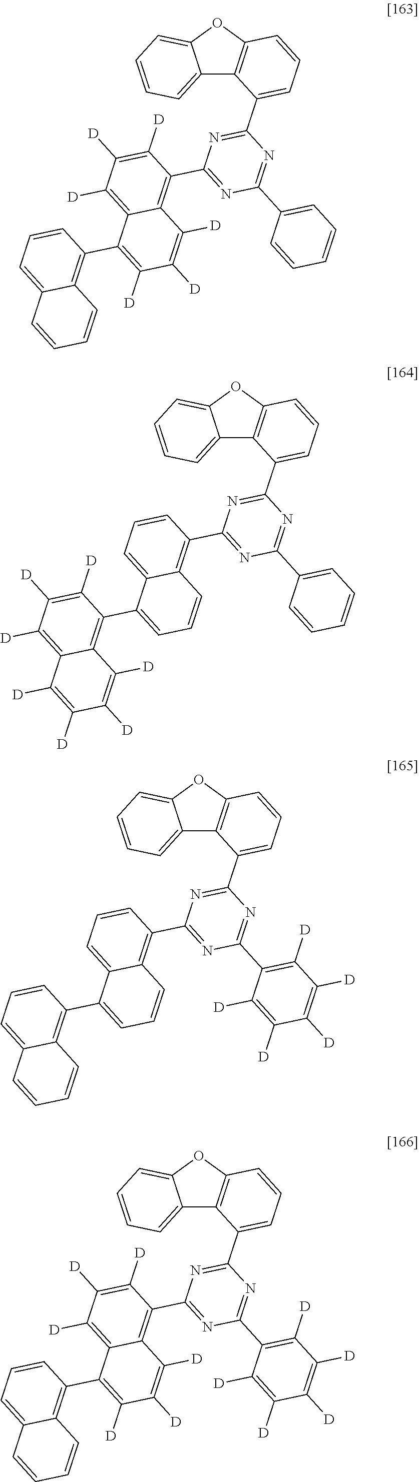 [SMILES: [2H]c1c([2H])c(-c2cccc3ccccc23)c2c([2H])c([2H])c([2H])c(-c3nc(-c4ccccc4)nc(-c4cccc5oc6ccccc6c45)n3)c2c1[2H] ; [2H]c1c([2H])c([2H])c2c(-c3cccc4c(-c5nc(-c6ccccc6)nc(-c6cccc7oc8ccccc8c67)n5)cccc34)c([2H])c([2H])c([2H])c2c1[2H] ; [2H]c1c([2H])c([2H])c(-c2nc(-c3cccc4c(-c5cccc6ccccc56)cccc34)nc(-c3cccc4oc5ccccc5c34)n2)c([2H])c1[2H] ; [2H]c1c([2H])c([2H])c(-c2nc(-c3c([2H])c([2H])c([2H])c4c(-c5cccc6ccccc56)c([2H])c([2H])c([2H])c34)nc(-c3cccc4oc5ccccc5c34)n2)c([2H])c1[2H]]

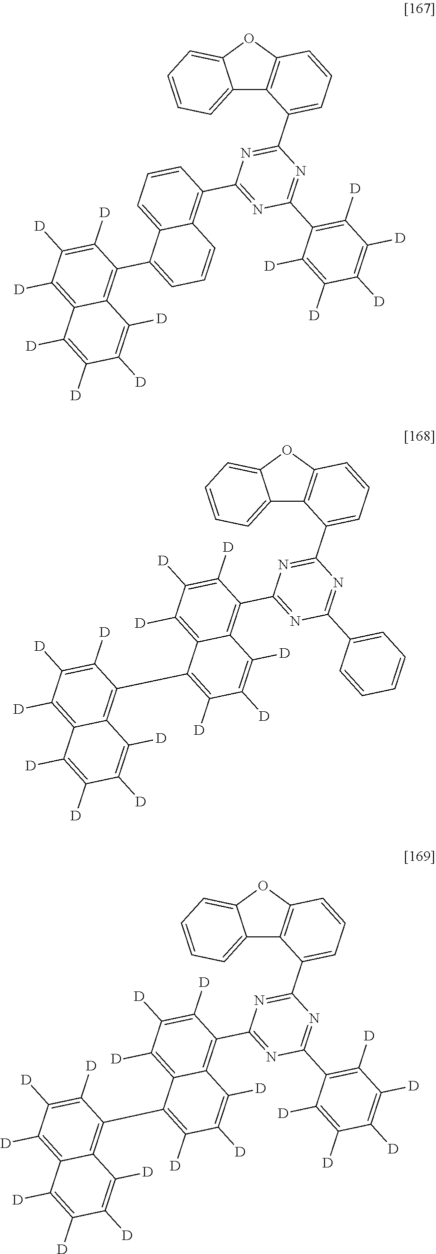 [SMILES: [2H]c1c([2H])c([2H])c(-c2nc(-c3cccc4c(-c5c([2H])c([2H])c([2H])c6c([2H])c([2H])c([2H])c([2H])c56)cccc34)nc(-c3cccc4oc5ccccc5c34)n2)c([2H])c1[2H] ; [2H]c1c([2H])c([2H])c2c(-c3c([2H])c([2H])c([2H])c4c(-c5nc(-c6ccccc6)nc(-c6cccc7oc8ccccc8c67)n5)c([2H])c([2H])c([2H])c34)c([2H])c([2H])c([2H])c2c1[2H] ; [2H]c1c([2H])c([2H])c(-c2nc(-c3c([2H])c([2H])c([2H])c4c(-c5c([2H])c([2H])c([2H])c6c([2H])c([2H])c([2H])c([2H])c56)c([2H])c([2H])c([2H])c34)nc(-c3cccc4oc5ccccc5c34)n2)c([2H])c1[2H]]

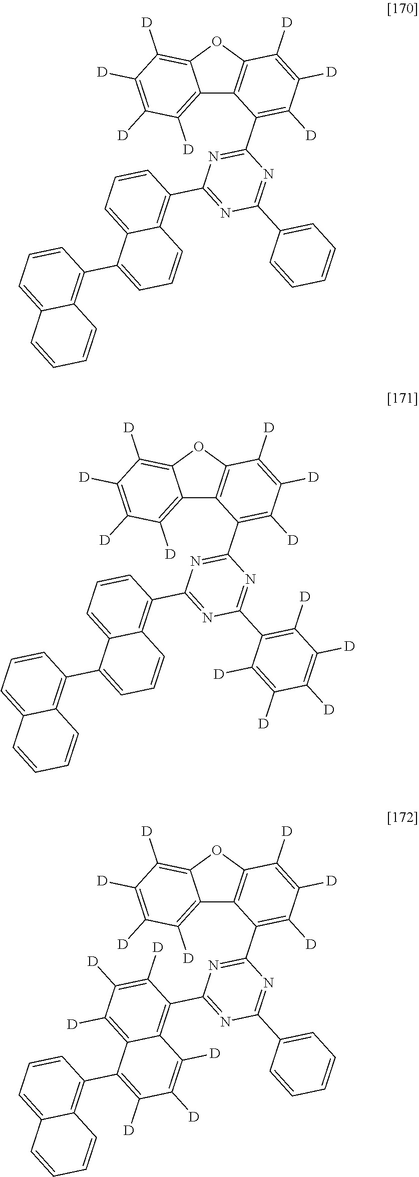 [SMILES: [2H]c1c([2H])c([2H])c2c(oc3c([2H])c([2H])c([2H])c(-c4nc(-c5ccccc5)nc(-c5cccc6c(-c7cccc8ccccc78)cccc56)n4)c32)c1[2H] ; [2H]c1c([2H])c([2H])c(-c2nc(-c3cccc4c(-c5cccc6ccccc56)cccc34)nc(-c3c([2H])c([2H])c([2H])c4oc5c([2H])c([2H])c([2H])c([2H])c5c34)n2)c([2H])c1[2H] ; [2H]c1c([2H])c([2H])c2c(oc3c([2H])c([2H])c([2H])c(-c4nc(-c5ccccc5)nc(-c5c([2H])c([2H])c([2H])c6c(-c7cccc8ccccc78)c([2H])c([2H])c([2H])c56)n4)c32)c1[2H]]

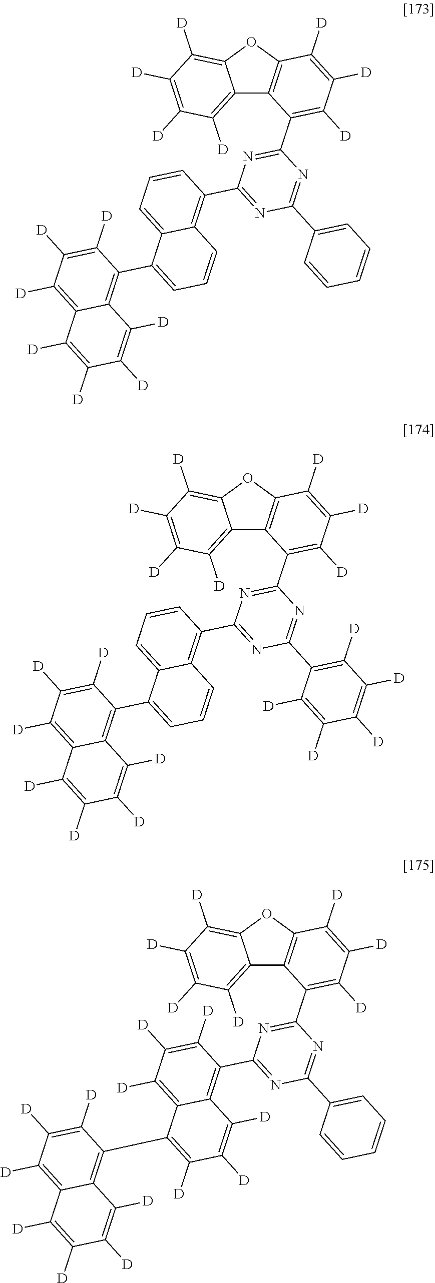

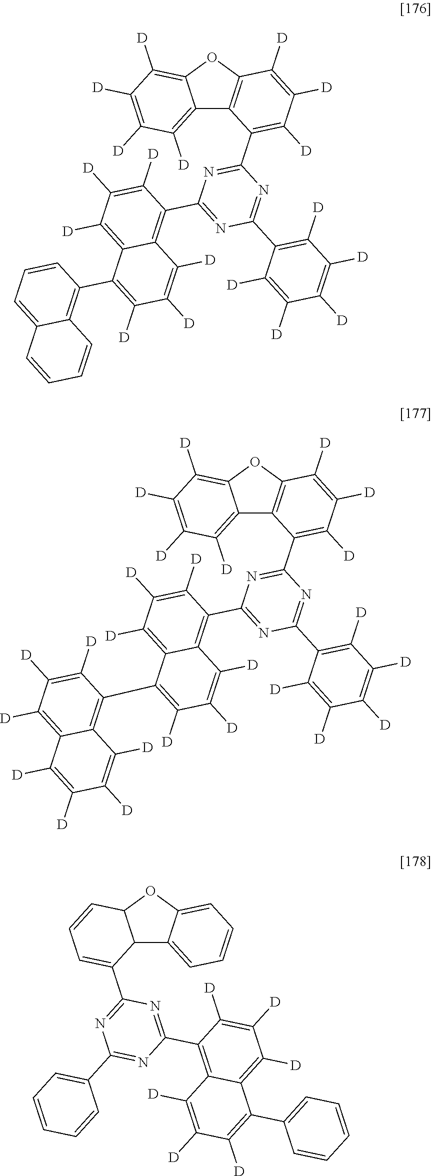

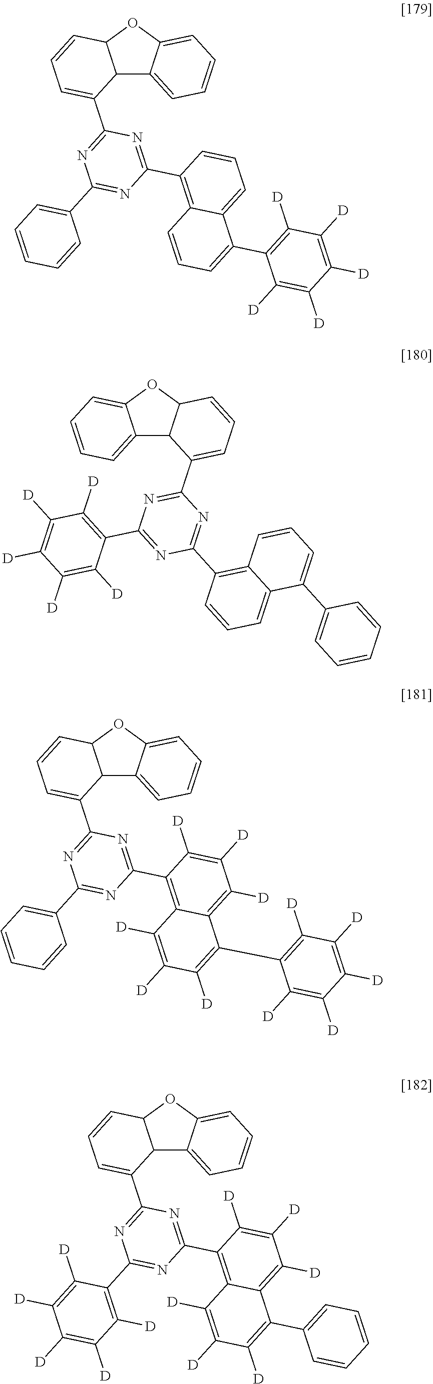 [SMILES: [2H]c1c([2H])c([2H])c(-c2cccc3c(-c4nc(C5=CC=CC6Oc7ccccc7C56)nc(-c5ccccc5)n4)cccc23)c([2H])c1[2H] ; [2H]c1c([2H])c([2H])c(-c2nc(C3=CC=CC4Oc5ccccc5C34)nc(-c3cccc4c(-c5ccccc5)cccc34)n2)c([2H])c1[2H] ; [2H]c1c([2H])c([2H])c(-c2c([2H])c([2H])c([2H])c3c(-c4nc(C5=CC=CC6Oc7ccccc7C56)nc(-c5ccccc5)n4)c([2H])c([2H])c([2H])c23)c([2H])c1[2H] ; [2H]c1c([2H])c([2H])c(-c2nc(C3=CC=CC4Oc5ccccc5C34)nc(-c3c([2H])c([2H])c([2H])c4c(-c5ccccc5)c([2H])c([2H])c([2H])c34)n2)c([2H])c1[2H]]

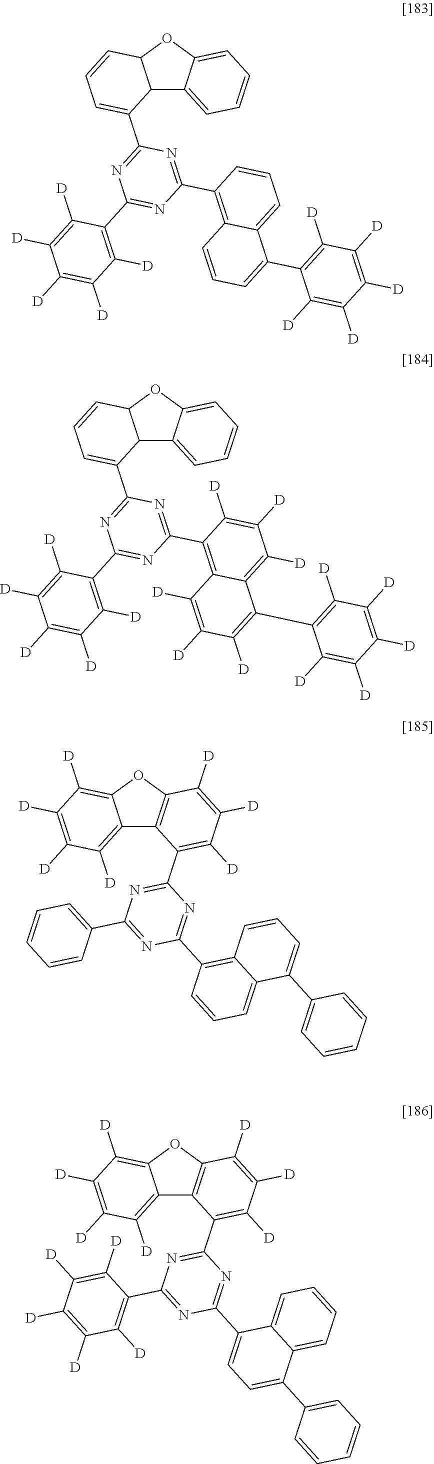

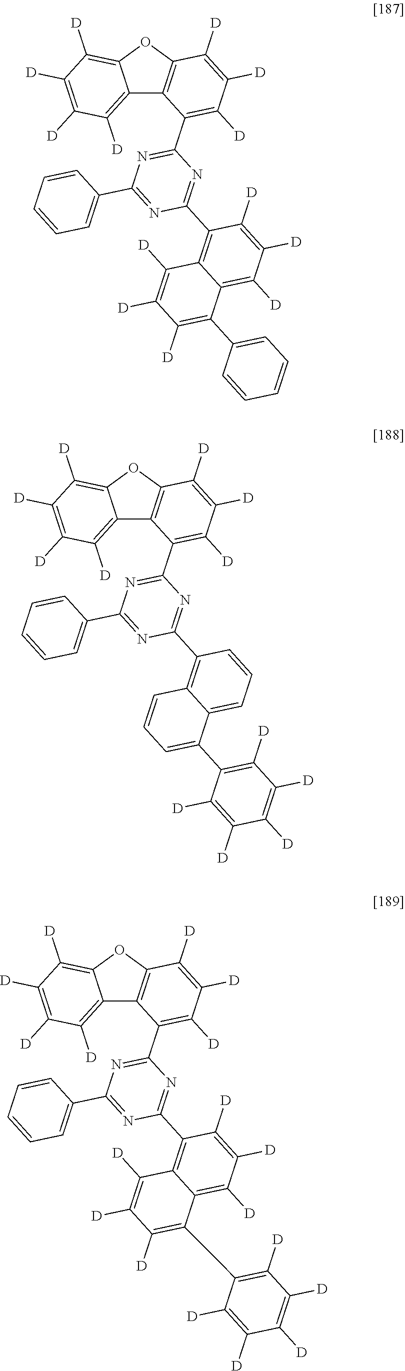 [SMILES: [2H]c1c([2H])c([2H])c2c(oc3c([2H])c([2H])c([2H])c(-c4nc(-c5ccccc5)nc(-c5c([2H])c([2H])c([2H])c6c(-c7ccccc7)c([2H])c([2H])c([2H])c56)n4)c32)c1[2H] ; [2H]c1c([2H])c([2H])c(-c2cccc3c(-c4nc(-c5ccccc5)nc(-c5c([2H])c([2H])c([2H])c6oc7c([2H])c([2H])c([2H])c([2H])c7c56)n4)cccc23)c([2H])c1[2H] ; [2H]c1c([2H])c([2H])c(-c2c([2H])c([2H])c([2H])c3c(-c4nc(-c5ccccc5)nc(-c5c([2H])c([2H])c([2H])c6oc7c([2H])c([2H])c([2H])c([2H])c7c56)n4)c([2H])c([2H])c([2H])c23)c([2H])c1[2H]]

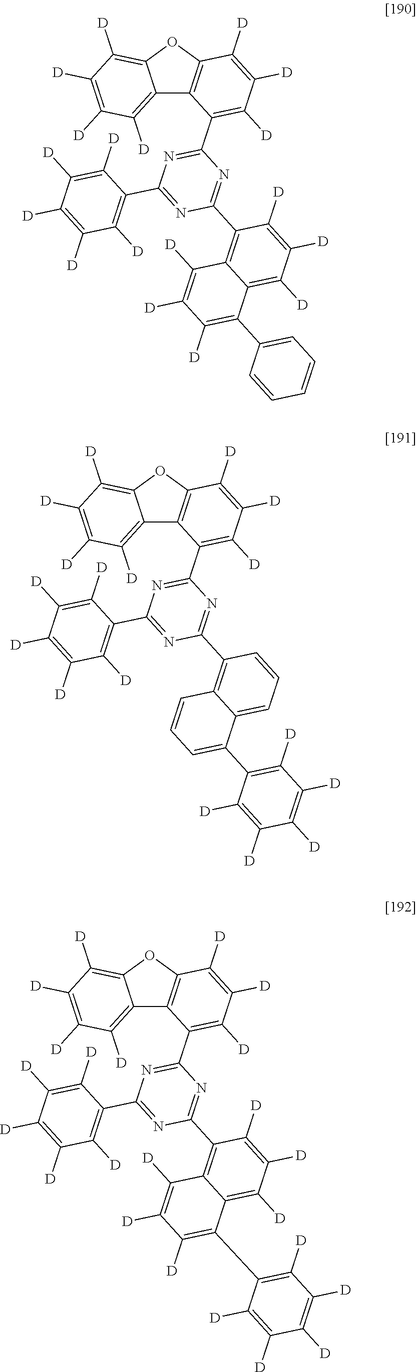

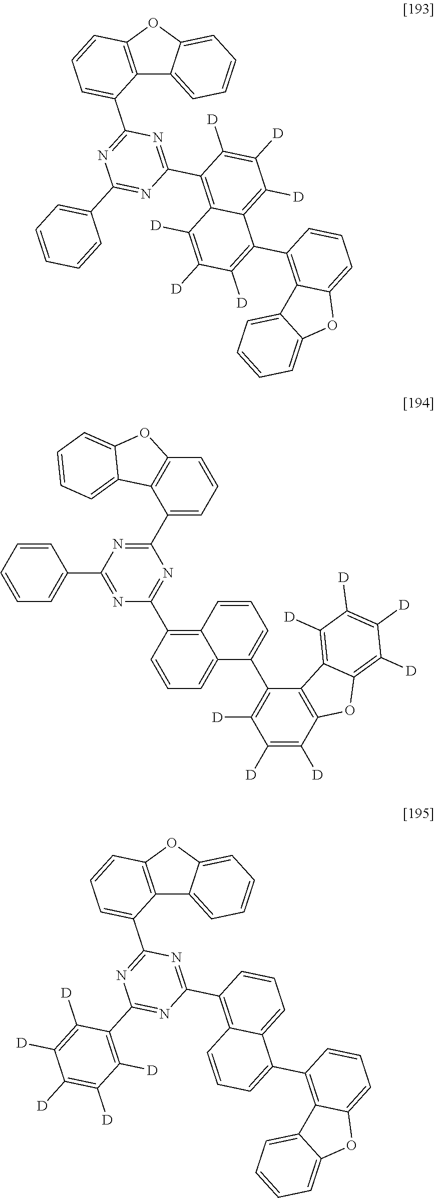 [SMILES: [2H]c1c([2H])c(-c2cccc3oc4ccccc4c23)c2c([2H])c([2H])c([2H])c(-c3nc(-c4ccccc4)nc(-c4cccc5oc6ccccc6c45)n3)c2c1[2H] ; [2H]c1c([2H])c([2H])c2c(oc3c([2H])c([2H])c([2H])c(-c4cccc5c(-c6nc(-c7ccccc7)nc(-c7cccc8oc9ccccc9c78)n6)cccc45)c32)c1[2H] ; [2H]c1c([2H])c([2H])c(-c2nc(-c3cccc4c(-c5cccc6oc7ccccc7c56)cccc34)nc(-c3cccc4oc5ccccc5c34)n2)c([2H])c1[2H]]

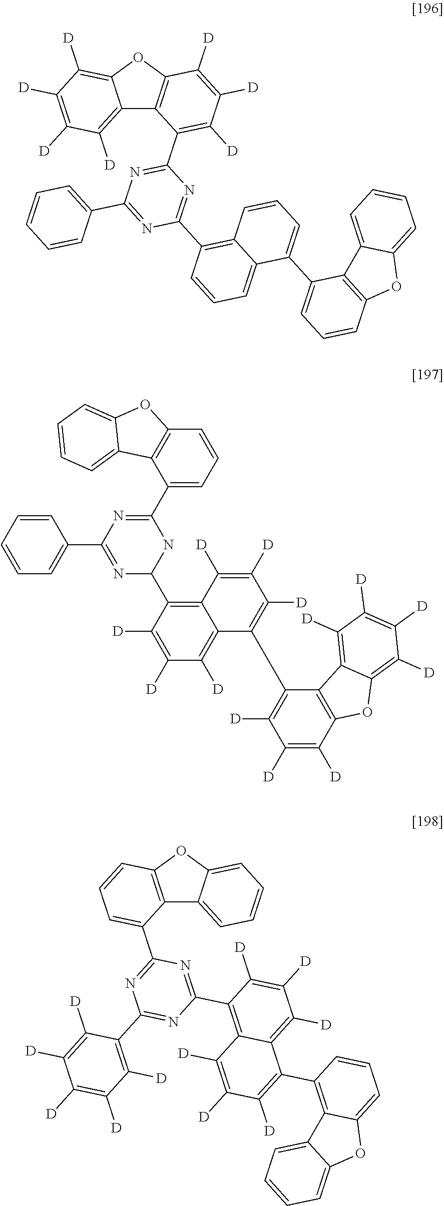

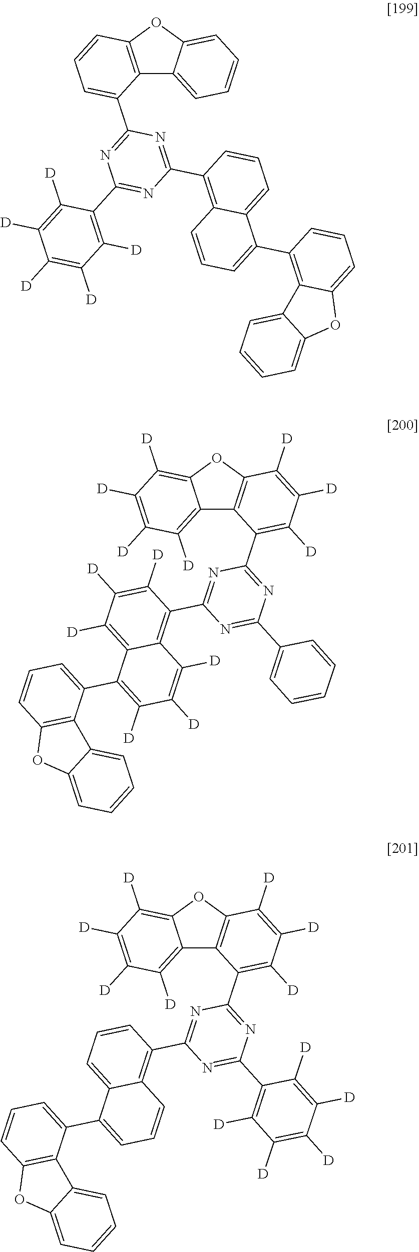 [SMILES: [2H]c1c([2H])c([2H])c(-c2nc(-c3cccc4c(-c5cccc6oc7ccccc7c56)cccc34)nc(-c3cccc4oc5ccccc5c34)n2)c([2H])c1[2H] ; [2H]c1c([2H])c([2H])c2c(oc3c([2H])c([2H])c([2H])c(-c4nc(-c5ccccc5)nc(-c5c([2H])c([2H])c([2H])c6c(-c7cccc8oc9ccccc9c78)c([2H])c([2H])c([2H])c56)n4)c32)c1[2H] ; [2H]c1c([2H])c([2H])c(-c2nc(-c3cccc4c(-c5cccc6oc7ccccc7c56)cccc34)nc(-c3c([2H])c([2H])c([2H])c4oc5c([2H])c([2H])c([2H])c([2H])c5c34)n2)c([2H])c1[2H]]

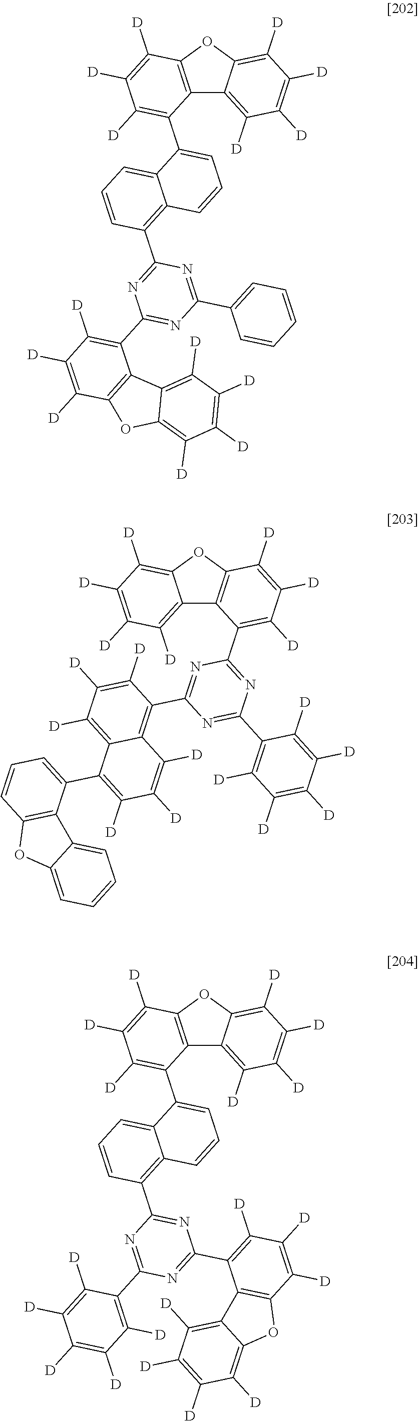

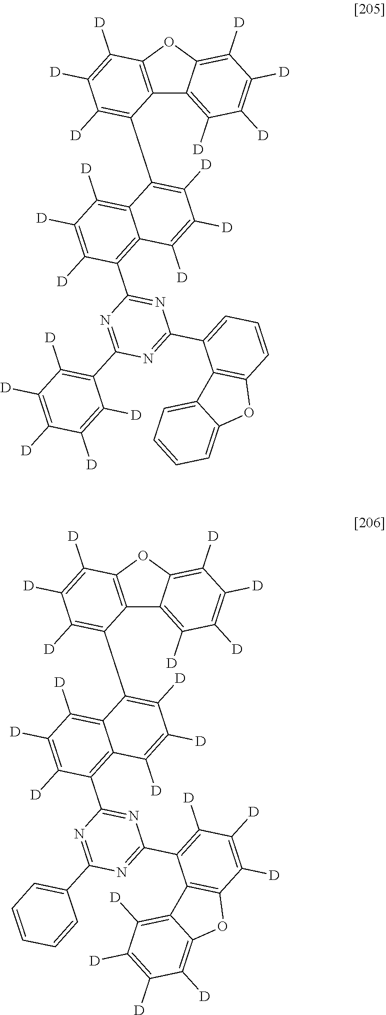 [SMILES: [2H]c1c([2H])c([2H])c(-c2nc(-c3c([2H])c([2H])c([2H])c4c(-c5c([2H])c([2H])c([2H])c6oc7c([2H])c([2H])c([2H])c([2H])c7c56)c([2H])c([2H])c([2H])c34)nc(-c3cccc4oc5ccccc5c34)n2)c([2H])c1[2H] ; [2H]c1c([2H])c([2H])c2c(oc3c([2H])c([2H])c([2H])c(-c4nc(-c5ccccc5)nc(-c5c([2H])c([2H])c([2H])c6c(-c7c([2H])c([2H])c([2H])c8oc9c([2H])c([2H])c([2H])c([2H])c9c78)c([2H])c([2H])c([2H])c56)n4)c32)c1[2H]]

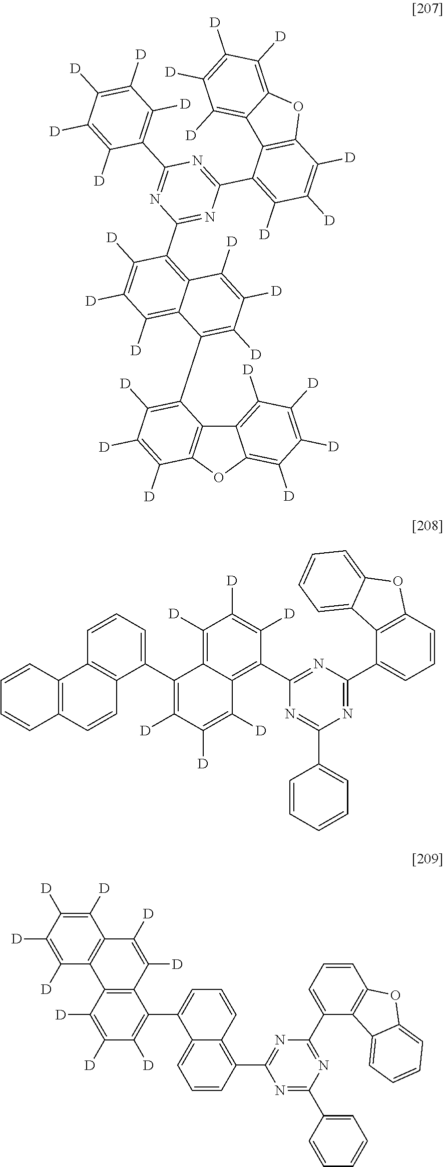 [SMILES: [2H]c1c([2H])c([2H])c(-c2nc(-c3c([2H])c([2H])c([2H])c4c(-c5c([2H])c([2H])c([2H])c6oc7c([2H])c([2H])c([2H])c([2H])c7c56)c([2H])c([2H])c([2H])c34)nc(-c3c([2H])c([2H])c([2H])c4oc5c([2H])c([2H])c([2H])c([2H])c5c34)n2)c([2H])c1[2H] ; [2H]c1c([2H])c(-c2cccc3c2ccc2ccccc23)c2c([2H])c([2H])c([2H])c(-c3nc(-c4ccccc4)nc(-c4cccc5oc6ccccc6c45)n3)c2c1[2H] ; [2H]c1c([2H])c([2H])c2c(c1[2H])c([2H])c([2H])c1c(-c3cccc4c(-c5nc(-c6ccccc6)nc(-c6cccc7oc8ccccc8c67)n5)cccc34)c([2H])c([2H])c([2H])c12]

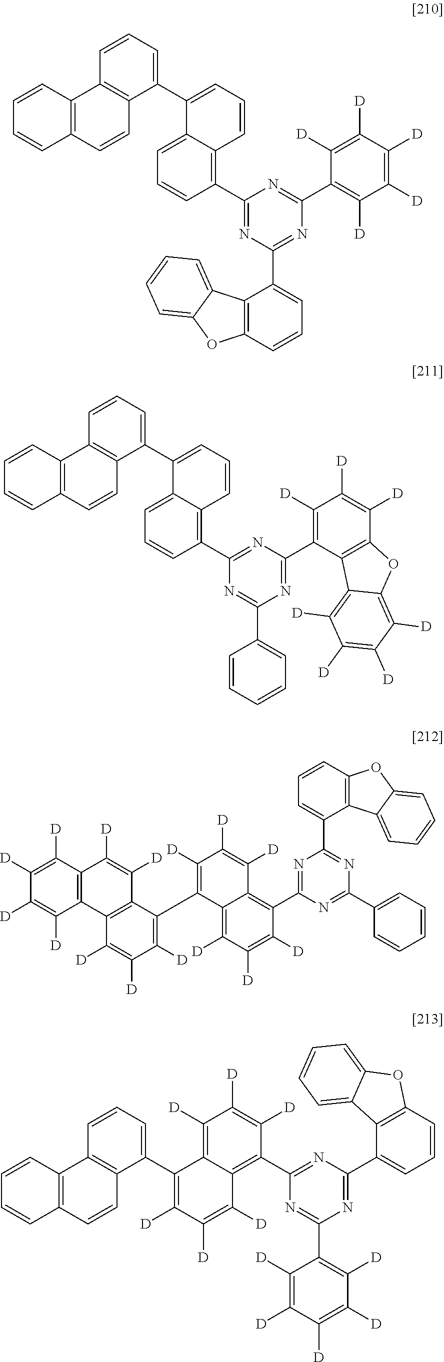

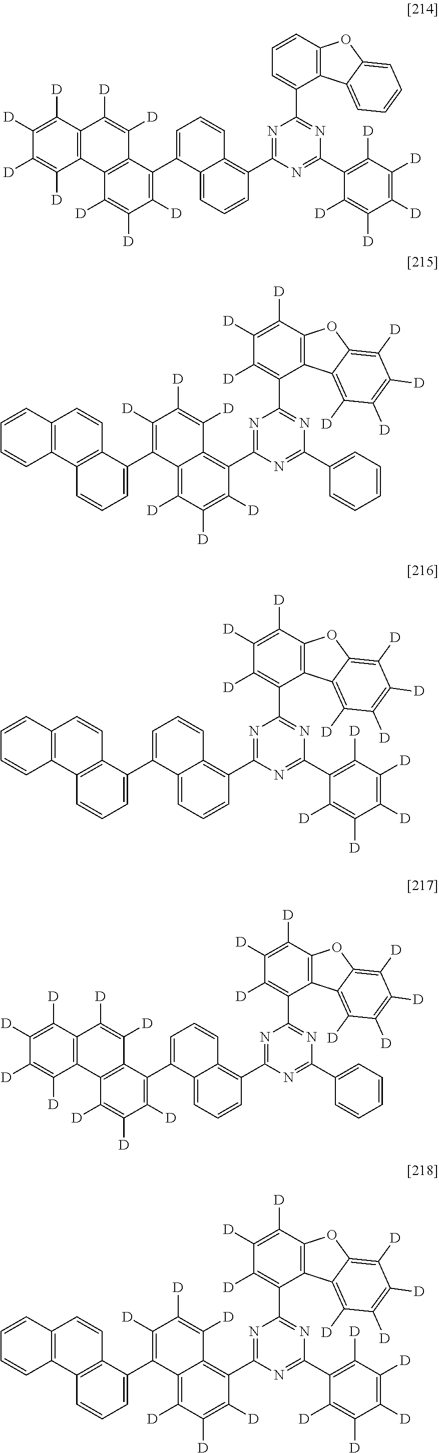

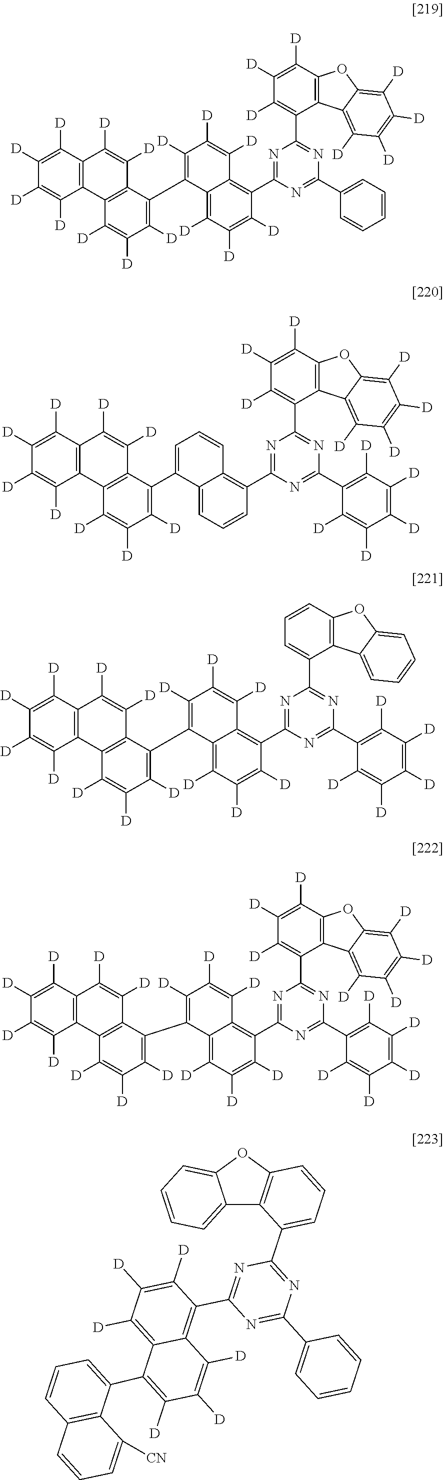

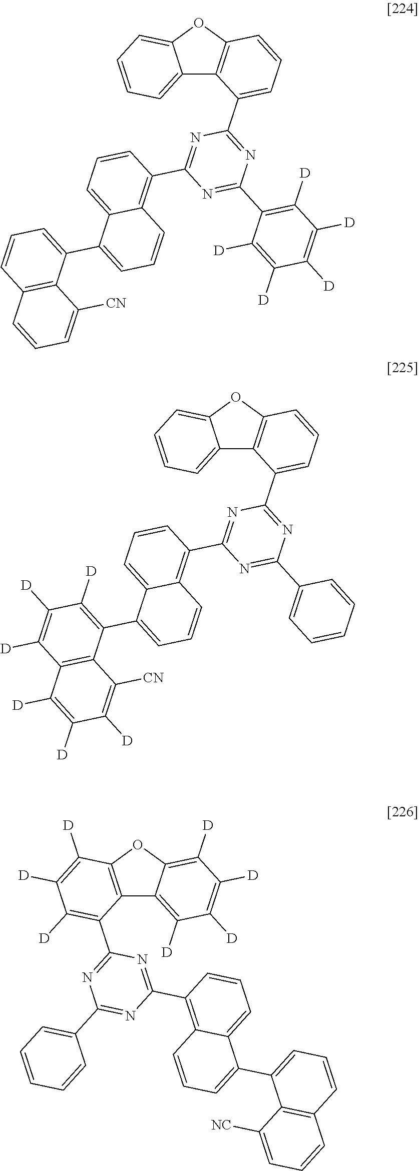 [SMILES: [2H]c1c([2H])c([2H])c(-c2nc(-c3cccc4c(-c5cccc6cccc(C#N)c56)cccc34)nc(-c3cccc4oc5ccccc5c34)n2)c([2H])c1[2H] ; [2H]c1c([2H])c(C#N)c2c(-c3cccc4c(-c5nc(-c6ccccc6)nc(-c6cccc7oc8ccccc8c67)n5)cccc34)c([2H])c([2H])c([2H])c2c1[2H] ; [2H]c1c([2H])c([2H])c2c(oc3c([2H])c([2H])c([2H])c(-c4nc(-c5ccccc5)nc(-c5cccc6c(-c7cccc8cccc(C#N)c78)cccc56)n4)c32)c1[2H]]

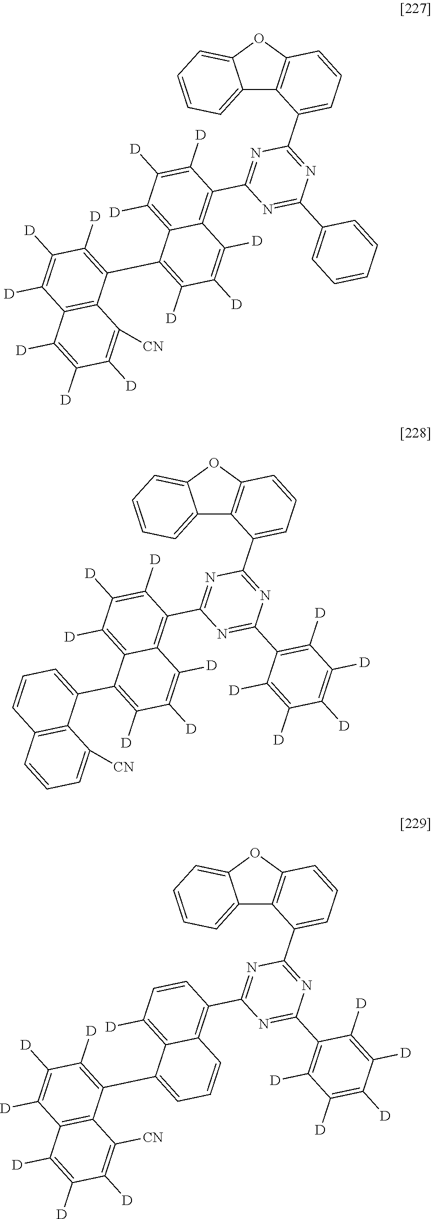 [SMILES: [2H]c1c([2H])c(C#N)c2c(-c3c([2H])c([2H])c([2H])c4c(-c5nc(-c6ccccc6)nc(-c6cccc7oc8ccccc8c67)n5)c([2H])c([2H])c([2H])c34)c([2H])c([2H])c([2H])c2c1[2H] ; [2H]c1c([2H])c([2H])c(-c2nc(-c3c([2H])c([2H])c([2H])c4c(-c5cccc6cccc(C#N)c56)c([2H])c([2H])c([2H])c34)nc(-c3cccc4oc5ccccc5c34)n2)c([2H])c1[2H] ; [2H]c1c([2H])c([2H])c(-c2nc(-c3ccc([2H])c4c(-c5c([2H])c([2H])c([2H])c6c([2H])c([2H])c([2H])c(C#N)c56)cccc34)nc(-c3cccc4oc5ccccc5c34)n2)c([2H])c1[2H]]

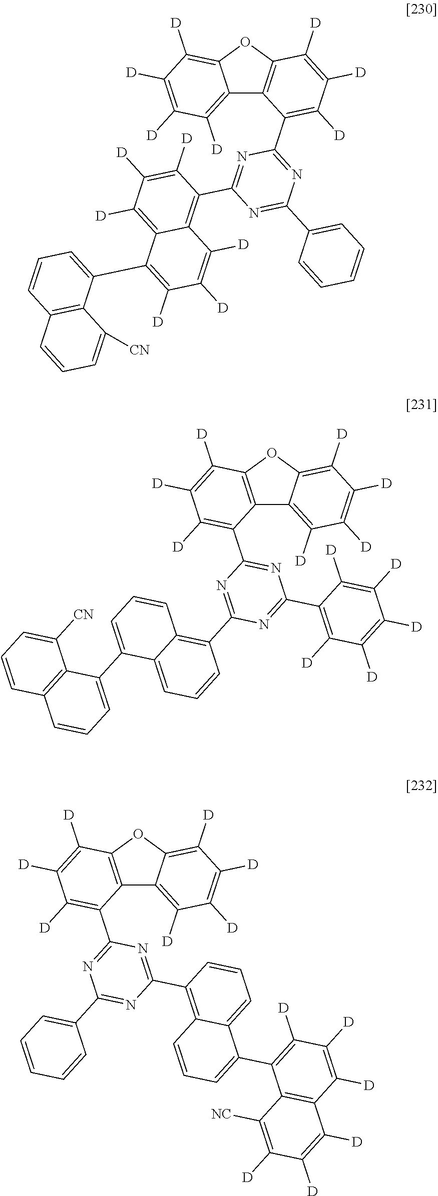 [SMILES: [2H]c1c([2H])c([2H])c2c(oc3c([2H])c([2H])c([2H])c(-c4nc(-c5ccccc5)nc(-c5c([2H])c([2H])c([2H])c6c(-c7cccc8cccc(C#N)c78)c([2H])c([2H])c([2H])c56)n4)c32)c1[2H] ; [2H]c1c([2H])c([2H])c(-c2nc(-c3cccc4c(-c5cccc6cccc(C#N)c56)cccc34)nc(-c3c([2H])c([2H])c([2H])c4oc5c([2H])c([2H])c([2H])c([2H])c5c34)n2)c([2H])c1[2H] ; [2H]c1c([2H])c([2H])c2c(oc3c([2H])c([2H])c([2H])c(-c4nc(-c5ccccc5)nc(-c5cccc6c(-c7c([2H])c([2H])c([2H])c8c([2H])c([2H])c([2H])c(C#N)c78)cccc56)n4)c32)c1[2H]]

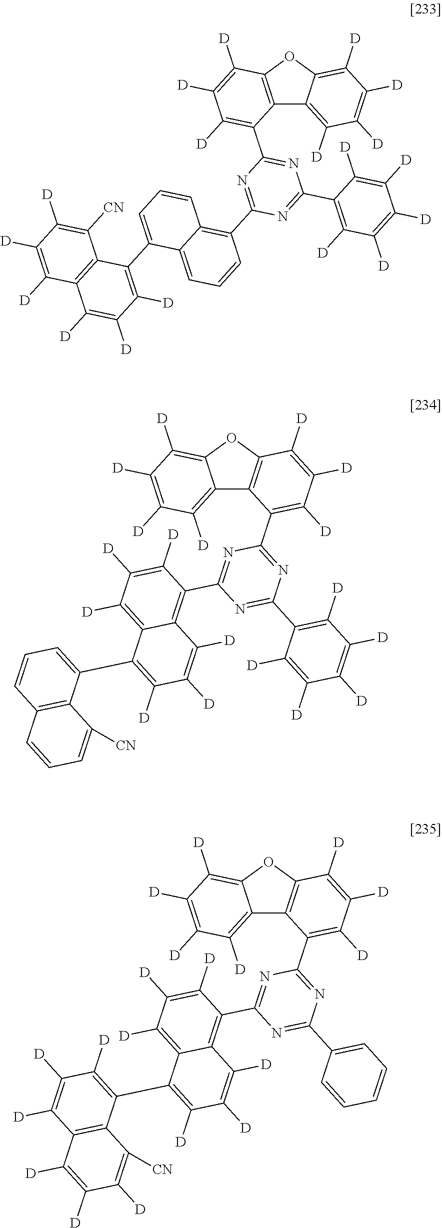

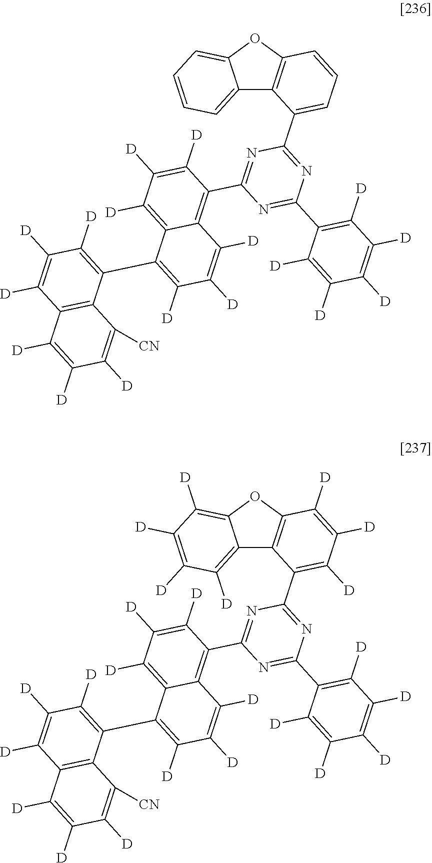 [SMILES: [2H]c1c([2H])c([2H])c(-c2nc(-c3c([2H])c([2H])c([2H])c4c(-c5c([2H])c([2H])c([2H])c6c([2H])c([2H])c([2H])c(C#N)c56)c([2H])c([2H])c([2H])c34)nc(-c3cccc4oc5ccccc5c34)n2)c([2H])c1[2H] ; [2H]c1c([2H])c([2H])c(-c2nc(-c3c([2H])c([2H])c([2H])c4c(-c5c([2H])c([2H])c([2H])c6c([2H])c([2H])c([2H])c(C#N)c56)c([2H])c([2H])c([2H])c34)nc(-c3c([2H])c([2H])c([2H])c4oc5c([2H])c([2H])c([2H])c([2H])c5c34)n2)c([2H])c1[2H]]

A composition for an organic optoelectronic device according to an embodiment may include, e.g., a first compound and a second compound. In an implementation, the first compound may be, e.g., the aforementioned compound for the organic optoelectronic device according to an embodiment. In an implementation, the second compound may be, e.g., represented by Chemical Formula 2.

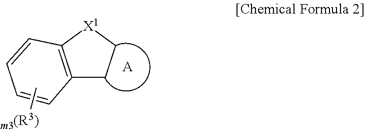

In Chemical Formula 2, X¹ may be, e.g., O, S, NR^{b}, CR^{c}R^{d}, or SiR^{e}R^{f}.

R^{b}, R^{c}, R^{d}, R^{e}, R^{f}, and R³ may each independently be or include, e.g., hydrogen, deuterium, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

m3 may be, e.g., an integer of 1 to 4.

ring A may be, e.g., a ring of Group II.

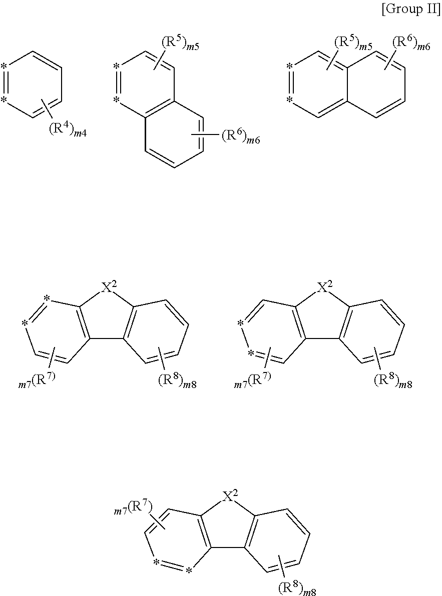

In Group II, * is a linking point, e.g., a shared carbon at which the ring of Group

II is fused/linked with the X¹-containing ring of Chemical Formula 2.

X² may be, e.g., O, S, NR^{g}, CR^{h}Rⁱ, or SiR^{j}R^{k}.

R^{g}, R^{h}, Rⁱ, R^{j}, R^{k}, and R⁴ to R⁸ may each independently be, e.g., hydrogen, deuterium, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

m4, m6, and m8 may each independently be, e.g., an integer of 1 to 4.

m5 and m7 may each independently be, e.g., 1 or 2.

In an implementation, at least one of R³ to R⁸ may be, e.g., a (e.g., substituted amine) group represented by Chemical Formula a,

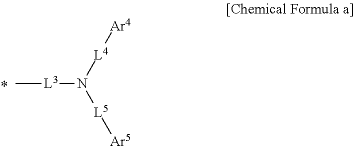

In Chemical Formula a, L³ to L⁵ may each independently be, e.g., a single bond or a substituted or unsubstituted C6 to C30 arylene group.

Ar⁴ and Ar⁵ may each independently be, e.g., a substituted or unsubstituted amine group, a substituted or unsubstituted C6 to C30 aryl group, or a substituted or unsubstituted C2 to C30 heterocyclic group.

* is a linking point.

In Chemical Formula 2, when m3 is 2 or more, each R³ may be the same or different from each other.

In Group II, when m4 is 2 or more, each R⁴ may be the same or different from each other.

In Group II, when m5 is 2 or more, each R⁵ may be the same or different from each other.

In Group II, when m6 is 2 or more, each R⁶ may be the same or different from each other.

In Group II, when m7 is 2 or more, each R⁷ may be the same or different from each other.

In Group II, when m8 is 2 or more, each R⁸ may be the same or different from each other.

The second compound may have a structure in which carbazole/fused carbazole/fused dibenzofuran/fused dibenzothiophene/fused dibenzosilole is substituted with an amine and may be represented by, e.g., one of Chemical Formula 2-I to Chemical Formula 2-IX, depending on the type and fusion position of the additional benzene ring.

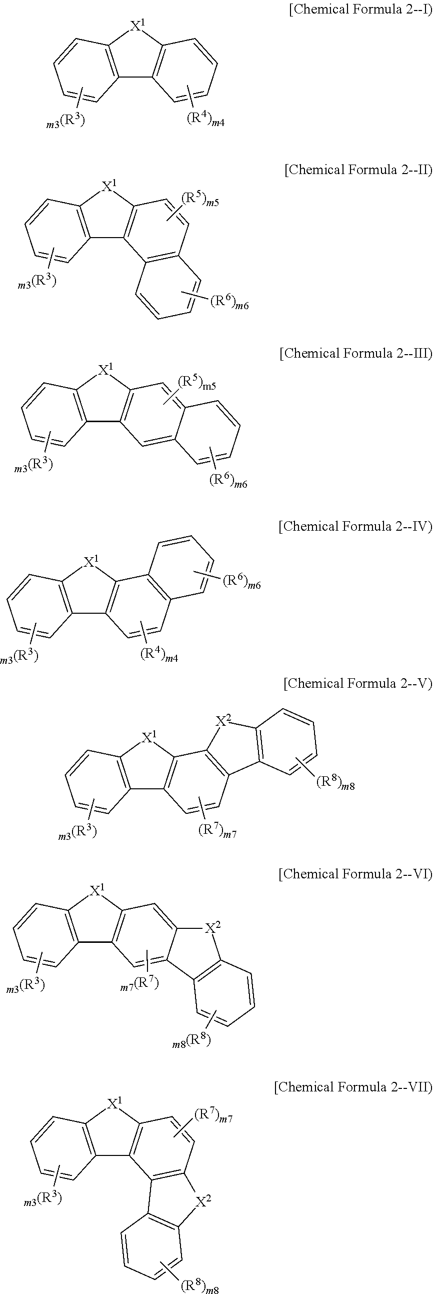

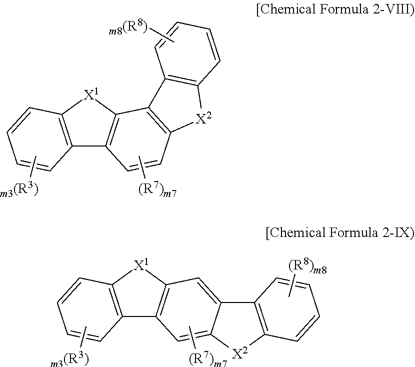

In Chemical Formula 2-I to Chemical Formula 2-IX, X¹, X², m3 to m8, and R³ to R⁸ may be defined the same as those described above.

In an implementation, the second compound may be represented by, e.g., one of Chemical Formula 2-IA to Chemical Formula 2-IXA, Chemical Formula 2-IB to Chemical Formula 2-IXB, and Chemical Formula 2-IIC to Chemical Formula 2-IVC, depending on a substitution direction or position of the amine group.

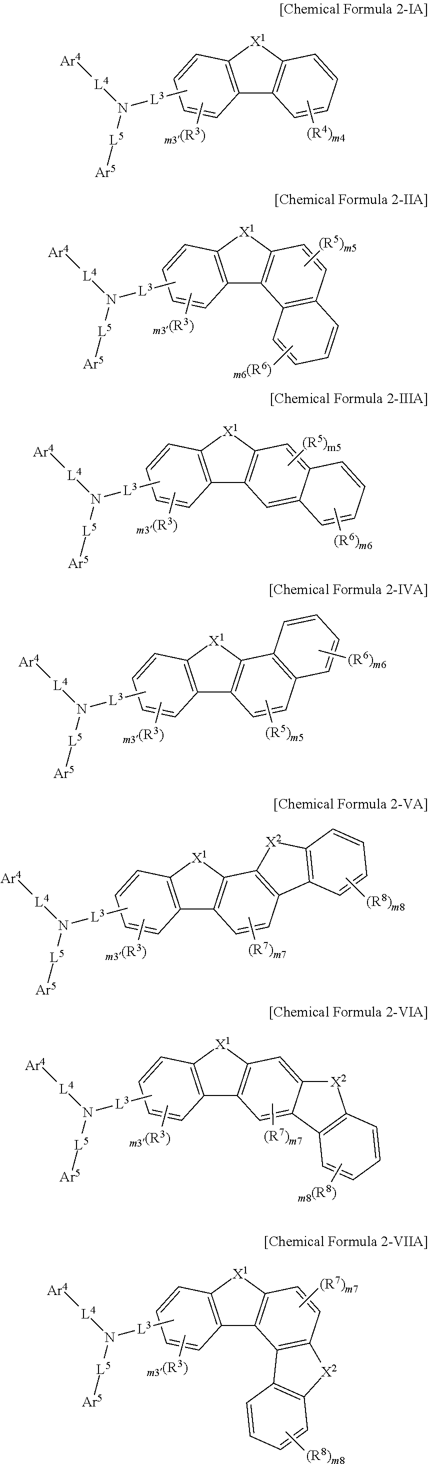

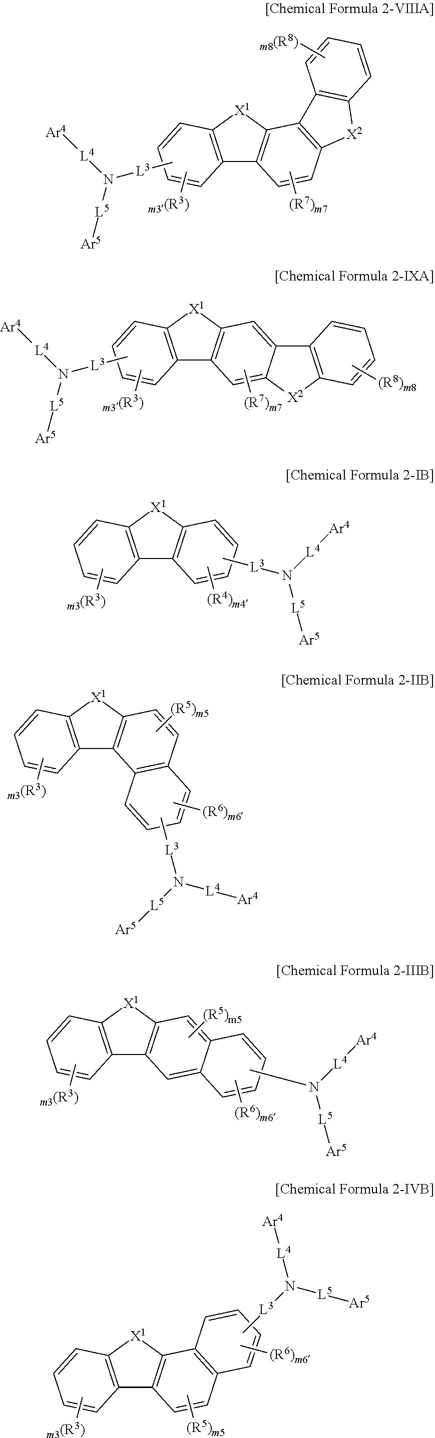

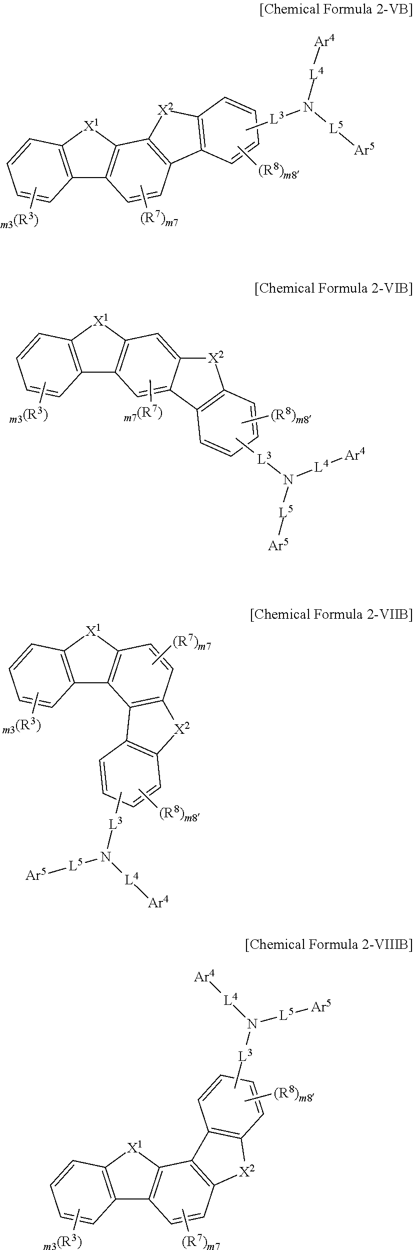

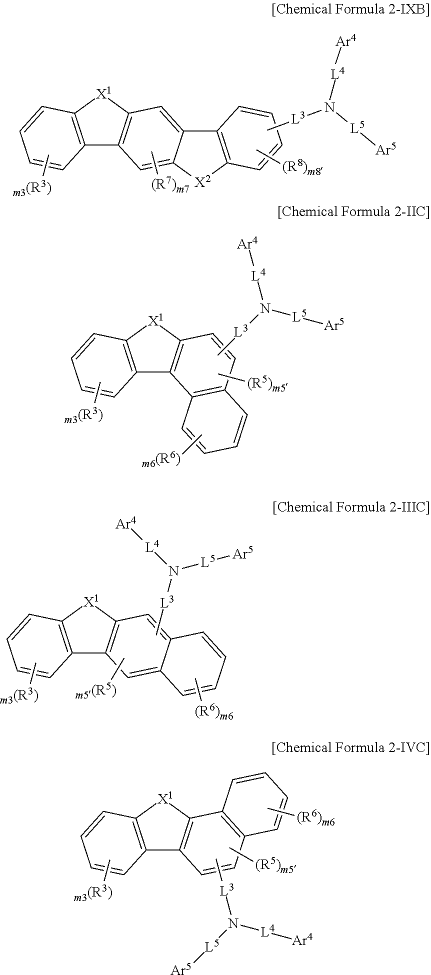

In Chemical Formula 2-IA to Chemical Formula 2-IXA, Chemical Formula 2-IB to Chemical Formula 2-IXB, and Chemical Formula 2-IIC to Chemical Formula 2-IVC, X¹, X², L³ to L⁵, m3 to m8, Ar⁴, and Ar⁵ may be defined the same as those described above.

R³ to R⁸ may each independently be, e.g., hydrogen, deuterium, a substituted or unsubstituted C1 to C30 alkyl group, or a substituted or unsubstituted C6 to C30 aryl group.

m3′, m4′, m6′, and m8′ may each independently be, e.g., an integer of 1 to 3.

m5′ may be, e.g., 1.

when m3′ is 2 or more, each R³ may be the same or different from each other.

when m4′ is 2 or more, each R⁴ may be the same or different from each other.

when m6′ is 2 or more, each R⁶ may be the same or different from each other.

when m8′ is 2 or more, each R⁸ may be the same or different from each other.

In an implementation, the second compound according to an embodiment may be represented by, e.g., Chemical Formula 2-IVB or Chemical Formula 2-VIIIB.

In an implementation, X² of Chemical Formula 2-IVB may be, e.g., NR^{b}.

In an implementation, in Chemical Formula X¹ may be, e.g., 0 or S, and X² may be, e.g., CR^{h}Rⁱ or SiR^{j}R^{k}.

In an implementation, R^{b}, R^{h}, Rⁱ, R^{j}, and R^{k} may each independently be, e.g., a substituted or unsubstituted C1 to C10 alkyl group or a substituted or unsubstituted C6 to C20 aryl group.

In an implementation, the second compound may be represented by, e.g., Chemical Formula 2-IVB-2 or Chemical Formula 2-VIIIB-2.

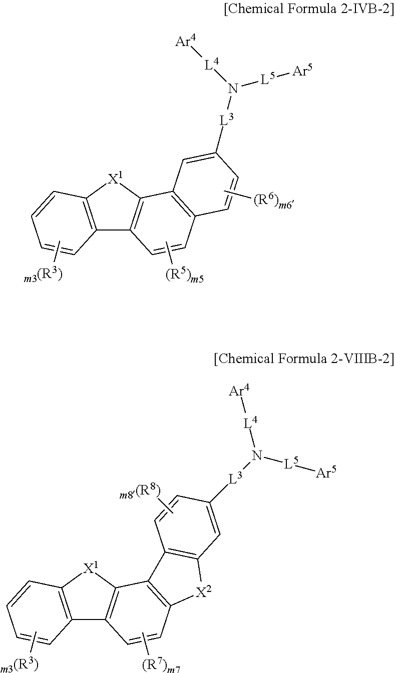

In Chemical Formula 2-IVB-2 and Chemical Formula 2-VIIIB-2, L³ to L⁵ may each independently be, e.g., a single bond or a substituted or unsubstituted phenylene group.

Ar⁴ and Ar⁵ may each independently be, e.g., a substituted or unsubstituted phenyl group, a substituted or unsubstituted biphenyl group, or a substituted or unsubstituted naphthyl group.

X¹ may be, e.g., NR^{b}, O, or S.

X² may be, e.g., CR^{h}Rⁱ or SiR^{j}R^{k}.

R^{b}, R^{h}, Rⁱ, R^{j}, and R^{k} may each independently be, e.g., a substituted or unsubstituted C1 to C10 alkyl group or a substituted or unsubstituted C6 to C20 aryl group.

R³, R⁵, R⁶, R⁷, and R⁸ may each independently be, e.g., hydrogen, deuterium, a substituted or unsubstituted C1 to C30 alkyl group, or a substituted or unsubstituted C6 to C30 aryl group.

m3 may be, e.g., an integer of 1 to 4.

m5 and m7 may each independently be, e.g., 1 or 2.

m6′ and m8′ may each independently be, e.g., an integer of 1 to 3.

In an implementation, L⁴ and L⁵ may each independently be, e.g., a single bond, a substituted or unsubstituted phenylene group, or a substituted or unsubstituted biphenylene group.

In an implementation, Ar⁴ and Ar^{y} may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group, a substituted or unsubstituted dibenzothiophenyl group, a substituted or unsubstituted benzofuranofluorenyl group, or a substituted or unsubstituted benzothiophenofluorenyl group.

In an implementation, the second compound may be, e.g., a compound of Group 2.

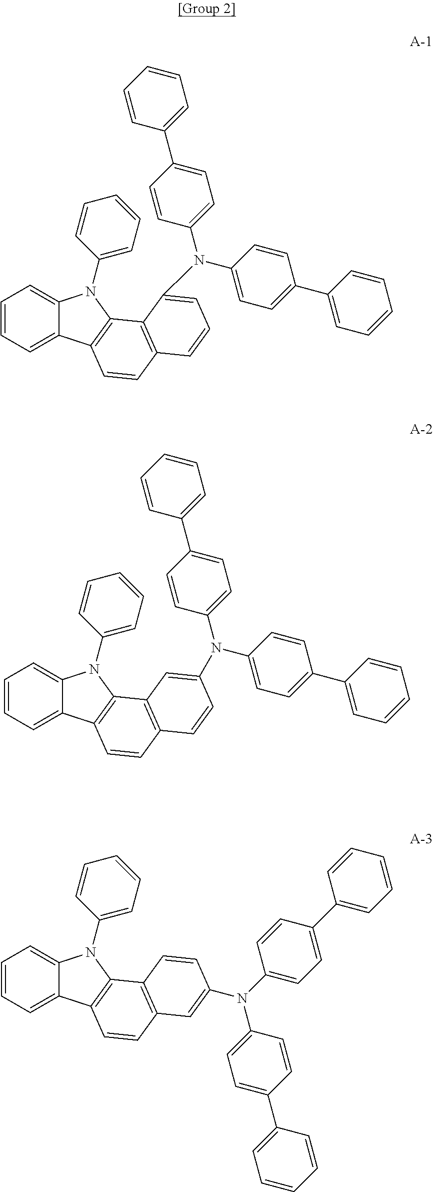 [SMILES: c1ccc(-c2ccc(N(c3ccc(-c4ccccc4)cc3)c3cccc4ccc5c6ccccc6n(-c6ccccc6)c5c34)cc2)cc1 ; c1ccc(-c2ccc(N(c3ccc(-c4ccccc4)cc3)c3ccc4ccc5c6ccccc6n(-c6ccccc6)c5c4c3)cc2)cc1 ; c1ccc(-c2ccc(N(c3ccc(-c4ccccc4)cc3)c3ccc4c(ccc5c6ccccc6n(-c6ccccc6)c45)c3)cc2)cc1]

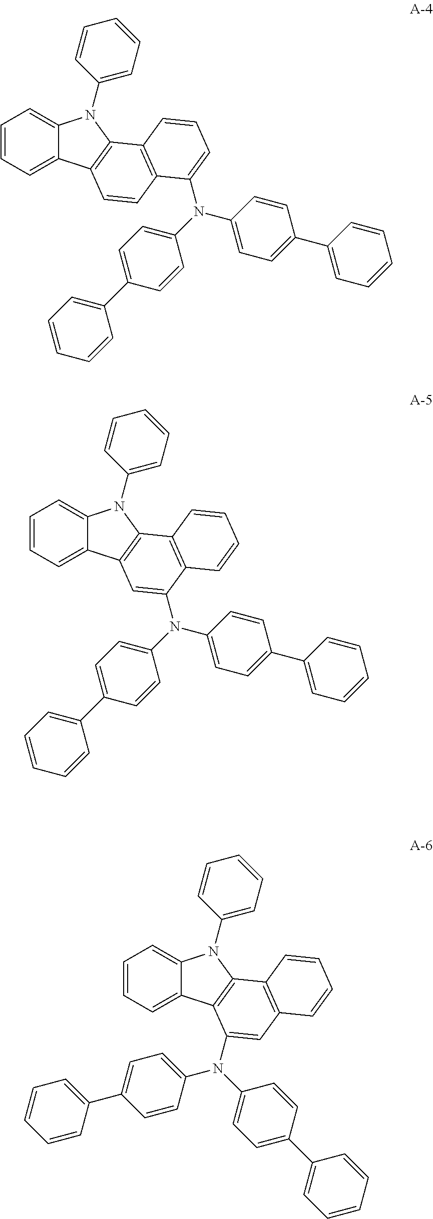 [SMILES: c1ccc(-c2ccc(N(c3ccc(-c4ccccc4)cc3)c3cccc4c3ccc3c5ccccc5n(-c5ccccc5)c43)cc2)cc1 ; c1ccc(-c2ccc(N(c3ccc(-c4ccccc4)cc3)c3cc4c5ccccc5n(-c5ccccc5)c4c4ccccc34)cc2)cc1 ; c1ccc(-c2ccc(N(c3ccc(-c4ccccc4)cc3)c3cc4ccccc4c4c3c3ccccc3n4-c3ccccc3)cc2)cc1]

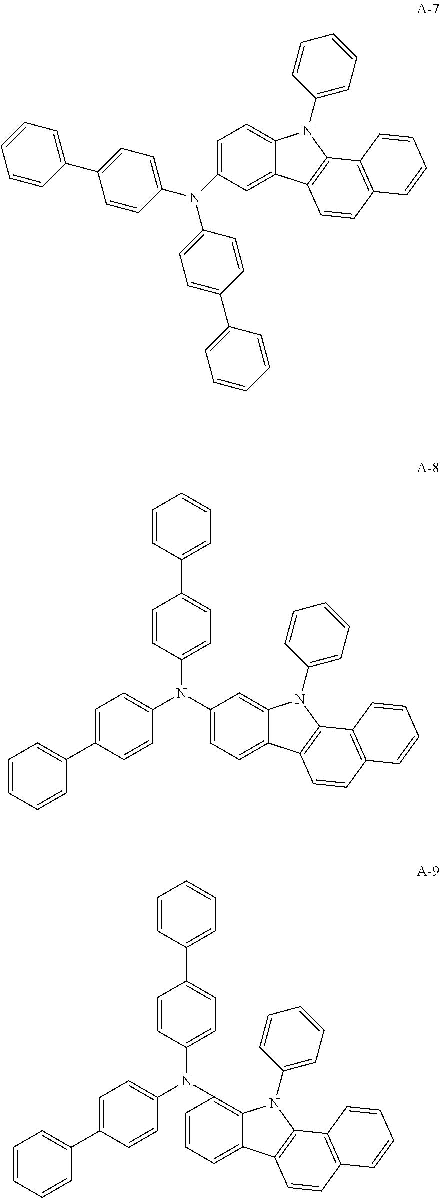 [SMILES: c1ccc(-c2ccc(N(c3ccc(-c4ccccc4)cc3)c3ccc4c(c3)c3ccc5ccccc5c3n4-c3ccccc3)cc2)cc1 ; c1ccc(-c2ccc(N(c3ccc(-c4ccccc4)cc3)c3ccc4c5ccc6ccccc6c5n(-c5ccccc5)c4c3)cc2)cc1 ; c1ccc(-c2ccc(N(c3ccc(-c4ccccc4)cc3)c3cccc4c5ccc6ccccc6c5n(-c5ccccc5)c34)cc2)cc1]

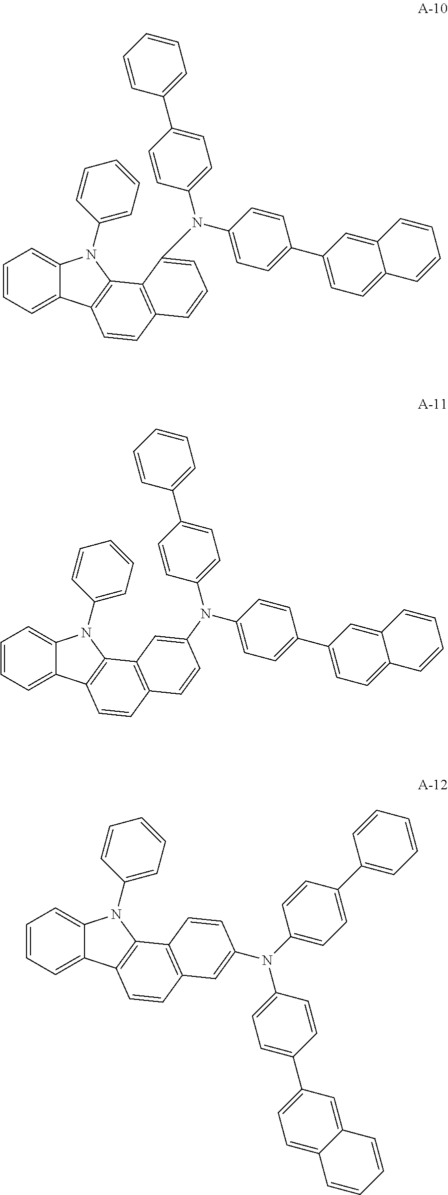 [SMILES: c1ccc(-c2ccc(N(c3ccc(-c4ccc5ccccc5c4)cc3)c3cccc4ccc5c6ccccc6n(-c6ccccc6)c5c34)cc2)cc1 ; c1ccc(-c2ccc(N(c3ccc(-c4ccc5ccccc5c4)cc3)c3ccc4ccc5c6ccccc6n(-c6ccccc6)c5c4c3)cc2)cc1 ; c1ccc(-c2ccc(N(c3ccc(-c4ccc5ccccc5c4)cc3)c3ccc4c(ccc5c6ccccc6n(-c6ccccc6)c45)c3)cc2)cc1]

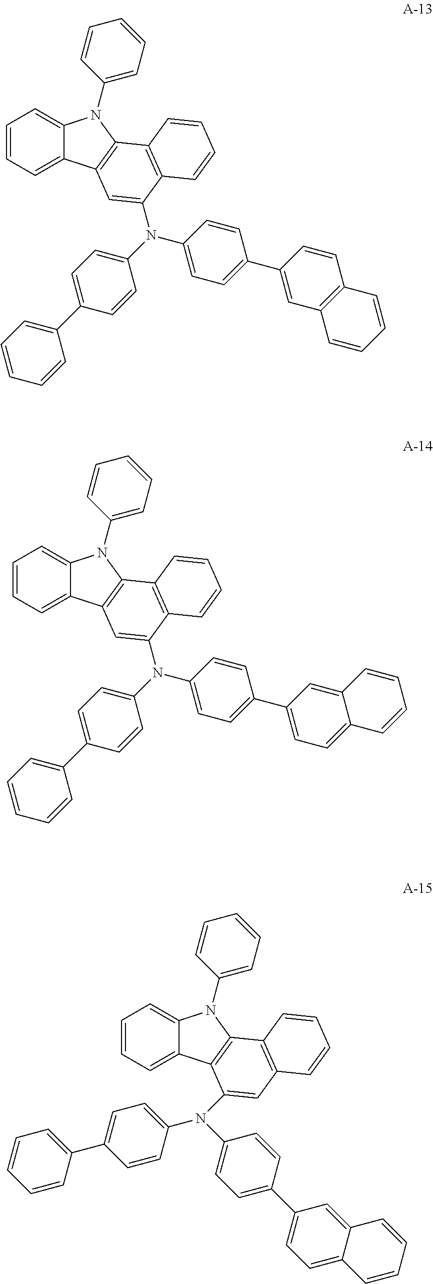 [SMILES: c1ccc(-c2ccc(N(c3ccc(-c4ccc5ccccc5c4)cc3)c3cc4c5ccccc5n(-c5ccccc5)c4c4ccccc34)cc2)cc1 ; c1ccc(-c2ccc(N(c3ccc(-c4ccc5ccccc5c4)cc3)c3cc4c5ccccc5n(-c5ccccc5)c4c4ccccc34)cc2)cc1 ; c1ccc(-c2ccc(N(c3ccc(-c4ccc5ccccc5c4)cc3)c3cc4ccccc4c4c3c3ccccc3n4-c3ccccc3)cc2)cc1]

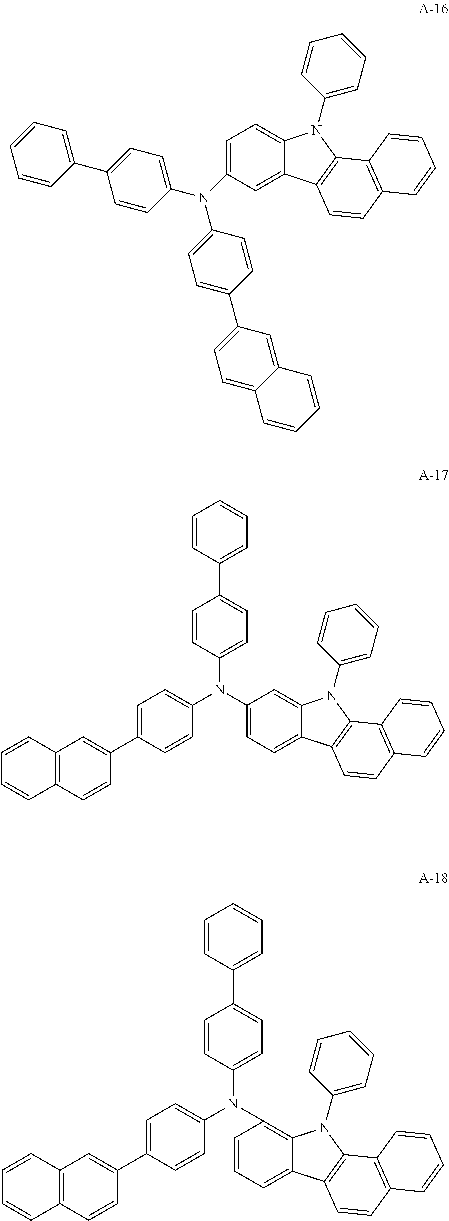 [SMILES: c1ccc(-c2ccc(N(c3ccc(-c4ccc5ccccc5c4)cc3)c3ccc4c(c3)c3ccc5ccccc5c3n4-c3ccccc3)cc2)cc1 ; c1ccc(-c2ccc(N(c3ccc(-c4ccc5ccccc5c4)cc3)c3ccc4c5ccc6ccccc6c5n(-c5ccccc5)c4c3)cc2)cc1 ; c1ccc(-c2ccc(N(c3ccc(-c4ccc5ccccc5c4)cc3)c3cccc4c5ccc6ccccc6c5n(-c5ccccc5)c34)cc2)cc1]

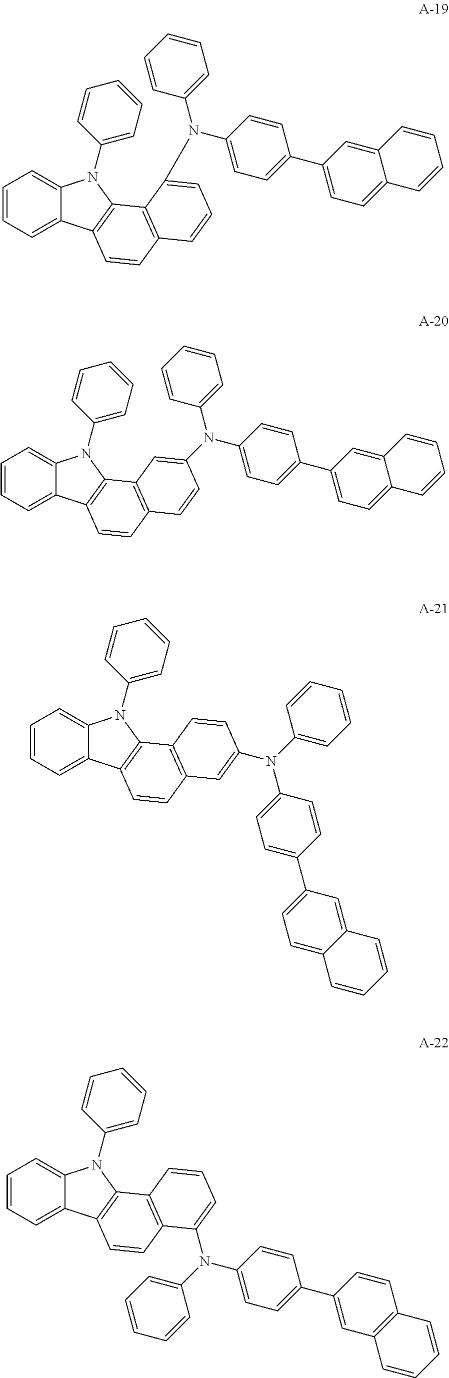 [SMILES: c1ccc(N(c2ccc(-c3ccc4ccccc4c3)cc2)c2cccc3ccc4c5ccccc5n(-c5ccccc5)c4c23)cc1 ; c1ccc(N(c2ccc(-c3ccc4ccccc4c3)cc2)c2ccc3ccc4c5ccccc5n(-c5ccccc5)c4c3c2)cc1 ; c1ccc(N(c2ccc(-c3ccc4ccccc4c3)cc2)c2ccc3c(ccc4c5ccccc5n(-c5ccccc5)c34)c2)cc1 ; c1ccc(N(c2ccc(-c3ccc4ccccc4c3)cc2)c2cccc3c2ccc2c4ccccc4n(-c4ccccc4)c32)cc1]

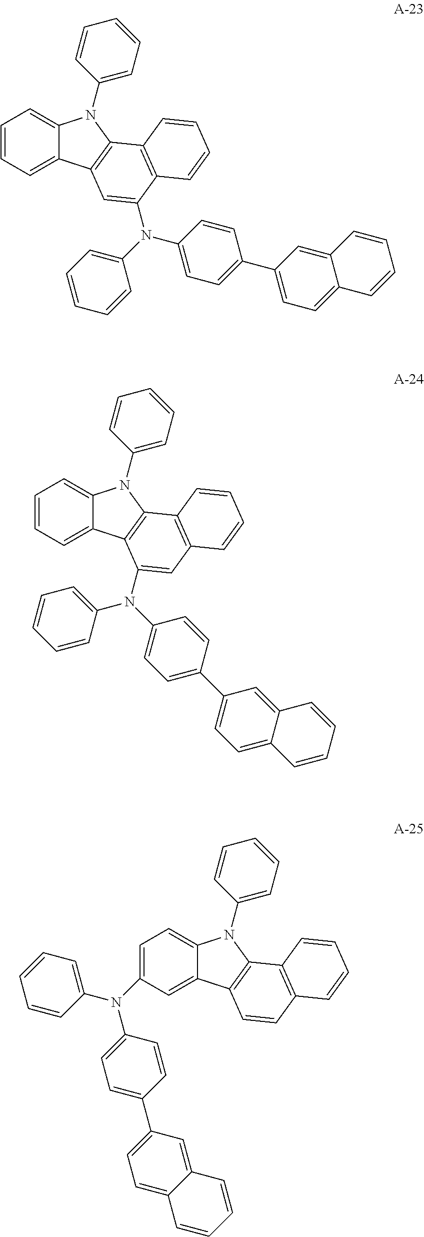 [SMILES: c1ccc(N(c2ccc(-c3ccc4ccccc4c3)cc2)c2cc3c4ccccc4n(-c4ccccc4)c3c3ccccc23)cc1 ; c1ccc(N(c2ccc(-c3ccc4ccccc4c3)cc2)c2cc3ccccc3c3c2c2ccccc2n3-c2ccccc2)cc1 ; c1ccc(N(c2ccc(-c3ccc4ccccc4c3)cc2)c2ccc3c(c2)c2ccc4ccccc4c2n3-c2ccccc2)cc1]

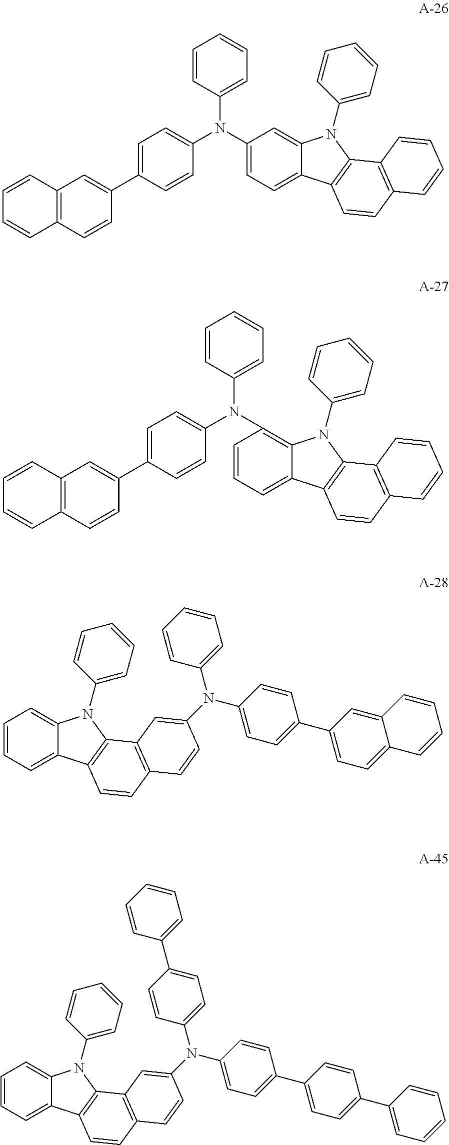 [SMILES: c1ccc(N(c2ccc(-c3ccc4ccccc4c3)cc2)c2ccc3c4ccc5ccccc5c4n(-c4ccccc4)c3c2)cc1 ; c1ccc(N(c2ccc(-c3ccc4ccccc4c3)cc2)c2cccc3c4ccc5ccccc5c4n(-c4ccccc4)c23)cc1 ; c1ccc(N(c2ccc(-c3ccc4ccccc4c3)cc2)c2ccc3ccc4c5ccccc5n(-c5ccccc5)c4c3c2)cc1 ; c1ccc(-c2ccc(-c3ccc(N(c4ccc(-c5ccccc5)cc4)c4ccc5ccc6c7ccccc7n(-c7ccccc7)c6c5c4)cc3)cc2)cc1]

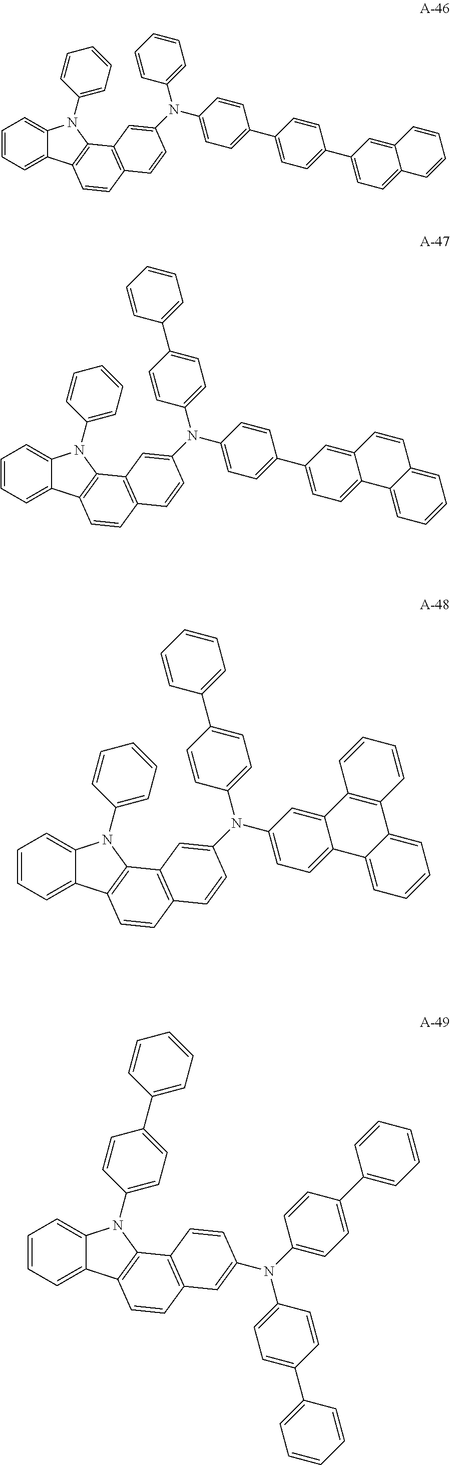 [SMILES: c1ccc(N(c2ccc(-c3ccc(-c4ccc5ccccc5c4)cc3)cc2)c2ccc3ccc4c5ccccc5n(-c5ccccc5)c4c3c2)cc1 ; c1ccc(-c2ccc(N(c3ccc(-c4ccc5c(ccc6ccccc65)c4)cc3)c3ccc4ccc5c6ccccc6n(-c6ccccc6)c5c4c3)cc2)cc1 ; c1ccc(-c2ccc(N(c3ccc4c5ccccc5c5ccccc5c4c3)c3ccc4ccc5c6ccccc6n(-c6ccccc6)c5c4c3)cc2)cc1 ; c1ccc(-c2ccc(N(c3ccc(-c4ccccc4)cc3)c3ccc4c(ccc5c6ccccc6n(-c6ccc(-c7ccccc7)cc6)c45)c3)cc2)cc1]

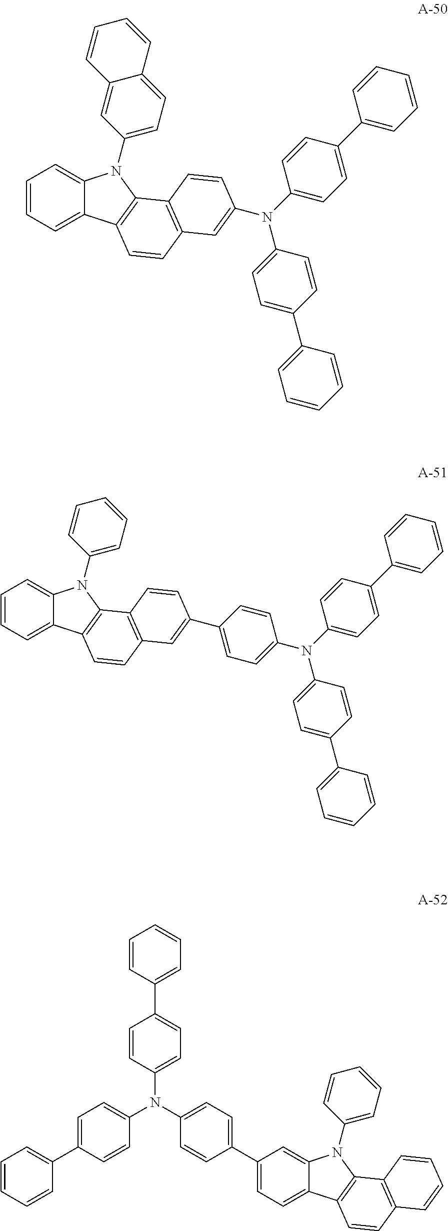 [SMILES: c1ccc(-c2ccc(N(c3ccc(-c4ccccc4)cc3)c3ccc4c(ccc5c6ccccc6n(-c6ccc7ccccc7c6)c45)c3)cc2)cc1 ; c1ccc(-c2ccc(N(c3ccc(-c4ccccc4)cc3)c3ccc(-c4ccc5c(ccc6c7ccccc7n(-c7ccccc7)c56)c4)cc3)cc2)cc1 ; c1ccc(-c2ccc(N(c3ccc(-c4ccccc4)cc3)c3ccc(-c4ccc5c6ccc7ccccc7c6n(-c6ccccc6)c5c4)cc3)cc2)cc1]

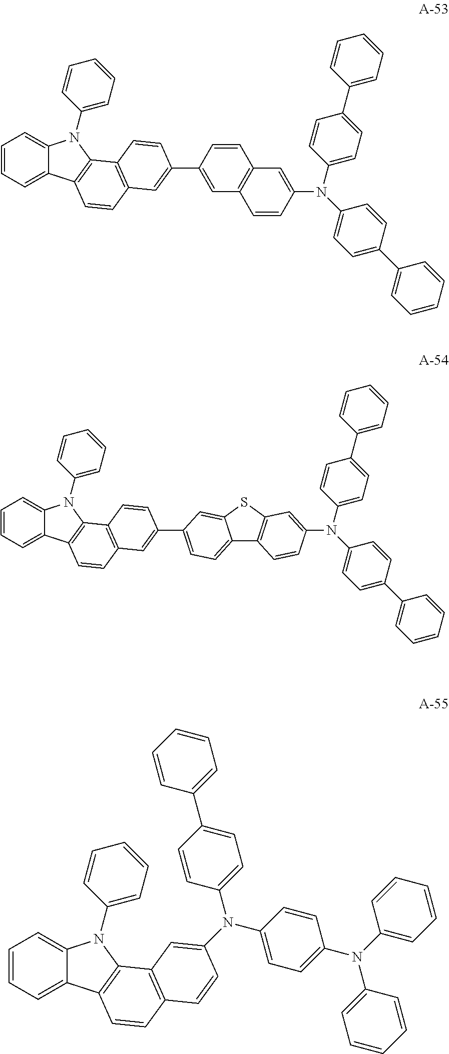 [SMILES: c1ccc(-c2ccc(N(c3ccc(-c4ccccc4)cc3)c3ccc4cc(-c5ccc6c(ccc7c8ccccc8n(-c8ccccc8)c67)c5)ccc4c3)cc2)cc1 ; c1ccc(-c2ccc(N(c3ccc(-c4ccccc4)cc3)c3ccc4c(c3)sc3cc(-c5ccc6c(ccc7c8ccccc8n(-c8ccccc8)c67)c5)ccc34)cc2)cc1 ; c1ccc(-c2ccc(N(c3ccc(N(c4ccccc4)c4ccccc4)cc3)c3ccc4ccc5c6ccccc6n(-c6ccccc6)c5c4c3)cc2)cc1]

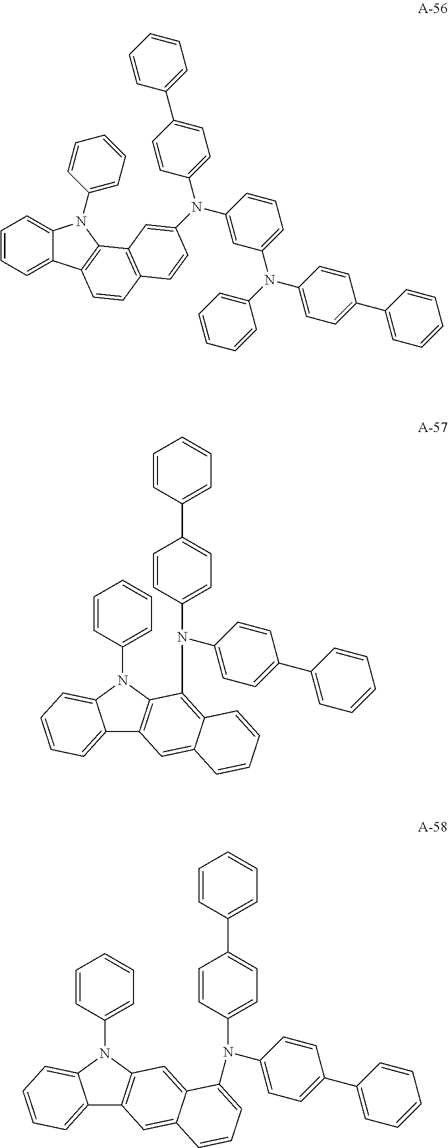 [SMILES: c1ccc(-c2ccc(N(c3ccccc3)c3cccc(N(c4ccc(-c5ccccc5)cc4)c4ccc5ccc6c7ccccc7n(-c7ccccc7)c6c5c4)c3)cc2)cc1 ; c1ccc(-c2ccc(N(c3ccc(-c4ccccc4)cc3)c3c4ccccc4cc4c5ccccc5n(-c5ccccc5)c34)cc2)cc1 ; c1ccc(-c2ccc(N(c3ccc(-c4ccccc4)cc3)c3cccc4cc5c6ccccc6n(-c6ccccc6)c5cc34)cc2)cc1]

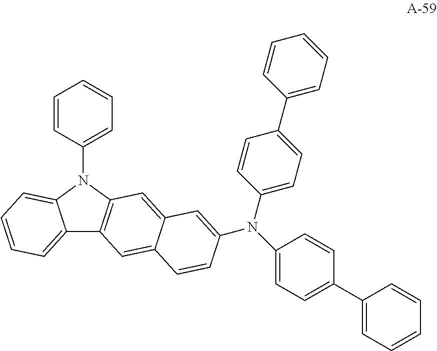 [SMILES: c1ccc(-c2ccc(N(c3ccc(-c4ccccc4)cc3)c3ccc4cc5c6ccccc6n(-c6ccccc6)c5cc4c3)cc2)cc1]

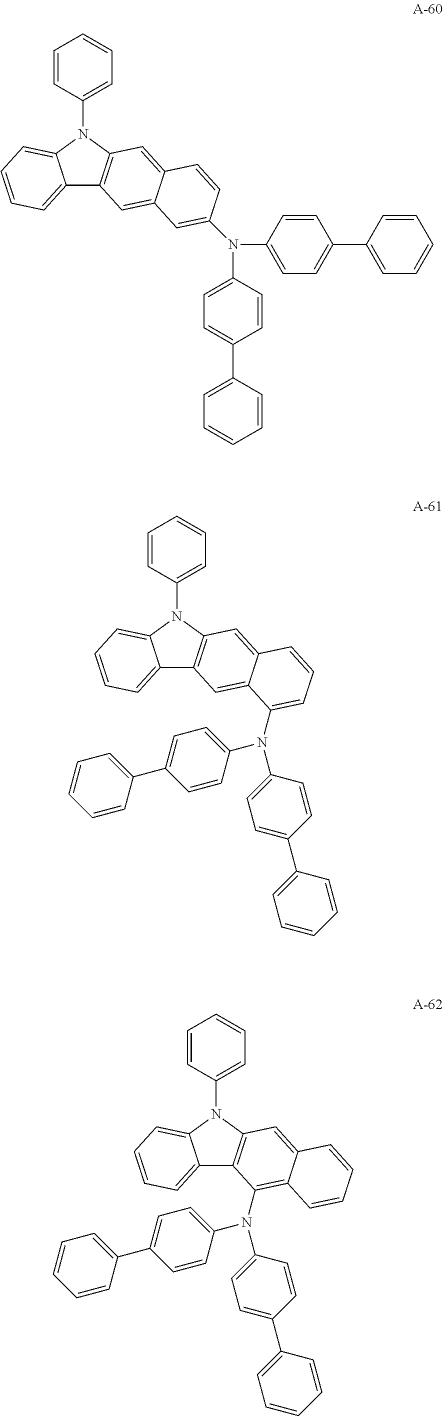 [SMILES: c1ccc(-c2ccc(N(c3ccc(-c4ccccc4)cc3)c3ccc4cc5c(cc4c3)c3ccccc3n5-c3ccccc3)cc2)cc1 ; c1ccc(-c2ccc(N(c3ccc(-c4ccccc4)cc3)c3cccc4cc5c(cc34)c3ccccc3n5-c3ccccc3)cc2)cc1 ; c1ccc(-c2ccc(N(c3ccc(-c4ccccc4)cc3)c3c4ccccc4cc4c3c3ccccc3n4-c3ccccc3)cc2)cc1]

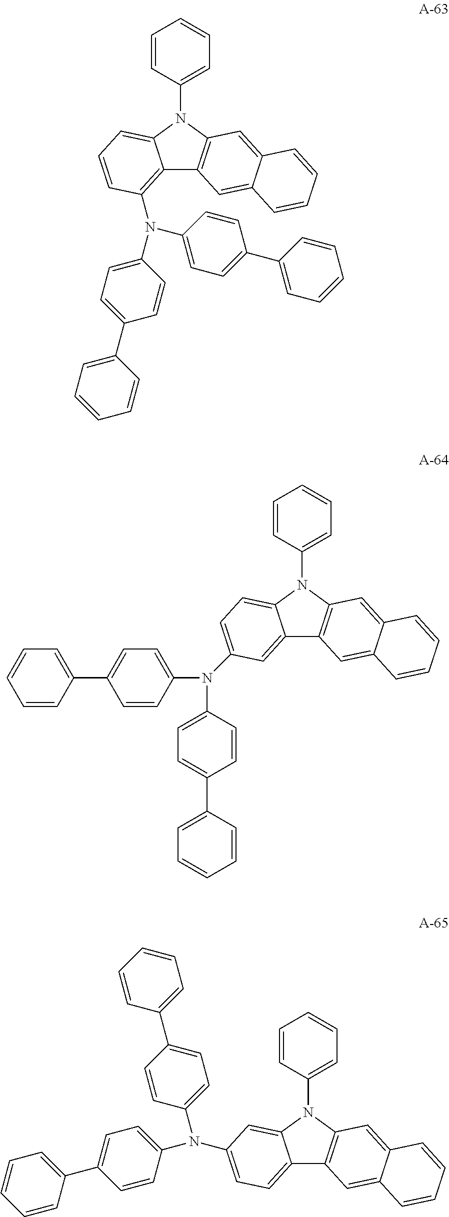 [SMILES: c1ccc(-c2ccc(N(c3ccc(-c4ccccc4)cc3)c3cccc4c3c3cc5ccccc5cc3n4-c3ccccc3)cc2)cc1 ; c1ccc(-c2ccc(N(c3ccc(-c4ccccc4)cc3)c3ccc4c(c3)c3cc5ccccc5cc3n4-c3ccccc3)cc2)cc1 ; c1ccc(-c2ccc(N(c3ccc(-c4ccccc4)cc3)c3ccc4c5cc6ccccc6cc5n(-c5ccccc5)c4c3)cc2)cc1]

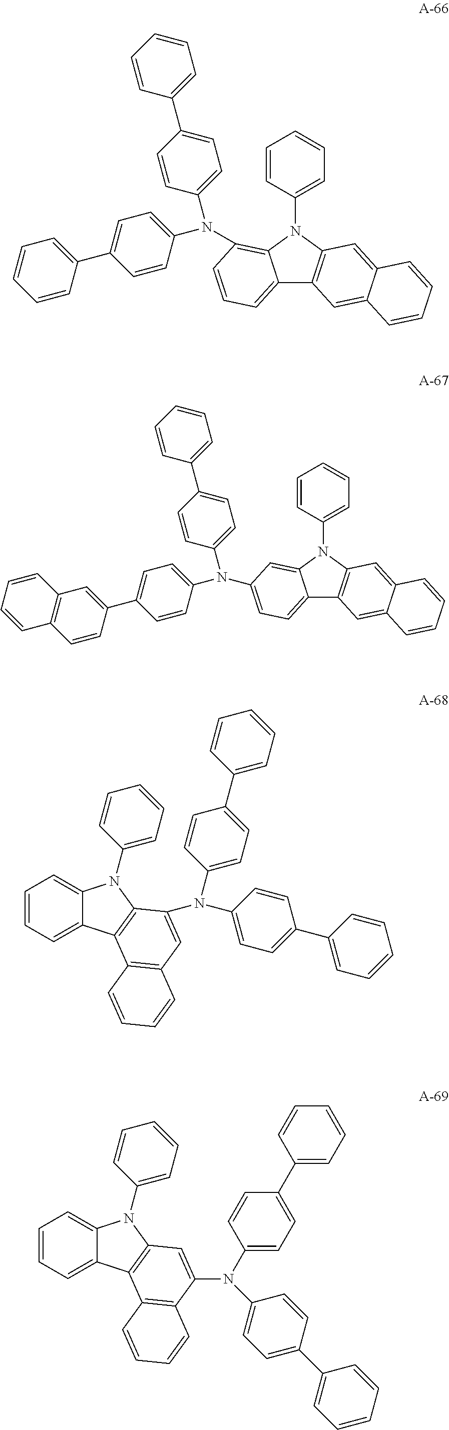 [SMILES: c1ccc(-c2ccc(N(c3ccc(-c4ccccc4)cc3)c3cccc4c5cc6ccccc6cc5n(-c5ccccc5)c34)cc2)cc1 ; c1ccc(-c2ccc(N(c3ccc(-c4ccc5ccccc5c4)cc3)c3ccc4c5cc6ccccc6cc5n(-c5ccccc5)c4c3)cc2)cc1 ; c1ccc(-c2ccc(N(c3ccc(-c4ccccc4)cc3)c3cc4ccccc4c4c5ccccc5n(-c5ccccc5)c34)cc2)cc1 ; c1ccc(-c2ccc(N(c3ccc(-c4ccccc4)cc3)c3cc4c(c5ccccc35)c3ccccc3n4-c3ccccc3)cc2)cc1]

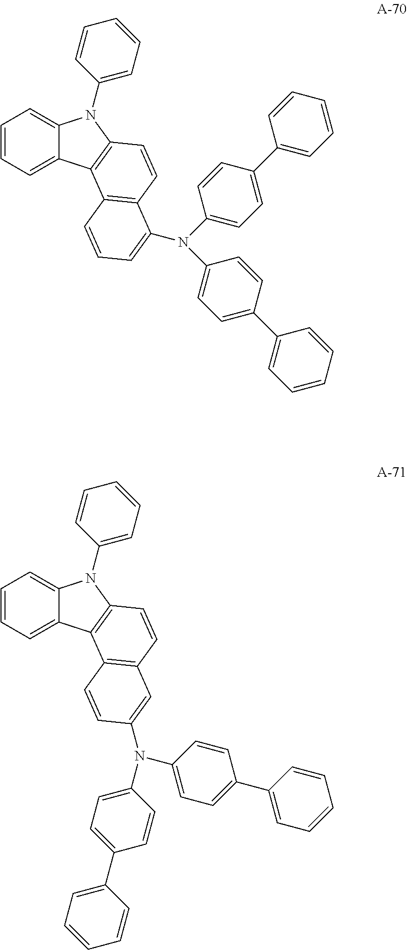 [SMILES: c1ccc(-c2ccc(N(c3ccc(-c4ccccc4)cc3)c3cccc4c3ccc3c4c4ccccc4n3-c3ccccc3)cc2)cc1 ; c1ccc(-c2ccc(N(c3ccc(-c4ccccc4)cc3)c3ccc4c(ccc5c4c4ccccc4n5-c4ccccc4)c3)cc2)cc1]

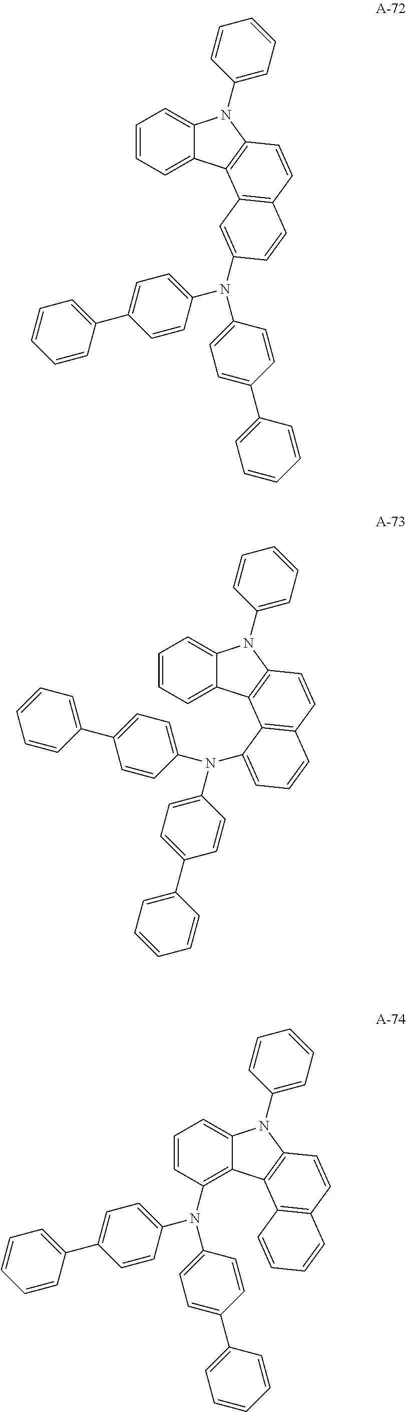 [SMILES: c1ccc(-c2ccc(N(c3ccc(-c4ccccc4)cc3)c3ccc4ccc5c(c4c3)c3ccccc3n5-c3ccccc3)cc2)cc1 ; c1ccc(-c2ccc(N(c3ccc(-c4ccccc4)cc3)c3cccc4ccc5c(c6ccccc6n5-c5ccccc5)c34)cc2)cc1 ; c1ccc(-c2ccc(N(c3ccc(-c4ccccc4)cc3)c3cccc4c3c3c5ccccc5ccc3n4-c3ccccc3)cc2)cc1]

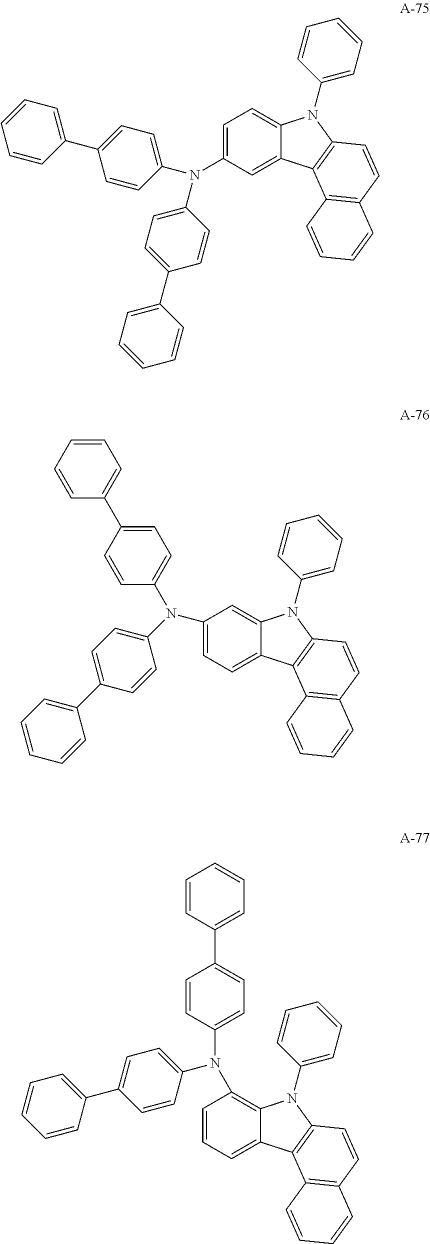 [SMILES: c1ccc(-c2ccc(N(c3ccc(-c4ccccc4)cc3)c3ccc4c(c3)c3c5ccccc5ccc3n4-c3ccccc3)cc2)cc1 ; c1ccc(-c2ccc(N(c3ccc(-c4ccccc4)cc3)c3ccc4c5c6ccccc6ccc5n(-c5ccccc5)c4c3)cc2)cc1 ; c1ccc(-c2ccc(N(c3ccc(-c4ccccc4)cc3)c3cccc4c5c6ccccc6ccc5n(-c5ccccc5)c34)cc2)cc1]

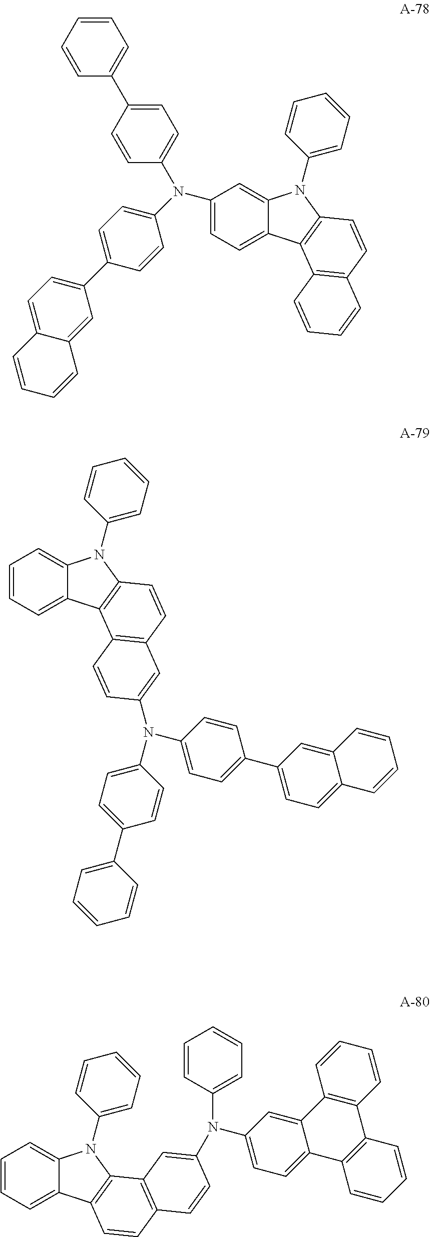 [SMILES: c1ccc(-c2ccc(N(c3ccc(-c4ccc5ccccc5c4)cc3)c3ccc4c5c6ccccc6ccc5n(-c5ccccc5)c4c3)cc2)cc1 ; c1ccc(-c2ccc(N(c3ccc(-c4ccc5ccccc5c4)cc3)c3ccc4c(ccc5c4c4ccccc4n5-c4ccccc4)c3)cc2)cc1 ; c1ccc(N(c2ccc3c4ccccc4c4ccccc4c3c2)c2ccc3ccc4c5ccccc5n(-c5ccccc5)c4c3c2)cc1]

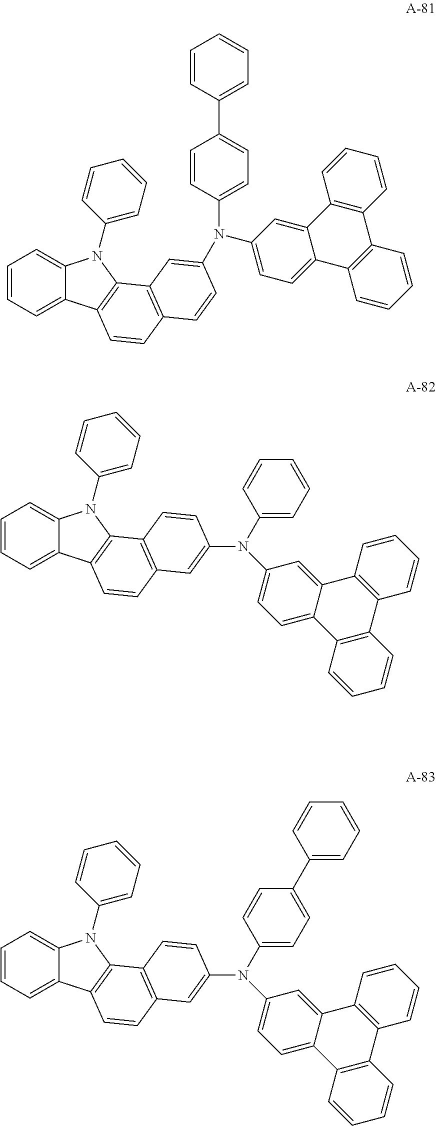 [SMILES: c1ccc(-c2ccc(N(c3ccc4c5ccccc5c5ccccc5c4c3)c3ccc4ccc5c6ccccc6n(-c6ccccc6)c5c4c3)cc2)cc1 ; c1ccc(N(c2ccc3c(ccc4c5ccccc5n(-c5ccccc5)c34)c2)c2ccc3c4ccccc4c4ccccc4c3c2)cc1 ; c1ccc(-c2ccc(N(c3ccc4c(ccc5c6ccccc6n(-c6ccccc6)c45)c3)c3ccc4c5ccccc5c5ccccc5c4c3)cc2)cc1]

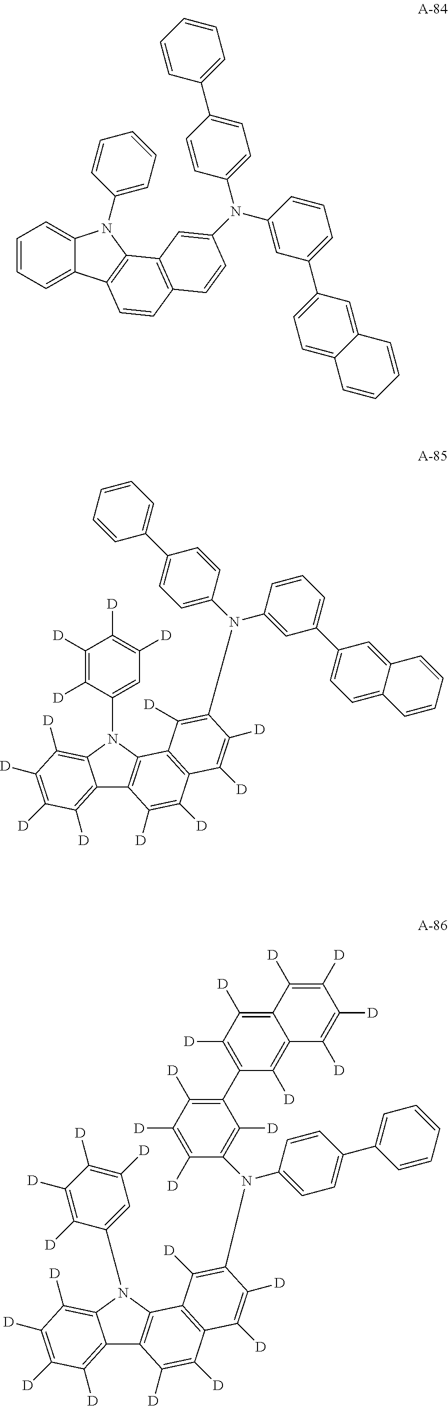 [SMILES: c1ccc(-c2ccc(N(c3cccc(-c4ccc5ccccc5c4)c3)c3ccc4ccc5c6ccccc6n(-c6ccccc6)c5c4c3)cc2)cc1 ; [2H]c1cc(-n2c3c([2H])c([2H])c([2H])c([2H])c3c3c([2H])c([2H])c4c([2H])c([2H])c(N(c5ccc(-c6ccccc6)cc5)c5cccc(-c6ccc7ccccc7c6)c5)c([2H])c4c32)c([2H])c([2H])c1[2H] ; [2H]c1cc(-n2c3c([2H])c([2H])c([2H])c([2H])c3c3c([2H])c([2H])c4c([2H])c([2H])c(N(c5ccc(-c6ccccc6)cc5)c5c([2H])c([2H])c([2H])c(-c6c([2H])c([2H])c7c([2H])c([2H])c([2H])c([2H])c7c6[2H])c5[2H])c([2H])c4c32)c([2H])c([2H])c1[2H]]

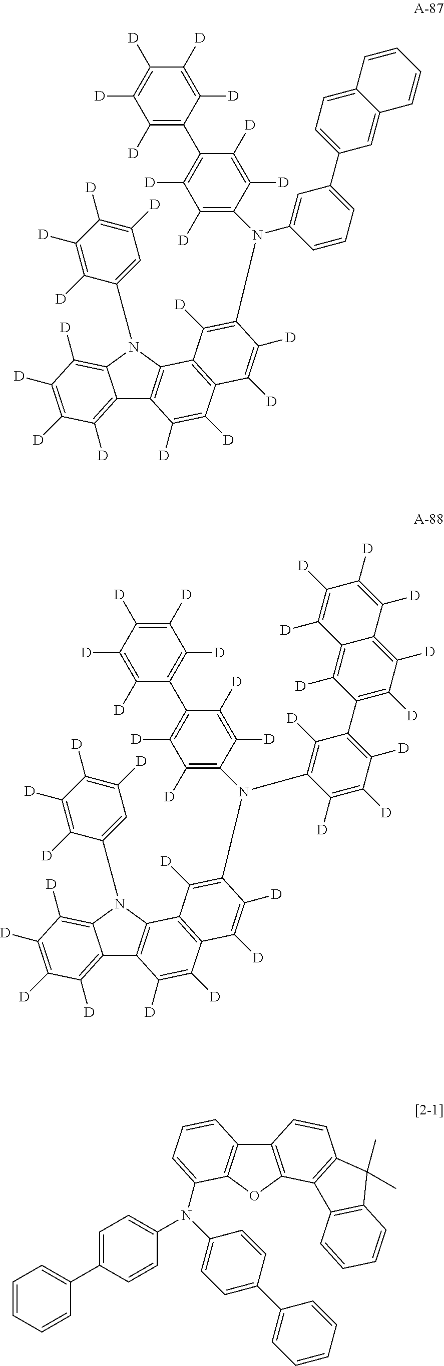

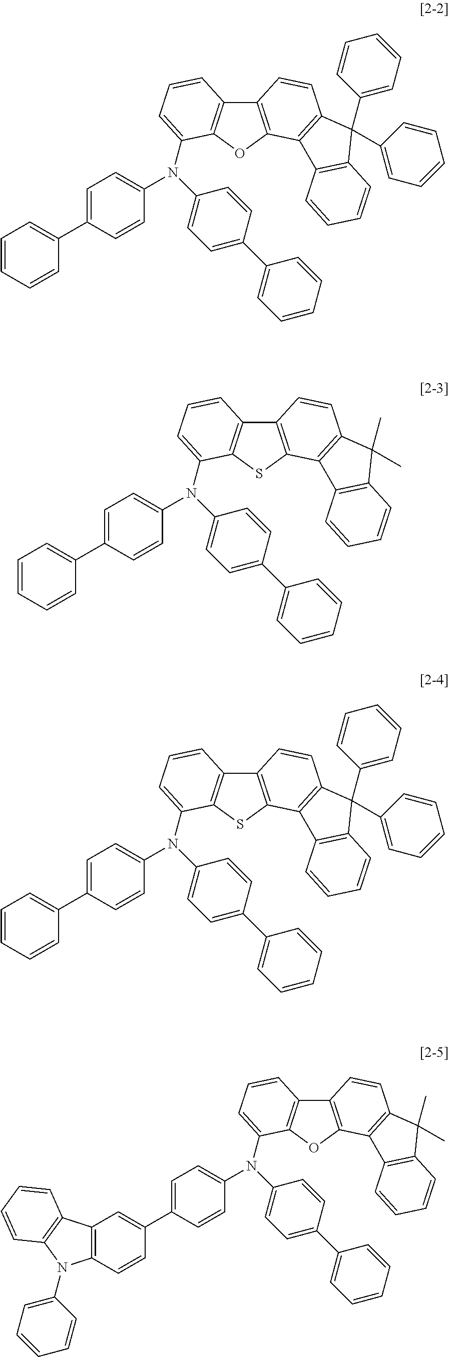 [SMILES: c1ccc(-c2ccc(N(c3ccc(-c4ccccc4)cc3)c3cccc4c3oc3c5c(ccc34)C(c3ccccc3)(c3ccccc3)c3ccccc3-5)cc2)cc1 ; CC1(C)c2ccccc2-c2c1ccc1c2sc2c(N(c3ccc(-c4ccccc4)cc3)c3ccc(-c4ccccc4)cc3)cccc21 ; c1ccc(-c2ccc(N(c3ccc(-c4ccccc4)cc3)c3cccc4c3sc3c5c(ccc34)C(c3ccccc3)(c3ccccc3)c3ccccc3-5)cc2)cc1 ; CC1(C)c2ccccc2-c2c1ccc1c2oc2c(N(c3ccc(-c4ccccc4)cc3)c3ccc(-c4ccc5c(c4)c4ccccc4n5-c4ccccc4)cc3)cccc21]

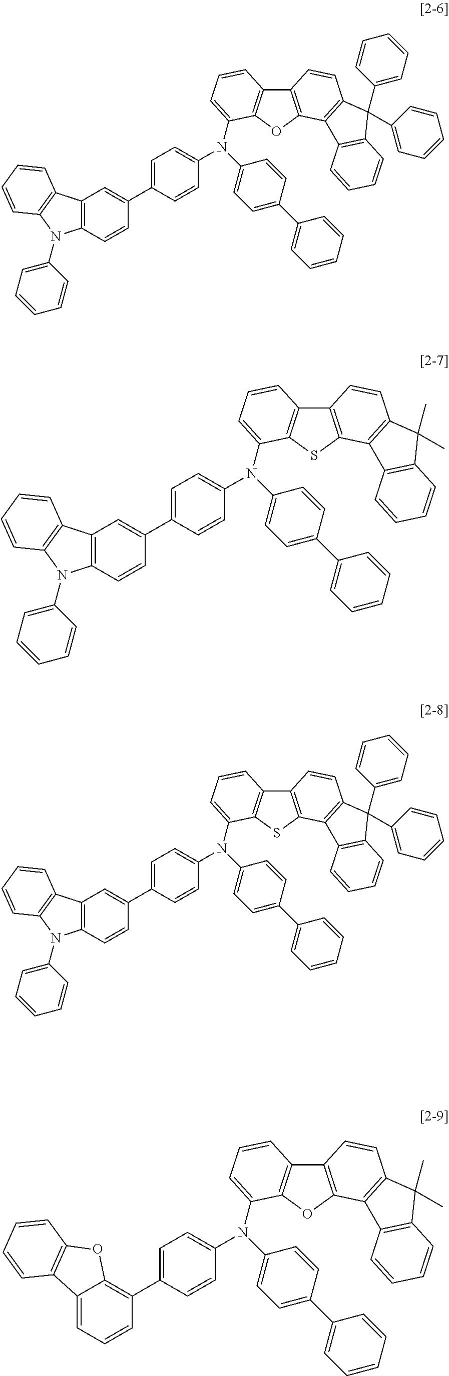 [SMILES: c1ccc(-c2ccc(N(c3ccc(-c4ccc5c(c4)c4ccccc4n5-c4ccccc4)cc3)c3cccc4c3oc3c5c(ccc34)C(c3ccccc3)(c3ccccc3)c3ccccc3-5)cc2)cc1 ; CC1(C)c2ccccc2-c2c1ccc1c2sc2c(N(c3ccc(-c4ccccc4)cc3)c3ccc(-c4ccc5c(c4)c4ccccc4n5-c4ccccc4)cc3)cccc21 ; c1ccc(-c2ccc(N(c3ccc(-c4ccc5c(c4)c4ccccc4n5-c4ccccc4)cc3)c3cccc4c3sc3c5c(ccc34)C(c3ccccc3)(c3ccccc3)c3ccccc3-5)cc2)cc1 ; CC1(C)c2ccccc2-c2c1ccc1c2oc2c(N(c3ccc(-c4ccccc4)cc3)c3ccc(-c4cccc5c4oc4ccccc45)cc3)cccc21]

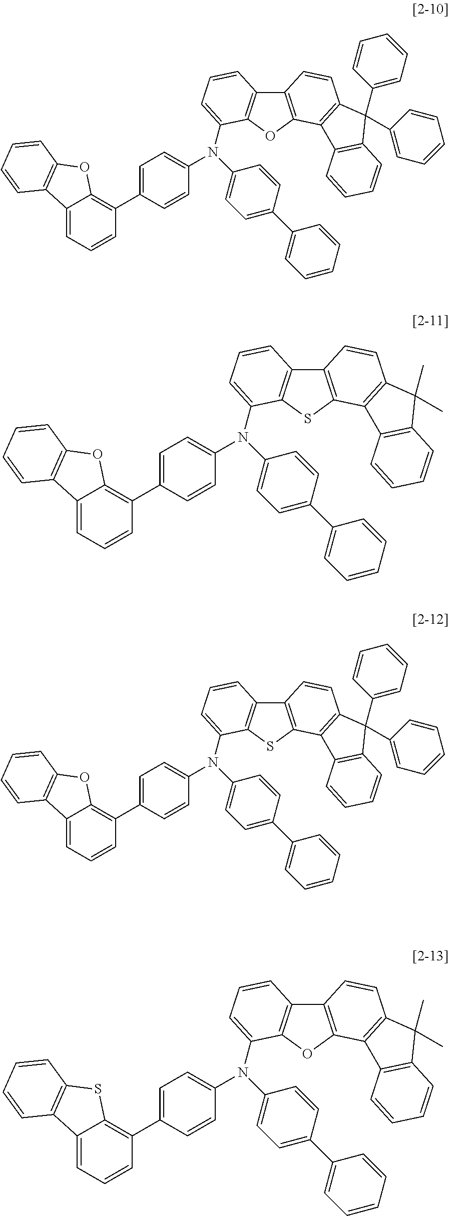 [SMILES: c1ccc(-c2ccc(N(c3ccc(-c4cccc5c4oc4ccccc45)cc3)c3cccc4c3oc3c5c(ccc34)C(c3ccccc3)(c3ccccc3)c3ccccc3-5)cc2)cc1 ; CC1(C)c2ccccc2-c2c1ccc1c2sc2c(N(c3ccc(-c4ccccc4)cc3)c3ccc(-c4cccc5c4oc4ccccc45)cc3)cccc21 ; c1ccc(-c2ccc(N(c3ccc(-c4cccc5c4oc4ccccc45)cc3)c3cccc4c3sc3c5c(ccc34)C(c3ccccc3)(c3ccccc3)c3ccccc3-5)cc2)cc1 ; CC1(C)c2ccccc2-c2c1ccc1c2oc2c(N(c3ccc(-c4ccccc4)cc3)c3ccc(-c4cccc5c4sc4ccccc45)cc3)cccc21]

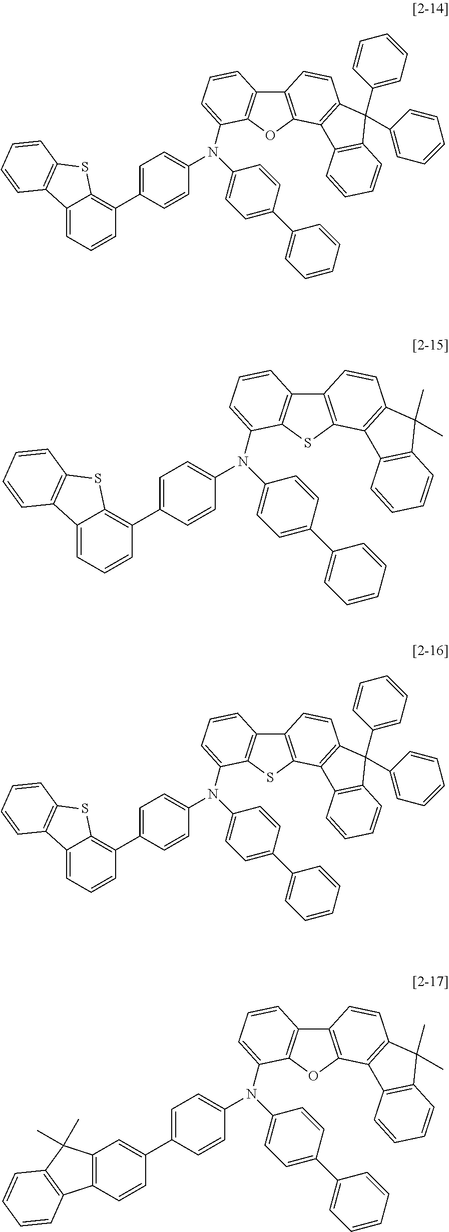 [SMILES: c1ccc(-c2ccc(N(c3ccc(-c4cccc5c4sc4ccccc45)cc3)c3cccc4c3oc3c5c(ccc34)C(c3ccccc3)(c3ccccc3)c3ccccc3-5)cc2)cc1 ; CC1(C)c2ccccc2-c2c1ccc1c2sc2c(N(c3ccc(-c4ccccc4)cc3)c3ccc(-c4cccc5c4sc4ccccc45)cc3)cccc21 ; c1ccc(-c2ccc(N(c3ccc(-c4cccc5c4sc4ccccc45)cc3)c3cccc4c3sc3c5c(ccc34)C(c3ccccc3)(c3ccccc3)c3ccccc3-5)cc2)cc1 ; CC1(C)c2ccccc2-c2ccc(-c3ccc(N(c4ccc(-c5ccccc5)cc4)c4cccc5c4oc4c6c(ccc45)C(C)(C)c4ccccc4-6)cc3)cc21]

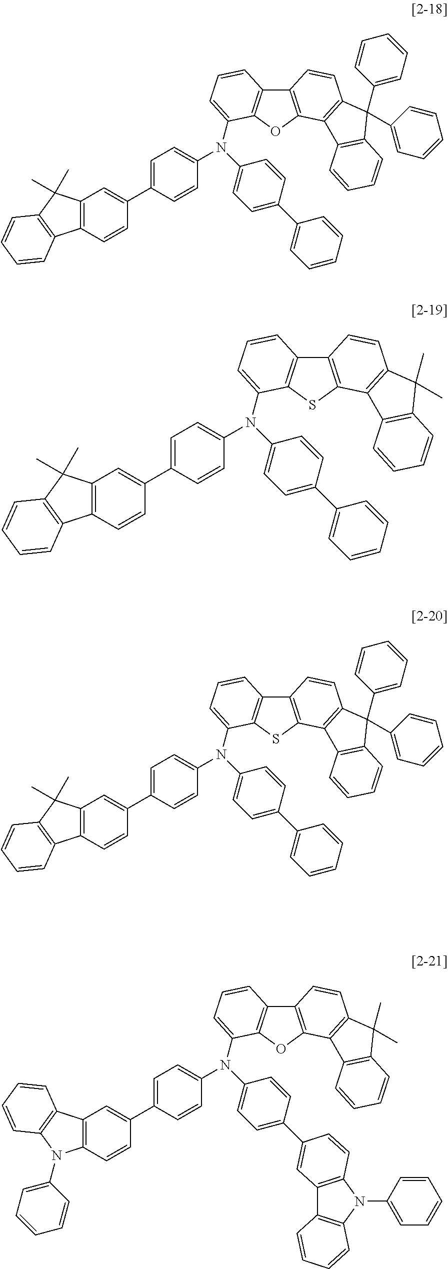 [SMILES: CC1(C)c2ccccc2-c2ccc(-c3ccc(N(c4ccc(-c5ccccc5)cc4)c4cccc5c4oc4c6c(ccc45)C(c4ccccc4)(c4ccccc4)c4ccccc4-6)cc3)cc21 ; CC1(C)c2ccccc2-c2ccc(-c3ccc(N(c4ccc(-c5ccccc5)cc4)c4cccc5c4sc4c6c(ccc45)C(C)(C)c4ccccc4-6)cc3)cc21 ; CC1(C)c2ccccc2-c2ccc(-c3ccc(N(c4ccc(-c5ccccc5)cc4)c4cccc5c4sc4c6c(ccc45)C(c4ccccc4)(c4ccccc4)c4ccccc4-6)cc3)cc21 ; CC1(C)c2ccccc2-c2c1ccc1c2oc2c(N(c3ccc(-c4ccc5c(c4)c4ccccc4n5-c4ccccc4)cc3)c3ccc(-c4ccc5c(c4)c4ccccc4n5-c4ccccc4)cc3)cccc21]

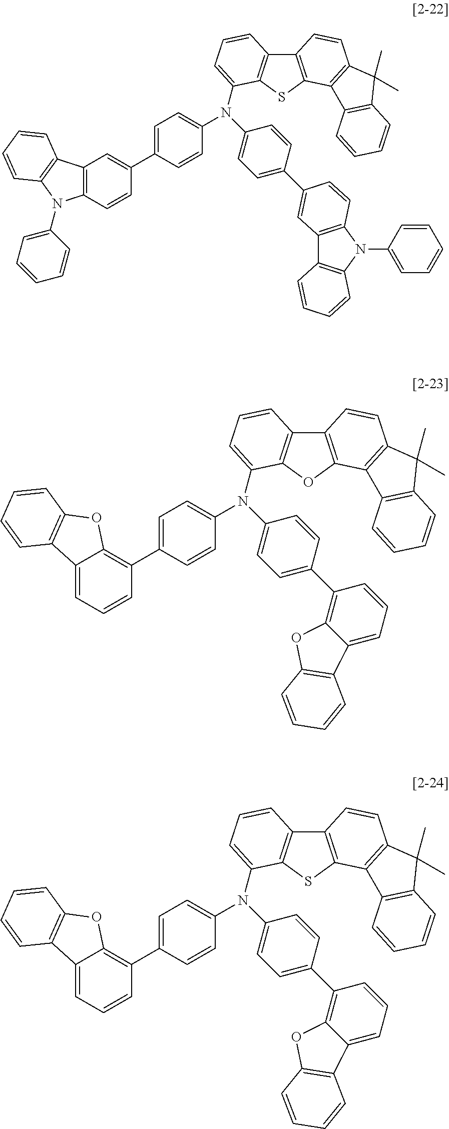 [SMILES: CC1(C)c2ccccc2-c2c1ccc1c2sc2c(N(c3ccc(-c4ccc5c(c4)c4ccccc4n5-c4ccccc4)cc3)c3ccc(-c4ccc5c(c4)c4ccccc4n5-c4ccccc4)cc3)cccc21 ; CC1(C)c2ccccc2-c2c1ccc1c2oc2c(N(c3ccc(-c4cccc5c4oc4ccccc45)cc3)c3ccc(-c4cccc5c4oc4ccccc45)cc3)cccc21 ; CC1(C)c2ccccc2-c2c1ccc1c2sc2c(N(c3ccc(-c4cccc5c4oc4ccccc45)cc3)c3ccc(-c4cccc5c4oc4ccccc45)cc3)cccc21]

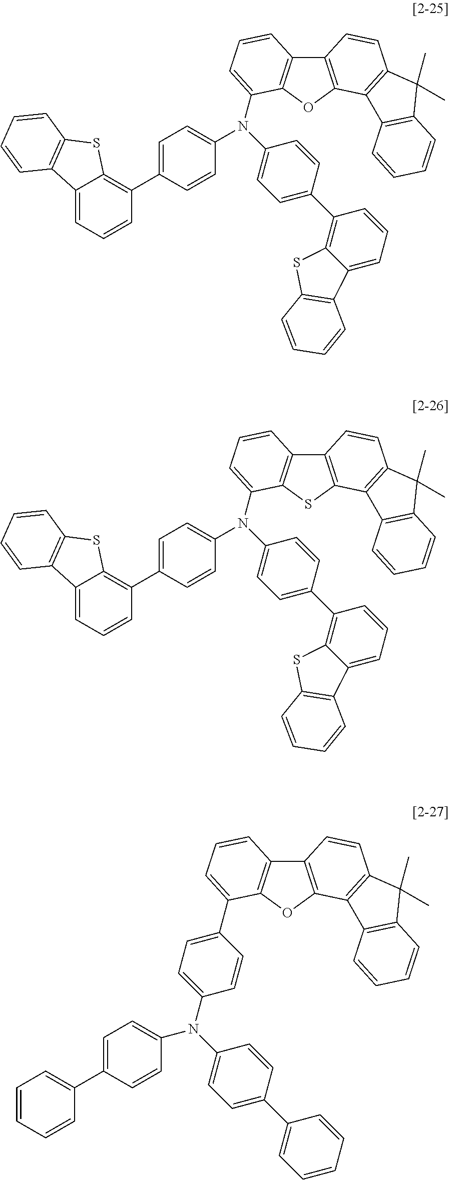 [SMILES: CC1(C)c2ccccc2-c2c1ccc1c2oc2c(N(c3ccc(-c4cccc5c4sc4ccccc45)cc3)c3ccc(-c4cccc5c4sc4ccccc45)cc3)cccc21 ; CC1(C)c2ccccc2-c2c1ccc1c2sc2c(N(c3ccc(-c4cccc5c4sc4ccccc45)cc3)c3ccc(-c4cccc5c4sc4ccccc45)cc3)cccc21 ; CC1(C)c2ccccc2-c2c1ccc1c2oc2c(-c3ccc(N(c4ccc(-c5ccccc5)cc4)c4ccc(-c5ccccc5)cc4)cc3)cccc21]

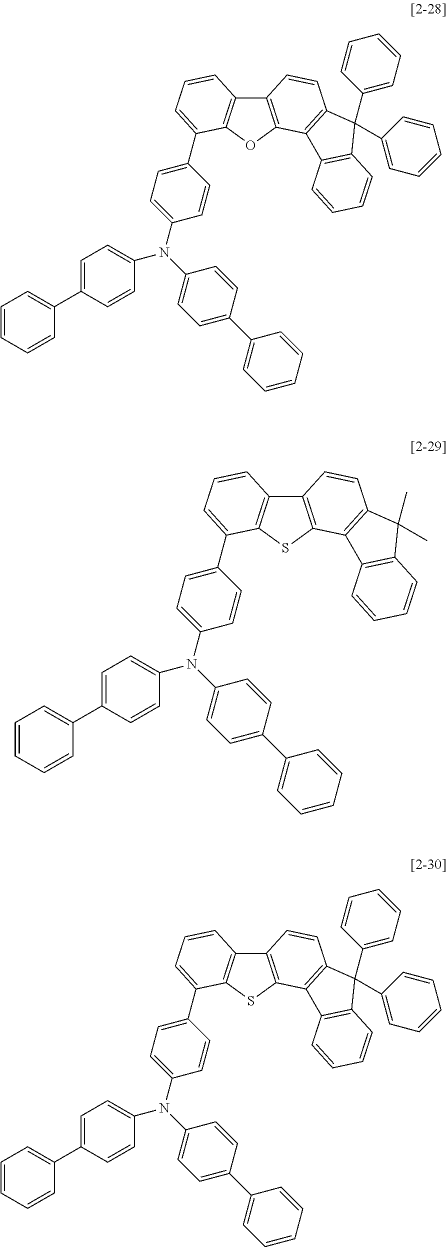 [SMILES: c1ccc(-c2ccc(N(c3ccc(-c4ccccc4)cc3)c3ccc(-c4cccc5c4oc4c6c(ccc45)C(c4ccccc4)(c4ccccc4)c4ccccc4-6)cc3)cc2)cc1 ; CC1(C)c2ccccc2-c2c1ccc1c2sc2c(-c3ccc(N(c4ccc(-c5ccccc5)cc4)c4ccc(-c5ccccc5)cc4)cc3)cccc21 ; c1ccc(-c2ccc(N(c3ccc(-c4ccccc4)cc3)c3ccc(-c4cccc5c4sc4c6c(ccc45)C(c4ccccc4)(c4ccccc4)c4ccccc4-6)cc3)cc2)cc1]

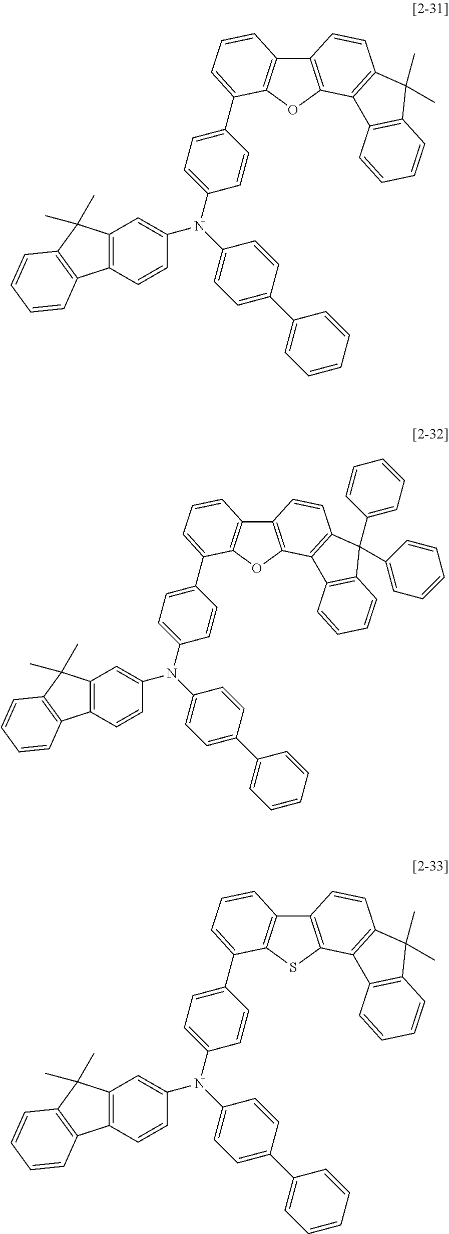 [SMILES: CC1(C)c2ccccc2-c2ccc(N(c3ccc(-c4ccccc4)cc3)c3ccc(-c4cccc5c4oc4c6c(ccc45)C(C)(C)c4ccccc4-6)cc3)cc21 ; CC1(C)c2ccccc2-c2ccc(N(c3ccc(-c4ccccc4)cc3)c3ccc(-c4cccc5c4oc4c6c(ccc45)C(c4ccccc4)(c4ccccc4)c4ccccc4-6)cc3)cc21 ; CC1(C)c2ccccc2-c2ccc(N(c3ccc(-c4ccccc4)cc3)c3ccc(-c4cccc5c4sc4c6c(ccc45)C(C)(C)c4ccccc4-6)cc3)cc21]

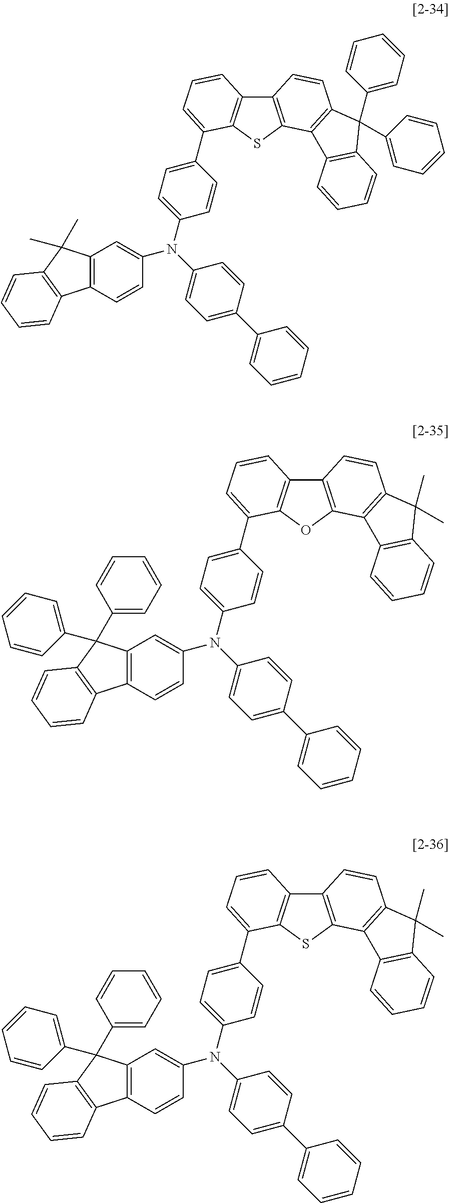 [SMILES: CC1(C)c2ccccc2-c2ccc(N(c3ccc(-c4ccccc4)cc3)c3ccc(-c4cccc5c4sc4c6c(ccc45)C(c4ccccc4)(c4ccccc4)c4ccccc4-6)cc3)cc21 ; CC1(C)c2ccccc2-c2c1ccc1c2oc2c(-c3ccc(N(c4ccc(-c5ccccc5)cc4)c4ccc5c(c4)C(c4ccccc4)(c4ccccc4)c4ccccc4-5)cc3)cccc21 ; CC1(C)c2ccccc2-c2c1ccc1c2sc2c(-c3ccc(N(c4ccc(-c5ccccc5)cc4)c4ccc5c(c4)C(c4ccccc4)(c4ccccc4)c4ccccc4-5)cc3)cccc21]

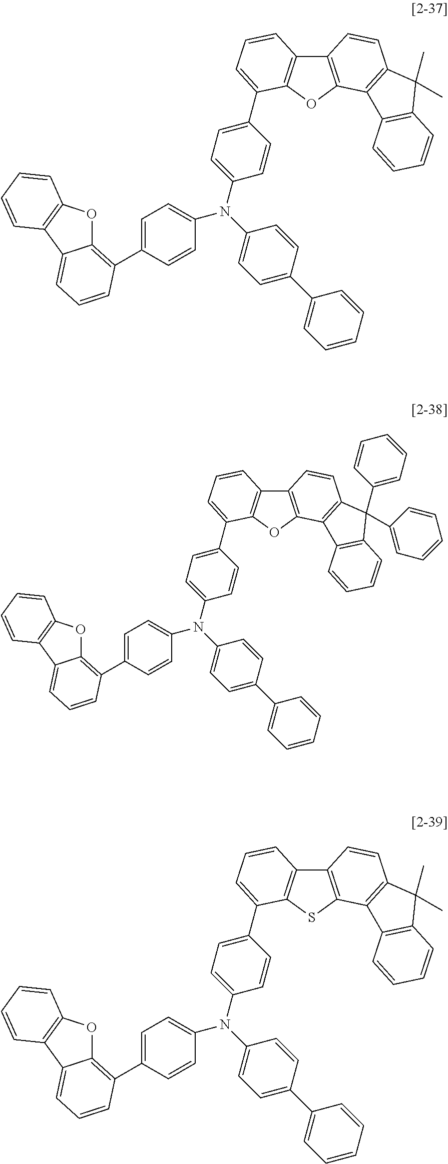 [SMILES: CC1(C)c2ccccc2-c2c1ccc1c2oc2c(-c3ccc(N(c4ccc(-c5ccccc5)cc4)c4ccc(-c5cccc6c5oc5ccccc56)cc4)cc3)cccc21 ; c1ccc(-c2ccc(N(c3ccc(-c4cccc5c4oc4ccccc45)cc3)c3ccc(-c4cccc5c4oc4c6c(ccc45)C(c4ccccc4)(c4ccccc4)c4ccccc4-6)cc3)cc2)cc1 ; CC1(C)c2ccccc2-c2c1ccc1c2sc2c(-c3ccc(N(c4ccc(-c5ccccc5)cc4)c4ccc(-c5cccc6c5oc5ccccc56)cc4)cc3)cccc21]

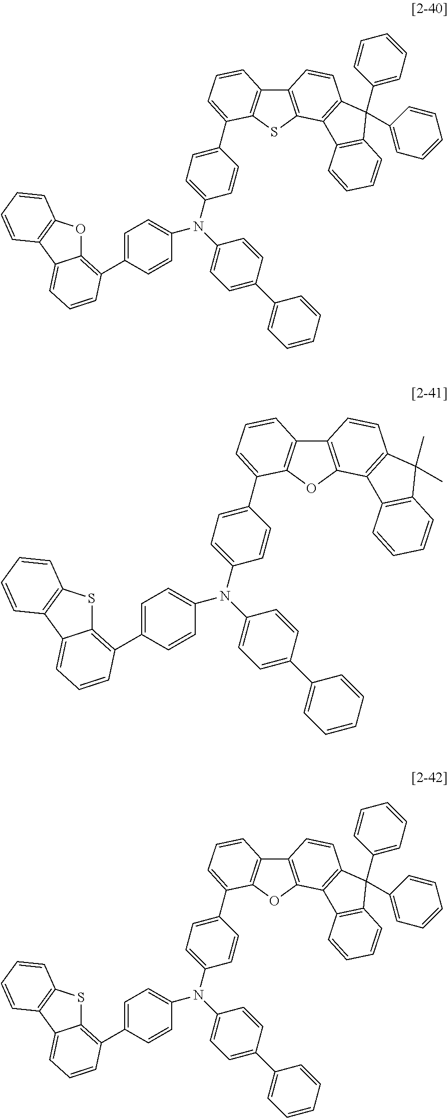 [SMILES: c1ccc(-c2ccc(N(c3ccc(-c4cccc5c4oc4ccccc45)cc3)c3ccc(-c4cccc5c4sc4c6c(ccc45)C(c4ccccc4)(c4ccccc4)c4ccccc4-6)cc3)cc2)cc1 ; CC1(C)c2ccccc2-c2c1ccc1c2oc2c(-c3ccc(N(c4ccc(-c5ccccc5)cc4)c4ccc(-c5cccc6c5sc5ccccc56)cc4)cc3)cccc21 ; c1ccc(-c2ccc(N(c3ccc(-c4cccc5c4oc4c6c(ccc45)C(c4ccccc4)(c4ccccc4)c4ccccc4-6)cc3)c3ccc(-c4cccc5c4sc4ccccc45)cc3)cc2)cc1]

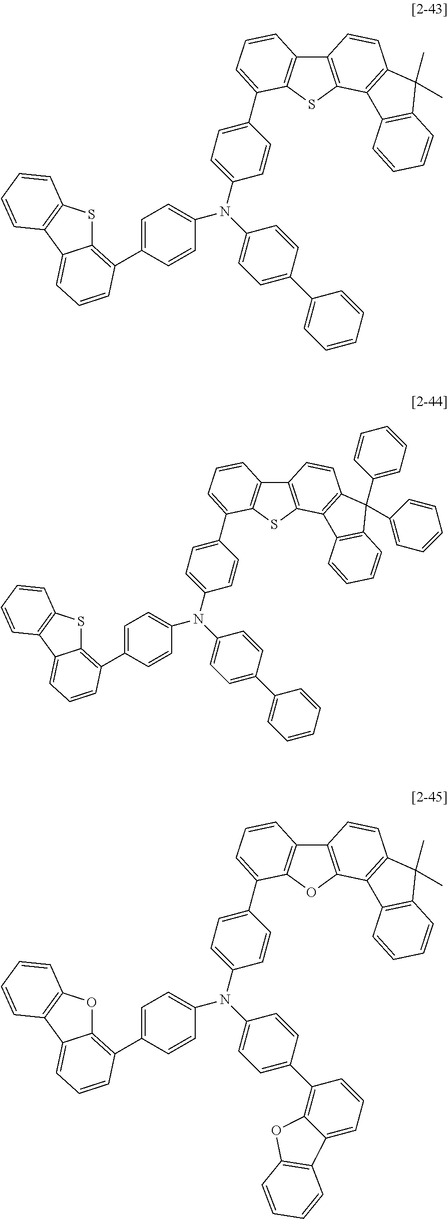 [SMILES: CC1(C)c2ccccc2-c2c1ccc1c2sc2c(-c3ccc(N(c4ccc(-c5ccccc5)cc4)c4ccc(-c5cccc6c5sc5ccccc56)cc4)cc3)cccc21 ; c1ccc(-c2ccc(N(c3ccc(-c4cccc5c4sc4ccccc45)cc3)c3ccc(-c4cccc5c4sc4c6c(ccc45)C(c4ccccc4)(c4ccccc4)c4ccccc4-6)cc3)cc2)cc1 ; CC1(C)c2ccccc2-c2c1ccc1c2oc2c(-c3ccc(N(c4ccc(-c5cccc6c5oc5ccccc56)cc4)c4ccc(-c5cccc6c5oc5ccccc56)cc4)cc3)cccc21]

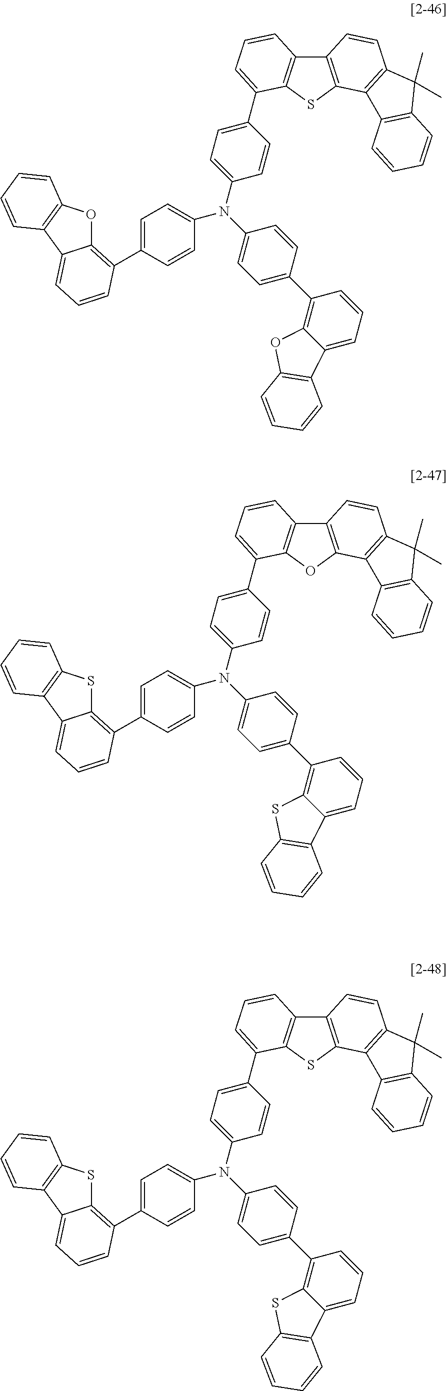 [SMILES: CC1(C)c2ccccc2-c2c1ccc1c2sc2c(-c3ccc(N(c4ccc(-c5cccc6c5oc5ccccc56)cc4)c4ccc(-c5cccc6c5oc5ccccc56)cc4)cc3)cccc21 ; CC1(C)c2ccccc2-c2c1ccc1c2oc2c(-c3ccc(N(c4ccc(-c5cccc6c5sc5ccccc56)cc4)c4ccc(-c5cccc6c5sc5ccccc56)cc4)cc3)cccc21 ; CC1(C)c2ccccc2-c2c1ccc1c2sc2c(-c3ccc(N(c4ccc(-c5cccc6c5sc5ccccc56)cc4)c4ccc(-c5cccc6c5sc5ccccc56)cc4)cc3)cccc21]

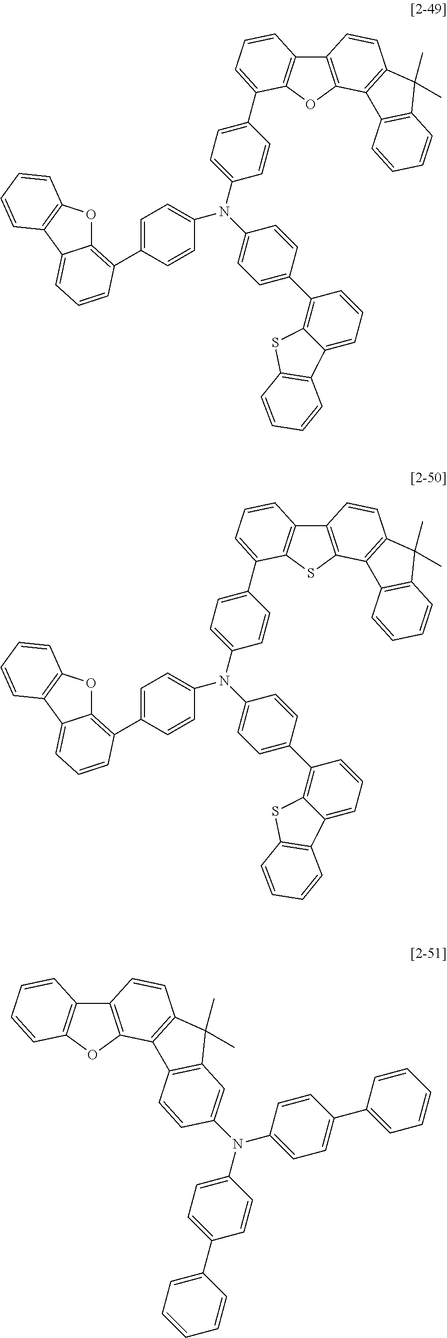 [SMILES: CC1(C)c2ccccc2-c2c1ccc1c2oc2c(-c3ccc(N(c4ccc(-c5cccc6c5oc5ccccc56)cc4)c4ccc(-c5cccc6c5sc5ccccc56)cc4)cc3)cccc21 ; CC1(C)c2ccccc2-c2c1ccc1c2sc2c(-c3ccc(N(c4ccc(-c5cccc6c5oc5ccccc56)cc4)c4ccc(-c5cccc6c5sc5ccccc56)cc4)cc3)cccc21 ; CC1(C)c2cc(N(c3ccc(-c4ccccc4)cc3)c3ccc(-c4ccccc4)cc3)ccc2-c2c1ccc1c2oc2ccccc21]

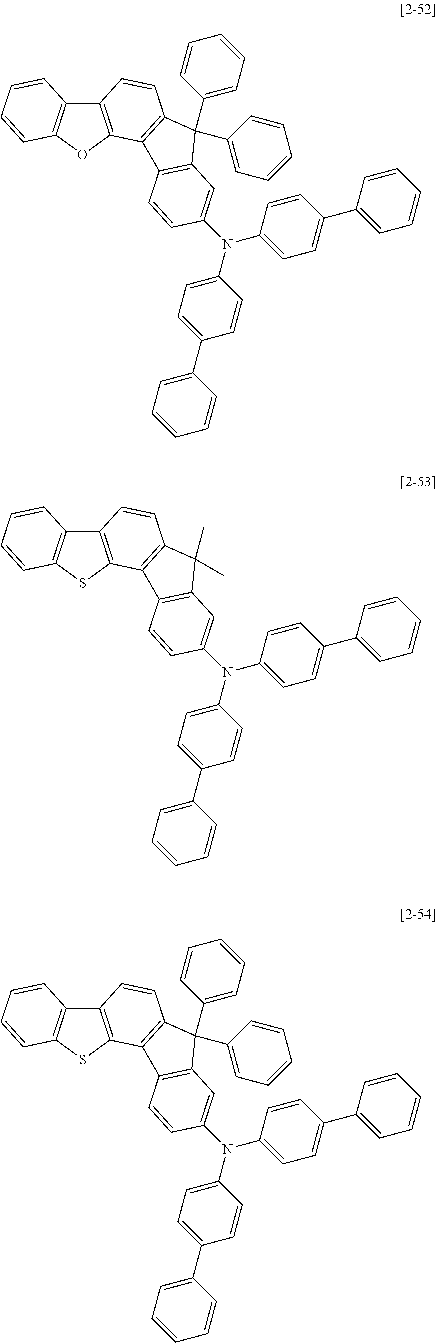 [SMILES: c1ccc(-c2ccc(N(c3ccc(-c4ccccc4)cc3)c3ccc4c(c3)C(c3ccccc3)(c3ccccc3)c3ccc5c(oc6ccccc65)c3-4)cc2)cc1 ; CC1(C)c2cc(N(c3ccc(-c4ccccc4)cc3)c3ccc(-c4ccccc4)cc3)ccc2-c2c1ccc1c2sc2ccccc21 ; c1ccc(-c2ccc(N(c3ccc(-c4ccccc4)cc3)c3ccc4c(c3)C(c3ccccc3)(c3ccccc3)c3ccc5c(sc6ccccc65)c3-4)cc2)cc1]

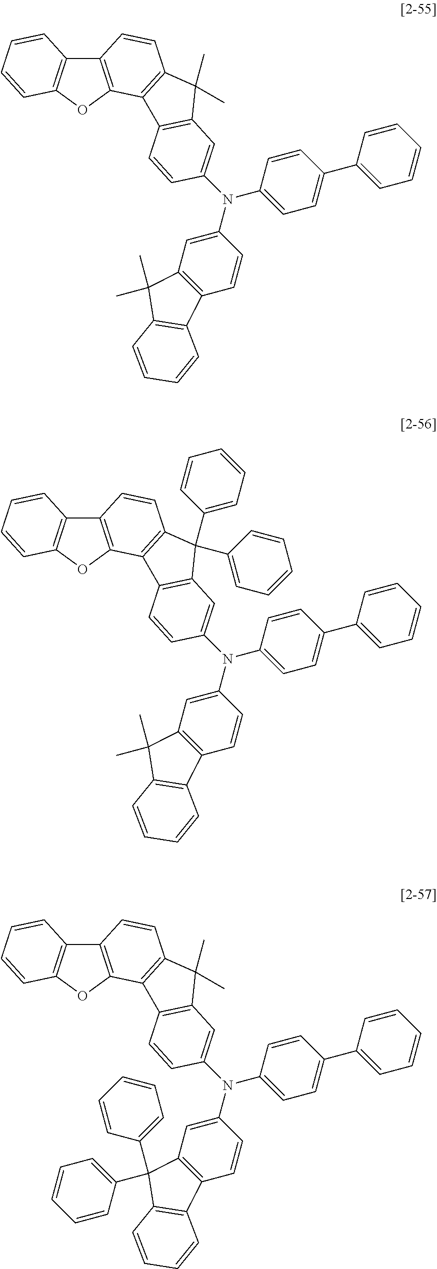 [SMILES: CC1(C)c2ccccc2-c2ccc(N(c3ccc(-c4ccccc4)cc3)c3ccc4c(c3)C(C)(C)c3ccc5c(oc6ccccc65)c3-4)cc21 ; CC1(C)c2ccccc2-c2ccc(N(c3ccc(-c4ccccc4)cc3)c3ccc4c(c3)C(c3ccccc3)(c3ccccc3)c3ccc5c(oc6ccccc65)c3-4)cc21 ; CC1(C)c2cc(N(c3ccc(-c4ccccc4)cc3)c3ccc4c(c3)C(c3ccccc3)(c3ccccc3)c3ccccc3-4)ccc2-c2c1ccc1c2oc2ccccc21]

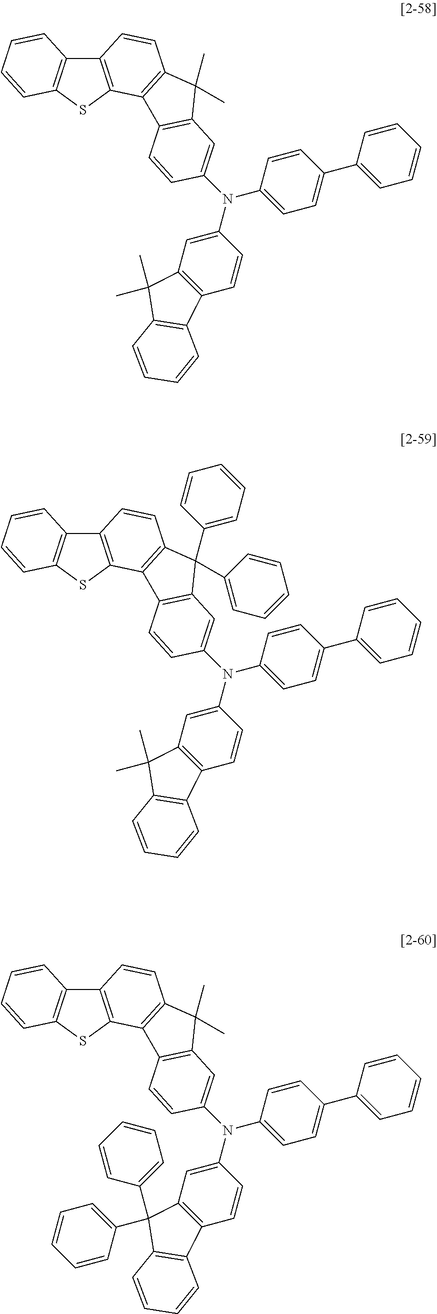 [SMILES: CC1(C)c2ccccc2-c2ccc(N(c3ccc(-c4ccccc4)cc3)c3ccc4c(c3)C(C)(C)c3ccc5c(sc6ccccc65)c3-4)cc21 ; CC1(C)c2ccccc2-c2ccc(N(c3ccc(-c4ccccc4)cc3)c3ccc4c(c3)C(c3ccccc3)(c3ccccc3)c3ccc5c(sc6ccccc65)c3-4)cc21 ; CC1(C)c2cc(N(c3ccc(-c4ccccc4)cc3)c3ccc4c(c3)C(c3ccccc3)(c3ccccc3)c3ccccc3-4)ccc2-c2c1ccc1c2sc2ccccc21]

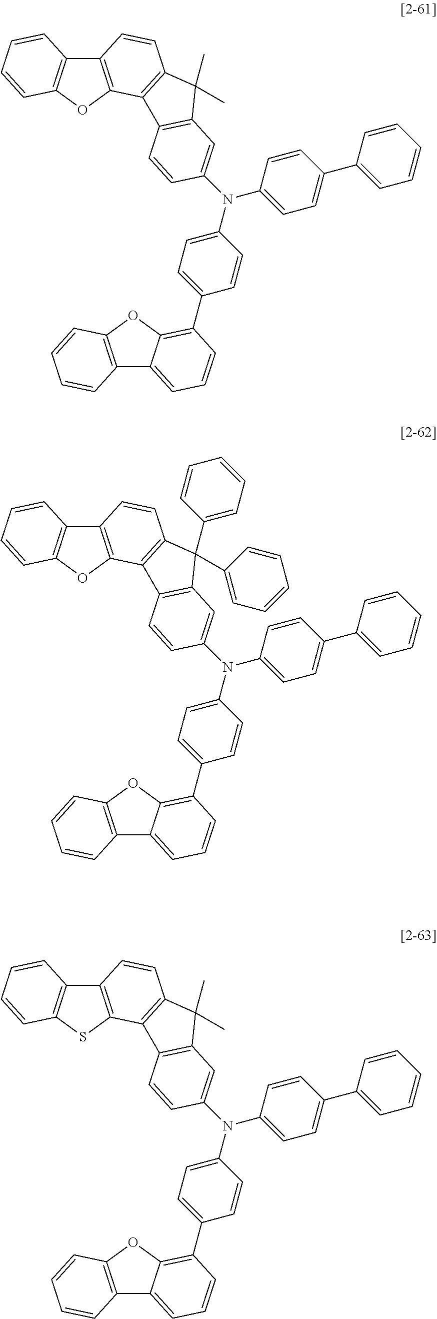 [SMILES: CC1(C)c2cc(N(c3ccc(-c4ccccc4)cc3)c3ccc(-c4cccc5c4oc4ccccc45)cc3)ccc2-c2c1ccc1c2oc2ccccc21 ; c1ccc(-c2ccc(N(c3ccc(-c4cccc5c4oc4ccccc45)cc3)c3ccc4c(c3)C(c3ccccc3)(c3ccccc3)c3ccc5c(oc6ccccc65)c3-4)cc2)cc1 ; CC1(C)c2cc(N(c3ccc(-c4ccccc4)cc3)c3ccc(-c4cccc5c4oc4ccccc45)cc3)ccc2-c2c1ccc1c2sc2ccccc21]

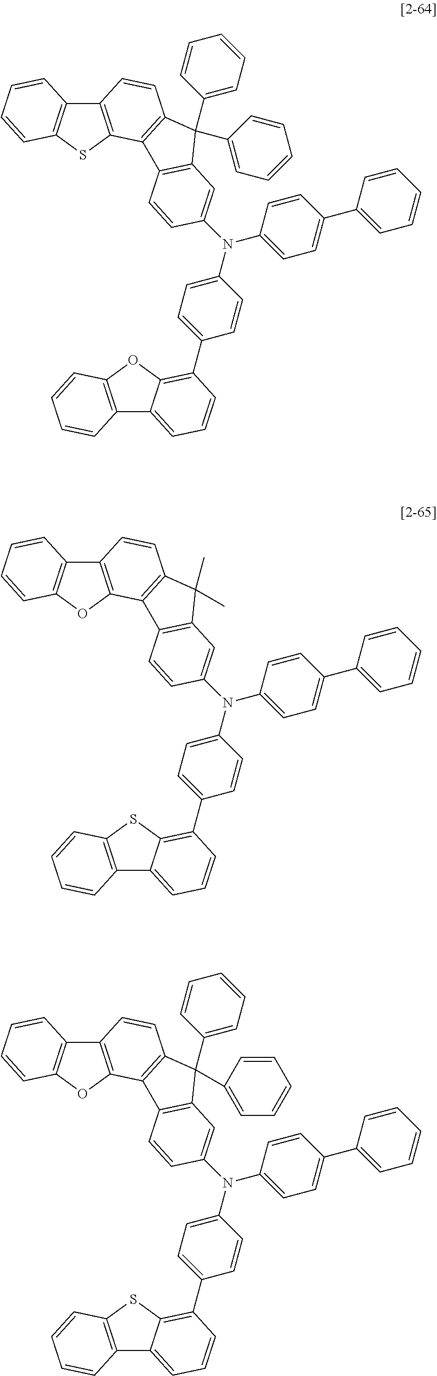 [SMILES: c1ccc(-c2ccc(N(c3ccc(-c4cccc5c4oc4ccccc45)cc3)c3ccc4c(c3)C(c3ccccc3)(c3ccccc3)c3ccc5c(sc6ccccc65)c3-4)cc2)cc1 ; CC1(C)c2cc(N(c3ccc(-c4ccccc4)cc3)c3ccc(-c4cccc5c4sc4ccccc45)cc3)ccc2-c2c1ccc1c2oc2ccccc21 ; c1ccc(-c2ccc(N(c3ccc(-c4cccc5c4sc4ccccc45)cc3)c3ccc4c(c3)C(c3ccccc3)(c3ccccc3)c3ccc5c(oc6ccccc65)c3-4)cc2)cc1]

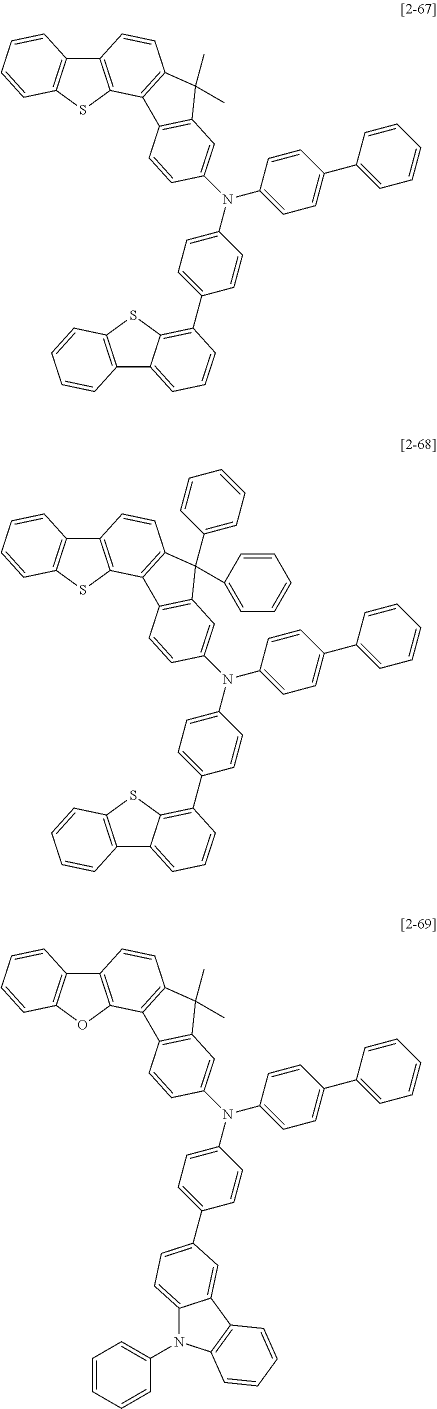 [SMILES: CC1(C)c2cc(N(c3ccc(-c4ccccc4)cc3)c3ccc(-c4cccc5c4sc4ccccc45)cc3)ccc2-c2c1ccc1c2sc2ccccc21 ; c1ccc(-c2ccc(N(c3ccc(-c4cccc5c4sc4ccccc45)cc3)c3ccc4c(c3)C(c3ccccc3)(c3ccccc3)c3ccc5c(sc6ccccc65)c3-4)cc2)cc1 ; CC1(C)c2cc(N(c3ccc(-c4ccccc4)cc3)c3ccc(-c4ccc5c(c4)c4ccccc4n5-c4ccccc4)cc3)ccc2-c2c1ccc1c2oc2ccccc21]

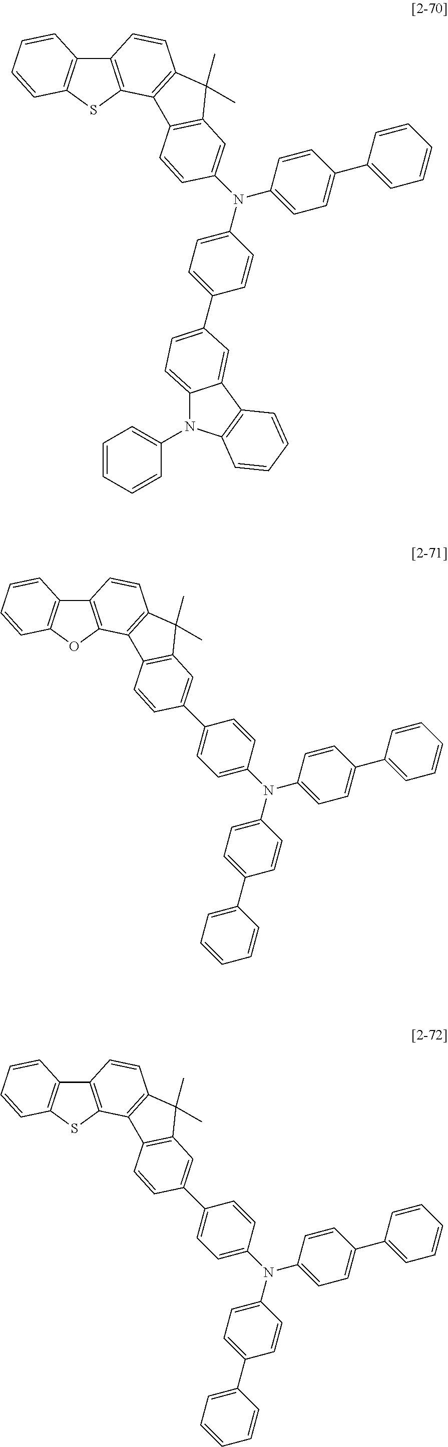 [SMILES: CC1(C)c2cc(N(c3ccc(-c4ccccc4)cc3)c3ccc(-c4ccc5c(c4)c4ccccc4n5-c4ccccc4)cc3)ccc2-c2c1ccc1c2sc2ccccc21 ; CC1(C)c2cc(-c3ccc(N(c4ccc(-c5ccccc5)cc4)c4ccc(-c5ccccc5)cc4)cc3)ccc2-c2c1ccc1c2oc2ccccc21 ; CC1(C)c2cc(-c3ccc(N(c4ccc(-c5ccccc5)cc4)c4ccc(-c5ccccc5)cc4)cc3)ccc2-c2c1ccc1c2sc2ccccc21]

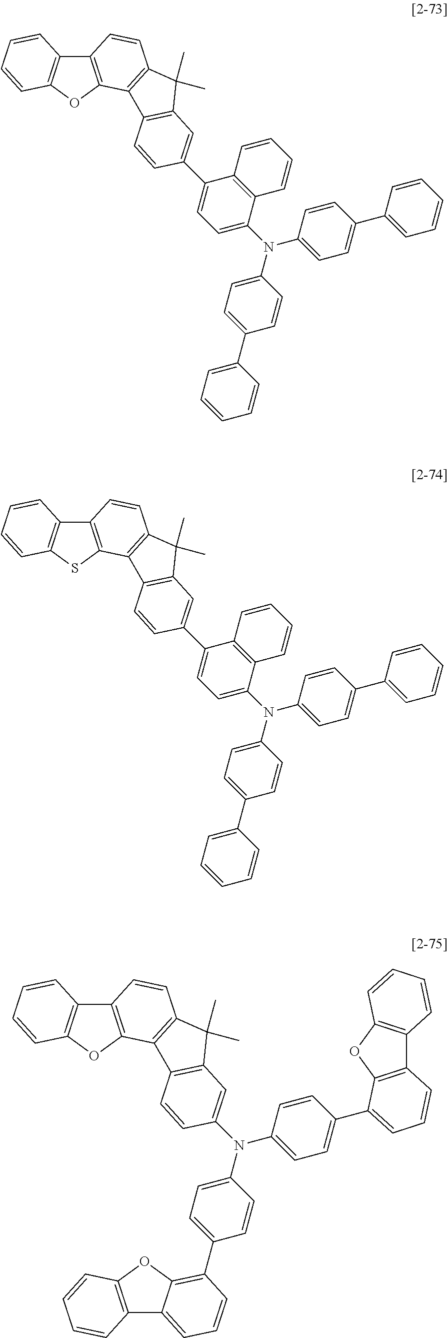 [SMILES: CC1(C)c2cc(-c3ccc(N(c4ccc(-c5ccccc5)cc4)c4ccc(-c5ccccc5)cc4)c4ccccc34)ccc2-c2c1ccc1c2oc2ccccc21 ; CC1(C)c2cc(-c3ccc(N(c4ccc(-c5ccccc5)cc4)c4ccc(-c5ccccc5)cc4)c4ccccc34)ccc2-c2c1ccc1c2sc2ccccc21 ; CC1(C)c2cc(N(c3ccc(-c4cccc5c4oc4ccccc45)cc3)c3ccc(-c4cccc5c4oc4ccccc45)cc3)ccc2-c2c1ccc1c2oc2ccccc21]

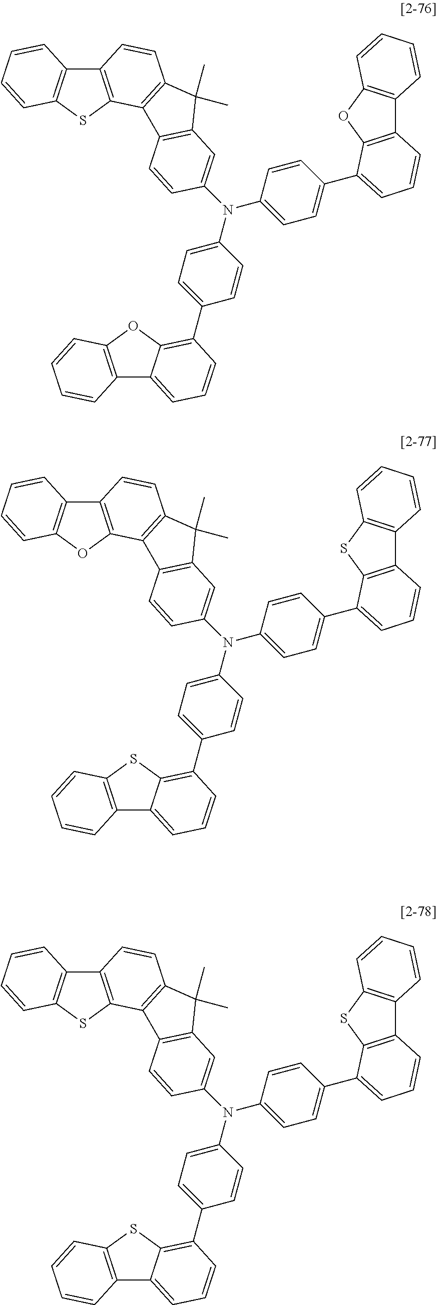 [SMILES: CC1(C)c2cc(N(c3ccc(-c4cccc5c4oc4ccccc45)cc3)c3ccc(-c4cccc5c4oc4ccccc45)cc3)ccc2-c2c1ccc1c2sc2ccccc21 ; CC1(C)c2cc(N(c3ccc(-c4cccc5c4sc4ccccc45)cc3)c3ccc(-c4cccc5c4sc4ccccc45)cc3)ccc2-c2c1ccc1c2oc2ccccc21 ; CC1(C)c2cc(N(c3ccc(-c4cccc5c4sc4ccccc45)cc3)c3ccc(-c4cccc5c4sc4ccccc45)cc3)ccc2-c2c1ccc1c2sc2ccccc21]

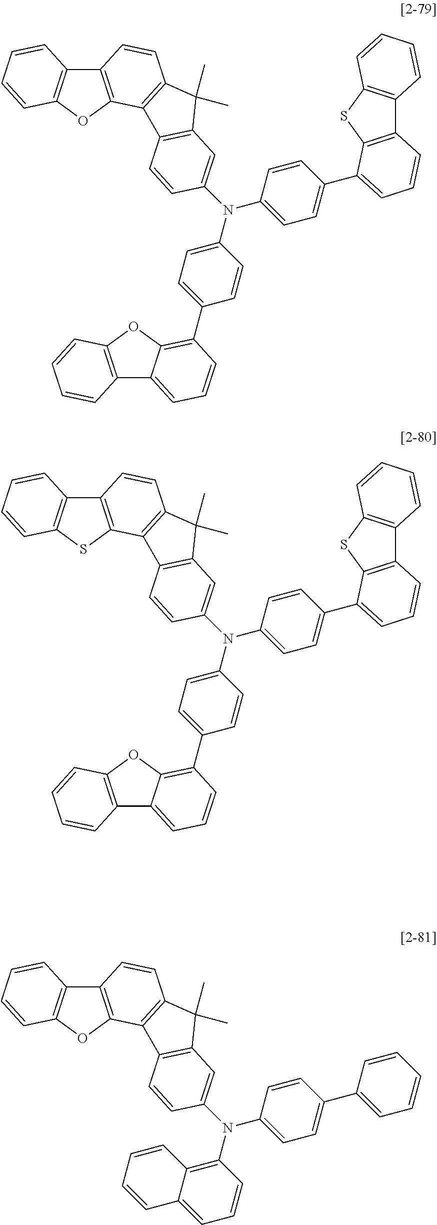 [SMILES: CC1(C)c2cc(N(c3ccc(-c4cccc5c4oc4ccccc45)cc3)c3ccc(-c4cccc5c4sc4ccccc45)cc3)ccc2-c2c1ccc1c2oc2ccccc21 ; CC1(C)c2cc(N(c3ccc(-c4cccc5c4oc4ccccc45)cc3)c3ccc(-c4cccc5c4sc4ccccc45)cc3)ccc2-c2c1ccc1c2sc2ccccc21 ; CC1(C)c2cc(N(c3ccc(-c4ccccc4)cc3)c3cccc4ccccc34)ccc2-c2c1ccc1c2oc2ccccc21]

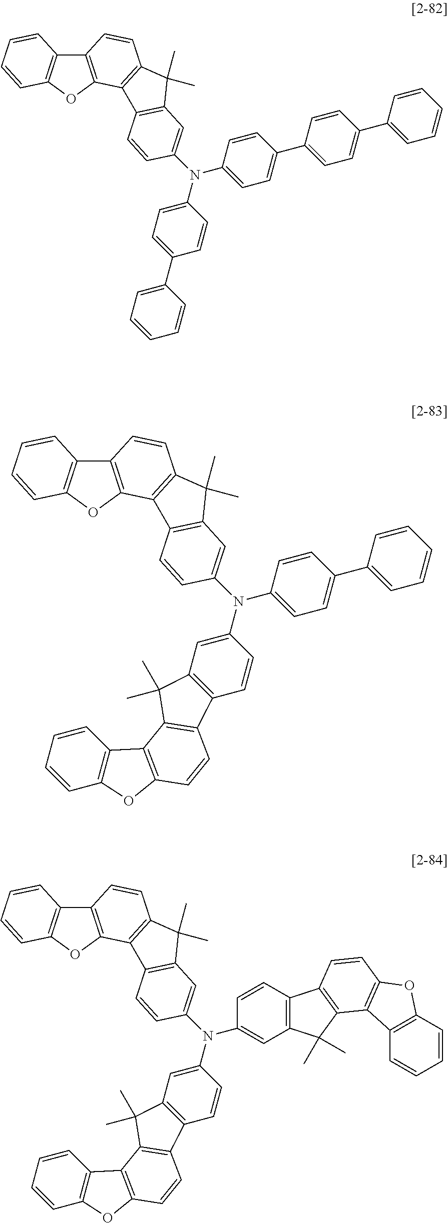 [SMILES: CC1(C)c2cc(N(c3ccc(-c4ccccc4)cc3)c3ccc(-c4ccc(-c5ccccc5)cc4)cc3)ccc2-c2c1ccc1c2oc2ccccc21 ; CC1(C)c2cc(N(c3ccc(-c4ccccc4)cc3)c3ccc4c(c3)C(C)(C)c3c-4ccc4oc5ccccc5c34)ccc2-c2c1ccc1c2oc2ccccc21 ; CC1(C)c2cc(N(c3ccc4c(c3)C(C)(C)c3c-4ccc4oc5ccccc5c34)c3ccc4c(c3)C(C)(C)c3c-4ccc4oc5ccccc5c34)ccc2-c2c1ccc1c2oc2ccccc21]

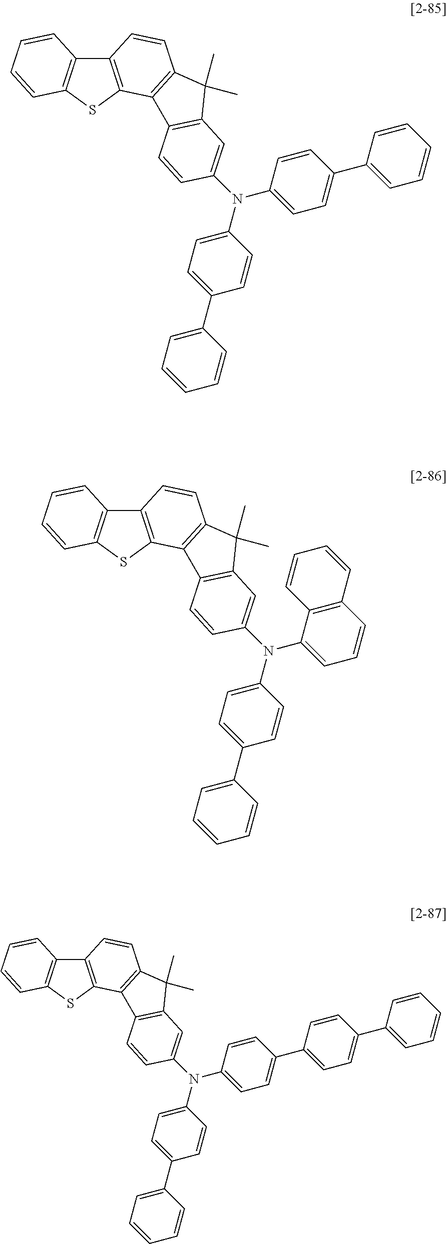 [SMILES: CC1(C)c2cc(N(c3ccc(-c4ccccc4)cc3)c3ccc(-c4ccccc4)cc3)ccc2-c2c1ccc1c2sc2ccccc21 ; CC1(C)c2cc(N(c3ccc(-c4ccccc4)cc3)c3cccc4ccccc34)ccc2-c2c1ccc1c2sc2ccccc21 ; CC1(C)c2cc(N(c3ccc(-c4ccccc4)cc3)c3ccc(-c4ccc(-c5ccccc5)cc4)cc3)ccc2-c2c1ccc1c2sc2ccccc21]

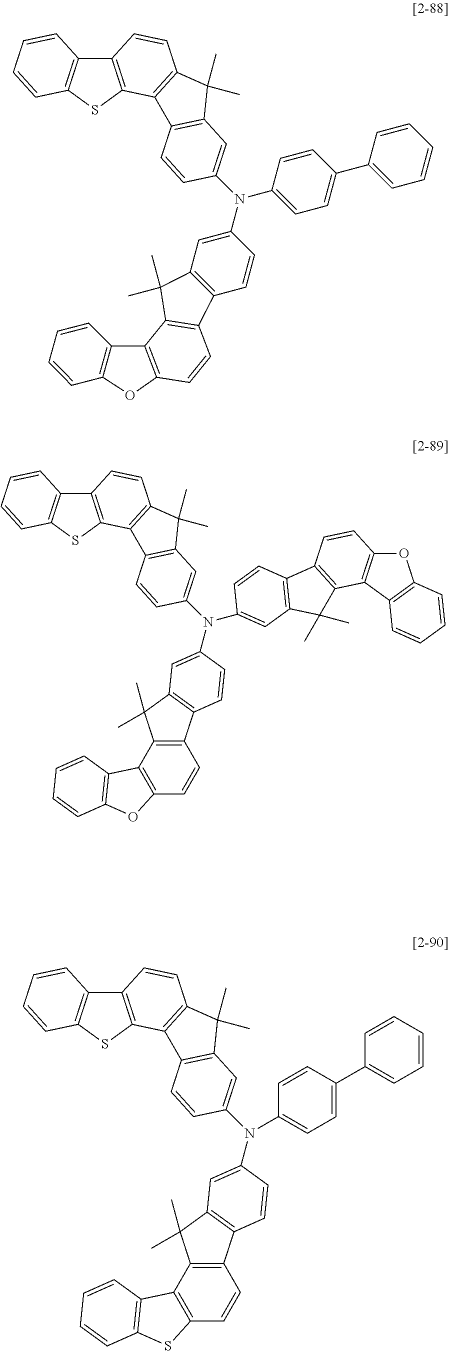 [SMILES: CC1(C)c2cc(N(c3ccc(-c4ccccc4)cc3)c3ccc4c(c3)C(C)(C)c3c-4ccc4oc5ccccc5c34)ccc2-c2c1ccc1c2sc2ccccc21 ; CC1(C)c2cc(N(c3ccc4c(c3)C(C)(C)c3c-4ccc4oc5ccccc5c34)c3ccc4c(c3)C(C)(C)c3c-4ccc4oc5ccccc5c34)ccc2-c2c1ccc1c2sc2ccccc21 ; CC1(C)c2cc(N(c3ccc(-c4ccccc4)cc3)c3ccc4c(c3)C(C)(C)c3c-4ccc4sc5ccccc5c34)ccc2-c2c1ccc1c2sc2ccccc21]

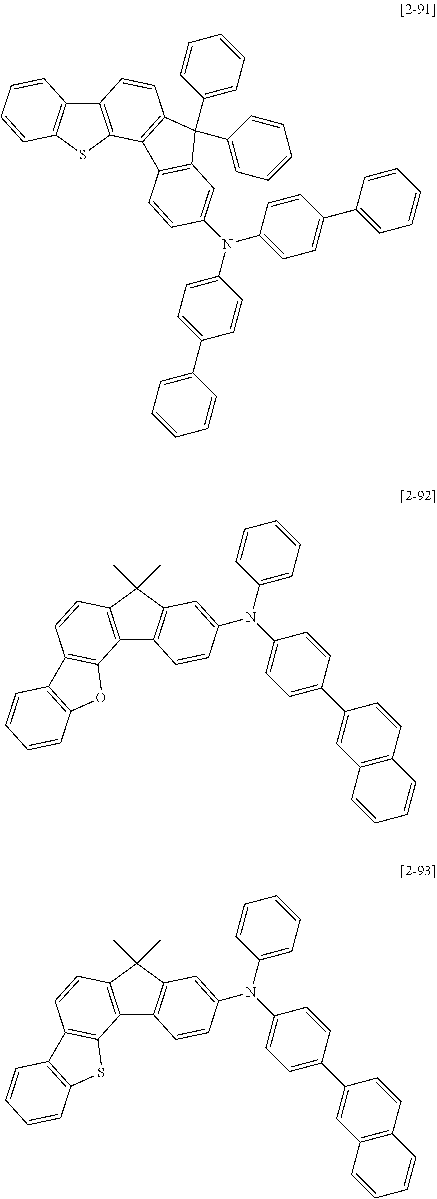 [SMILES: c1ccc(-c2ccc(N(c3ccc(-c4ccccc4)cc3)c3ccc4c(c3)C(c3ccccc3)(c3ccccc3)c3ccc5c(sc6ccccc65)c3-4)cc2)cc1 ; CC1(C)c2cc(N(c3ccccc3)c3ccc(-c4ccc5ccccc5c4)cc3)ccc2-c2c1ccc1c2oc2ccccc21 ; CC1(C)c2cc(N(c3ccccc3)c3ccc(-c4ccc5ccccc5c4)cc3)ccc2-c2c1ccc1c2sc2ccccc21]

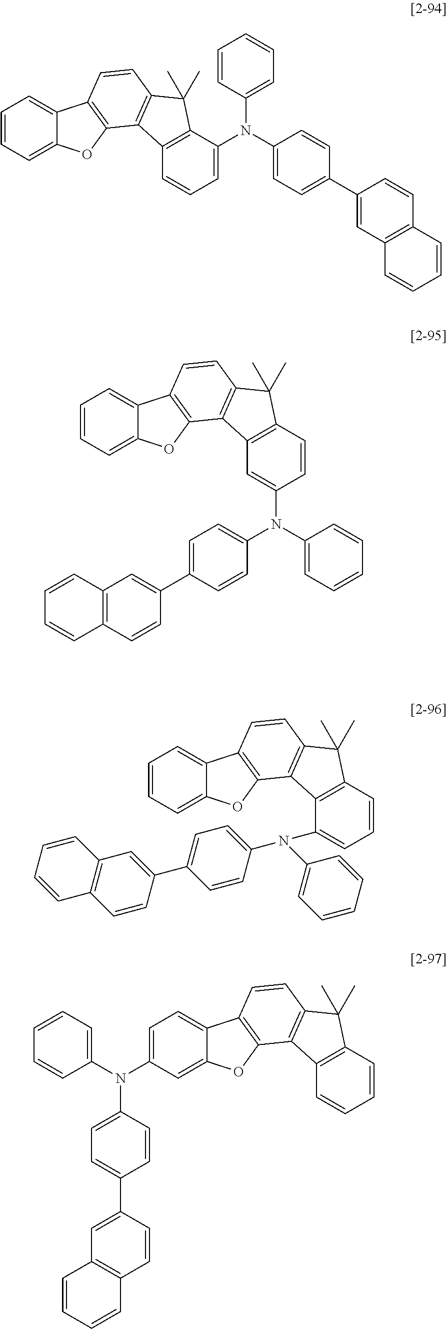 [SMILES: CC1(C)c2ccc3c(oc4ccccc43)c2-c2cccc(N(c3ccccc3)c3ccc(-c4ccc5ccccc5c4)cc3)c21 ; CC1(C)c2ccc(N(c3ccccc3)c3ccc(-c4ccc5ccccc5c4)cc3)cc2-c2c1ccc1c2oc2ccccc21 ; CC1(C)c2cccc(N(c3ccccc3)c3ccc(-c4ccc5ccccc5c4)cc3)c2-c2c1ccc1c2oc2ccccc21 ; CC1(C)c2ccccc2-c2c1ccc1c2oc2cc(N(c3ccccc3)c3ccc(-c4ccc5ccccc5c4)cc3)ccc21]

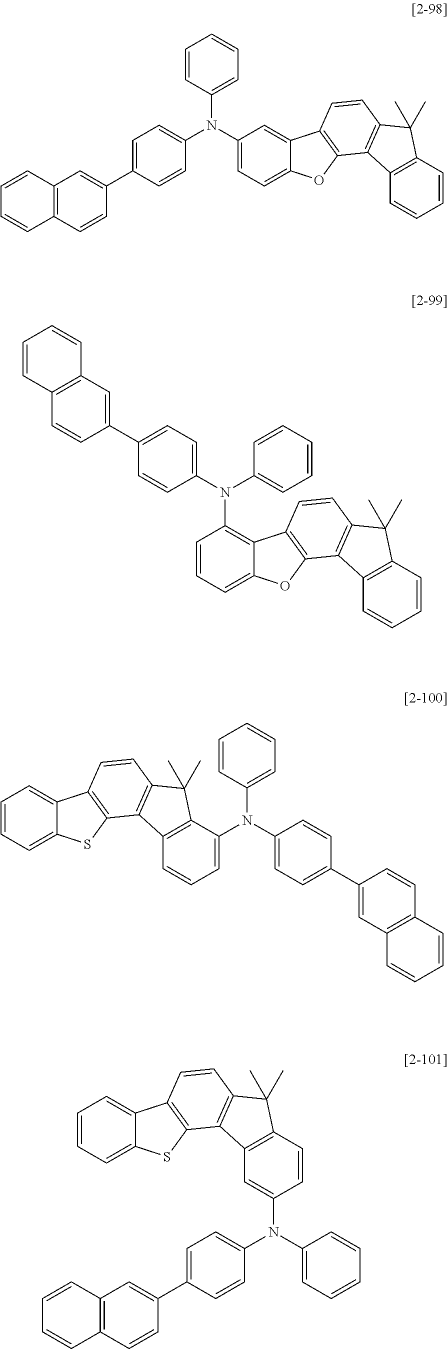 [SMILES: CC1(C)c2ccccc2-c2c1ccc1c2oc2ccc(N(c3ccccc3)c3ccc(-c4ccc5ccccc5c4)cc3)cc21 ; CC1(C)c2ccccc2-c2c1ccc1c2oc2cccc(N(c3ccccc3)c3ccc(-c4ccc5ccccc5c4)cc3)c21 ; CC1(C)c2ccc3c(sc4ccccc43)c2-c2cccc(N(c3ccccc3)c3ccc(-c4ccc5ccccc5c4)cc3)c21 ; CC1(C)c2ccc(N(c3ccccc3)c3ccc(-c4ccc5ccccc5c4)cc3)cc2-c2c1ccc1c2sc2ccccc21]

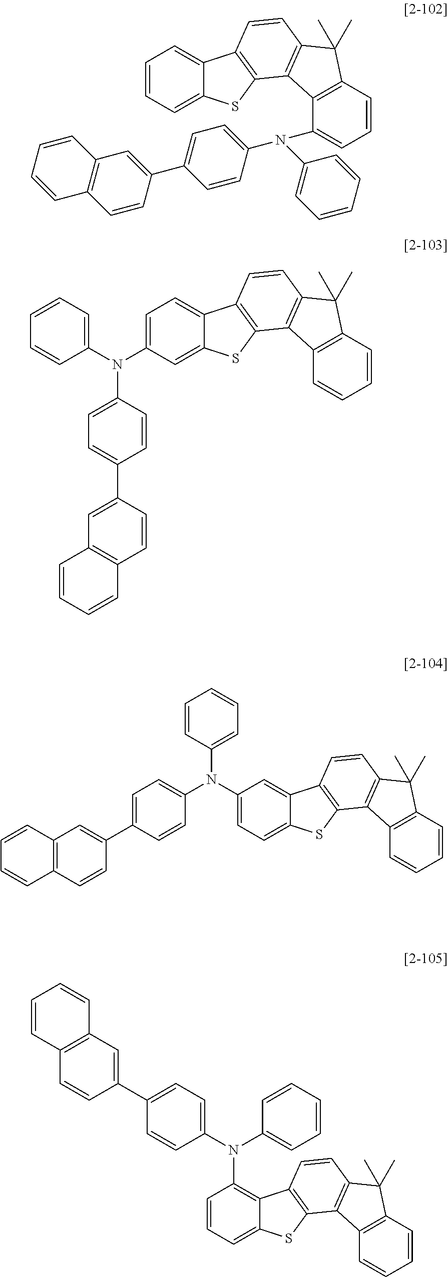 [SMILES: CC1(C)c2cccc(N(c3ccccc3)c3ccc(-c4ccc5ccccc5c4)cc3)c2-c2c1ccc1c2sc2ccccc21 ; CC1(C)c2ccccc2-c2c1ccc1c2sc2cc(N(c3ccccc3)c3ccc(-c4ccc5ccccc5c4)cc3)ccc21 ; CC1(C)c2ccccc2-c2c1ccc1c2sc2ccc(N(c3ccccc3)c3ccc(-c4ccc5ccccc5c4)cc3)cc21 ; CC1(C)c2ccccc2-c2c1ccc1c2sc2cccc(N(c3ccccc3)c3ccc(-c4ccc5ccccc5c4)cc3)c21]

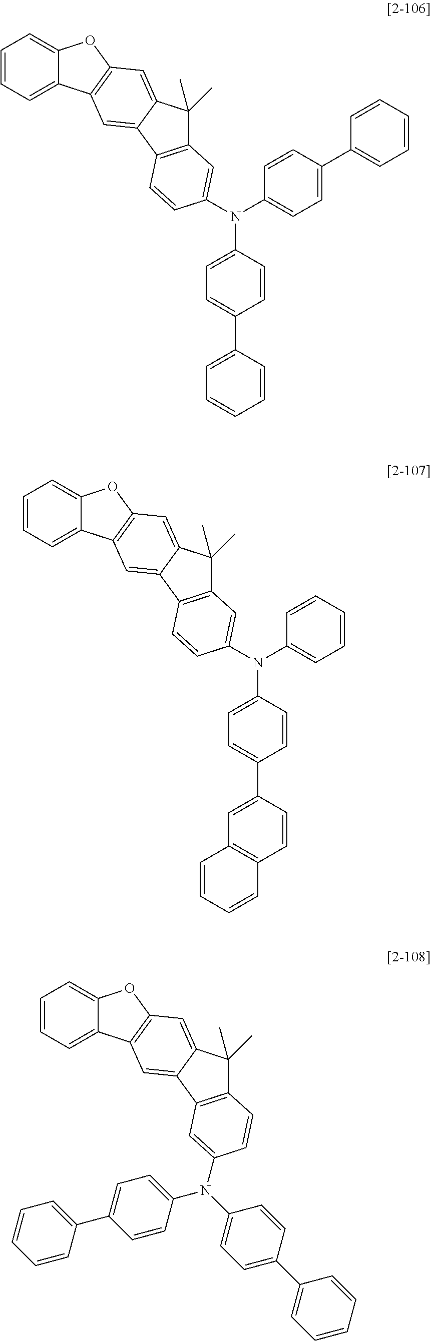 [SMILES: CC1(C)c2cc(N(c3ccc(-c4ccccc4)cc3)c3ccc(-c4ccccc4)cc3)ccc2-c2cc3c(cc21)oc1ccccc13 ; CC1(C)c2cc(N(c3ccccc3)c3ccc(-c4ccc5ccccc5c4)cc3)ccc2-c2cc3c(cc21)oc1ccccc13 ; CC1(C)c2ccc(N(c3ccc(-c4ccccc4)cc3)c3ccc(-c4ccccc4)cc3)cc2-c2cc3c(cc21)oc1ccccc13]

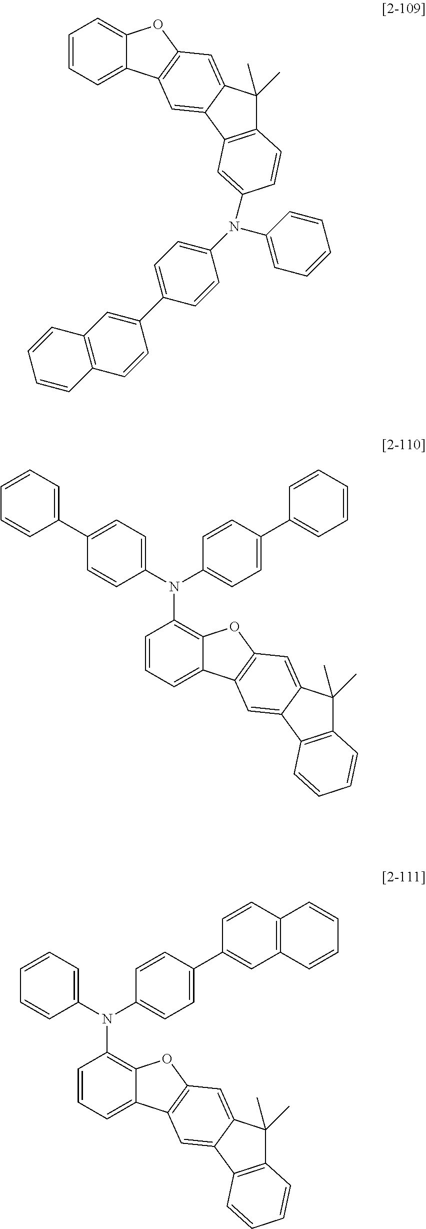 [SMILES: CC1(C)c2ccc(N(c3ccccc3)c3ccc(-c4ccc5ccccc5c4)cc3)cc2-c2cc3c(cc21)oc1ccccc13 ; CC1(C)c2ccccc2-c2cc3c(cc21)oc1c(N(c2ccc(-c4ccccc4)cc2)c2ccc(-c4ccccc4)cc2)cccc13 ; CC1(C)c2ccccc2-c2cc3c(cc21)oc1c(N(c2ccccc2)c2ccc(-c4ccc5ccccc5c4)cc2)cccc13]

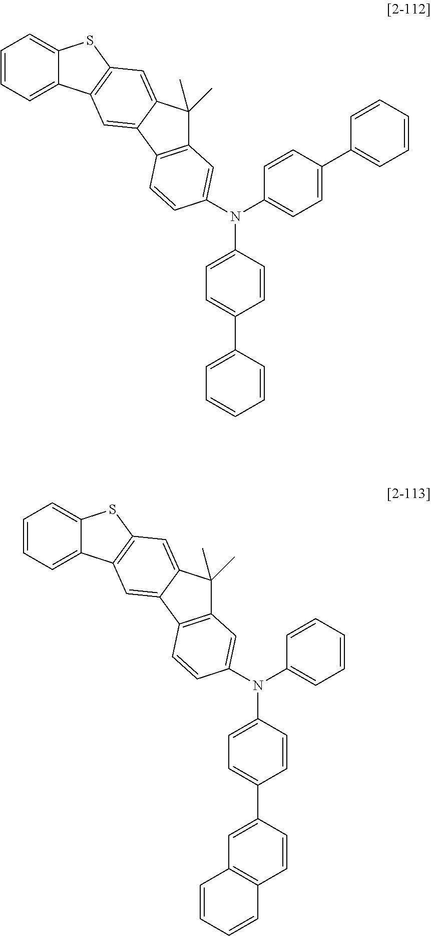 [SMILES: CC1(C)c2cc(N(c3ccc(-c4ccccc4)cc3)c3ccc(-c4ccccc4)cc3)ccc2-c2cc3c(cc21)sc1ccccc13 ; CC1(C)c2cc(N(c3ccccc3)c3ccc(-c4ccc5ccccc5c4)cc3)ccc2-c2cc3c(cc21)sc1ccccc13]

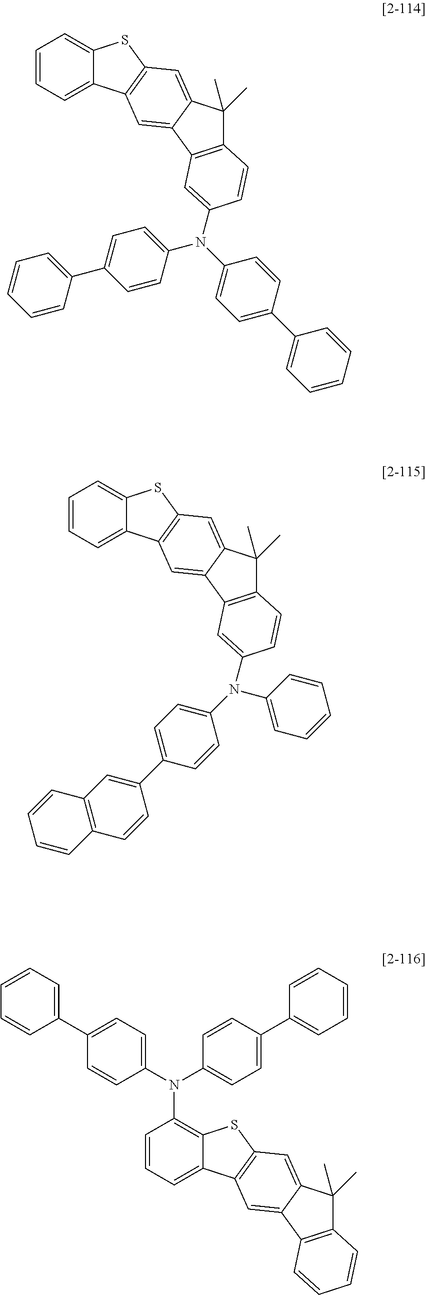 [SMILES: CC1(C)c2ccc(N(c3ccc(-c4ccccc4)cc3)c3ccc(-c4ccccc4)cc3)cc2-c2cc3c(cc21)sc1ccccc13 ; CC1(C)c2ccc(N(c3ccccc3)c3ccc(-c4ccc5ccccc5c4)cc3)cc2-c2cc3c(cc21)sc1ccccc13 ; CC1(C)c2ccccc2-c2cc3c(cc21)sc1c(N(c2ccc(-c4ccccc4)cc2)c2ccc(-c4ccccc4)cc2)cccc13]

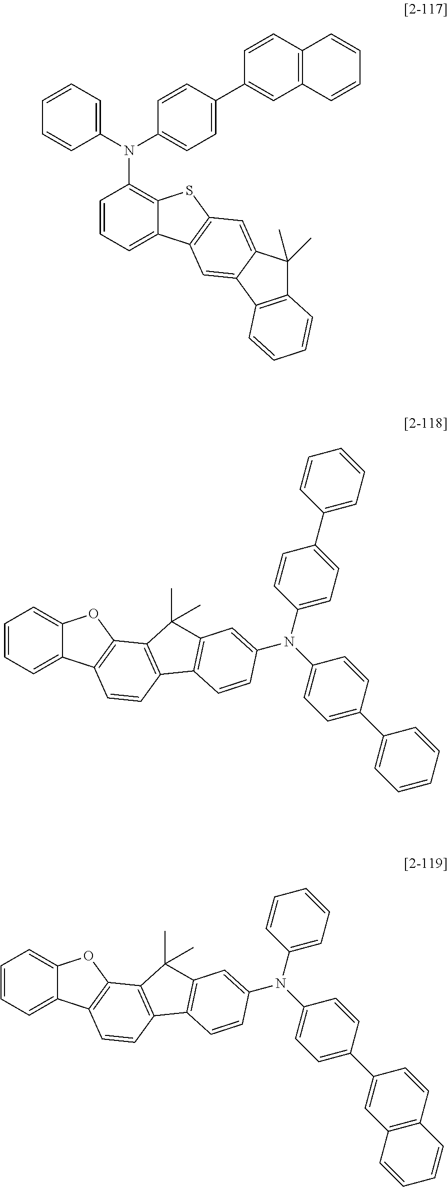 [SMILES: CC1(C)c2ccccc2-c2cc3c(cc21)sc1c(N(c2ccccc2)c2ccc(-c4ccc5ccccc5c4)cc2)cccc13 ; CC1(C)c2cc(N(c3ccc(-c4ccccc4)cc3)c3ccc(-c4ccccc4)cc3)ccc2-c2ccc3c(oc4ccccc43)c21 ; CC1(C)c2cc(N(c3ccccc3)c3ccc(-c4ccc5ccccc5c4)cc3)ccc2-c2ccc3c(oc4ccccc43)c21]

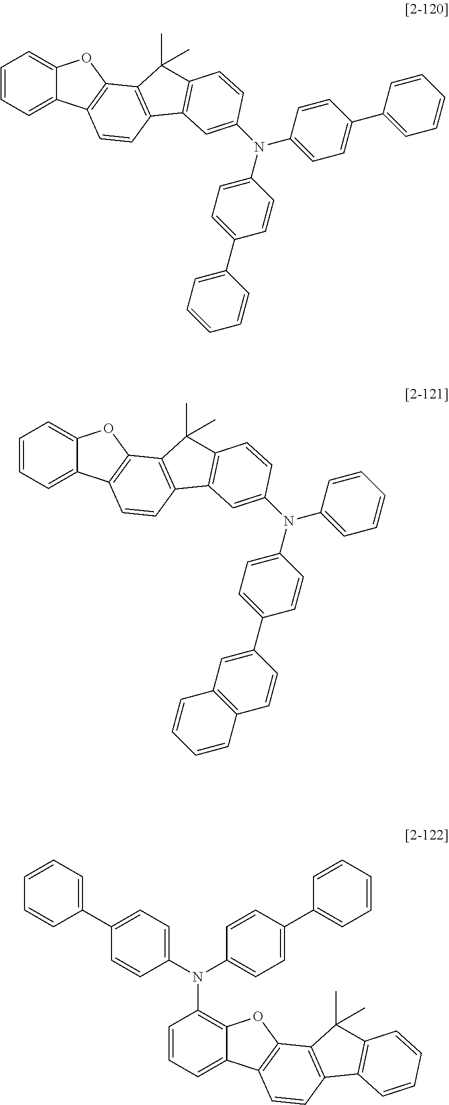 [SMILES: CC1(C)c2ccc(N(c3ccc(-c4ccccc4)cc3)c3ccc(-c4ccccc4)cc3)cc2-c2ccc3c(oc4ccccc43)c21 ; CC1(C)c2ccc(N(c3ccccc3)c3ccc(-c4ccc5ccccc5c4)cc3)cc2-c2ccc3c(oc4ccccc43)c21 ; CC1(C)c2ccccc2-c2ccc3c(oc4c(N(c5ccc(-c6ccccc6)cc5)c5ccc(-c6ccccc6)cc5)cccc43)c21]

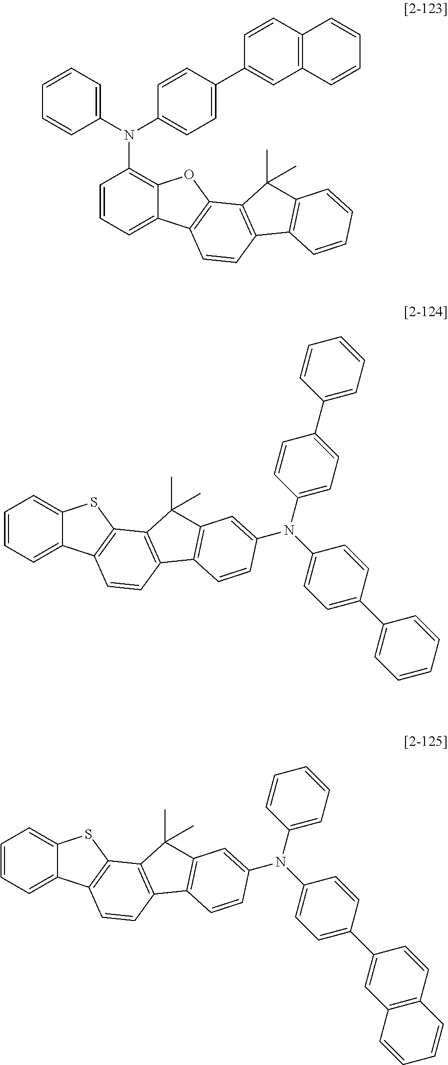 [SMILES: CC1(C)c2ccccc2-c2ccc3c(oc4c(N(c5ccccc5)c5ccc(-c6ccc7ccccc7c6)cc5)cccc43)c21 ; CC1(C)c2cc(N(c3ccc(-c4ccccc4)cc3)c3ccc(-c4ccccc4)cc3)ccc2-c2ccc3c(sc4ccccc43)c21 ; CC1(C)c2cc(N(c3ccccc3)c3ccc(-c4ccc5ccccc5c4)cc3)ccc2-c2ccc3c(sc4ccccc43)c21]

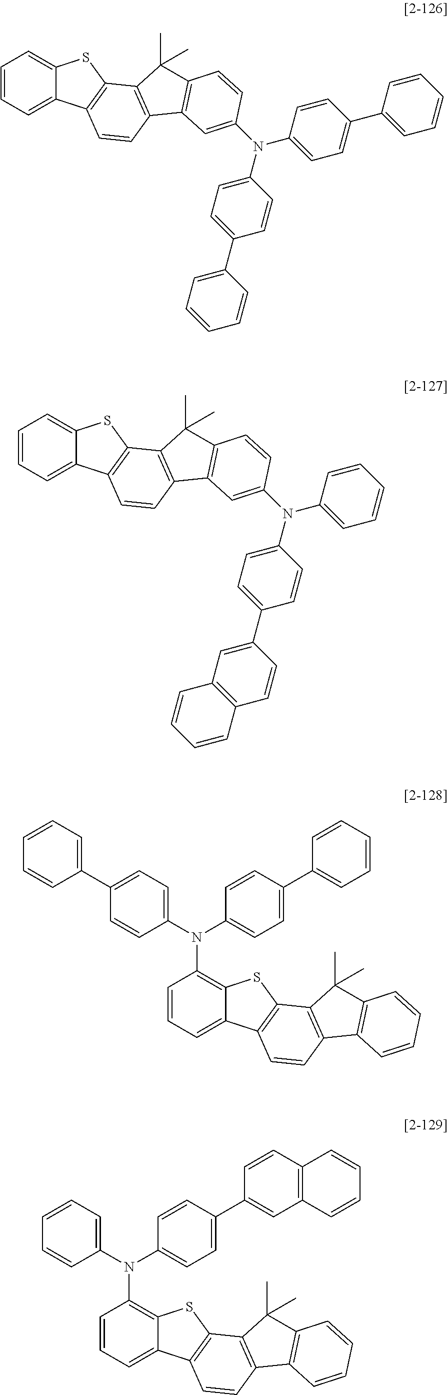 [SMILES: CC1(C)c2ccc(N(c3ccc(-c4ccccc4)cc3)c3ccc(-c4ccccc4)cc3)cc2-c2ccc3c(sc4ccccc43)c21 ; CC1(C)c2ccc(N(c3ccccc3)c3ccc(-c4ccc5ccccc5c4)cc3)cc2-c2ccc3c(sc4ccccc43)c21 ; CC1(C)c2ccccc2-c2ccc3c(sc4c(N(c5ccc(-c6ccccc6)cc5)c5ccc(-c6ccccc6)cc5)cccc43)c21 ; CC1(C)c2ccccc2-c2ccc3c(sc4c(N(c5ccccc5)c5ccc(-c6ccc7ccccc7c6)cc5)cccc43)c21]

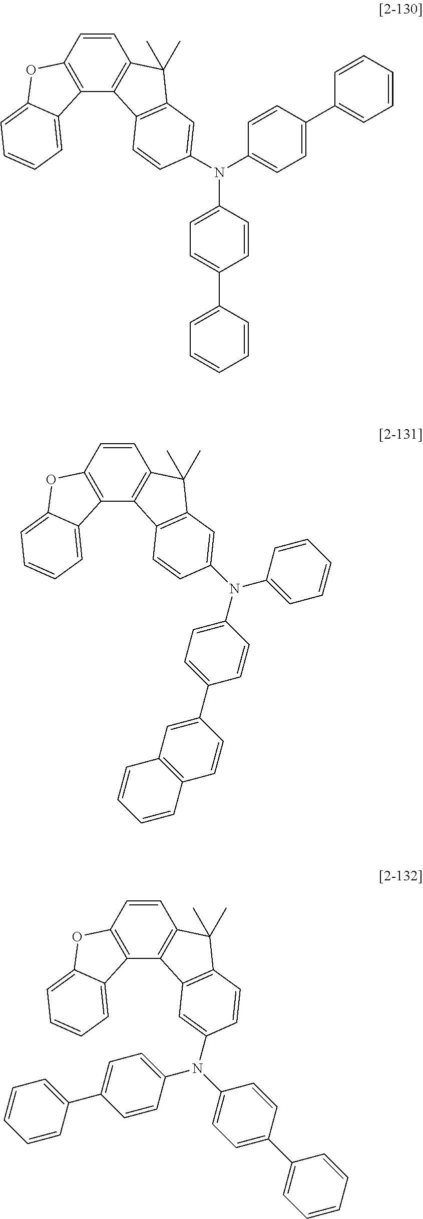 [SMILES: CC1(C)c2cc(N(c3ccc(-c4ccccc4)cc3)c3ccc(-c4ccccc4)cc3)ccc2-c2c1ccc1oc3ccccc3c21 ; CC1(C)c2cc(N(c3ccccc3)c3ccc(-c4ccc5ccccc5c4)cc3)ccc2-c2c1ccc1oc3ccccc3c21 ; CC1(C)c2ccc(N(c3ccc(-c4ccccc4)cc3)c3ccc(-c4ccccc4)cc3)cc2-c2c1ccc1oc3ccccc3c21]

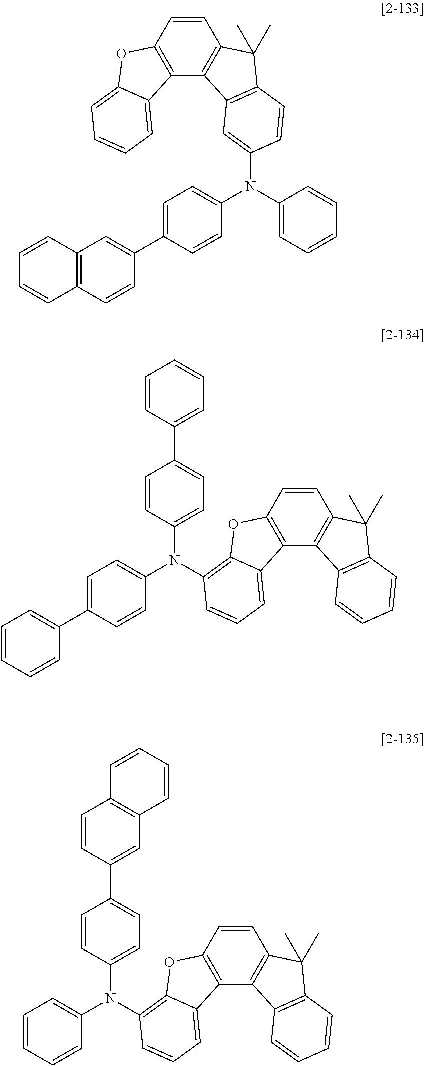 [SMILES: CC1(C)c2ccc(N(c3ccccc3)c3ccc(-c4ccc5ccccc5c4)cc3)cc2-c2c1ccc1oc3ccccc3c21 ; CC1(C)c2ccccc2-c2c1ccc1oc3c(N(c4ccc(-c5ccccc5)cc4)c4ccc(-c5ccccc5)cc4)cccc3c21 ; CC1(C)c2ccccc2-c2c1ccc1oc3c(N(c4ccccc4)c4ccc(-c5ccc6ccccc6c5)cc4)cccc3c21]

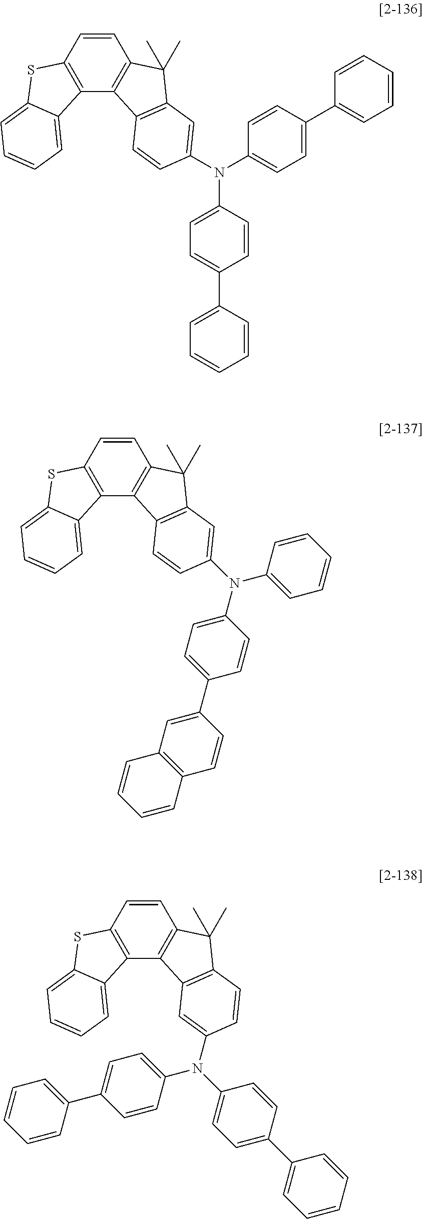 [SMILES: CC1(C)c2cc(N(c3ccc(-c4ccccc4)cc3)c3ccc(-c4ccccc4)cc3)ccc2-c2c1ccc1sc3ccccc3c21 ; CC1(C)c2cc(N(c3ccccc3)c3ccc(-c4ccc5ccccc5c4)cc3)ccc2-c2c1ccc1sc3ccccc3c21 ; CC1(C)c2ccc(N(c3ccc(-c4ccccc4)cc3)c3ccc(-c4ccccc4)cc3)cc2-c2c1ccc1sc3ccccc3c21]

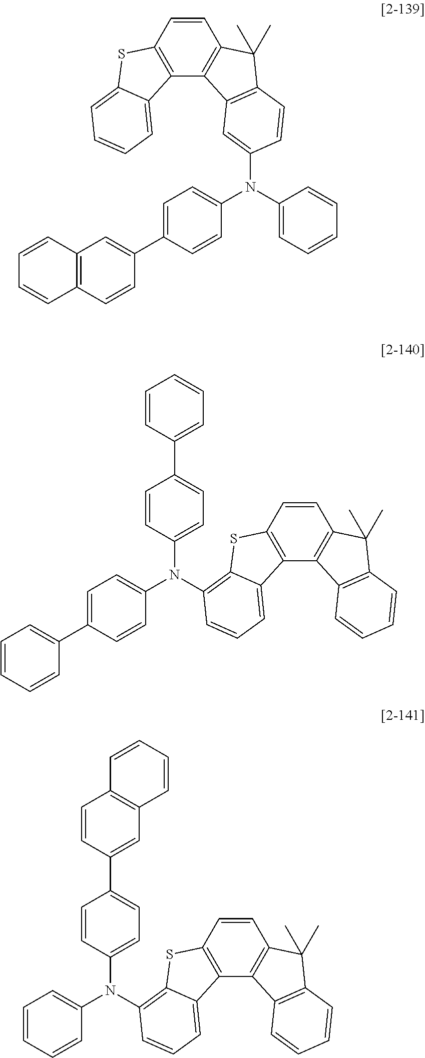 [SMILES: CC1(C)c2ccc(N(c3ccccc3)c3ccc(-c4ccc5ccccc5c4)cc3)cc2-c2c1ccc1sc3ccccc3c21 ; CC1(C)c2ccccc2-c2c1ccc1sc3c(N(c4ccc(-c5ccccc5)cc4)c4ccc(-c5ccccc5)cc4)cccc3c21 ; CC1(C)c2ccccc2-c2c1ccc1sc3c(N(c4ccccc4)c4ccc(-c5ccc6ccccc6c5)cc4)cccc3c21]

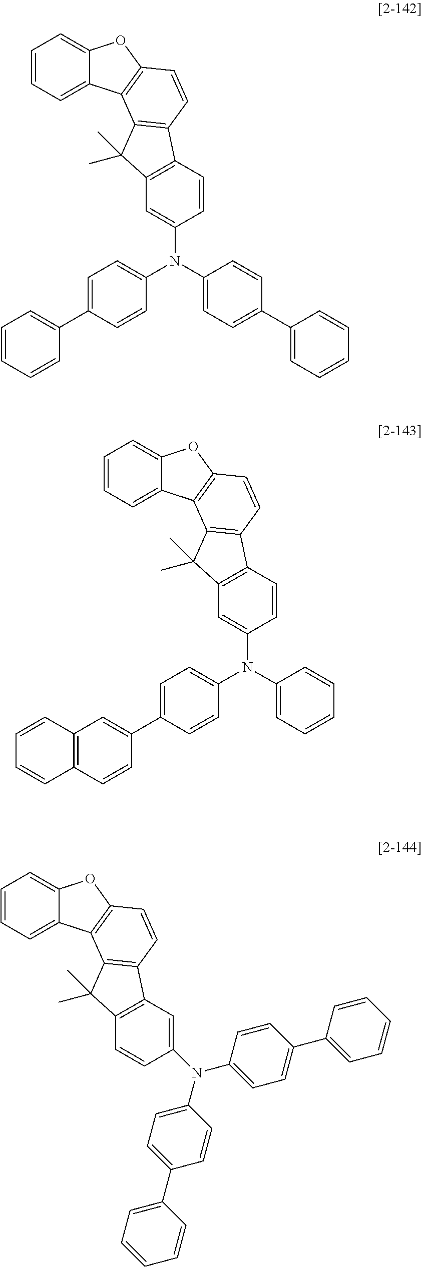 [SMILES: CC1(C)c2cc(N(c3ccc(-c4ccccc4)cc3)c3ccc(-c4ccccc4)cc3)ccc2-c2ccc3oc4ccccc4c3c21 ; CC1(C)c2cc(N(c3ccccc3)c3ccc(-c4ccc5ccccc5c4)cc3)ccc2-c2ccc3oc4ccccc4c3c21 ; CC1(C)c2ccc(N(c3ccc(-c4ccccc4)cc3)c3ccc(-c4ccccc4)cc3)cc2-c2ccc3oc4ccccc4c3c21]

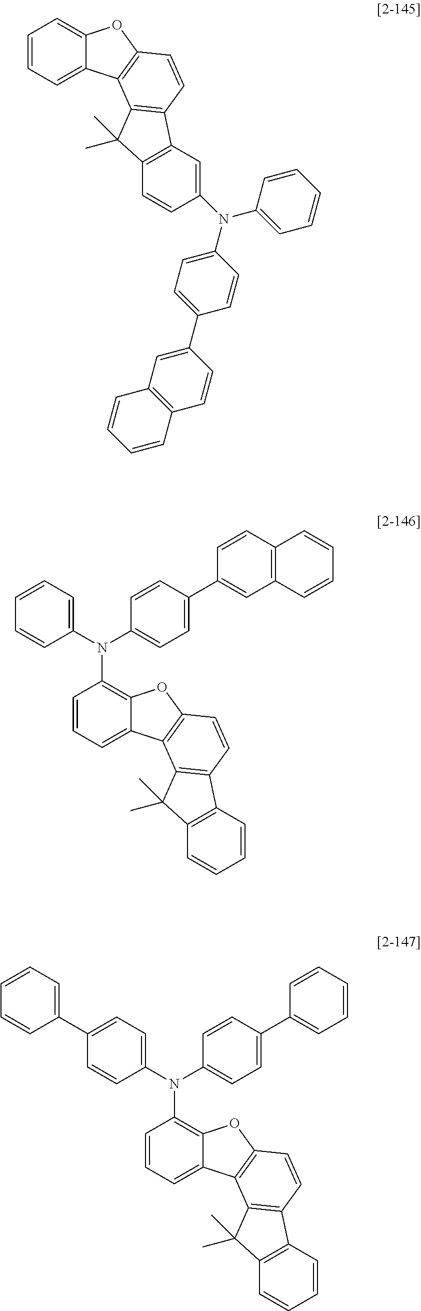 [SMILES: CC1(C)c2ccc(N(c3ccccc3)c3ccc(-c4ccc5ccccc5c4)cc3)cc2-c2ccc3oc4ccccc4c3c21 ; CC1(C)c2ccccc2-c2ccc3oc4c(N(c5ccccc5)c5ccc(-c6ccc7ccccc7c6)cc5)cccc4c3c21 ; CC1(C)c2ccccc2-c2ccc3oc4c(N(c5ccc(-c6ccccc6)cc5)c5ccc(-c6ccccc6)cc5)cccc4c3c21]

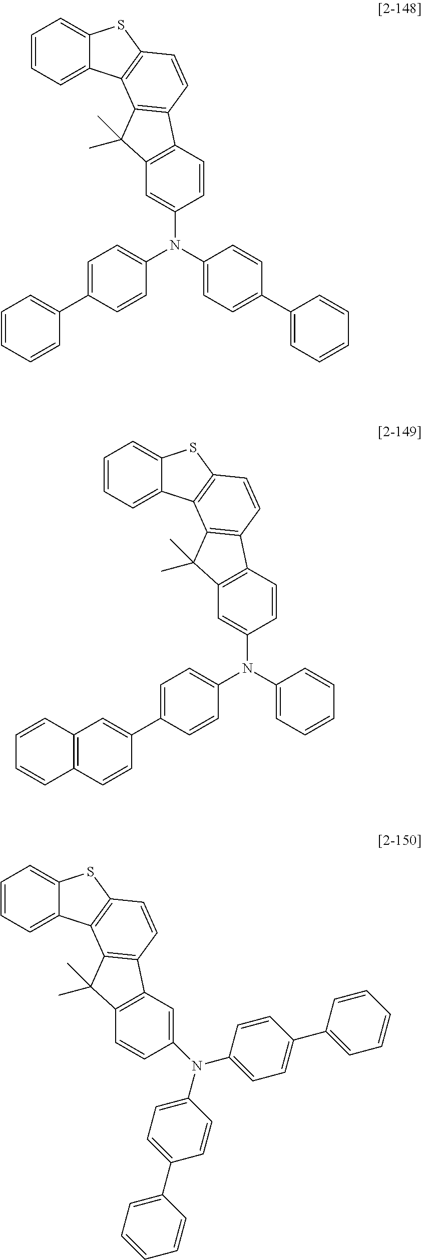 [SMILES: CC1(C)c2cc(N(c3ccc(-c4ccccc4)cc3)c3ccc(-c4ccccc4)cc3)ccc2-c2ccc3sc4ccccc4c3c21 ; CC1(C)c2cc(N(c3ccccc3)c3ccc(-c4ccc5ccccc5c4)cc3)ccc2-c2ccc3sc4ccccc4c3c21 ; CC1(C)c2ccc(N(c3ccc(-c4ccccc4)cc3)c3ccc(-c4ccccc4)cc3)cc2-c2ccc3sc4ccccc4c3c21]

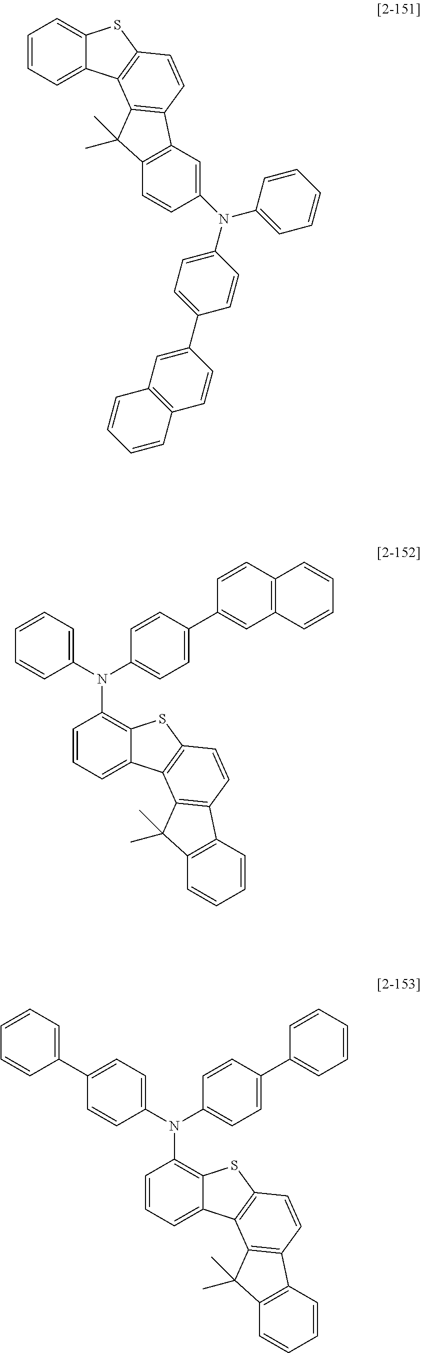 [SMILES: CC1(C)c2ccc(N(c3ccccc3)c3ccc(-c4ccc5ccccc5c4)cc3)cc2-c2ccc3sc4ccccc4c3c21 ; CC1(C)c2ccccc2-c2ccc3sc4c(N(c5ccccc5)c5ccc(-c6ccc7ccccc7c6)cc5)cccc4c3c21 ; CC1(C)c2ccccc2-c2ccc3sc4c(N(c5ccc(-c6ccccc6)cc5)c5ccc(-c6ccccc6)cc5)cccc4c3c21]

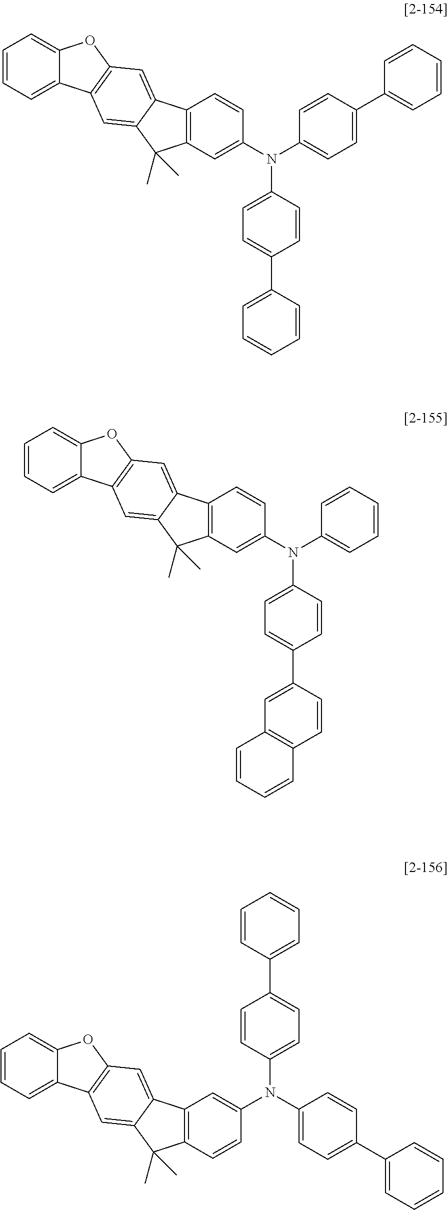 [SMILES: CC1(C)c2cc(N(c3ccc(-c4ccccc4)cc3)c3ccc(-c4ccccc4)cc3)ccc2-c2cc3oc4ccccc4c3cc21 ; CC1(C)c2cc(N(c3ccccc3)c3ccc(-c4ccc5ccccc5c4)cc3)ccc2-c2cc3oc4ccccc4c3cc21 ; CC1(C)c2ccc(N(c3ccc(-c4ccccc4)cc3)c3ccc(-c4ccccc4)cc3)cc2-c2cc3oc4ccccc4c3cc21]

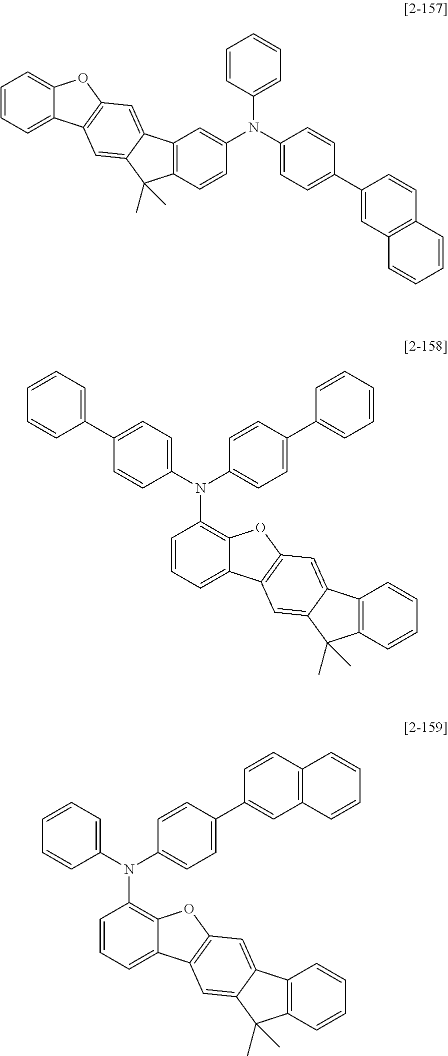 [SMILES: CC1(C)c2ccc(N(c3ccccc3)c3ccc(-c4ccc5ccccc5c4)cc3)cc2-c2cc3oc4ccccc4c3cc21 ; CC1(C)c2ccccc2-c2cc3oc4c(N(c5ccc(-c6ccccc6)cc5)c5ccc(-c6ccccc6)cc5)cccc4c3cc21 ; CC1(C)c2ccccc2-c2cc3oc4c(N(c5ccccc5)c5ccc(-c6ccc7ccccc7c6)cc5)cccc4c3cc21]

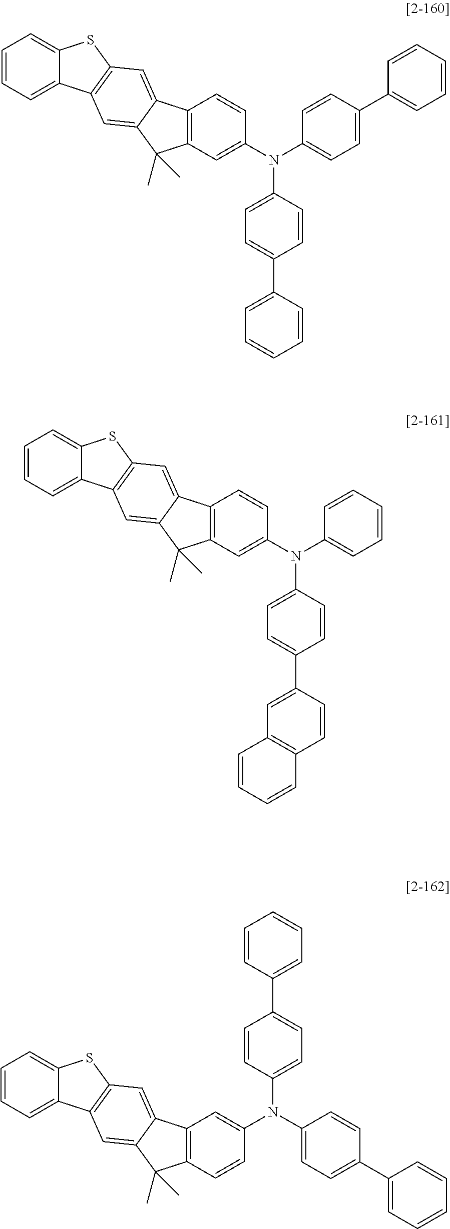 [SMILES: CC1(C)c2cc(N(c3ccc(-c4ccccc4)cc3)c3ccc(-c4ccccc4)cc3)ccc2-c2cc3sc4ccccc4c3cc21 ; CC1(C)c2cc(N(c3ccccc3)c3ccc(-c4ccc5ccccc5c4)cc3)ccc2-c2cc3sc4ccccc4c3cc21 ; CC1(C)c2ccc(N(c3ccc(-c4ccccc4)cc3)c3ccc(-c4ccccc4)cc3)cc2-c2cc3sc4ccccc4c3cc21]

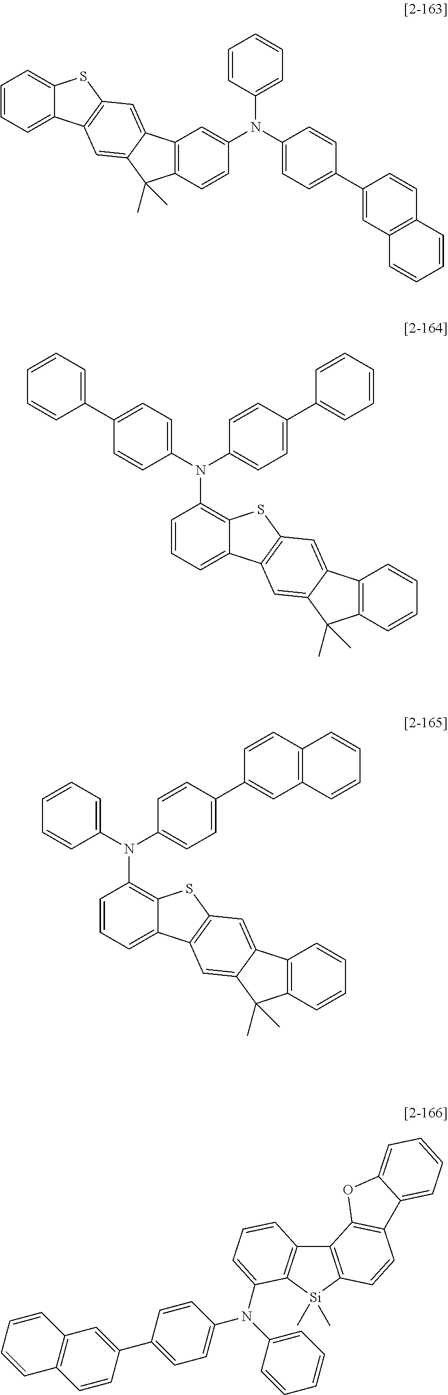 [SMILES: CC1(C)c2ccc(N(c3ccccc3)c3ccc(-c4ccc5ccccc5c4)cc3)cc2-c2cc3sc4ccccc4c3cc21 ; CC1(C)c2ccccc2-c2cc3sc4c(N(c5ccc(-c6ccccc6)cc5)c5ccc(-c6ccccc6)cc5)cccc4c3cc21 ; CC1(C)c2ccccc2-c2cc3sc4c(N(c5ccccc5)c5ccc(-c6ccc7ccccc7c6)cc5)cccc4c3cc21 ; C[Si]1(C)c2ccc3c(oc4ccccc43)c2-c2cccc(N(c3ccccc3)c3ccc(-c4ccc5ccccc5c4)cc3)c21]

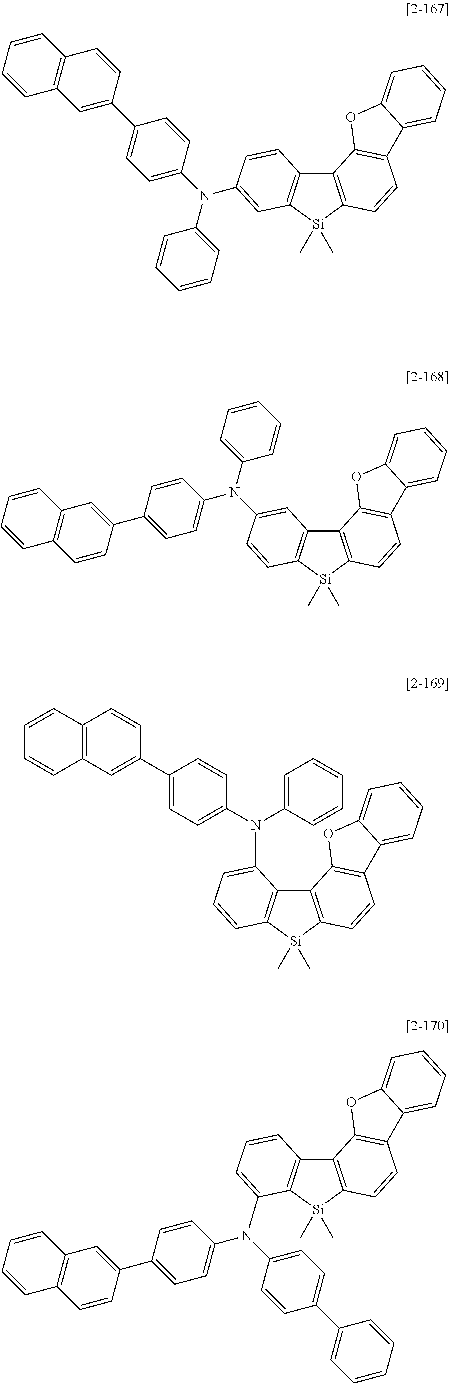 [SMILES: C[Si]1(C)c2cc(N(c3ccccc3)c3ccc(-c4ccc5ccccc5c4)cc3)ccc2-c2c1ccc1c2oc2ccccc21 ; C[Si]1(C)c2ccc(N(c3ccccc3)c3ccc(-c4ccc5ccccc5c4)cc3)cc2-c2c1ccc1c2oc2ccccc21 ; C[Si]1(C)c2cccc(N(c3ccccc3)c3ccc(-c4ccc5ccccc5c4)cc3)c2-c2c1ccc1c2oc2ccccc21 ; C[Si]1(C)c2ccc3c(oc4ccccc43)c2-c2cccc(N(c3ccc(-c4ccccc4)cc3)c3ccc(-c4ccc5ccccc5c4)cc3)c21]

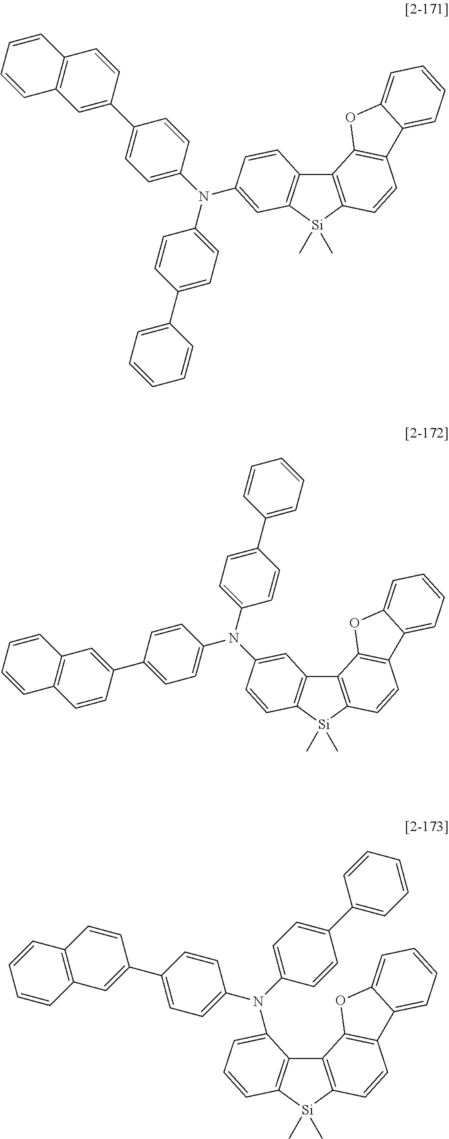 [SMILES: C[Si]1(C)c2cc(N(c3ccc(-c4ccccc4)cc3)c3ccc(-c4ccc5ccccc5c4)cc3)ccc2-c2c1ccc1c2oc2ccccc21 ; C[Si]1(C)c2ccc(N(c3ccc(-c4ccccc4)cc3)c3ccc(-c4ccc5ccccc5c4)cc3)cc2-c2c1ccc1c2oc2ccccc21 ; C[Si]1(C)c2cccc(N(c3ccc(-c4ccccc4)cc3)c3ccc(-c4ccc5ccccc5c4)cc3)c2-c2c1ccc1c2oc2ccccc21]

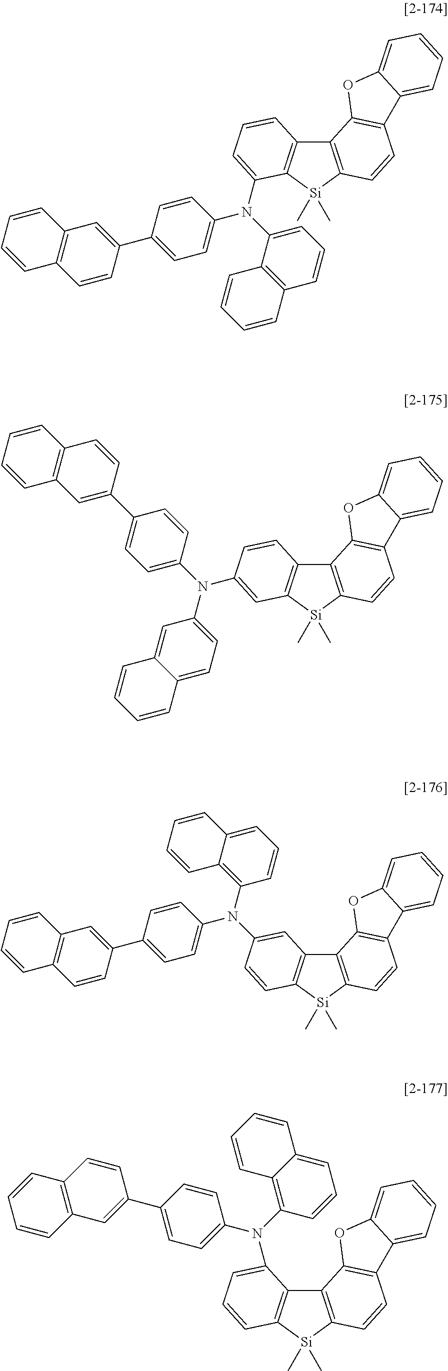 [SMILES: C[Si]1(C)c2ccc3c(oc4ccccc43)c2-c2cccc(N(c3ccc(-c4ccc5ccccc5c4)cc3)c3cccc4ccccc34)c21 ; C[Si]1(C)c2cc(N(c3ccc(-c4ccc5ccccc5c4)cc3)c3ccc4ccccc4c3)ccc2-c2c1ccc1c2oc2ccccc21 ; C[Si]1(C)c2ccc(N(c3ccc(-c4ccc5ccccc5c4)cc3)c3cccc4ccccc34)cc2-c2c1ccc1c2oc2ccccc21 ; C[Si]1(C)c2cccc(N(c3ccc(-c4ccc5ccccc5c4)cc3)c3cccc4ccccc34)c2-c2c1ccc1c2oc2ccccc21]

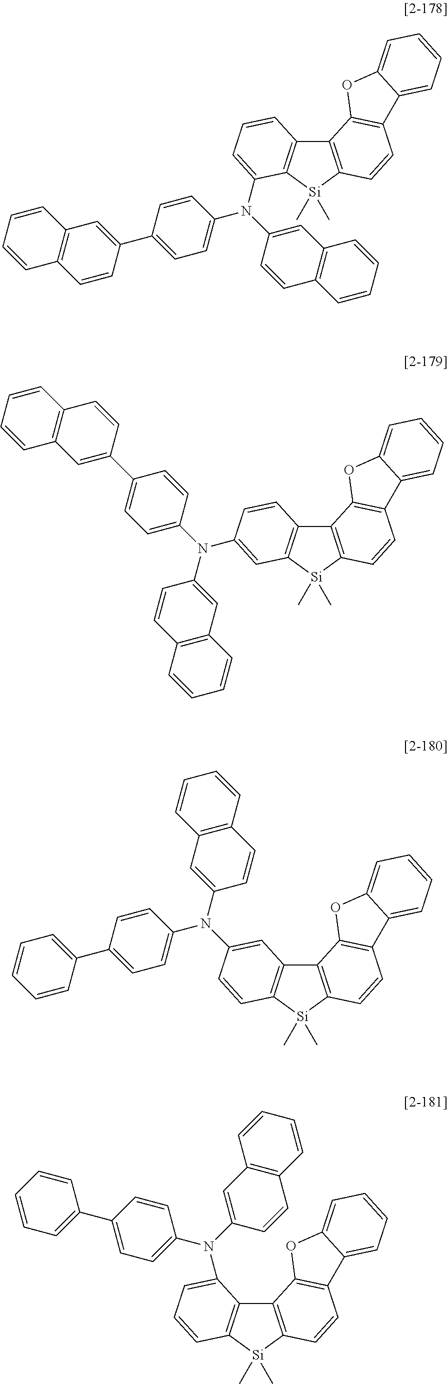 [SMILES: C[Si]1(C)c2ccc3c(oc4ccccc43)c2-c2cccc(N(c3ccc(-c4ccc5ccccc5c4)cc3)c3ccc4ccccc4c3)c21 ; C[Si]1(C)c2cc(N(c3ccc(-c4ccc5ccccc5c4)cc3)c3ccc4ccccc4c3)ccc2-c2c1ccc1c2oc2ccccc21 ; C[Si]1(C)c2ccc(N(c3ccc(-c4ccccc4)cc3)c3ccc4ccccc4c3)cc2-c2c1ccc1c2oc2ccccc21 ; C[Si]1(C)c2cccc(N(c3ccc(-c4ccccc4)cc3)c3ccc4ccccc4c3)c2-c2c1ccc1c2oc2ccccc21]

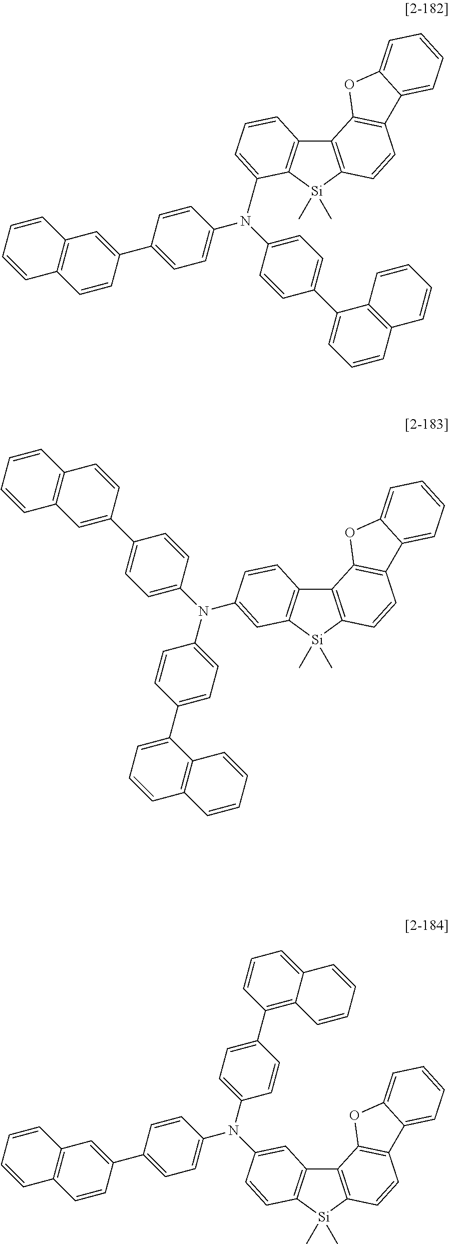 [SMILES: C[Si]1(C)c2ccc3c(oc4ccccc43)c2-c2cccc(N(c3ccc(-c4ccc5ccccc5c4)cc3)c3ccc(-c4cccc5ccccc45)cc3)c21 ; C[Si]1(C)c2cc(N(c3ccc(-c4ccc5ccccc5c4)cc3)c3ccc(-c4cccc5ccccc45)cc3)ccc2-c2c1ccc1c2oc2ccccc21 ; C[Si]1(C)c2ccc(N(c3ccc(-c4ccc5ccccc5c4)cc3)c3ccc(-c4cccc5ccccc45)cc3)cc2-c2c1ccc1c2oc2ccccc21]

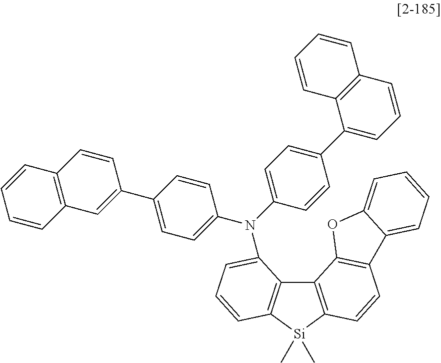 [SMILES: C[Si]1(C)c2cccc(N(c3ccc(-c4ccc5ccccc5c4)cc3)c3ccc(-c4cccc5ccccc45)cc3)c2-c2c1ccc1c2oc2ccccc21]

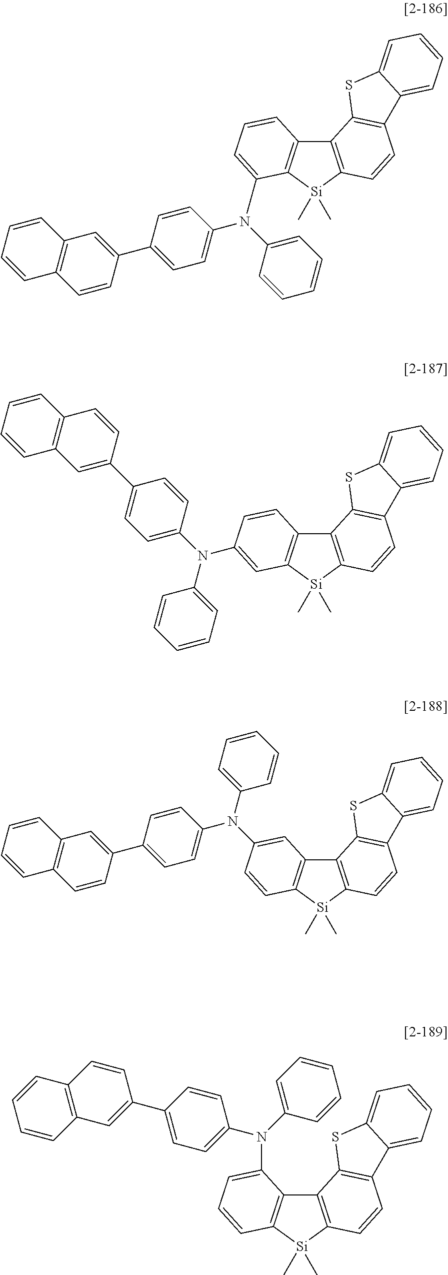 [SMILES: C[Si]1(C)c2ccc3c(sc4ccccc43)c2-c2cccc(N(c3ccccc3)c3ccc(-c4ccc5ccccc5c4)cc3)c21 ; C[Si]1(C)c2cc(N(c3ccccc3)c3ccc(-c4ccc5ccccc5c4)cc3)ccc2-c2c1ccc1c2sc2ccccc21 ; C[Si]1(C)c2ccc(N(c3ccccc3)c3ccc(-c4ccc5ccccc5c4)cc3)cc2-c2c1ccc1c2sc2ccccc21 ; C[Si]1(C)c2cccc(N(c3ccccc3)c3ccc(-c4ccc5ccccc5c4)cc3)c2-c2c1ccc1c2sc2ccccc21]

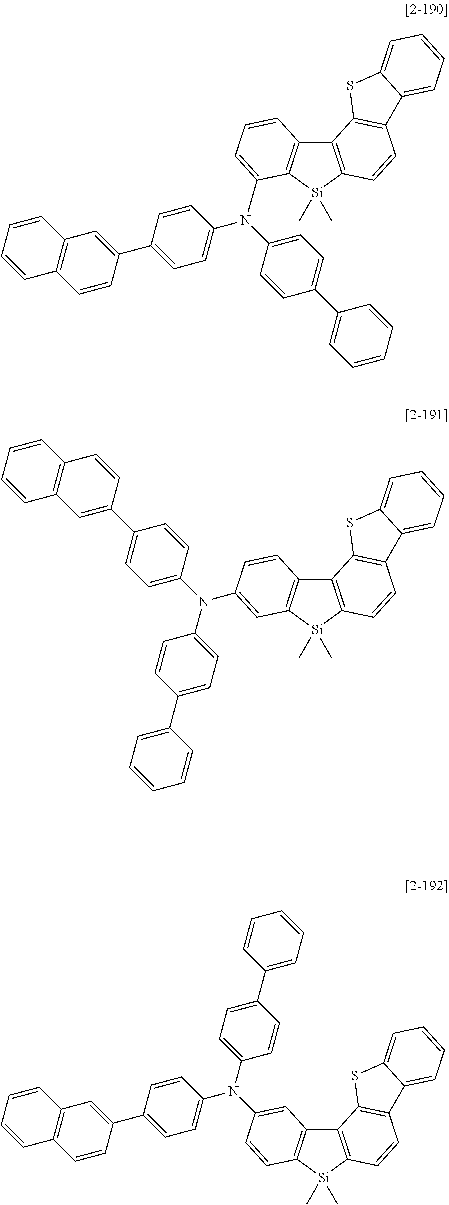 [SMILES: C[Si]1(C)c2ccc3c(sc4ccccc43)c2-c2cccc(N(c3ccc(-c4ccccc4)cc3)c3ccc(-c4ccc5ccccc5c4)cc3)c21 ; C[Si]1(C)c2cc(N(c3ccc(-c4ccccc4)cc3)c3ccc(-c4ccc5ccccc5c4)cc3)ccc2-c2c1ccc1c2sc2ccccc21 ; C[Si]1(C)c2ccc(N(c3ccc(-c4ccccc4)cc3)c3ccc(-c4ccc5ccccc5c4)cc3)cc2-c2c1ccc1c2sc2ccccc21]

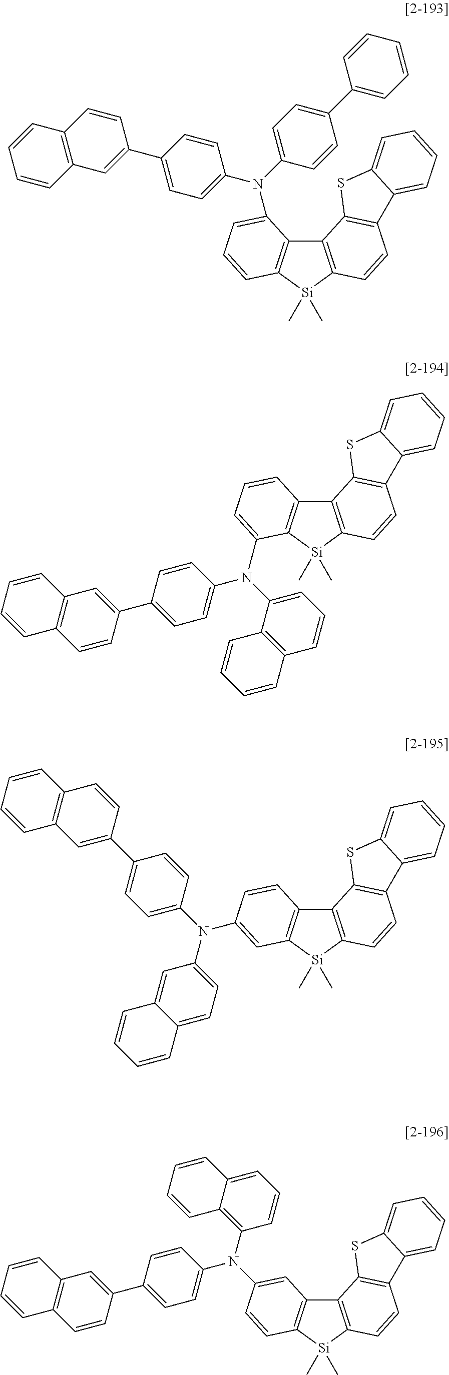 [SMILES: C[Si]1(C)c2cccc(N(c3ccc(-c4ccccc4)cc3)c3ccc(-c4ccc5ccccc5c4)cc3)c2-c2c1ccc1c2sc2ccccc21 ; C[Si]1(C)c2ccc3c(sc4ccccc43)c2-c2cccc(N(c3ccc(-c4ccc5ccccc5c4)cc3)c3cccc4ccccc34)c21 ; C[Si]1(C)c2cc(N(c3ccc(-c4ccc5ccccc5c4)cc3)c3ccc4ccccc4c3)ccc2-c2c1ccc1c2sc2ccccc21 ; C[Si]1(C)c2ccc(N(c3ccc(-c4ccc5ccccc5c4)cc3)c3cccc4ccccc34)cc2-c2c1ccc1c2sc2ccccc21]

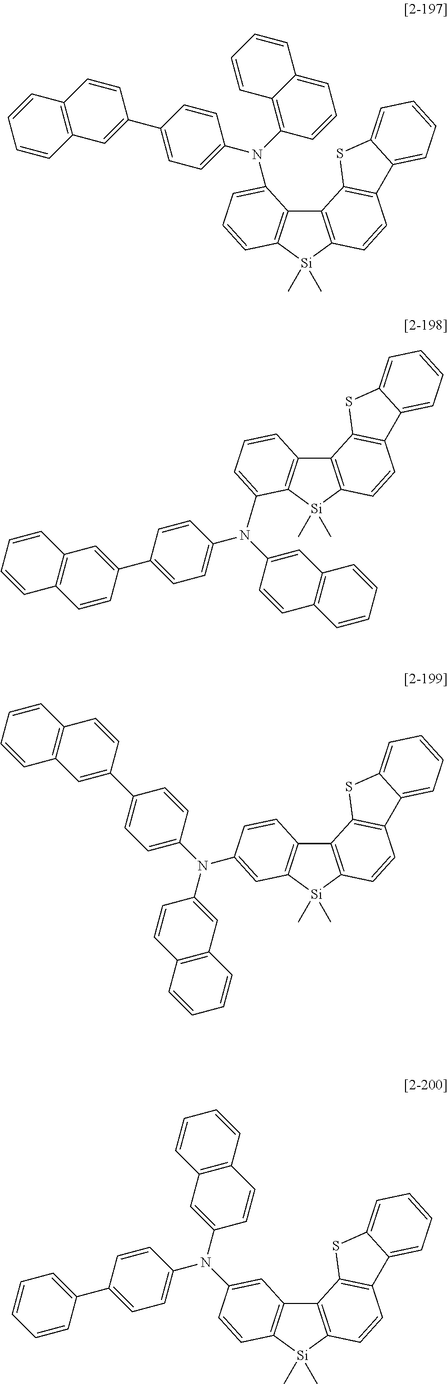 [SMILES: C[Si]1(C)c2cccc(N(c3ccc(-c4ccc5ccccc5c4)cc3)c3cccc4ccccc34)c2-c2c1ccc1c2sc2ccccc21 ; C[Si]1(C)c2ccc3c(sc4ccccc43)c2-c2cccc(N(c3ccc(-c4ccc5ccccc5c4)cc3)c3ccc4ccccc4c3)c21 ; C[Si]1(C)c2cc(N(c3ccc(-c4ccc5ccccc5c4)cc3)c3ccc4ccccc4c3)ccc2-c2c1ccc1c2sc2ccccc21 ; C[Si]1(C)c2ccc(N(c3ccc(-c4ccccc4)cc3)c3ccc4ccccc4c3)cc2-c2c1ccc1c2sc2ccccc21]

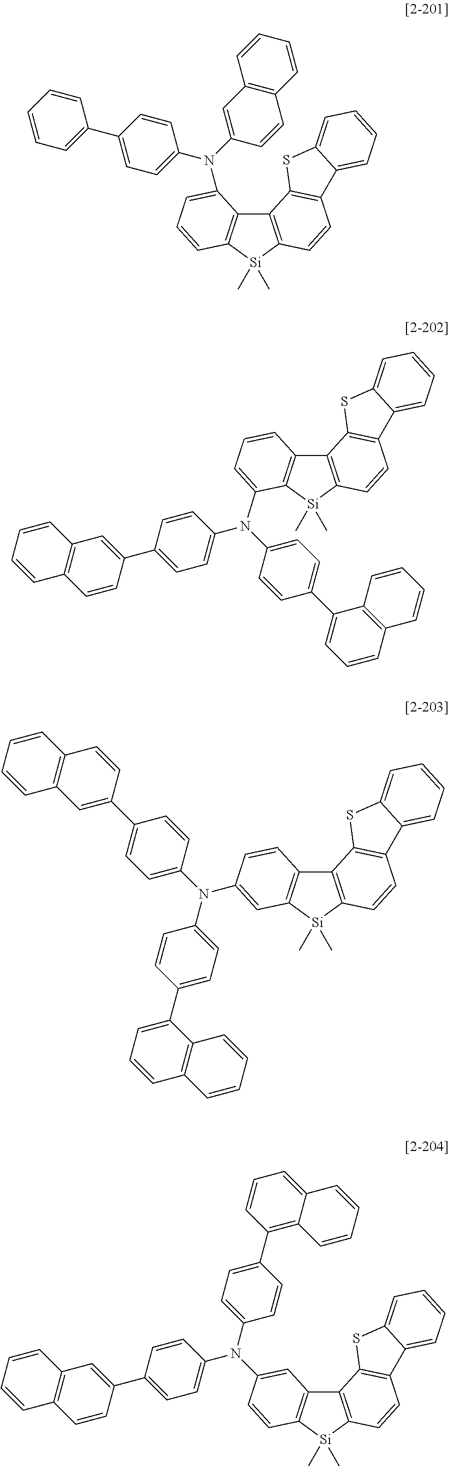 [SMILES: C[Si]1(C)c2cccc(N(c3ccc(-c4ccccc4)cc3)c3ccc4ccccc4c3)c2-c2c1ccc1c2sc2ccccc21 ; C[Si]1(C)c2ccc3c(sc4ccccc43)c2-c2cccc(N(c3ccc(-c4ccc5ccccc5c4)cc3)c3ccc(-c4cccc5ccccc45)cc3)c21 ; C[Si]1(C)c2cc(N(c3ccc(-c4ccc5ccccc5c4)cc3)c3ccc(-c4cccc5ccccc45)cc3)ccc2-c2c1ccc1c2sc2ccccc21 ; C[Si]1(C)c2ccc(N(c3ccc(-c4ccc5ccccc5c4)cc3)c3ccc(-c4cccc5ccccc45)cc3)cc2-c2c1ccc1c2sc2ccccc21]

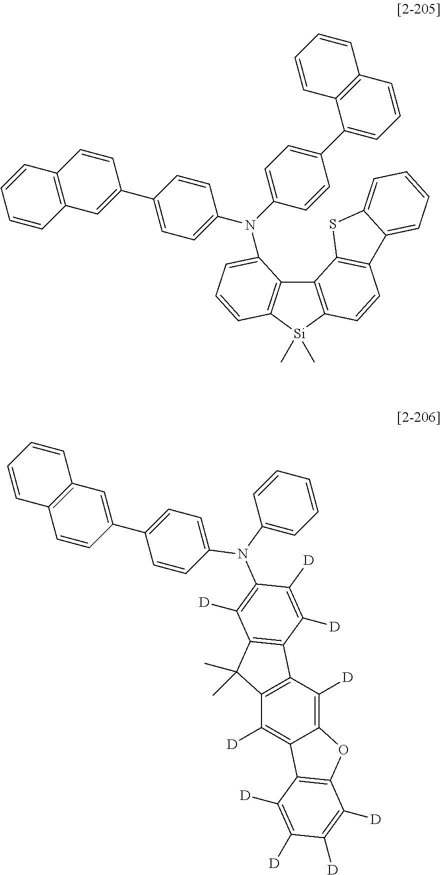 [SMILES: C[Si]1(C)c2cccc(N(c3ccc(-c4ccc5ccccc5c4)cc3)c3ccc(-c4cccc5ccccc45)cc3)c2-c2c1ccc1c2sc2ccccc21 ; [2H]c1c([2H])c(N(c2ccccc2)c2ccc(-c3ccc4ccccc4c3)cc2)c([2H])c2c1-c1c(c([2H])c3c(oc4c([2H])c([2H])c([2H])c([2H])c43)c1[2H])C2(C)C]

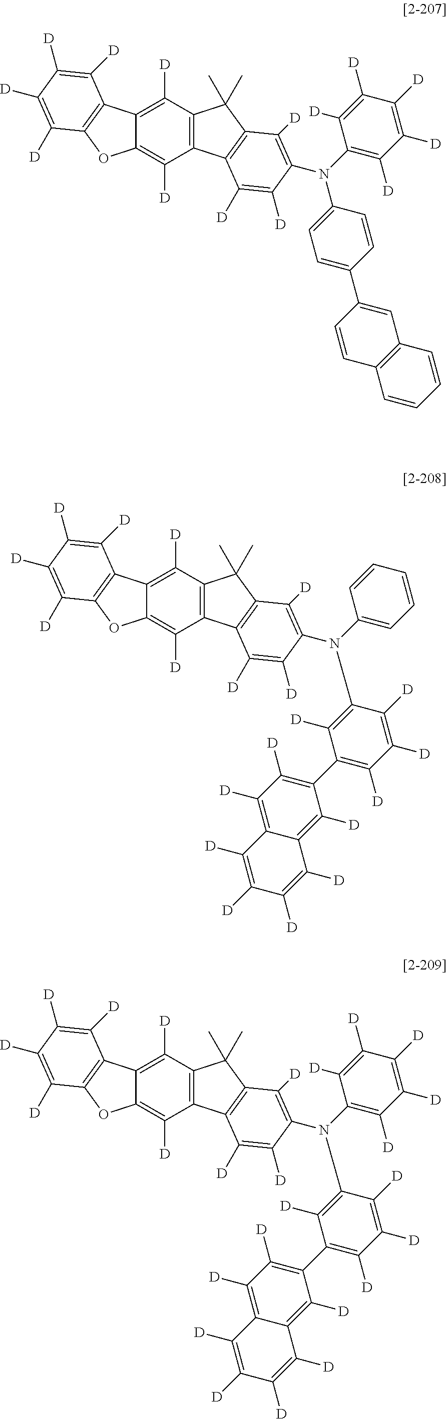

The first compound and the second compound may be included or mixed, e.g., in a weight ratio of about 1:99 to about 99:1. Within the range, a desirable weight ratio may be adjusted using an electron transport capability of the first compound and a hole transport capability of the second compound to realize bipolar characteristics and thus to improve efficiency and life-span. In an implementation, they may be, e.g., included in a weight ratio of about 10:90 to about 90:10, about 20:80 to about 80:20, about 20:80 to about 70:30, about 20:80 to about 60:40, or about 30:70 to about 60:40. In an implementation, they may be included in a weight ratio of, e.g., about 40:60, about 50:50, or about 60:40.

In an implementation, at least one compound may be further included in addition to the aforementioned first compound and second compound.

The aforementioned compound for the organic optoelectronic device or composition for the organic optoelectronic device may be a composition further including a dopant.

The dopant may be, e.g., a phosphorescent dopant, such as a red, green, or blue phosphorescent dopant, and may be, e.g., a red or green phosphorescent dopant.

The dopant is a material mixed with the compound or composition for an organic optoelectronic device in a small amount to cause light emission and may be generally a material such as a metal complex that emits light by multiple excitation into a triplet or more. The dopant may be, e.g., an inorganic, organic, or organic-inorganic compound, and one or more types thereof may be used.

Examples of the dopant may be a phosphorescent dopant and examples of the phosphorescent dopant may be an organic metal compound including, e.g., Ir, Pt, Os, Ti, Zr, Hf, Eu, Tb, Tm, Fe, Co, Ni, Ru, Rh, Pd, or a combination thereof. In an implementation, the phosphorescent dopant may be, e.g., a compound represented by Chemical Formula Z.

L⁶MX³ [Chemical Formula Z]

In Chemical Formula Z, M may be, e.g., a metal, L⁶ and X³ may each independently be, e.g., ligands forming a complex compound with M.

The M may be, e.g., Ir, Pt, Os, Ti, Zr, Hf, Eu, Tb, Tm, Fe, Co, Ni, Ru, Rh, Pd, or a combination thereof, and L⁶ and X³ may be, e.g., a bidentate ligand.

In an implementation, the ligand represented by L⁶ and X³ may be, e.g., a ligand of Group A.

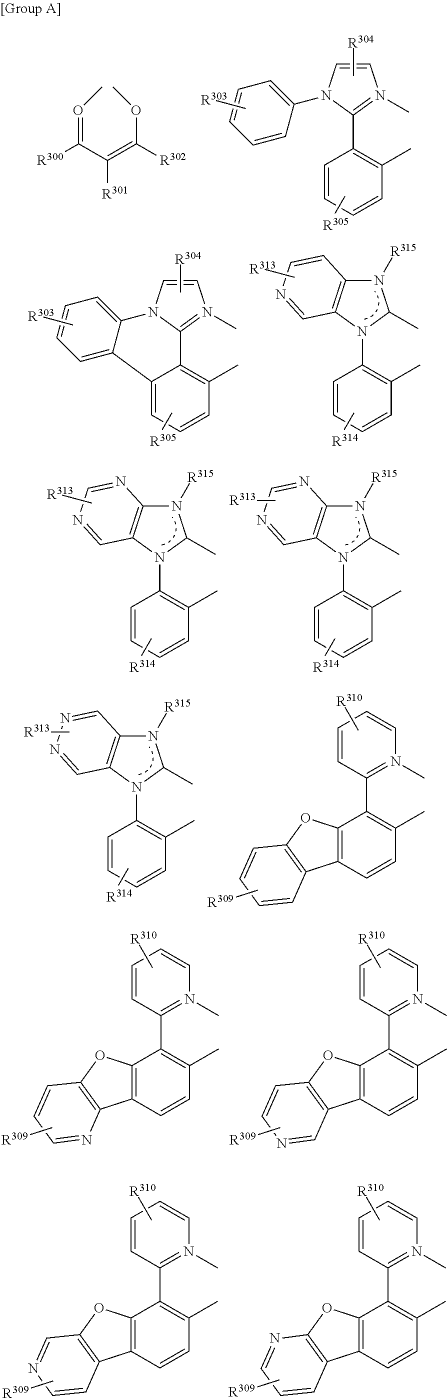

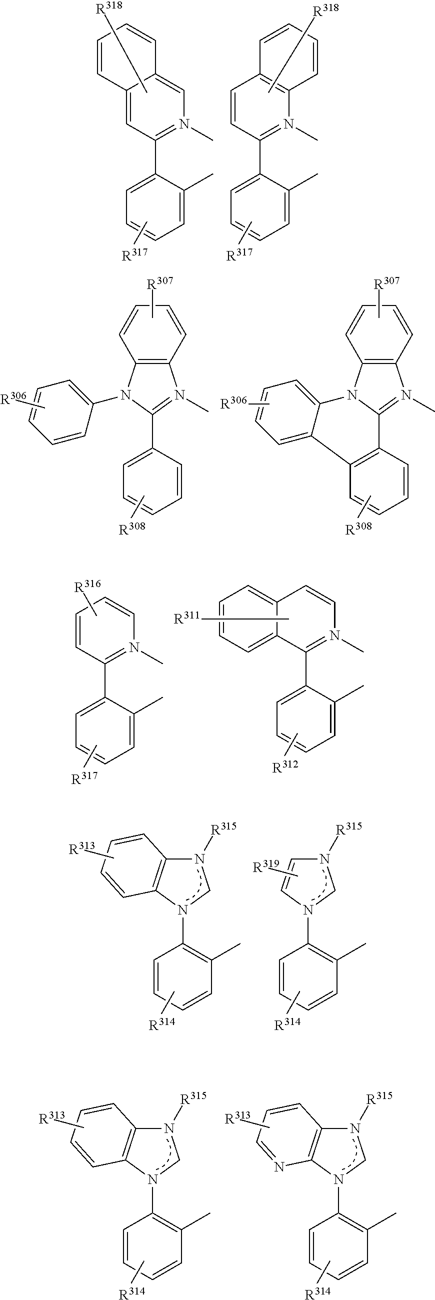

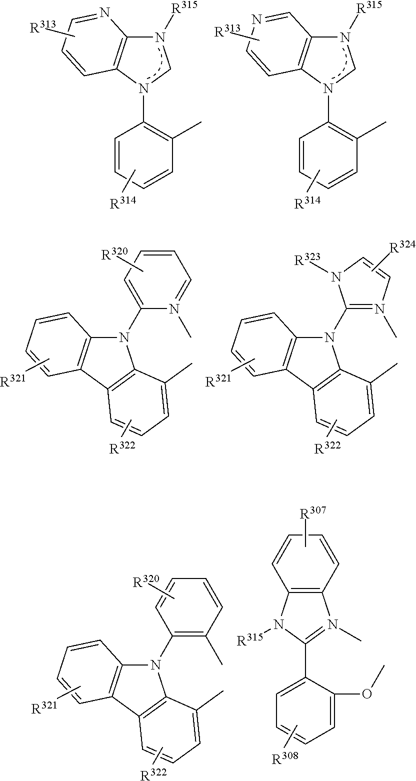

In Group A, R³⁰⁰ to R³⁰² may each independently be, e.g., hydrogen, deuterium a C1 to C30 alkyl group that is substituted or unsubstituted with a halogen, a C6 to C30 aryl group that is substituted or unsubstituted with a C1 to C30 alkyl, or a halogen.

R³⁰³ to R³²⁴ may each independently be, e.g., hydrogen, deuterium, a halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group and C6 to C30 aryl group, or a triarylsilyl group having a substituted or unsubstituted C6 to C30 aryl group.

In an implementation, a dopant represented by Chemical Formula I may be included.

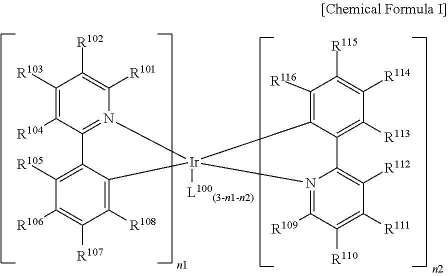

In Chemical Formula I, R¹⁰¹ to R¹¹⁶ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

R¹³² to R¹³⁴ may each independently be, e.g., a C1 to C6 alkyl group.

In an implementation, at least one of R¹⁰¹ to R¹¹⁶ may be, e.g., a functional group represented by Chemical Formula I-1.

L¹⁰⁰ may be, e.g., a bidentate ligand of a monovalent anion, and is a ligand that coordinates to iridium through a lone pair of carbons or heteroatoms.

n1 and n2 may each independently be, e.g., an integer of 0 to 3, and n1+n2 may e, e.g., an integer of 1 to 3.

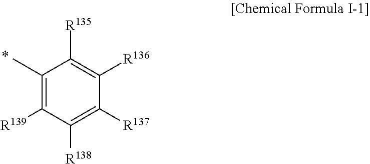

In Chemical Formula I-1, R¹³⁵ to R¹³⁹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

R¹³² to R¹³⁴ may each independently be, e.g., a C1 to C6 alkyl group.

* indicates a portion linked to a carbon atom.

In an implementation, a dopant represented by Chemical Formula Z-1 may be included.

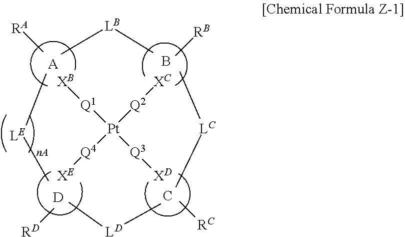

In Chemical Formula Z-1, rings A, B, C, and D may each independently be, e.g., a 5-membered or 6-membered carbocyclic or heterocyclic ring.

R^{A}, R^{B}, R^{C}, and R^{D} may each independently be, e.g., mono-, di-, tri-, or tetra-substitution, or unsubstitution.

L^{B}, L^{C}, and L^{D} may each independently be, e.g., a direct bond, BR, NR, PR, O, S, Se, C═O, S═O, SO₂, CRR′, SiRR′, GeRR′, or a combination thereof.

In an implementation, when nA is 1, L^{E} may be, e.g., a direct bond, BR, NR, PR, O, S, Se, C═O, S═O, SO₂, CRR′, SiRR′, GeRR′, or a combination thereof; and when nA is 0, L^{E} does not exist.

R^{A}, R^{B}, R^{C}, R^{D}, R, and R′ may each independently be, e.g., hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof; any adjacent R^{A}, R^{B}, R^{C}, R^{D}, R, and R′ may be optionally linked to each other to provide a ring; X^{B}, X^{C}, X^{D}, and X^{E} may each independently be, e.g., carbon and nitrogen; and Q¹, Q², Q³, and Q⁴ may each independently be, e.g., oxygen or a direct bond.

The dopant according to an embodiment may be a platinum complex, and may be, e.g., represented by Chemical Formula II.

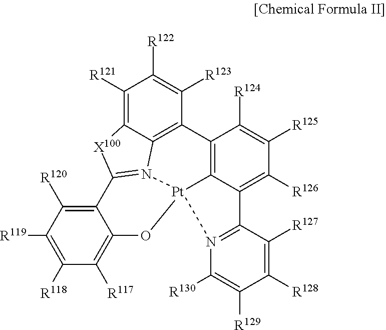

In Chemical Formula II, X¹⁰⁰ may be, e.g., O, S, or NR¹³¹.

R¹¹⁷ to R¹³¹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

R¹³² to R¹³⁴ may each independently be, e.g., a C1 to C6 alkyl group.

In an implementation, at least one of R¹¹⁷ to R¹³¹ may be, e.g., —SiR¹³²R¹³³R¹³⁴ or a tert-butyl group.

Hereinafter, an organic optoelectronic device including the aforementioned compound for an organic optoelectronic device or composition for an organic optoelectronic device is described.

The organic optoelectronic device may be a suitable device to convert electrical energy into photoenergy and vice versa, e.g., an organic photoelectric device, an organic light emitting diode, an organic solar cell, or an organic photo-conductor drum.

Herein, an organic light emitting diode as one example of an organic optoelectronic device is described referring to drawings.

The FIGURE a cross-sectional view illustrating an organic light emitting diode according to an embodiment.

Referring to the FIGURE, an organic light emitting diode 100 according to an embodiment may include an anode 120 and a cathode 110 facing each other and an organic layer 105 between the anode 120 and cathode 110.

The anode 120 may be made of a conductor having a large work function to help hole injection, and may be, e.g., a metal, a metal oxide, or a conductive polymer. The anode 120 may be, e.g., a metal such as nickel, platinum, vanadium, chromium, copper, zinc, gold, or the like or an alloy thereof; a metal oxide such as zinc oxide, indium oxide, indium tin oxide (ITO), indium zinc oxide (IZO), or the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, or polyaniline.

The cathode 110 may be made of a conductor having a small work function to help electron injection, and may be, e.g., a metal, a metal oxide, or a conductive polymer. The cathode 110 may be, e.g., a metal such as magnesium, calcium, sodium, potassium, titanium, indium, yttrium, lithium, gadolinium, aluminum silver, tin, lead, cesium, barium, or the like, or an alloy thereof; or a multi-layer structure material such as LiF/Al, LiO₂/Al, LiF/Ca, or BaF₂/Ca.

The organic layer 105 may include the aforementioned compound for an organic optoelectronic device or composition for an organic optoelectronic device.

The organic layer 105 may include a light emitting layer 130 and the light emitting layer 130 may include the aforementioned compound for an organic optoelectronic device or composition for an organic optoelectronic device.

The composition for an organic optoelectronic device further including the dopant may be, e.g., a red light emitting composition.

The light emitting layer 130 may include, e.g., the aforementioned first compound and second compound as a phosphorescent host.

The organic layer may further include a charge transport region in addition to the light emitting layer.

The charge transport region may be, e.g., the hole transport region 140.

The hole transport region 140 may further increase hole injection or hole mobility between the anode 120 and the light emitting layer 130 and block electrons. In an implementation, the hole transport region 140 may include a hole transport layer between the anode 120 and the light emitting layer 130, and a hole transport auxiliary layer between the light emitting layer 130 and the hole transport layer, and a compound of Group B may be included in at least one of the hole transport layer and the hole transport auxiliary layer.

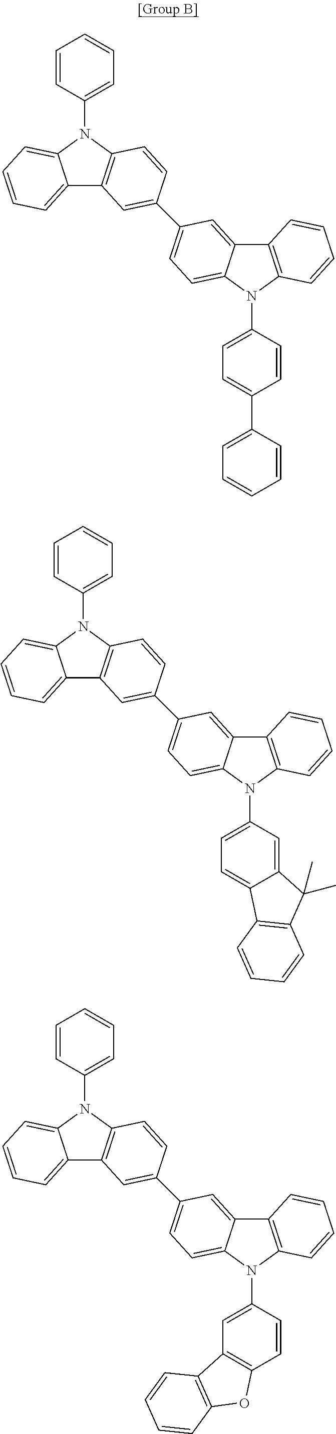 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

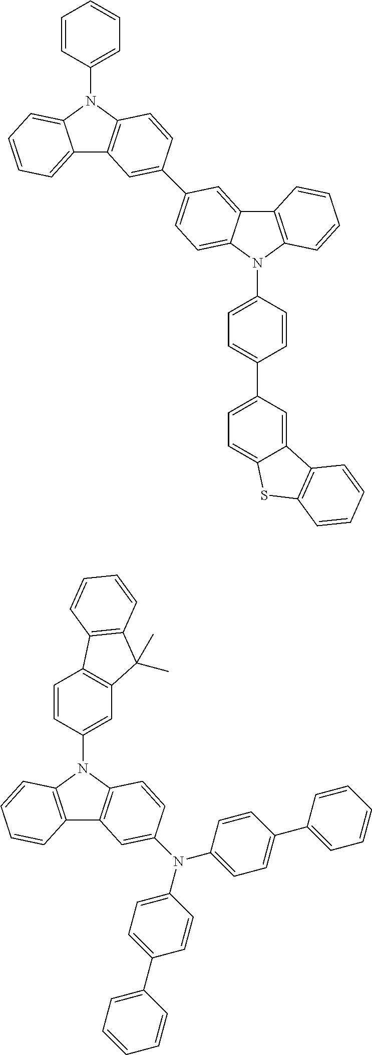 [SMILES: c1ccc(-n2c3ccccc3c3cc(-c4ccc5c(c4)c4ccccc4n5-c4ccc(-c5ccc6sc7ccccc7c6c5)cc4)ccc32)cc1 ; CC1(C)c2ccccc2-c2ccc(-n3c4ccccc4c4cc(N(c5ccc(-c6ccccc6)cc5)c5ccc(-c6ccccc6)cc5)ccc43)cc21]

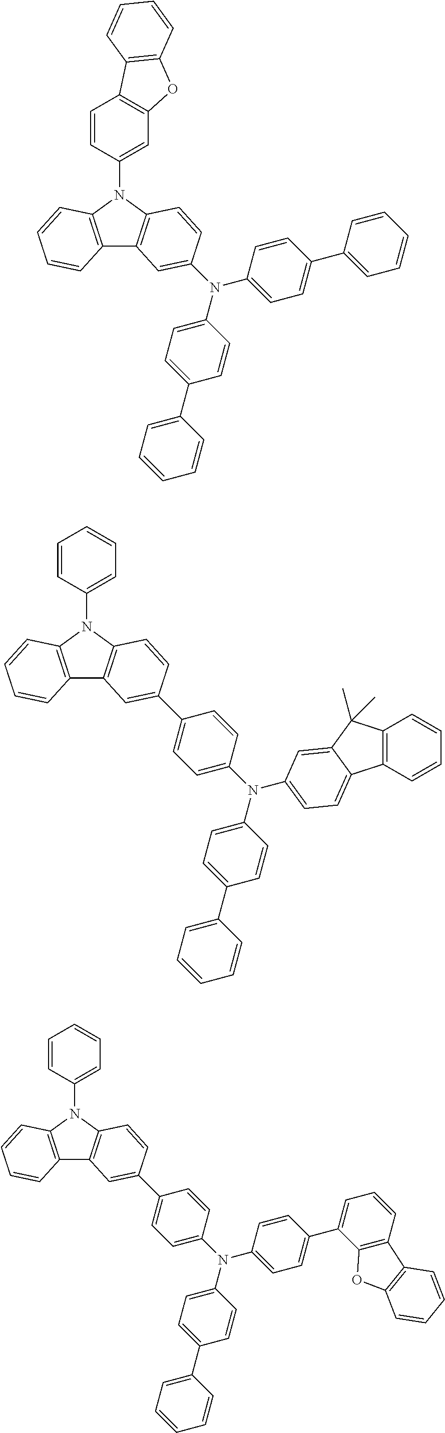 [SMILES: c1ccc(-c2ccc(N(c3ccc(-c4ccccc4)cc3)c3ccc4c(c3)c3ccccc3n4-c3ccc4c(c3)oc3ccccc34)cc2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1]

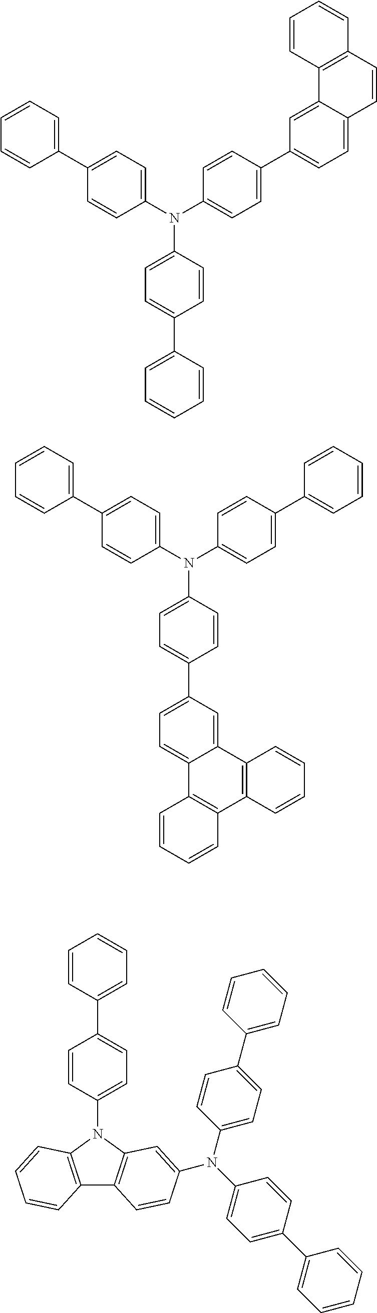 [SMILES: c1ccc(-c2ccc(N(c3ccc(-c4ccccc4)cc3)c3ccc(-c4ccc5ccc6ccccc6c5c4)cc3)cc2)cc1 ; c1ccc(-c2ccc(N(c3ccc(-c4ccccc4)cc3)c3ccc(-c4ccc5c6ccccc6c6ccccc6c5c4)cc3)cc2)cc1 ; c1ccc(-c2ccc(N(c3ccc(-c4ccccc4)cc3)c3ccc4c5ccccc5n(-c5ccc(-c6ccccc6)cc5)c4c3)cc2)cc1]

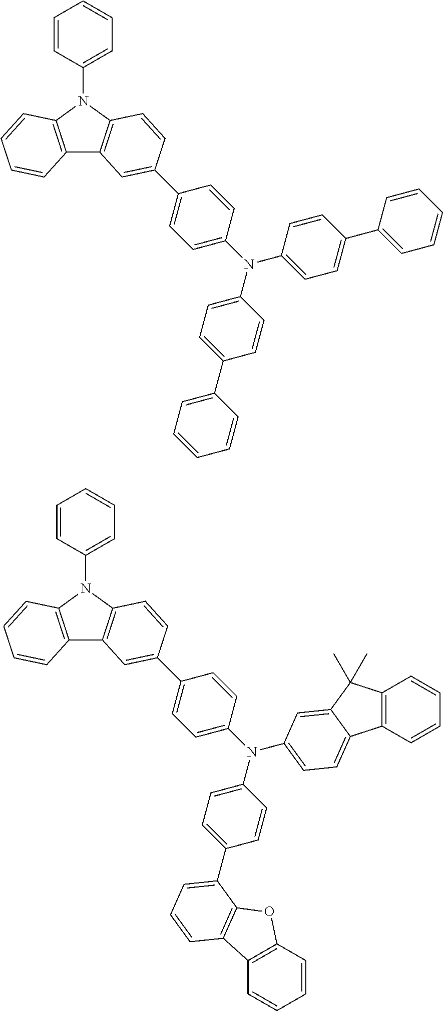 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21]

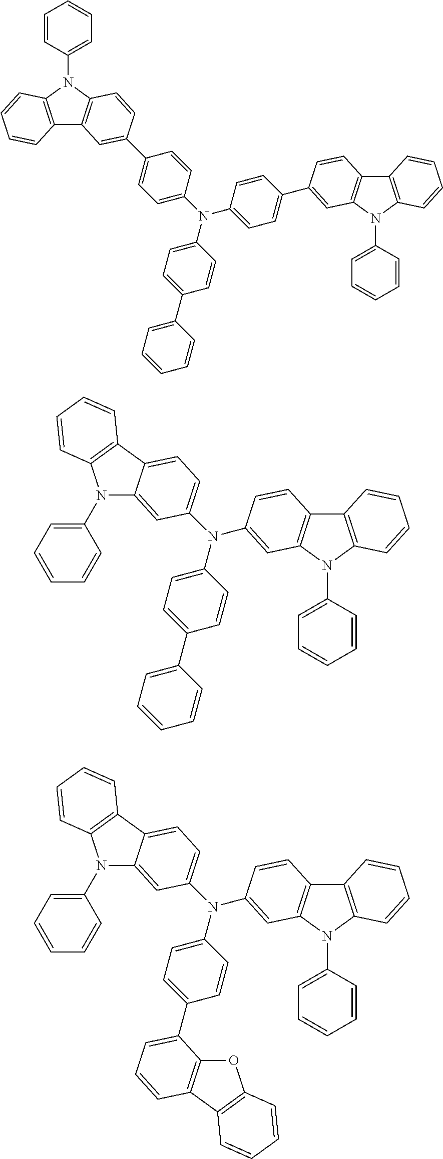 [SMILES: c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1 ; c1ccc(-c2ccc(N(c3ccc4c5ccccc5n(-c5ccccc5)c4c3)c3ccc4c5ccccc5n(-c5ccccc5)c4c3)cc2)cc1 ; c1ccc(-n2c3ccccc3c3ccc(N(c4ccc(-c5cccc6c5oc5ccccc56)cc4)c4ccc5c6ccccc6n(-c6ccccc6)c5c4)cc32)cc1]

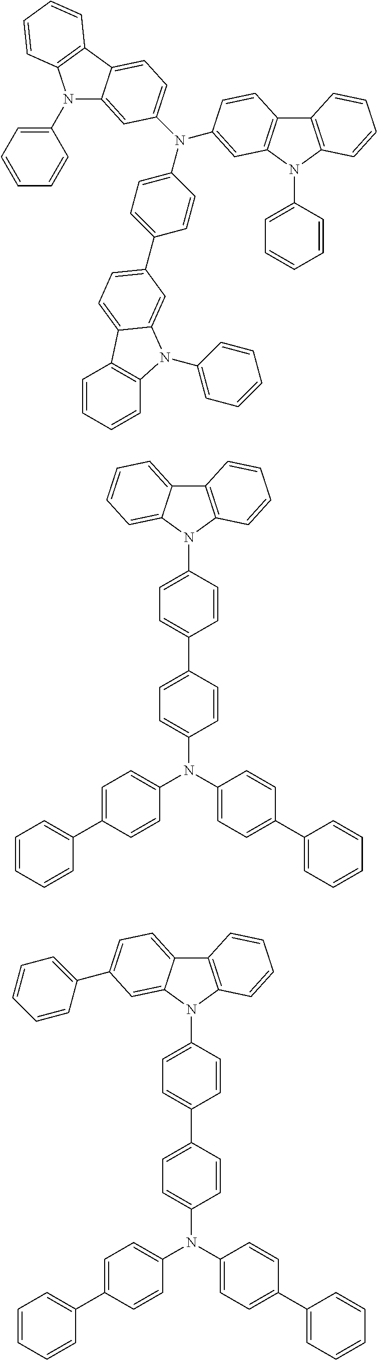

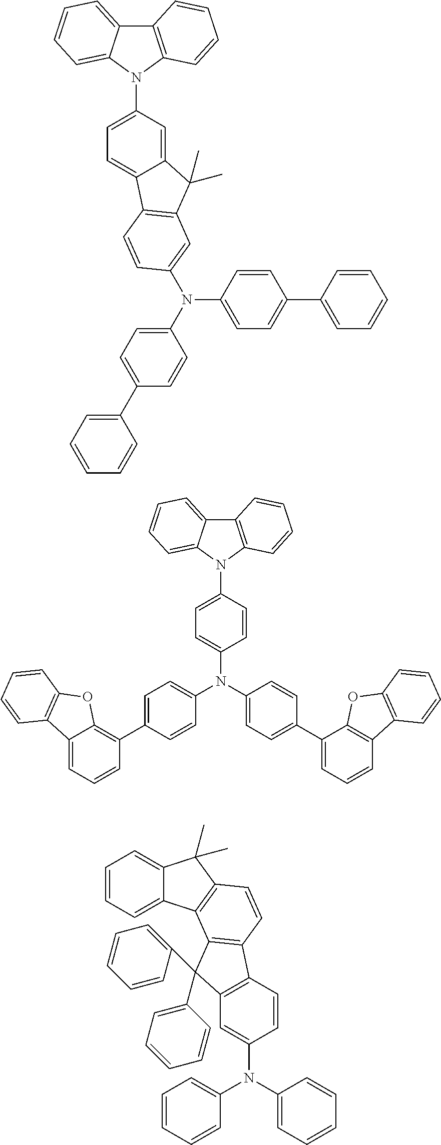 [SMILES: CC1(C)c2cc(N(c3ccc(-c4ccccc4)cc3)c3ccc(-c4ccccc4)cc3)ccc2-c2ccc(-n3c4ccccc4c4ccccc43)cc21 ; c1ccc2c(c1)oc1c(-c3ccc(N(c4ccc(-c5cccc6c5oc5ccccc56)cc4)c4ccc(-n5c6ccccc6c6ccccc65)cc4)cc3)cccc12 ; CC1(C)c2ccccc2-c2c1ccc1c2C(c2ccccc2)(c2ccccc2)c2cc(N(c3ccccc3)c3ccccc3)ccc2-1]

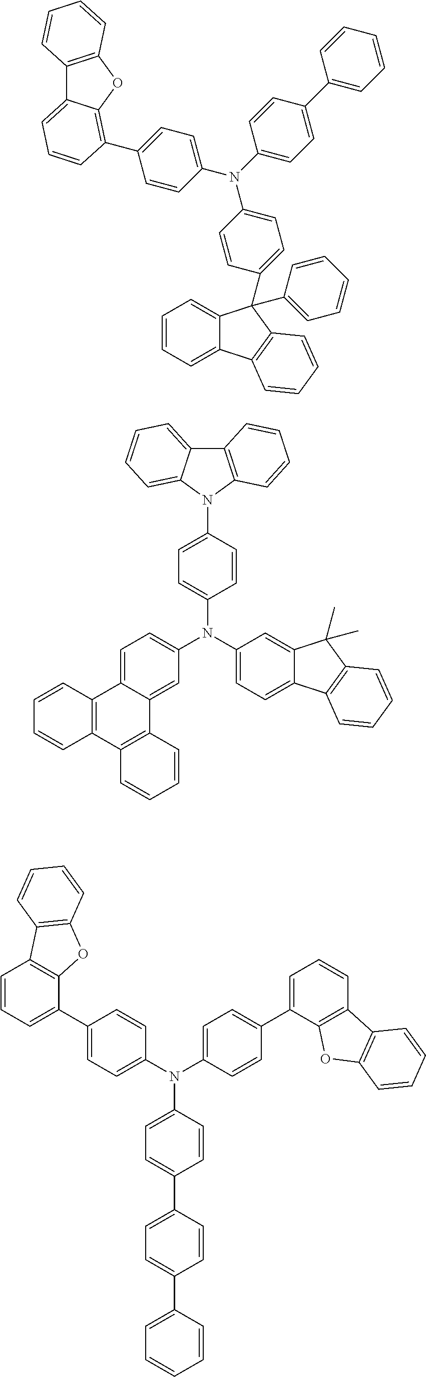

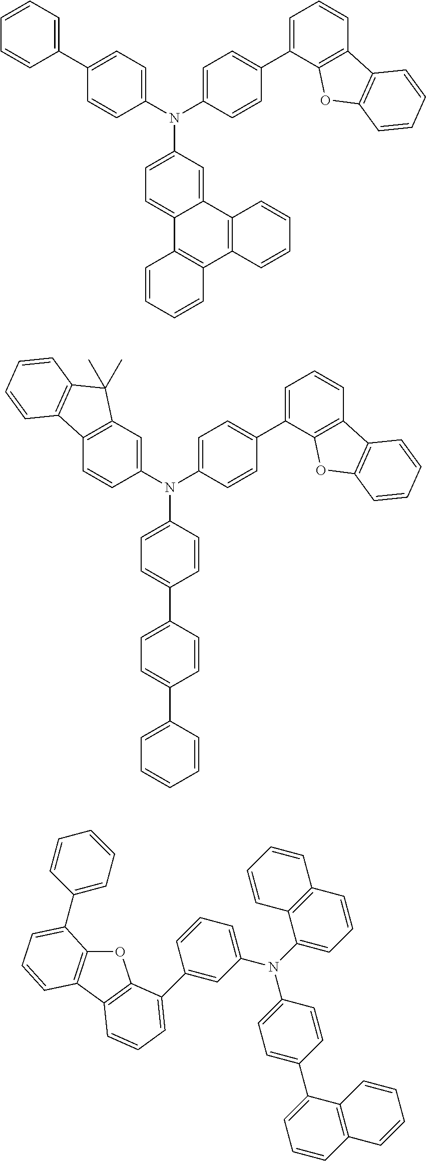 [SMILES: c1ccc(-c2ccc(N(c3ccc(-c4cccc5c4oc4ccccc45)cc3)c3ccc4c5ccccc5c5ccccc5c4c3)cc2)cc1 ; CC1(C)c2ccccc2-c2ccc(N(c3ccc(-c4ccc(-c5ccccc5)cc4)cc3)c3ccc(-c4cccc5c4oc4ccccc45)cc3)cc21 ; c1ccc(-c2cccc3c2oc2c(-c4cccc(N(c5ccc(-c6cccc7ccccc67)cc5)c5cccc6ccccc56)c4)cccc23)cc1]

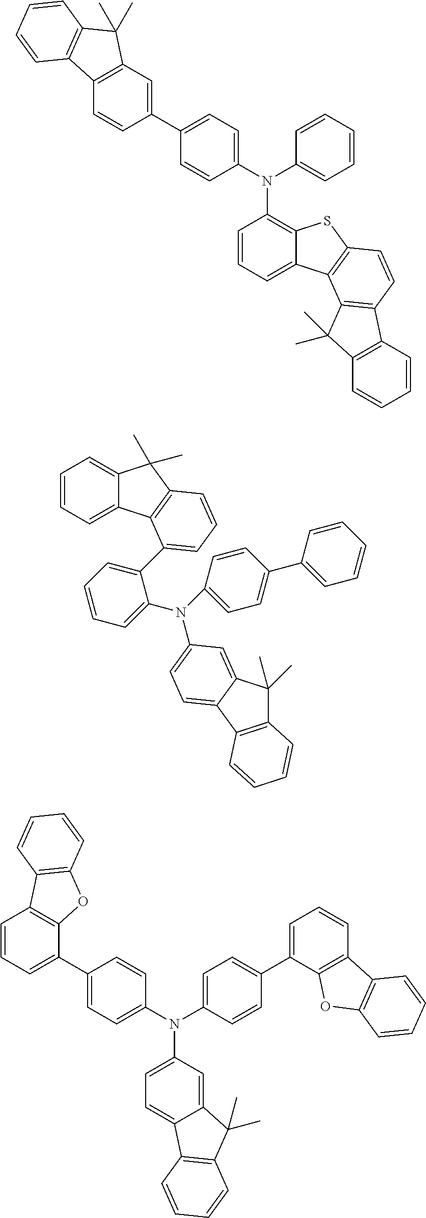 [SMILES: CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21 ; CC1(C)c2ccccc2-c2ccc(N(c3ccc(-c4cccc5c4oc4ccccc45)cc3)c3ccc(-c4cccc5c4oc4ccccc45)cc3)cc21]

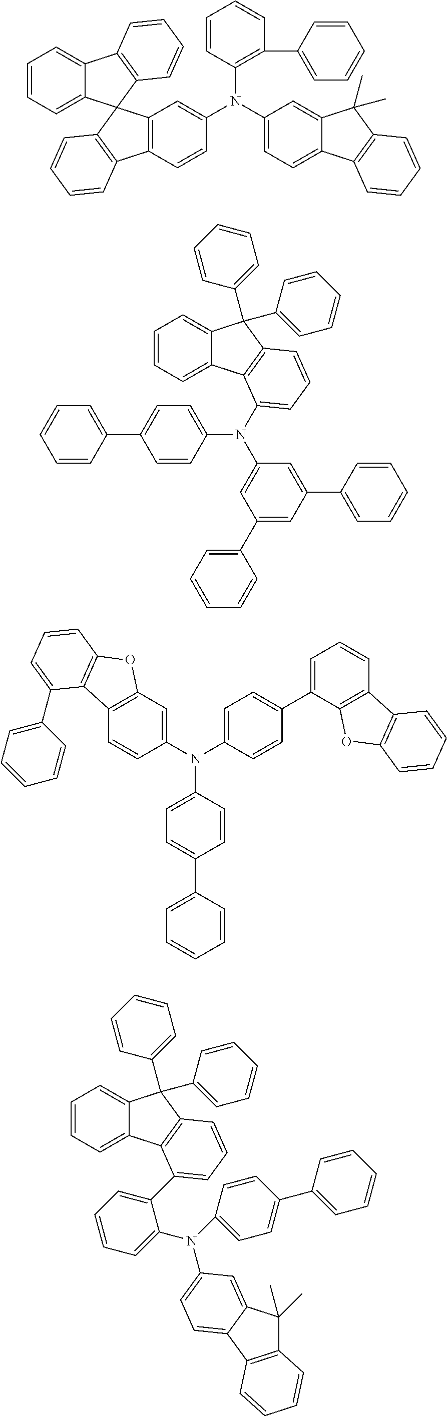 [SMILES: CC1(C)c2ccccc2-c2ccc(N(c3ccc4c(c3)C3(c5ccccc5-c5ccccc53)c3ccccc3-4)c3ccccc3-c3ccccc3)cc21 ; c1ccc(-c2ccc(N(c3cc(-c4ccccc4)cc(-c4ccccc4)c3)c3cccc4c3-c3ccccc3C4(c3ccccc3)c3ccccc3)cc2)cc1 ; c1ccc(-c2ccc(N(c3ccc(-c4cccc5c4oc4ccccc45)cc3)c3ccc4c(c3)oc3cccc(-c5ccccc5)c34)cc2)cc1 ; CC1(C)c2ccccc2-c2ccc(N(c3ccc(-c4ccccc4)cc3)c3ccccc3-c3cccc4c3-c3ccccc3C4(c3ccccc3)c3ccccc3)cc21]

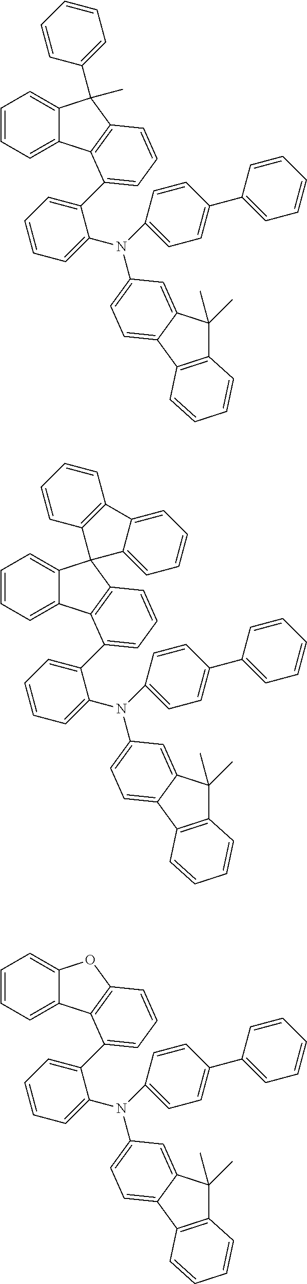 [SMILES: CC1(C)c2ccccc2-c2ccc(N(c3ccc(-c4ccccc4)cc3)c3ccccc3-c3cccc4c3-c3ccccc3C4(C)c3ccccc3)cc21 ; CC1(C)c2ccccc2-c2ccc(N(c3ccc(-c4ccccc4)cc3)c3ccccc3-c3cccc4c3-c3ccccc3C43c4ccccc4-c4ccccc43)cc21 ; CC1(C)c2ccccc2-c2ccc(N(c3ccc(-c4ccccc4)cc3)c3ccccc3-c3cccc4oc5ccccc5c34)cc21]

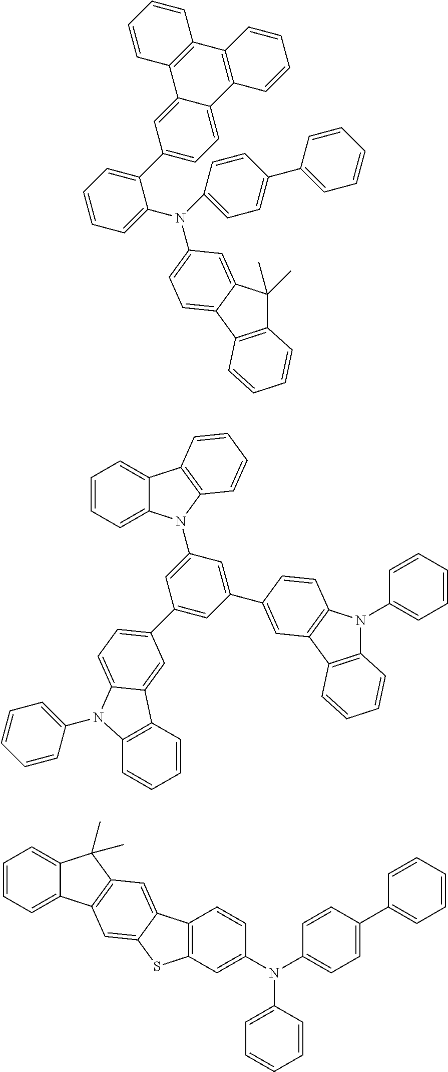 [SMILES: CC1(C)c2ccccc2-c2ccc(N(c3ccc(-c4ccccc4)cc3)c3ccccc3-c3ccc4c5ccccc5c5ccccc5c4c3)cc21 ; c1ccc(-n2c3ccccc3c3cc(-c4cc(-c5ccc6c(c5)c5ccccc5n6-c5ccccc5)cc(-n5c6ccccc6c6ccccc65)c4)ccc32)cc1 ; CC1(C)c2ccccc2-c2cc3sc4cc(N(c5ccccc5)c5ccc(-c6ccccc6)cc5)ccc4c3cc21]

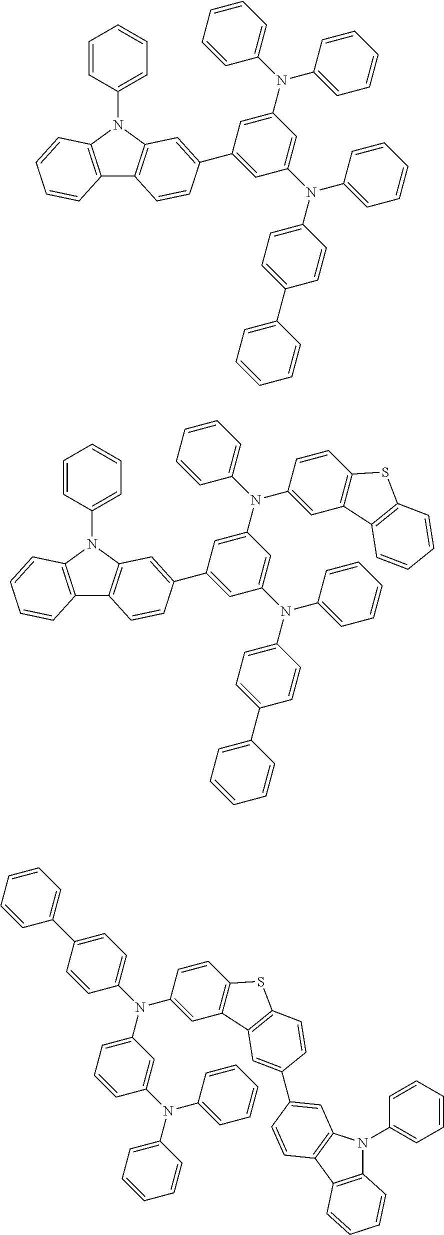 [SMILES: c1ccc(-c2ccc(N(c3ccccc3)c3cc(-c4ccc5c6ccccc6n(-c6ccccc6)c5c4)cc(N(c4ccccc4)c4ccccc4)c3)cc2)cc1 ; c1ccc(-c2ccc(N(c3ccccc3)c3cc(-c4ccc5c6ccccc6n(-c6ccccc6)c5c4)cc(N(c4ccccc4)c4ccc5sc6ccccc6c5c4)c3)cc2)cc1 ; c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1]

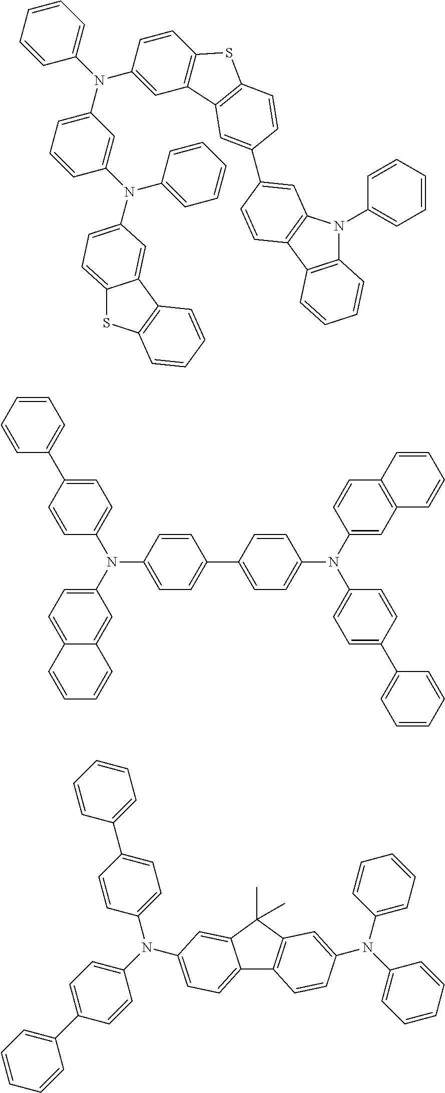 [SMILES: c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1 ; c1ccc(-c2ccc(N(c3ccc(-c4ccc(N(c5ccc(-c6ccccc6)cc5)c5ccc6ccccc6c5)cc4)cc3)c3ccc4ccccc4c3)cc2)cc1 ; CC1(C)c2cc(N(c3ccccc3)c3ccccc3)ccc2-c2ccc(N(c3ccc(-c4ccccc4)cc3)c3ccc(-c4ccccc4)cc3)cc21]

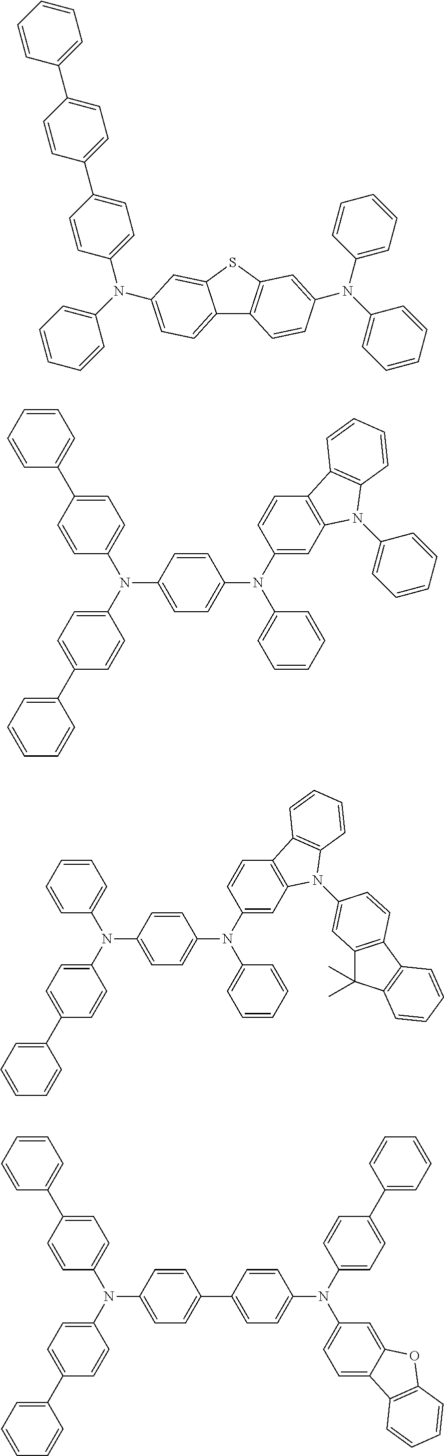

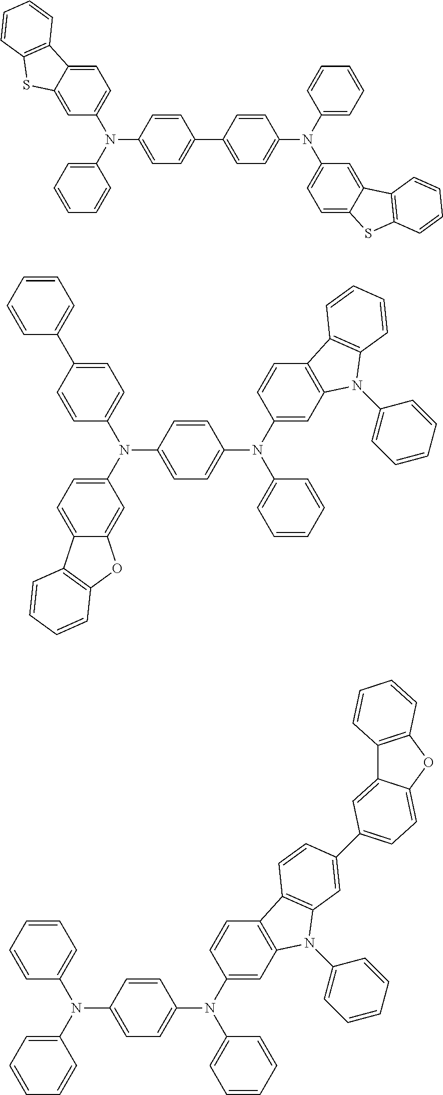 [SMILES: c1ccc(N(c2ccc(-c3ccc(N(c4ccccc4)c4ccc5sc6ccccc6c5c4)cc3)cc2)c2ccc3c(c2)sc2ccccc23)cc1 ; c1ccc(-c2ccc(N(c3ccc(N(c4ccccc4)c4ccc5c6ccccc6n(-c6ccccc6)c5c4)cc3)c3ccc4c(c3)oc3ccccc34)cc2)cc1 ; c1ccc(N(c2ccccc2)c2ccc(N(c3ccccc3)c3ccc4c5ccc(-c6ccc7oc8ccccc8c7c6)cc5n(-c5ccccc5)c4c3)cc2)cc1]

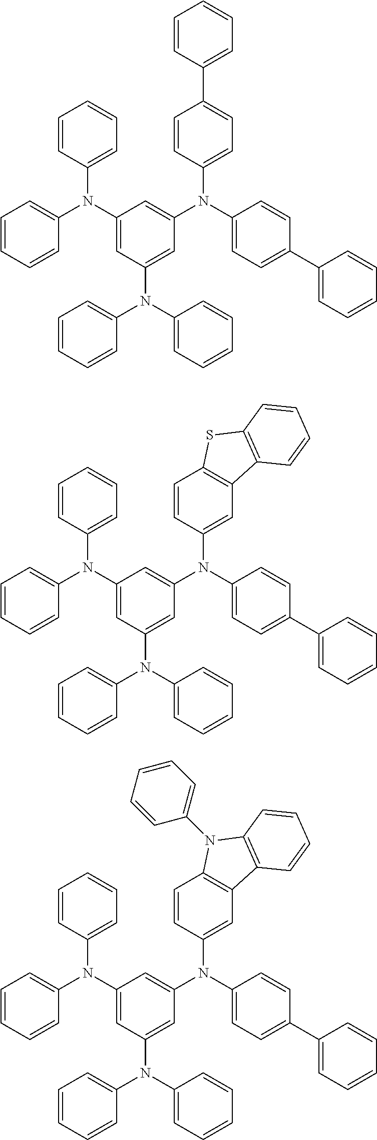

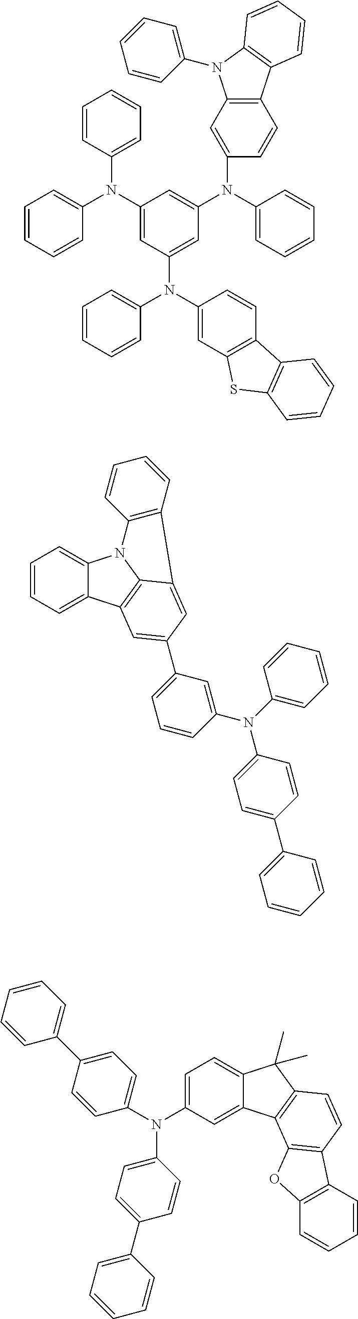 [SMILES: c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1 ; c1ccc(-c2ccc(N(c3ccccc3)c3cccc(-c4cc5c6ccccc6n6c7ccccc7c(c4)c56)c3)cc2)cc1 ; CC1(C)c2ccc(N(c3ccc(-c4ccccc4)cc3)c3ccc(-c4ccccc4)cc3)cc2-c2c1ccc1c2oc2ccccc21]

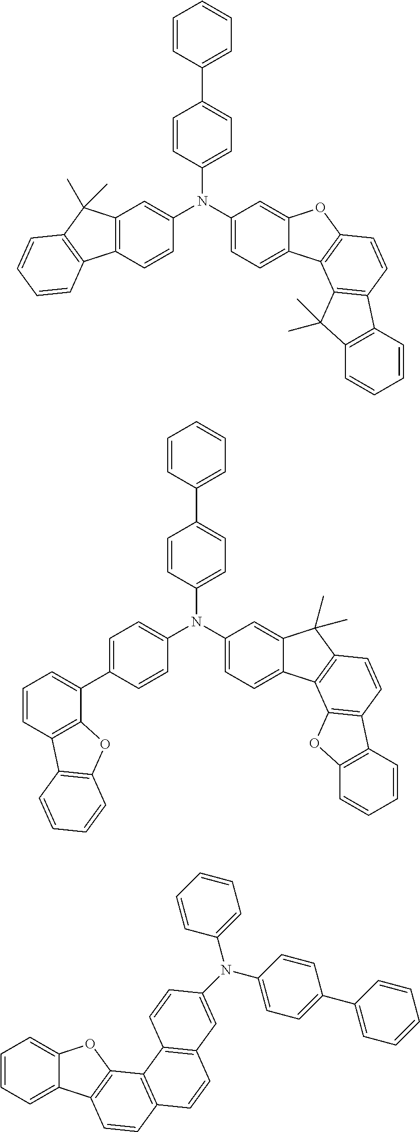 [SMILES: CC1(C)c2ccccc2-c2ccc(N(c3ccc(-c4ccccc4)cc3)c3ccc4c(c3)oc3ccc5c(c34)C(C)(C)c3ccccc3-5)cc21 ; CC1(C)c2cc(N(c3ccc(-c4ccccc4)cc3)c3ccc(-c4cccc5c4oc4ccccc45)cc3)ccc2-c2c1ccc1c2oc2ccccc21 ; c1ccc(-c2ccc(N(c3ccccc3)c3ccc4c(ccc5ccc6c7ccccc7oc6c54)c3)cc2)cc1]

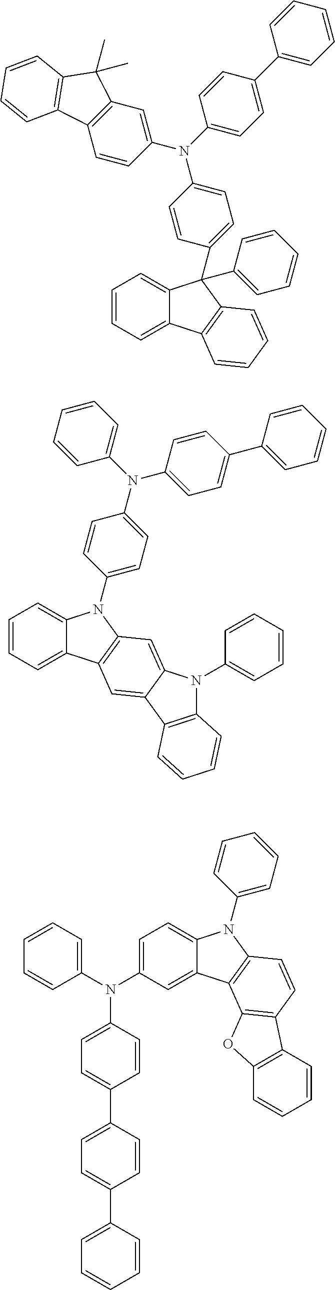 [SMILES: CC1(C)c2ccccc2-c2ccc(N(c3ccc(-c4ccccc4)cc3)c3ccc(C4(c5ccccc5)c5ccccc5-c5ccccc54)cc3)cc21 ; c1ccc(-c2ccc(N(c3ccccc3)c3ccc(-n4c5ccccc5c5cc6c7ccccc7n(-c7ccccc7)c6cc54)cc3)cc2)cc1 ; c1ccc(-c2ccc(-c3ccc(N(c4ccccc4)c4ccc5c(c4)c4c6oc7ccccc7c6ccc4n5-c4ccccc4)cc3)cc2)cc1]

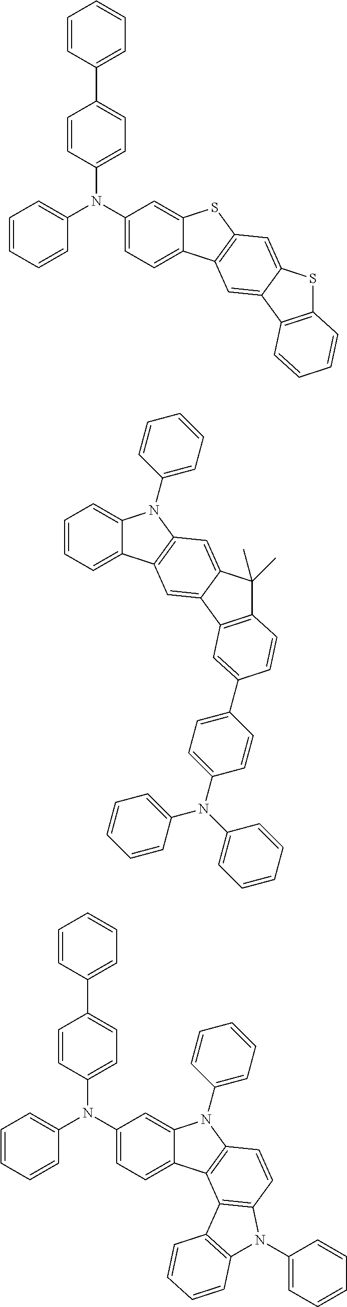 [SMILES: c1ccc(-c2ccc(N(c3ccccc3)c3ccc4c(c3)sc3cc5sc6ccccc6c5cc34)cc2)cc1 ; CC1(C)c2ccc(-c3ccc(N(c4ccccc4)c4ccccc4)cc3)cc2-c2cc3c4ccccc4n(-c4ccccc4)c3cc21 ; c1ccc(-c2ccc(N(c3ccccc3)c3ccc4c5c6c7ccccc7n(-c7ccccc7)c6ccc5n(-c5ccccc5)c4c3)cc2)cc1]

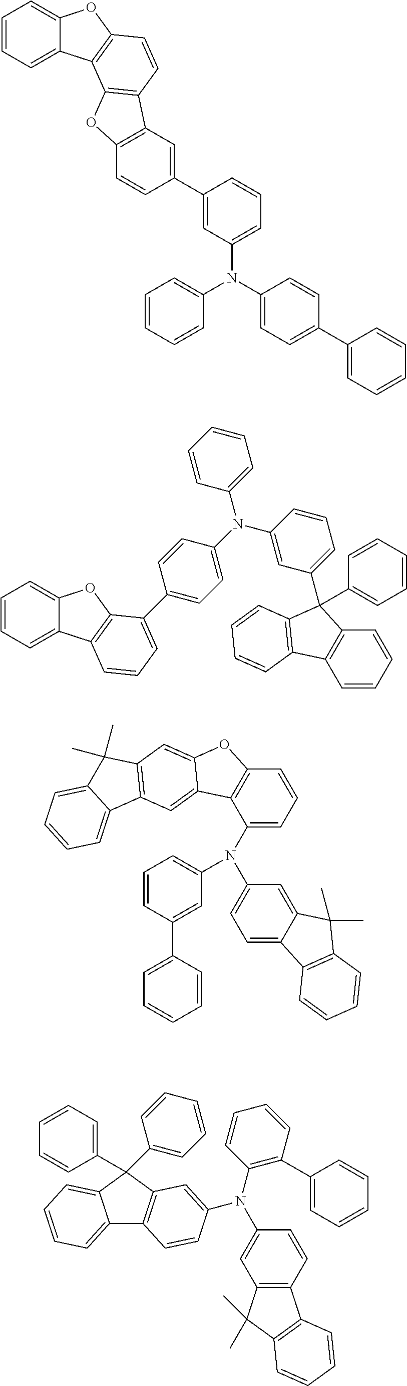 [SMILES: c1ccc(-c2ccc(N(c3ccccc3)c3cccc(-c4ccc5oc6c(ccc7oc8ccccc8c76)c5c4)c3)cc2)cc1 ; c1ccc(N(c2ccc(-c3cccc4c3oc3ccccc34)cc2)c2cccc(C3(c4ccccc4)c4ccccc4-c4ccccc43)c2)cc1 ; CC1(C)c2ccccc2-c2ccc(N(c3cccc(-c4ccccc4)c3)c3cccc4oc5cc6c(cc5c34)-c3ccccc3C6(C)C)cc21 ; CC1(C)c2ccccc2-c2ccc(N(c3ccc4c(c3)C(c3ccccc3)(c3ccccc3)c3ccccc3-4)c3ccccc3-c3ccccc3)cc21]

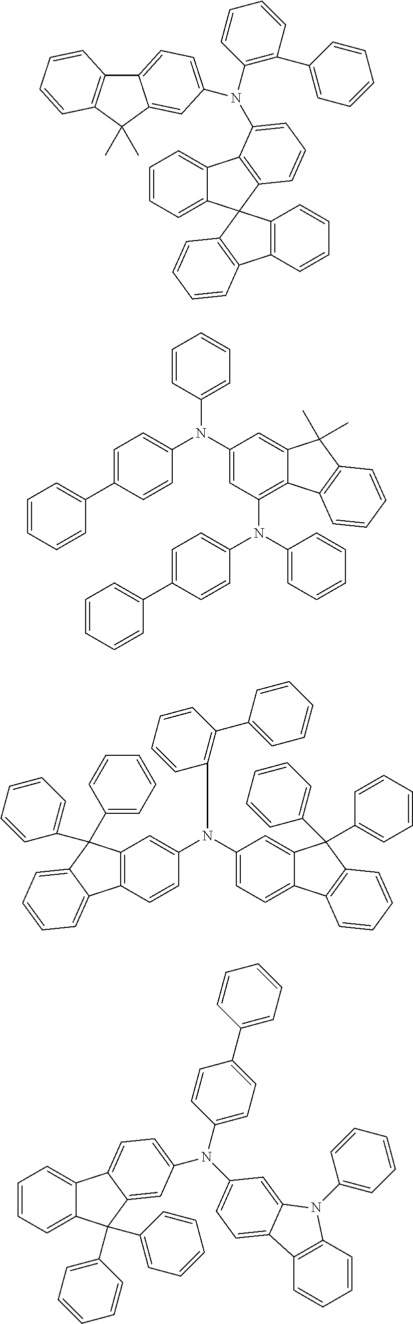

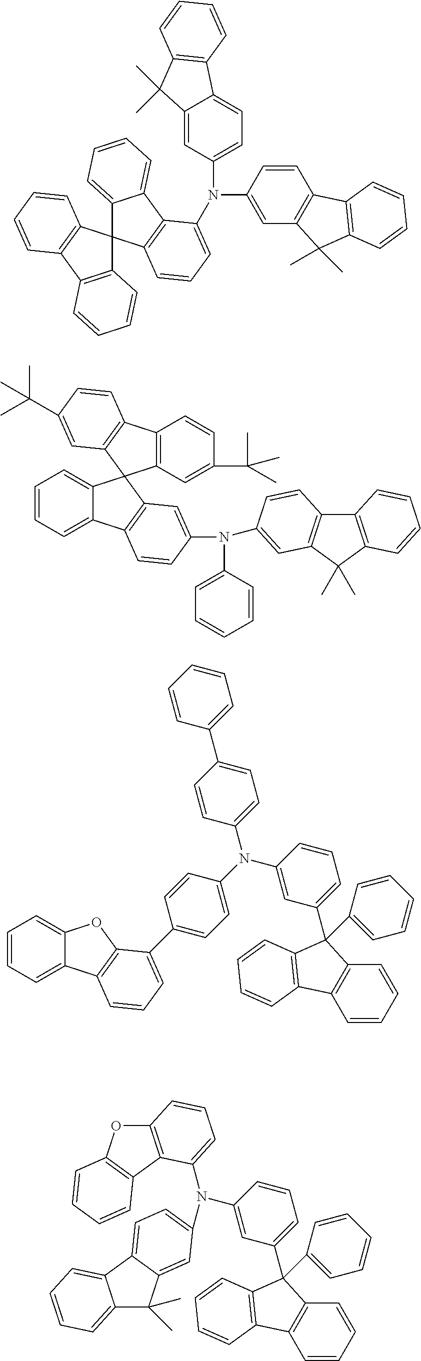

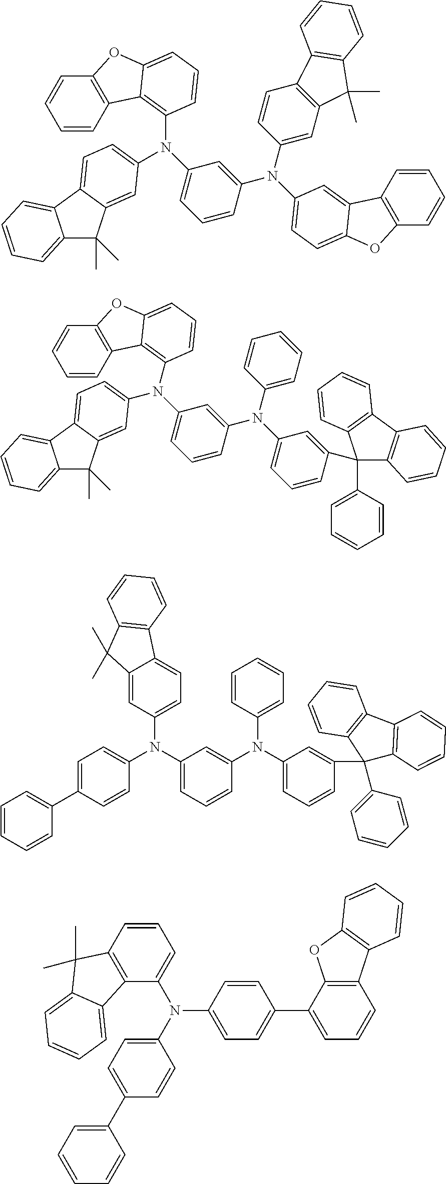

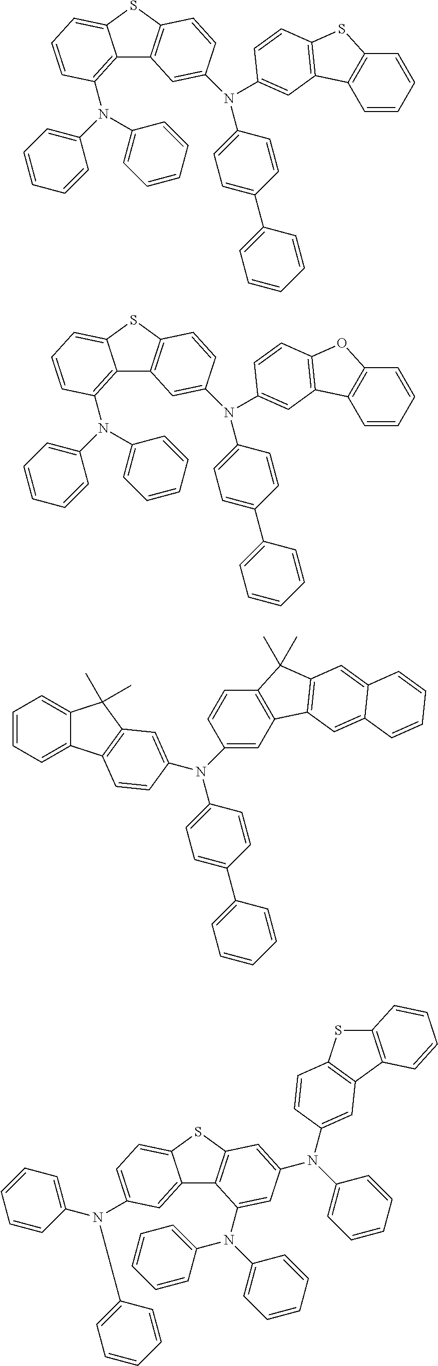

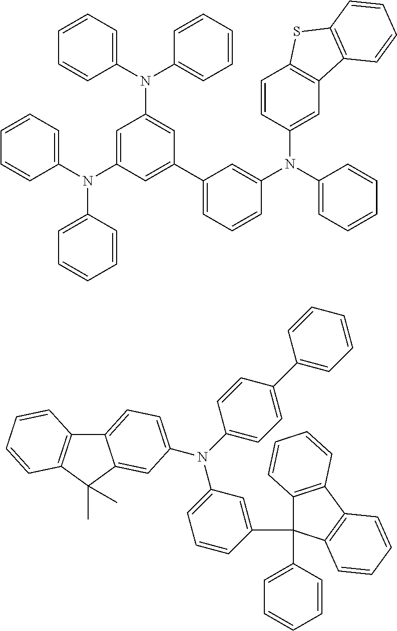 [SMILES: c1ccc(N(c2ccccc2)c2cc(-c3cccc(N(c4ccccc4)c4ccc5sc6ccccc6c5c4)c3)cc(N(c3ccccc3)c3ccccc3)c2)cc1 ; CC1(C)c2ccccc2-c2ccc(N(c3ccc(-c4ccccc4)cc3)c3cccc(C4(c5ccccc5)c5ccccc5-c5ccccc54)c3)cc21]

In the hole transport region 140, other suitable may be used in addition to the compounds.

In an implementation, the charge transport region may be, e.g., the electron transport region 150.

The electron transport region 150 may further increase electron injection and/or electron mobility between the cathode 110 and the light emitting layer 130 and block holes.

In an implementation, the electron transport region 150 may include an electron transport layer between the cathode 110 and the light emitting layer 130, and an electron transport auxiliary layer between the light emitting layer 130 and the electron transport layer and a compound of Group C may be included in at least one of the electron transport layer and the electron transport auxiliary layer.

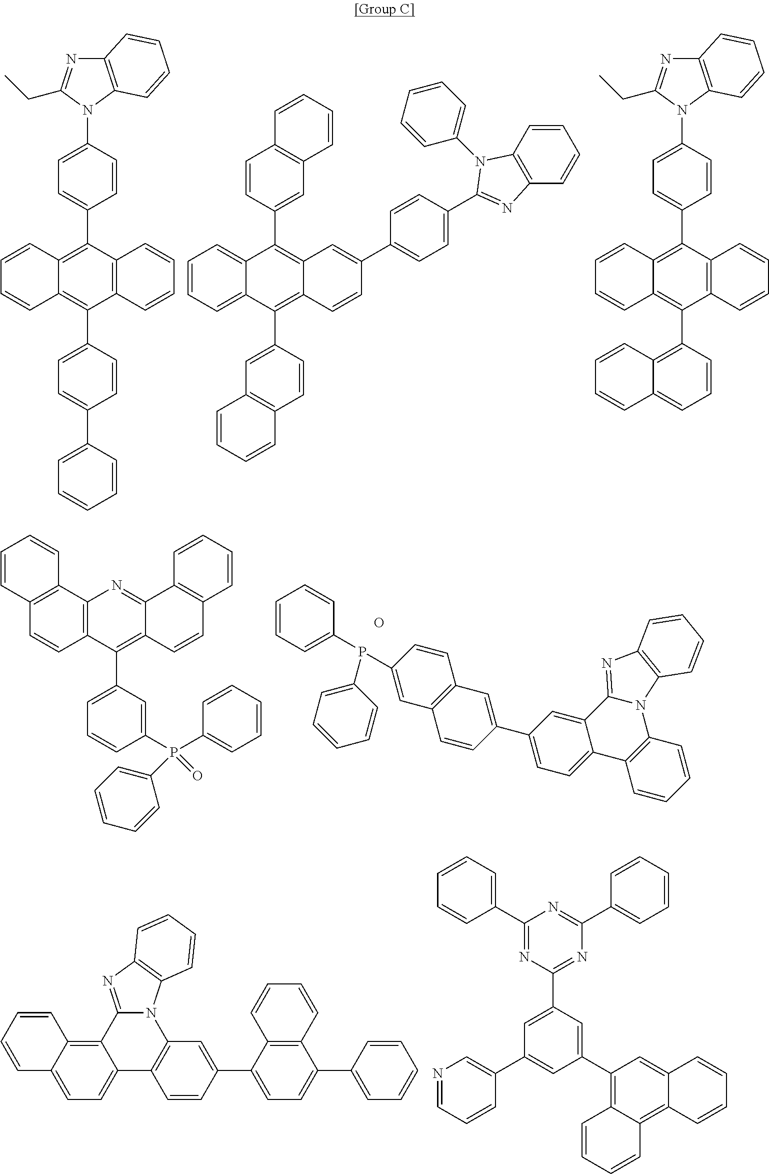

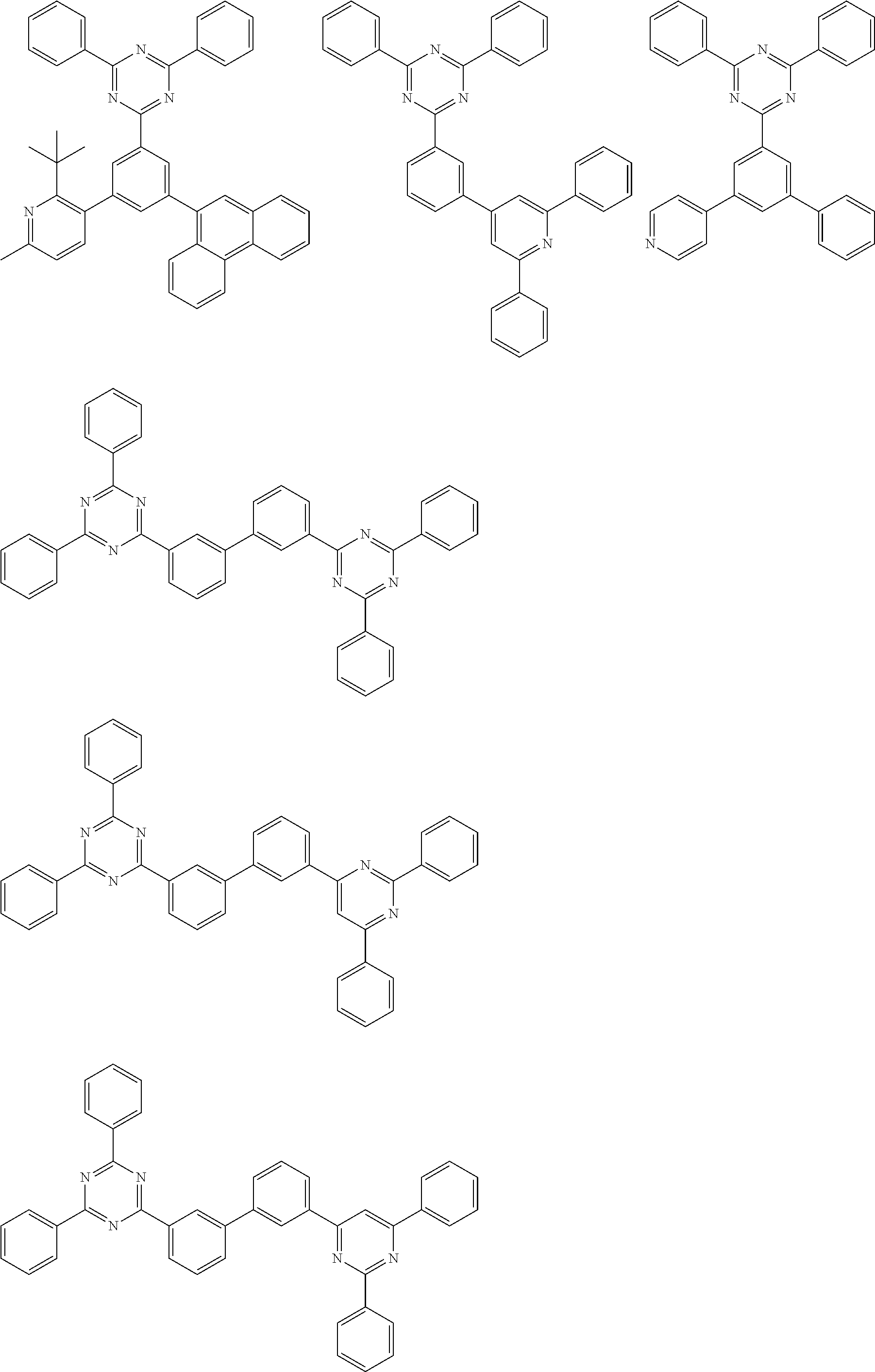

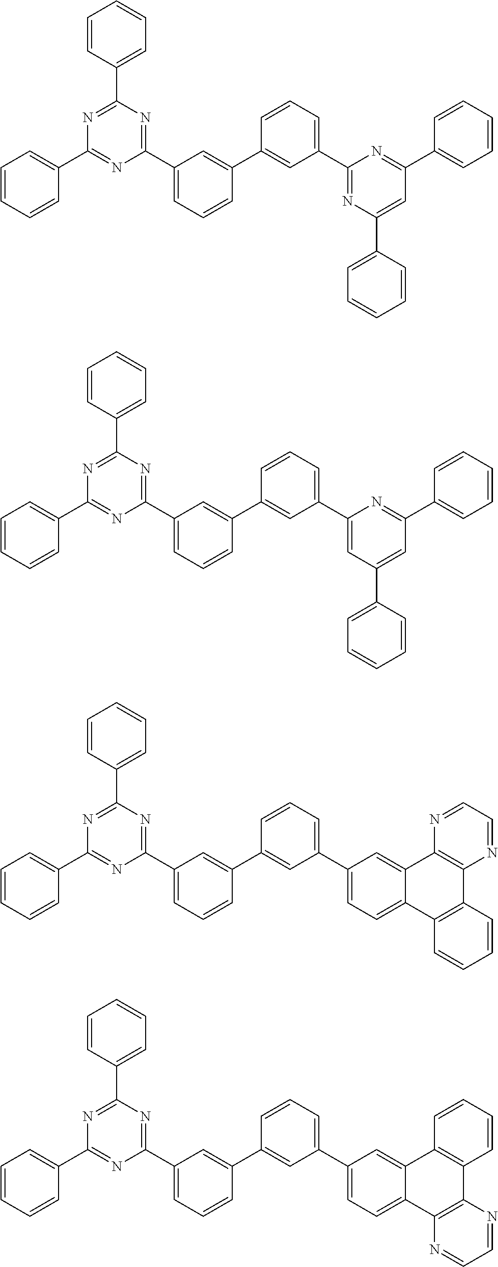 [SMILES: c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

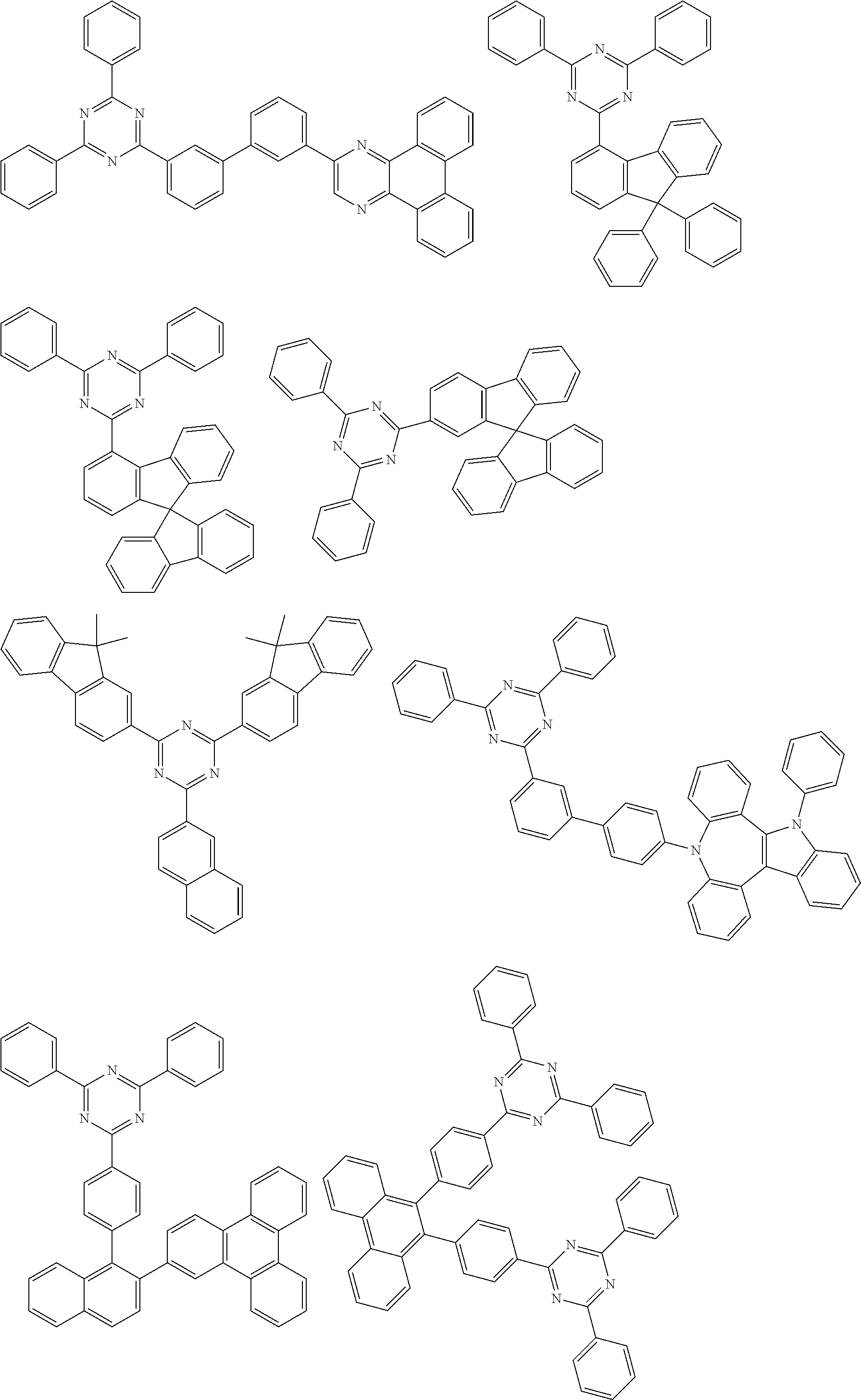

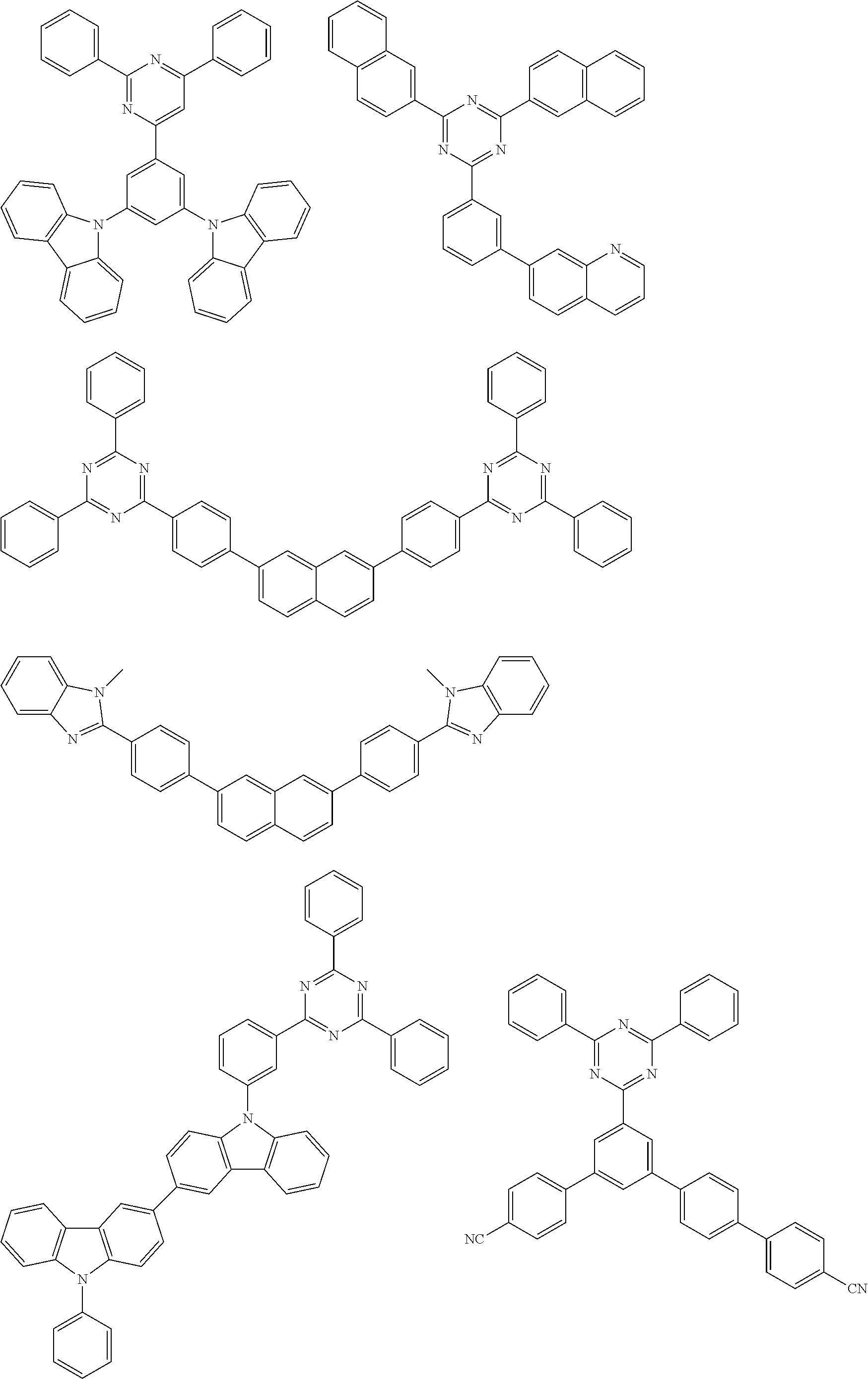

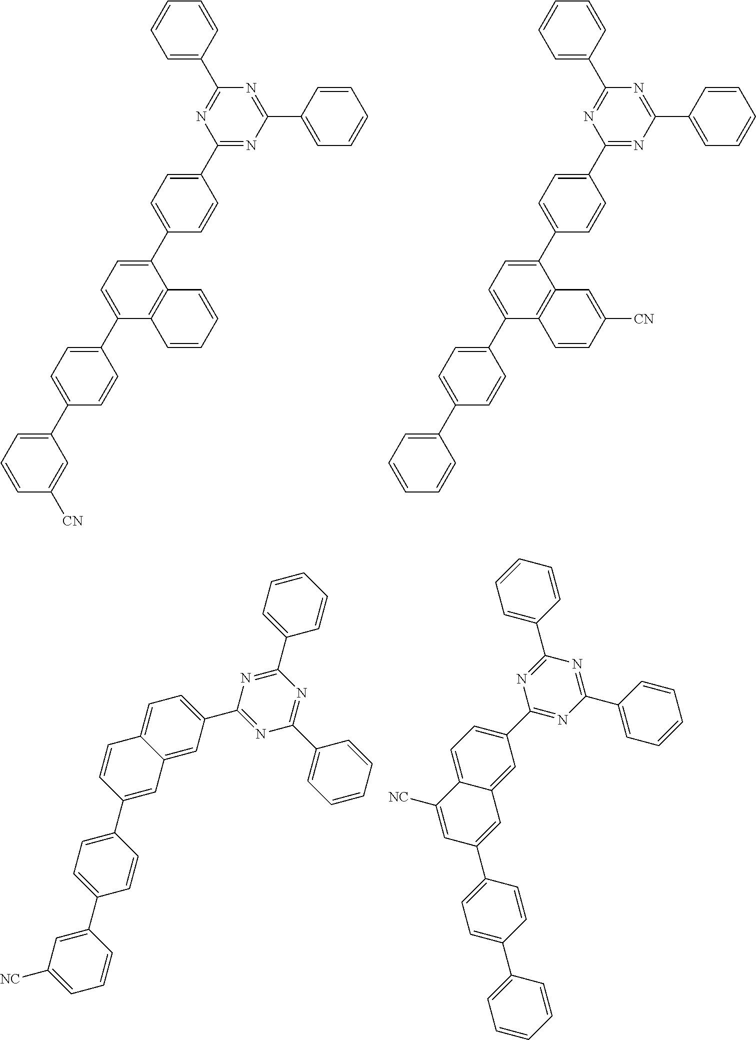

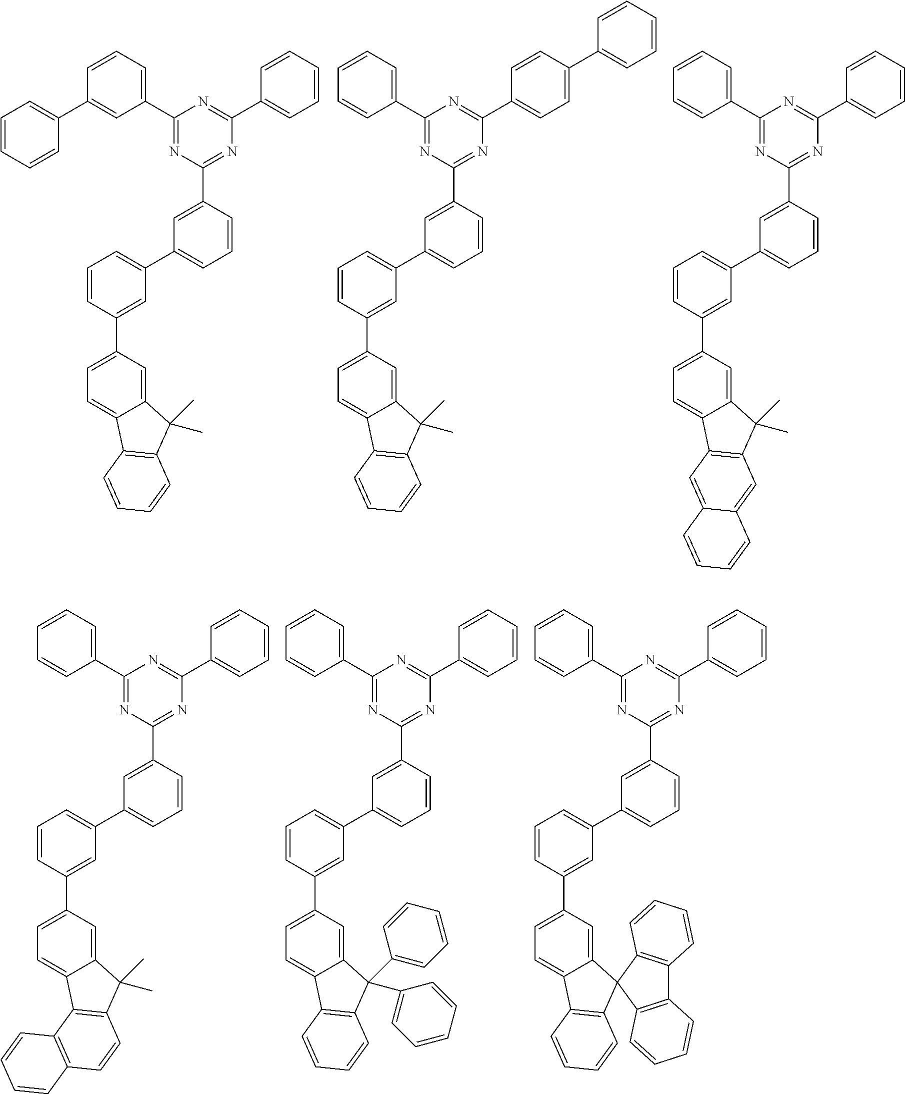

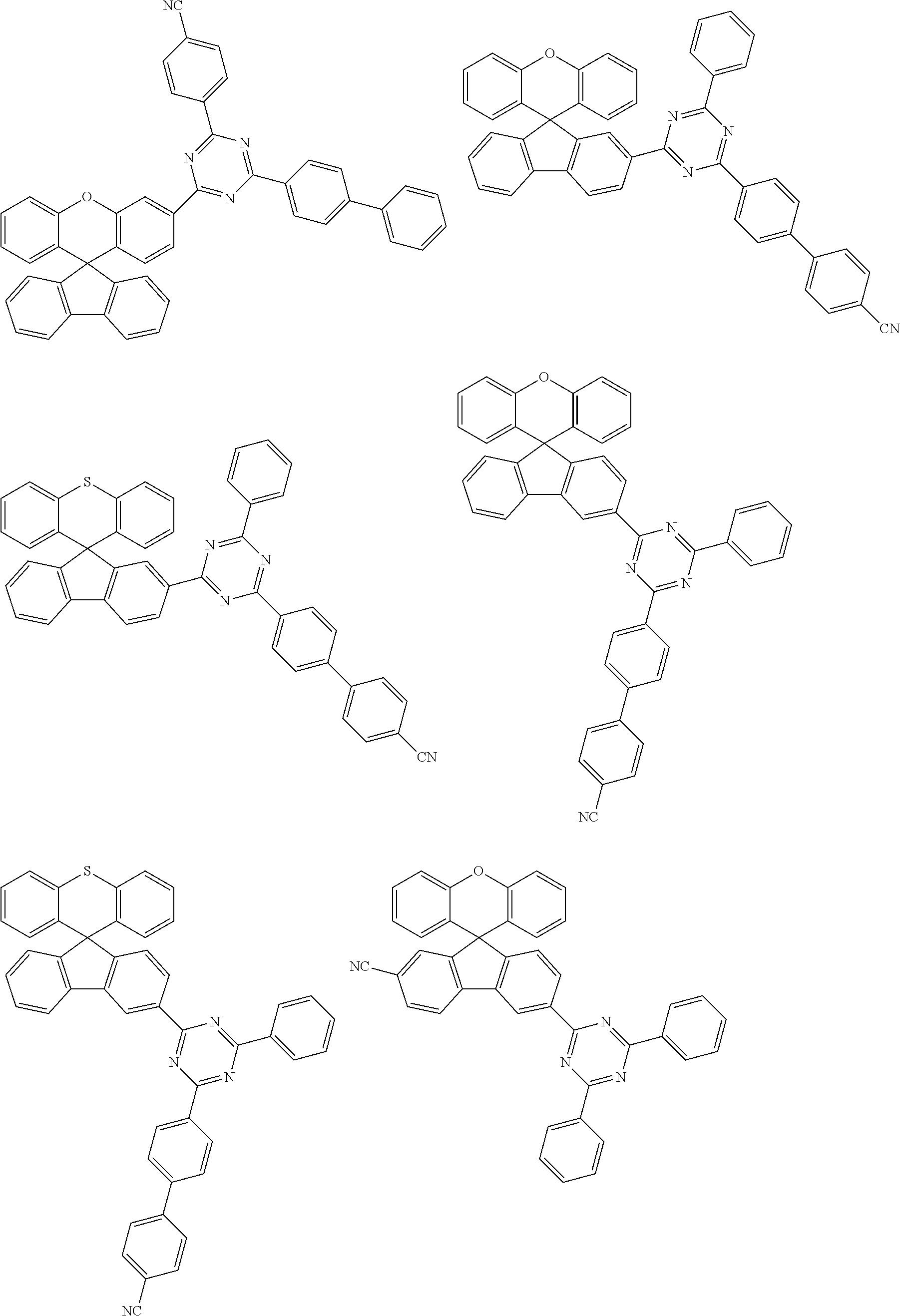

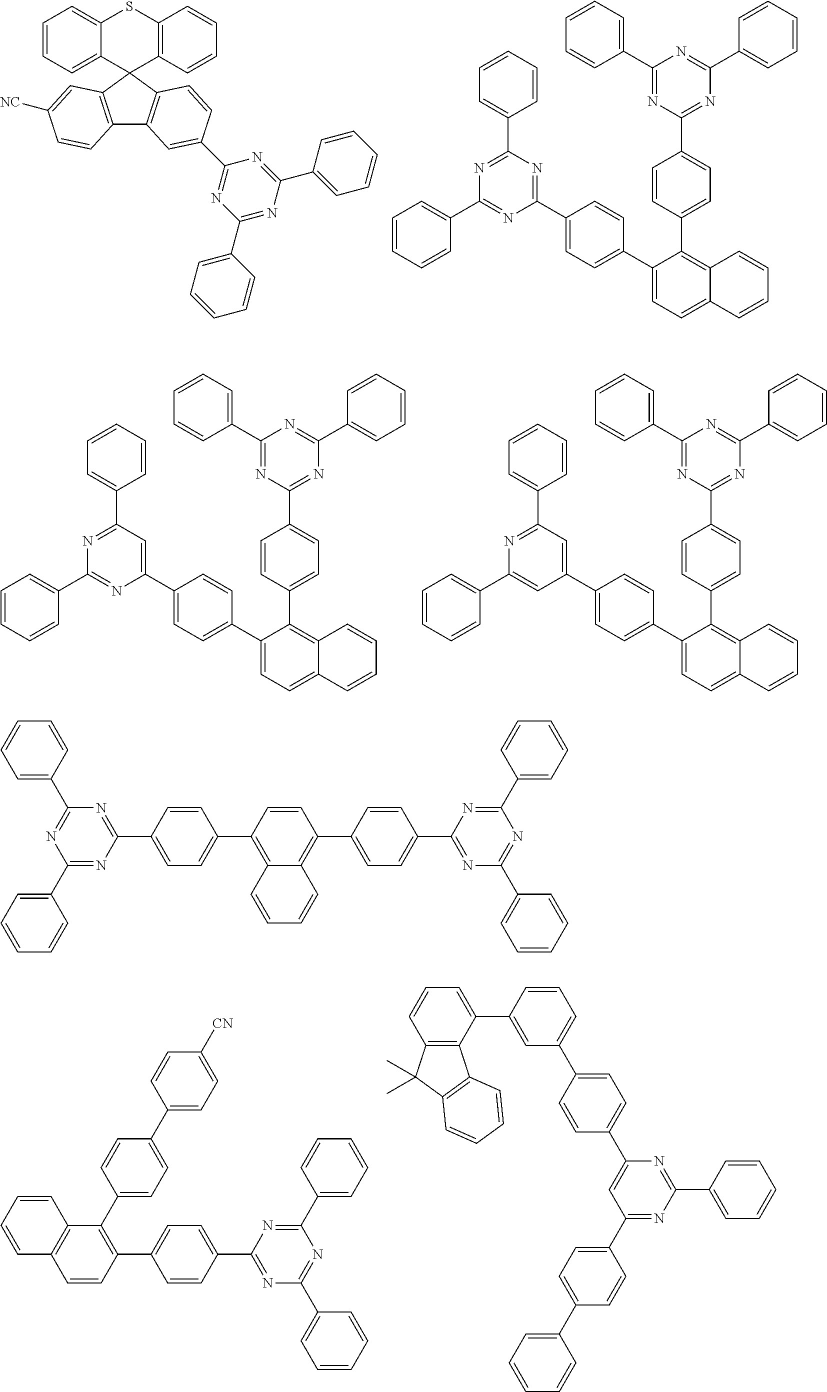

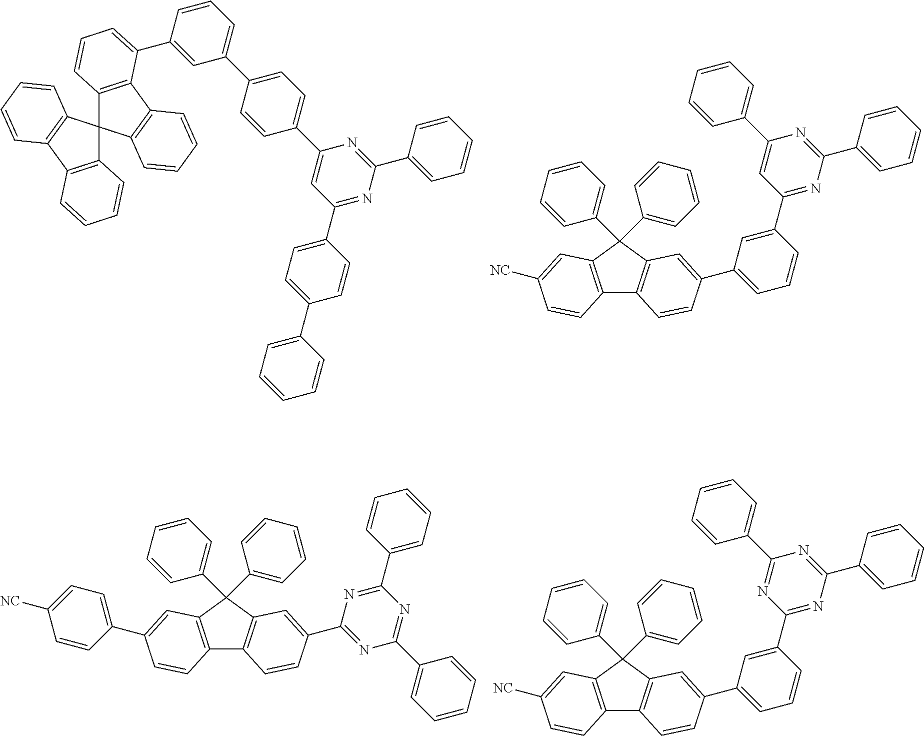

An embodiment may provide an organic light emitting diode including a light emitting layer as an organic layer.

Another embodiment may provide an organic light emitting diode including a light emitting layer and a hole transport region as an organic layer.

Another embodiment may provide an organic light emitting diode including a light emitting layer and an electron transport region as an organic layer.

The organic light emitting diode according to an embodiment may include a hole transport region 140 and an electron transport region 150 in addition to the light emitting layer 130 as the organic layer 105, as shown in the FIGURE.

In an implementation, the organic light emitting diode may further include an electron injection layer, a hole injection layer, or the like, in addition to the light emitting layer as the aforementioned organic layer.

The organic light emitting diode 100 may be manufactured by forming an anode or a cathode on a substrate, forming an organic layer using a dry film formation method such as a vacuum deposition method (evaporation), sputtering, plasma plating, and ion plating, and forming a cathode or an anode thereon.

The organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in examples and synthesis examples were purchased from Sigma-Aldrich Co. Ltd., TCI Inc., Tokyo Chemical Industry, or P&H Tech, as far as there in no particular comment or were synthesized by suitable methods.

(Preparation of Compounds for Organic Optoelectronic Device)

Compounds were synthesized through the following steps.

### Synthesis Example 1: Synthesis of Compound 1

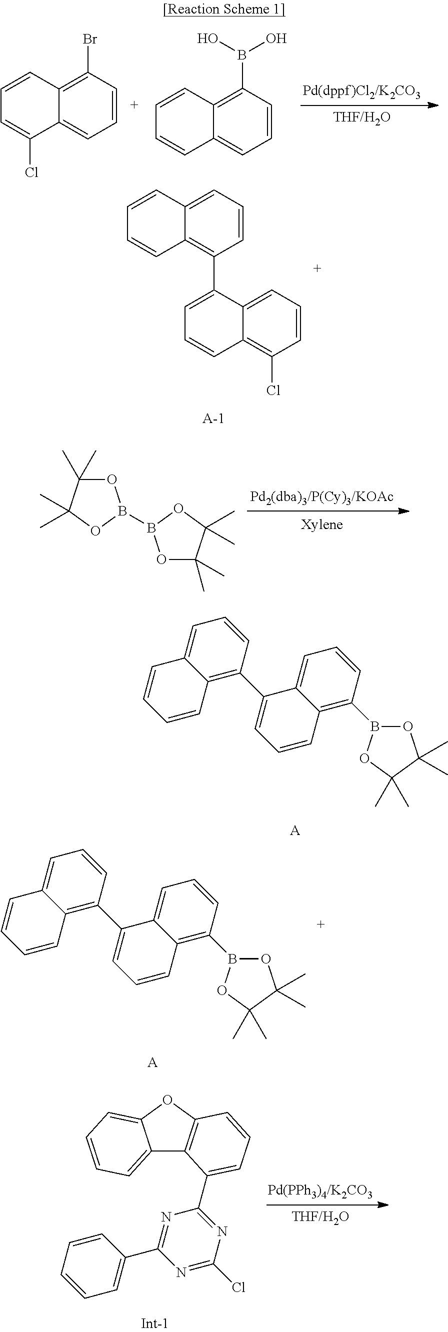

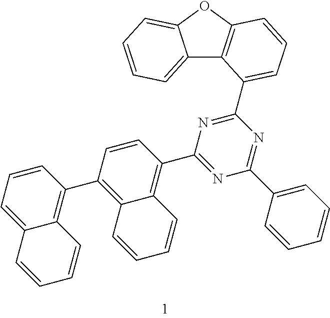 [SMILES: c1ccc(-c2nc(-c3ccc(-c4cccc5ccccc45)c4ccccc34)nc(-c3cccc4oc5ccccc5c34)n2)cc1]

1st step: Synthesis of Intermediate A-1

In a round-bottomed flask, 60.0 g (248.48 mmol) of 1-bromo-5-chloronaphthalene, 42.7 g (248.44 mmol) of 1-naphthaleneboronic acid, 12.17 g (14.91 mmol) of Pd(dppf)Cl₂, and 85.84 g (621.09 mmol) of K₂CO₃ were dissolved in 600 mL of THF and 300 mL of distilled water and then, heated under reflux under a nitrogen atmosphere. After 12 hours, the reaction solution was cooled, and after removing an aqueous layer, an organic layer therefrom was dried under a reduced pressure. The obtained solid was washed with water and methanol and recrystallized with 200 mL of toluene, obtaining 55.0 g (Yield: 77%) of Intermediate A-1.

2nd step: Synthesis of Intermediate A

55.0 g (190.46 mmol) of Intermediate A-1, 48.37 g (190.46 mmol) of bis(pinacolato) diboron, 10.46 g (11.43 mmol) of Pd₂(dba)₃, 12.82 g (45.71 mmol) of P(Cy)₃, and 56.08 g (571.39 mmol) of KOAc were dissolved in 600 m1 of xylene and then, stirred under reflux for 12 hours. When a reaction was completed, after removing a reaction solvent with a rotary evaporator, an organic layer was extracted therefrom with methylene chloride and columned with hexane:EA=4:1 (v/v), obtaining 55.0 g (Yield: 76%) of Intermediate A.

LC/MS calculated for: C26H25BO2 Exact Mass 380.29 found for 381.14 [M+H]

3rd step: Synthesis of Compound 1

In a round-bottomed flask, 13.92 g (30.74 mmol) of Intermediate A, 10.0 g (27.95 mmol) of 2-chloro-4-(1-dibenzofuranyl)-6-phenyl-1,3,5-triazine (Int-1), 0.97 g (0.84 mmol) of Pd(PPh₃)₄, and 7.73 g (55.90 mmol) of K₂CO₃ were dissolved in 150 mL of THF and 70 mL of distilled water and then, heated under reflux under a nitrogen atmosphere. After 12 hours, the reaction solution was cooled, and after removing an aqueous layer, an organic layer therefrom was dried under a reduced pressure. The obtained solid was washed with water and methanol and then, twice recrystallized with 200 mL of toluene, obtaining 12.0 g (Yield: 81%) of Compound 1.

LC/MS calculated for: C41H25N3O Exact Mass 575.67 found for 576.32 [M+H]

Synthesis of Intermediate B to Intermediate E

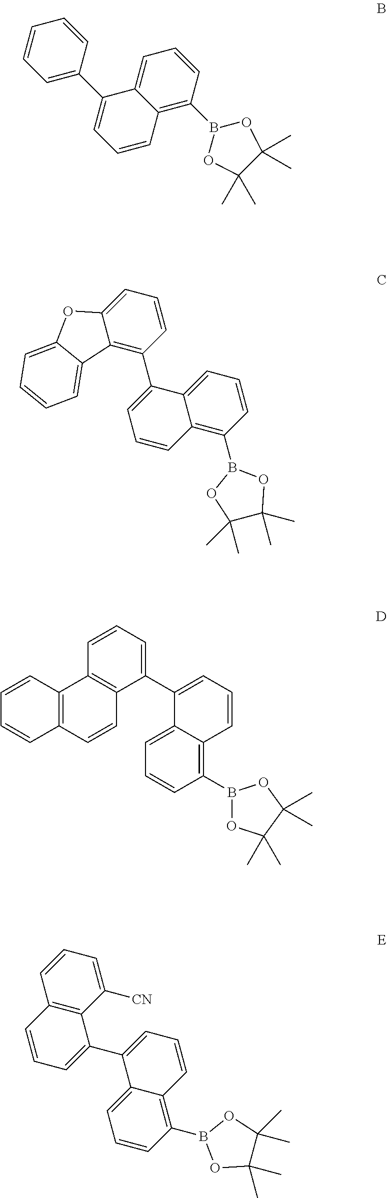 [SMILES: CC1(C)OB(c2cccc3c(-c4ccccc4)cccc23)OC1(C)C ; CC1(C)OB(c2cccc3c(-c4cccc5oc6ccccc6c45)cccc23)OC1(C)C ; CC1(C)OB(c2cccc3c(-c4cccc5c4ccc4ccccc45)cccc23)OC1(C)C ; CC1(C)OB(c2cccc3c(-c4cccc5cccc(C#N)c45)cccc23)OC1(C)C]

### Synthesis Example 2: Synthesis of Intermediate B

61.0 g (Yield: 80%) of Intermediate B was synthesized in the same manner as in the 1st and 2nd steps of Synthesis Example 1 except that phenylboronic acid instead of the 1-naphthaleneboronic acid was used in the same equivalent in the 1st step of Synthesis Example 1.

LC/MS calculated for: C22H23BO2 Exact Mass 330.23 found for 330.85 [M+H]

### Synthesis Example 3: Synthesis of Intermediate C

55.0 g (Yield: 78%) of Intermediate C was synthesized in the same manner as in the 1st and 2nd steps of Synthesis Example 1 except that dibenzo[b,d]furan-1-ylboronic acid instead of the 1-naphthaleneboronic acid was used in the same equivalent in the 1st step of Synthesis Example 1.

LC/MS calculated for: C28H25BO3 Exact Mass 420.32 found for 420.65 [M+H]

### Synthesis Example 4: Synthesis of Intermediate D

52.0 g (Yield: 74%) of Intermediate D was synthesized in the same manner as in the 1st and 2nd steps of Synthesis Example 1 except that phenanthren-1-ylboronic acid instead of the 1-naphthaleneboronic acid was used in the same equivalent in the 1st step of Synthesis Example 1.

LC/MS calculated for: C30H27BO2 Exact Mass 430.35 found for 431.11 [M+H]

### Synthesis Example 5: Synthesis of Intermediate E

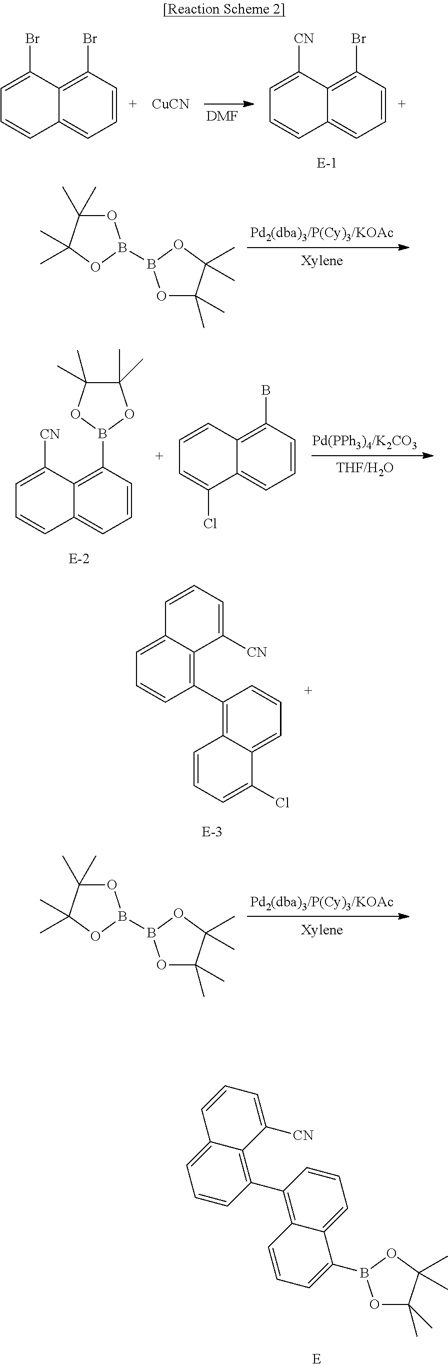

1st step: Synthesis of Intermediate E-1

50.0 g (174.84 mmol) of 1,8-dibromonaphthalene and 15.66 g (174.84 mmol) of copper(I) cyanide were dissolved in 1,000 m1 of DMF and heated under reflux for 12 hours at 150° C. under a nitrogen atmosphere. When a reaction was completed, after removing the reaction solvent with a rotary evaporator, an organic layer was extracted therefrom with methylene chloride and columned with hexane:EA=4:1 (v/v), obtaining 30.0 g (Yield: 74%) of Intermediate E-1.

2nd step: Synthesis of Intermediate E-2

In a round-bottomed flask, 30.0 g (129.27 mmol) of Intermediate E-1, 32.38 g (129.27 mmol) of bis(pinacolato) diboron, 7.10 g (7.76 mmol) of Pd₂(dba)₃, 8.70 g (31.08 mmol) of P(Cy)3, and 38.06 g (387.80 mmol) of KOAc were dissolved in 350 m1 of xylene and then, stirred under reflux for 12 hours. When a reaction was completed, after removing the reaction solvent with a rotary evaporator, an organic layer was extracted therefrom with methylene chloride and columned with hexane:EA=4:1 (v/v), obtaining 45.0 g (Yield: 77%) of Intermediate E-2.

3rd step: Synthesis of Intermediate E-3

In a round-bottomed flask, 32.0 g (132.50 mmol) of 1-bromo-5-chloronaphthalene, 44 g (132.50 mmol) of Intermediate E-2, 4.59 g (3.97 mmol) of Pd(PPh₃)₄, and 36.63 g (265.0 mmol) of K₂CO₃ were dissolved in 600 mL of THF and 300 mL of distilled water and then, heated under reflux under a nitrogen atmosphere. After 12 hours, the reaction solution was cooled, and after removing an aqueous layer, an organic layer therefrom was dried under a reduced pressure. The obtained solid was washed with water and methanol and recrystallized with 200 mL of toluene, obtaining 38.0 g (Yield: 76%) of Intermediate E-3.

4th step: Synthesis of Intermediate E

38.0 g (121.10 mmol) of Intermediate E-3, 30.75 g (121.10 mmol) of bis(pinacolato) diboron, 6.65 g (7.27 mmol) of Pd₂(dba)₃, 8.15 g (29.06 mmol) of P(Cy)₃, and 35.66 g (363.31 mmol) of KOAc were dissolved in 350 m1 of xylene and then, stirred under reflux for 12 hours. When a reaction was completed, after removing the reaction solvent with a rotary evaporator, an organic layer was extracted therefrom with methylene chloride and columned with hexane:EA=4:1 (v/v), obtaining 35.0 g (Yield: 71%) of Intermediate E.

LC/MS calculated for: C27H24BNO2 Exact Mass 405.30 found for 405.77 [M+H]

### Synthesis Examples 6 to 9

The compounds according to Table 1 were respectively synthesized in the same manner as in Synthesis Example 1 except that Intermediate B to Intermediate E were respectively used instead of Intermediate A, and Int-1 or Int-2 was used in the 3rd step of Synthesis Example 1.

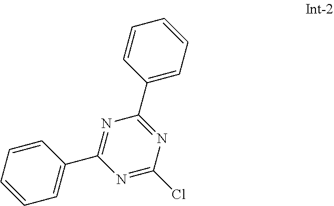 [SMILES: Clc1nc(-c2ccccc2)nc(-c2ccccc2)n1]

### Comparative Synthesis Example 1: Synthesis of Intermediate F

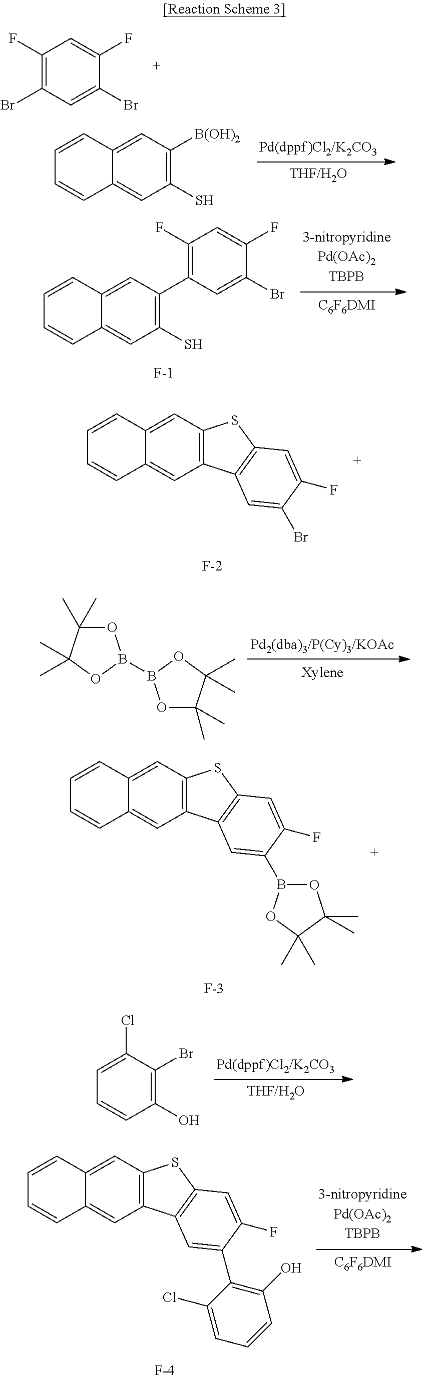

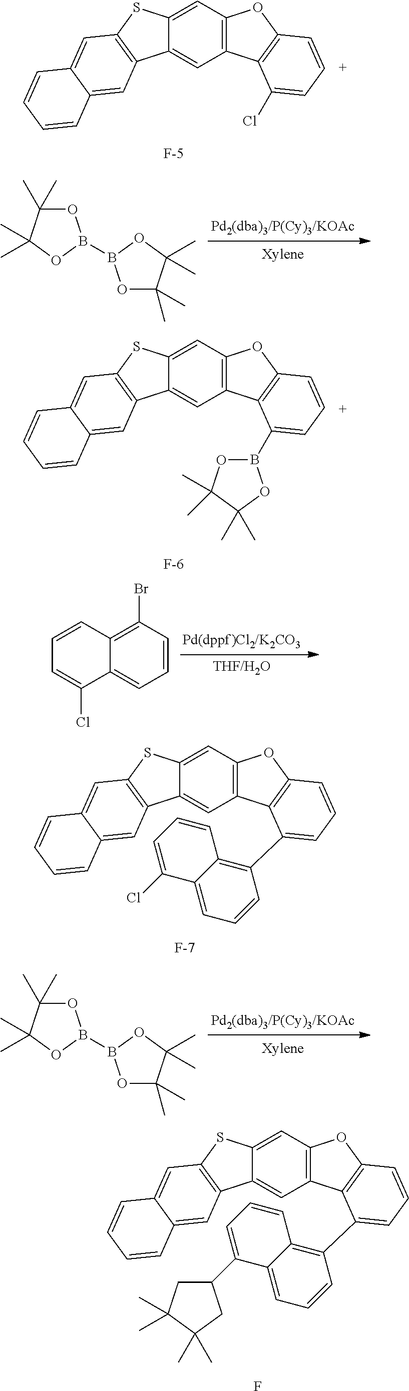

1st step: Synthesis of Intermediate F-1

9.0 g (Yield: 63%) of Intermediate F-1 was synthesized in the same manner as in the 1st step of Synthesis Example 1 except that (3-mercaptonaphthalen-2-yl)boronic acid (6.88 g, 28.32 mmol), Pd(PPh₃)₄ (1.4 g, 1.21 mmol), K₂CO₃ (11.18 g, 80.92 mmol), THF (200 ml), and H₂O (100 ml) were added to 1,5-dibromo-2,4-difluorobenzene (10.0 g, 36.78 mmol).

2nd step: Synthesis of Intermediate F-2

3-nitropyridine (0.30 g, 2.05 mmol), Pd(OAc)₂ (0.46 g, 2.05 mmol), tert-butyl peroxybenzoate (18.66 g, 46.13 mmol), C₆F₆ (100 ml), and 1,3-dimethyl-2-imidazolidinone (100 ml) were added to Intermediate F-1 (9.00 g, 25.63 mmol) and then, heated under reflux at 90° C. for 12 hours. When a reaction was completed, after removing the reaction solvent with a rotary evaporator, an organic layer was extracted therefrom with methylene chloride, dried with MgSO₄, and concentrated, and then, an organic material produced therefrom was separated through silica gel column, obtaining 7.0 g (Yield: 78%) of Intermediate F-2.

3rd step: Synthesis of Intermediate F-3

7.0 g (21.14 mmol) of Intermediate F-2, 5.37 g (21.14 mmol) of bis(pinacolato) diboron, 1.16 g (1.27 mmol) of Pd₂(dba)₃, 1.42 g (5.07 mmol) of P(Cy)₃, and 6.22 g (63.41 mmol) of KOAc were dissolved in 100 m1 of xylene and then, stirred under reflux for 12 hours. When a reaction was completed, after removing the reaction solvent with a rotary evaporator, an organic layer was extracted therefrom with methylene chloride and columned with hexane:EA=4:1 (v/v), obtaining 7.0 g (Yield: 88%) of Intermediate F-3.

4th step: Synthesis of Intermediate F-4

5.3 g (Yield: 90%) of Intermediate F-4 was synthesized in the same method as in the 1st step of Synthesis Example 1 except that Intermediate F-3 (7.0 g, 15.43 mmol), Pd(PPh₃)₄ (0.53 g, 0.46 mmol), K₂CO₃ (4.26 g, 30.85 mmol), THF (80 ml), and H₂O (40 m1) were added to 2-bromo-3-chlorophenol (3.2 g, 15.43 mmol).

5th step: Synthesis of Intermediate F-5

Intermediate F-5 4.0 g (Yield: 80%) was synthesized in the 2^{nd} step of Comparative Synthesis Example 1 except that 3-nitropyridine (0.17 g, 1.12 mmol), Pd(OAc)₂ (0.25 g, 1.12 mmol), tert-butyl peroxybenzoate (10.19 g, 25.18 mmol), C₆F₆ (50 ml), and 1,3-dimethyl-2-imidazolidinone (50 ml) were added to Intermediate F-4 (5.30 g, 13.99 mmol).

6th step: Synthesis of Intermediate F-6

4.2 g (Yield: 84%) of Intermediate F-6 was synthesized in the 3^{rd} step of Comparative Synthesis Example 1 except that 4.0 g (11.15 mmol) of Intermediate F-5, 2.83 g (11.15 mmol) of bis(pinacolato) diboron, 0.61 g (0.67 mmol) of Pd₂(dba)₃, 0.75 g (2.68 mmol) of P(Cy)₃, and 3.28 g (33.44 mmol) of KOAc were dissolved in 40 m1 of xylene.

LC/MS calculated for: C28H23BO3S Exact Mass 450.36 found for 450.81 [M+H]

7th step: Synthesis of Intermediate F-7

In a round-bottomed flask, 4.2 g (17.39 mmol) of Intermediate F-6, 7.83 g (17.39 mmol) of 1-bromo-5-chloronaphthalene, 0.6 g (0.52 mmol) of Pd(PPh₃)₄, and 4.81 g (34.78 mmol) of K₂CO₃ were dissolved in 90 m1 of THF and 45 ml of H₂O and then, heated under reflux under a nitrogen atmosphere. After 12 hours, the reaction solution was cooled, and after removing an aqueous layer, an organic layer therefrom was dried under a reduced pressure. The obtained solid was washed with MeOH and recrystallized with toluene, obtaining 7.5 g (Yield: 89%) of Intermediate F-7.

8th step: Synthesis of Intermediate F

7.5 g (15.46 mmol) of Intermediate F-7, 4.12 g (16.24 mmol) of bis(pinacolato) diboron, 0.85 g (0.06 mmol) of Pd₂(dba)₃, 4.55 g (46.39 mmol) of P(Cy)₃, and 4.55 g (46.39 mmol) of KOAc were dissolved in 50 m1 of xylene and then, stirred under reflux for 12 hours. When a reaction was completed, after removing the reaction solvent with a rotary evaporator, an organic layer was extracted therefrom with methylene chloride and columned with hexane:EA=4:1 (v/v), obtaining 7.7 g (Yield: 89%) of Intermediate F.

LC/MS calculated for: C38H29BO3S Exact Mass 576.19 found for 576.54 [M+H]

Synthesis of Intermediate G to Intermediate J

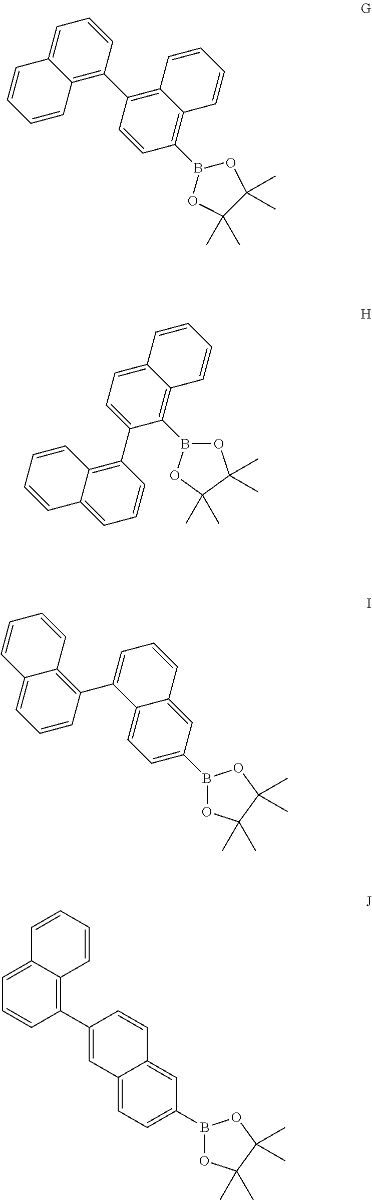 [SMILES: CC1(C)OB(c2ccc(-c3cccc4ccccc34)c3ccccc23)OC1(C)C ; CC1(C)OB(c2c(-c3cccc4ccccc34)ccc3ccccc23)OC1(C)C ; CC1(C)OB(c2ccc3c(-c4cccc5ccccc45)cccc3c2)OC1(C)C ; CC1(C)OB(c2ccc3cc(-c4cccc5ccccc45)ccc3c2)OC1(C)C]

### Comparative Synthesis Example 2: Synthesis of Intermediate G

55.0 g (Yield: 84%) of Intermediate G was synthesized in the same manner as in the 1st and 2nd steps of Synthesis Example 1 except that 1-bromo-4-chloronaphthalene instead of the 1-bromo-5-chloronaphthalene was used in the same equivalent in the 1st step of Synthesis Example 1.

LC/MS calculated for: C26H25BO2 Exact Mass 380.29 found for 381.11 [M+H]

### Comparative Synthesis Example 3: Synthesis of Intermediate H

58.0 g (Yield: 88%) of Intermediate H was synthesized in the same manner as in the 1st and 2nd steps of Synthesis Example 1 except that 2-bromo-1-chloronaphthalene instead of the 1-bromo-5-chloronaphthalene was used in the same equivalent in the 1st step of Synthesis Example 1.

LC/MS calculated for: C26H25BO2 Exact Mass 380.29 found for 380.87 [M+H]

### Comparative Synthesis Example 4: Synthesis of Intermediate I

57.0 g (Yield: 87%) of Intermediate I was synthesized in the same manner as in the 1st and 2nd steps of Synthesis Example 1 except that 1-bromo-6-chloronaphthalene instead of the 1-bromo-5-chloronaphthalene was used in the same equivalent in the 1st step of Synthesis Example 1.

LC/MS calculated for: C26H25BO2 Exact Mass 380.29 found for 380.58 [M+H]

### Comparative Synthesis Example 5: Synthesis of Intermediate J

55.0 g (Yield: 84%) of Intermediate J was synthesized in the same manner as in the 1st and 2nd steps of Synthesis Example 1 except that 2-bromo-6-chloronaphthalene instead of the 1-bromo-5-chloronaphthalene was used in the same equivalent, in the 1st step of Synthesis Example 1.

LC/MS calculated for: C26H25BO2 Exact Mass 380.29 found for 380.84 [M+H]

### Comparative Synthesis Examples 6 to 10

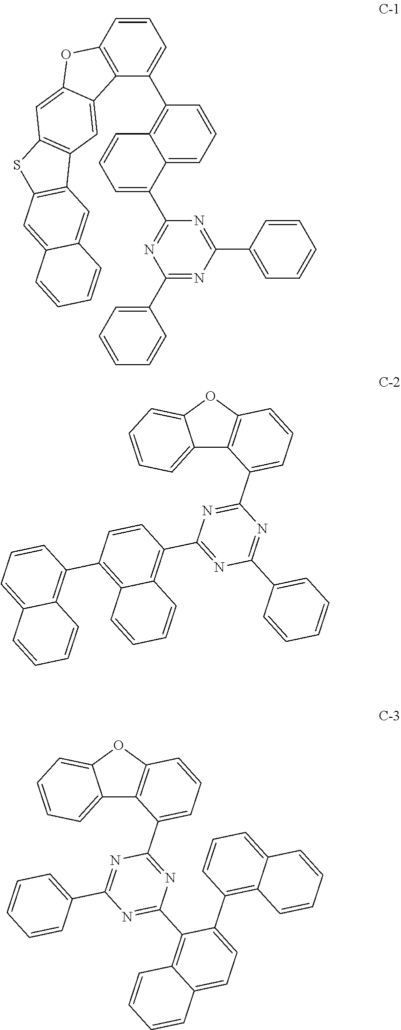

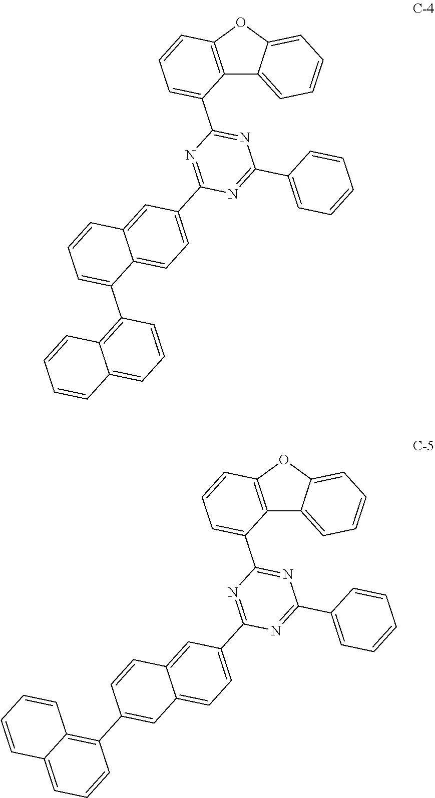 [SMILES: c1ccc(-c2nc(-c3ccc4c(-c5cccc6ccccc56)cccc4c3)nc(-c3cccc4oc5ccccc5c34)n2)cc1 ; c1ccc(-c2nc(-c3ccc4cc(-c5cccc6ccccc56)ccc4c3)nc(-c3cccc4oc5ccccc5c34)n2)cc1]

In the 3rd step of Synthesis Example 1, each reaction was performed in the same manner as in Synthesis Example 1, except that Intermediate G was used instead of Intermediate A, and Int-1 or Int-2 was used to synthesize the following compounds of Table 1.

TABLE 1

| Synthesis | | | Final | Amount | |
|---|---|---|---|---|---|
| Examples | Int A | Int-1 | product | (yield) | Property data of final products |
| Synthesis | Intermediate B | Int-1 | Compound 3 | 12.0 g, (81%) | LC/MS calculated for: |
| Example 6 | | | | | C37H25N3O Exact Mass: 527.63 |
| | | | | | found for 528.15 [M + H] |
| Synthesis | Intermediate C | Int-1 | Compound 5 | 14.0 g, (81%) | LC/MS calculated for: |
| Example 7 | | | | | C43H25N3O2 Exact Mass: |
| | | | | | 615.69 found for 616.21 [M + H] |
| Synthesis | Intermediate D | Int-1 | Compound 6 | 13.0 g, (74%) | LC/MS calculated for: |
| Example 8 | | | | | C45H27N30 Exact Mass: 625.73 |
| | | | | | found for 626.22 [M + H] |
| Synthesis | Intermediate E | Int-1 | Compound 9 | 12.8 g, (76%) | LC/MS calculated for: |
| Example 9 | | | | | C42H24N4O Exact Mass: 600.68 |
| | | | | | found for 600.83 [M + H] |
| Comparative | Intermediate F | Int-2 | Compound | 6.0 g (86%) | LC/MS calculated for: |
| Synthesis | | | C-1 | | C47H27N3OS Exact Mass: |
| Example 6 | | | | | 681.19 found for 681.42 [M + H] |
| Comparative | Intermediate G | Int-1 | Compound | 12.8 g, (80%) | LC/MS calculated for: |
| Synthesis | | | C-2 | | C41H25N3O Exact Mass: 575.67 |
| Example 7 | | | | | found for 576.33 [M + H] |
| Comparative | Intermediate H | Int-1 | Compound | 11.5 g, (71%) | LC/MS calculated for: |
| Synthesis | | | C-3 | | C41H25N30 Exact Mass: 575.67 |
| Example 8 | | | | | found for 576.28 [M + H] |
| Comparative | Intermediate I | Int-1 | Compound | 12.5 g, (78%) | LC/MS calculated for: |
| Synthesis | | | C-4 | | C41H25N3O Exact Mass: 575.67 |
| Example 9 | | | | | found for 576.12[M + H] |
| Comparative | Intermediate J | Int-1 | Compound | 13.2 g, (82%) | LC/MS calculated for: |
| Synthesis | | | C-5 | | C41H25N3O Exact Mass: 575.67 |
| Example 10 | | | | | found for 576.94 [M + H] |

### Synthesis Example 10: Synthesis of Compound A-84

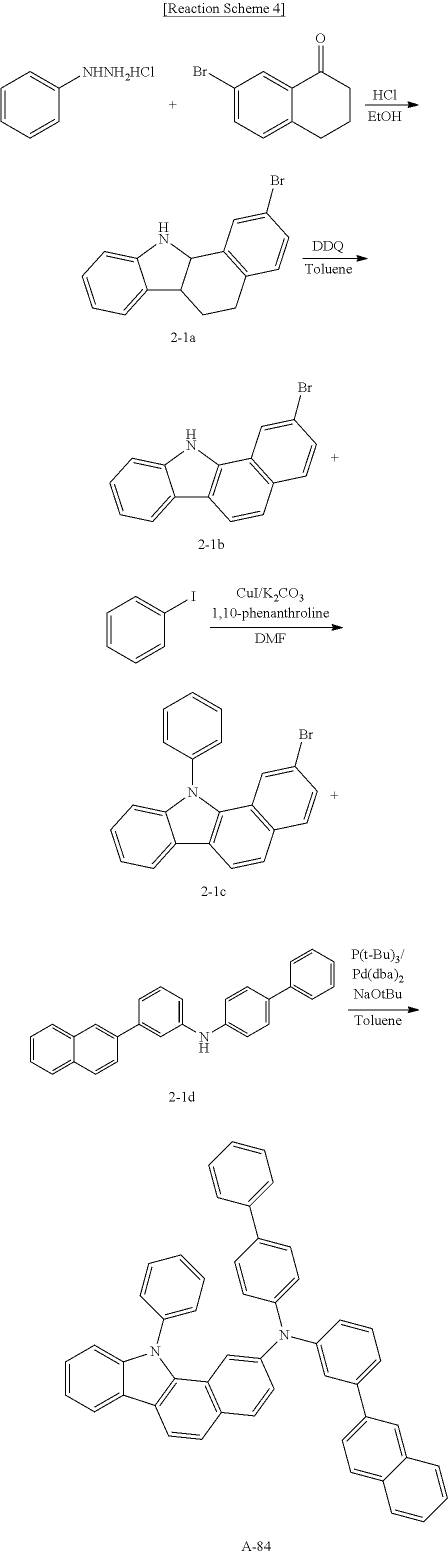

1st step: Synthesis of Intermediate 2-1a

Phenylhydrazine hydrochloride (70.0 g, 484.1 mmol) and 7-bromo-3,4-dihydro-2H-naphthalen-1-one (108.9 g, 484.1 mmol) were put in a round-bottomed flask and dissolved in ethanol (1,200 ml). Subsequently, 60 mL of hydrochloric acid was slowly added thereto in a dropwise fashion at ambient temperature and then, stirred at 90° C. for 12 hours. When a reaction was completed, after removing the solvent under a reduced pressure, an excess of EA was used for extraction. After removing an organic solvent under a reduced pressure, the residue was stirred in a small amount of methanol and then, filtered, obtaining 95.2 g (66%) of Intermediate 2-1a.

2nd step: Synthesis of Intermediate 2-1b

Intermediate 2-1a (95.2 g, 319.3 mmol) and 2,3-dichloro-5,6-dicyano-1,4-benzoquinone (108.7 g, 478.9 mmol) were put in a round-bottomed flask and dissolved in 600 m1 of toluene. The solution was stirred at 80° C. for 12 hours. When a reaction was completed, after removing the reaction solvent, the residue was treated through column chromatography, obtaining 41.3 g (44%) of Intermediate 2-1b.

3rd step: Synthesis of Intermediate 2-1c

Intermediate 2-1b (41.3 g, 139.0 mmol), iodobenzene (199.2 g, 976.0 mmol), CuI (5.31 g, 28.0 mmol), K₂CO₃ (28.9 g, 209.0 mmol), and 1,10-phenanthroline (5.03 g, 28.0 mmol) were dissolved in 500 m1 of DMF in a round-bottomed flask. The solution was stirred at 180° C. for 12 hours. When a reaction was completed, after removing the reaction solvent under a reduced pressure, a product therefrom was dissolved in methylene chloride and then, silica gel-filtered. The product was concentrated with methylene chloride and recrystallized with hexane, obtaining 39.0 g (75%) of Intermediate 2-1c.

4th step: Synthesis of Compound A-84

5.0 g (13.46 mmol) of Intermediate 2-1c, 4.41 g (13.46 mmol) of an amine intermediate 2-1d, 1.94 g (20.19 mmol) of sodium t-butoxide, and 0.54 g (1.35 mmol) of tri-tert-butylphosphine were dissolved in 100 m1 of toluene, and 0.37 g (0.4 mmol) of Pd(dba)₂ was added thereto and then, stirred under reflux at nitrogen atmosphere for 12 hours. After the reaction was completed, an organic layer was extracted therefrom with toluene and distilled water, dried with anhydrous magnesium sulfate, filtered, and the filtrate was concentrated under reduced pressure. A product therefrom was purified by silica gel column chromatography with normal hexane/methylene chloride (a volume ratio of 2:1) to obtain 6.4 g (Yield: 82.0%) of Compound A-84.

### Synthesis Example 11: Synthesis of Compound 2-92

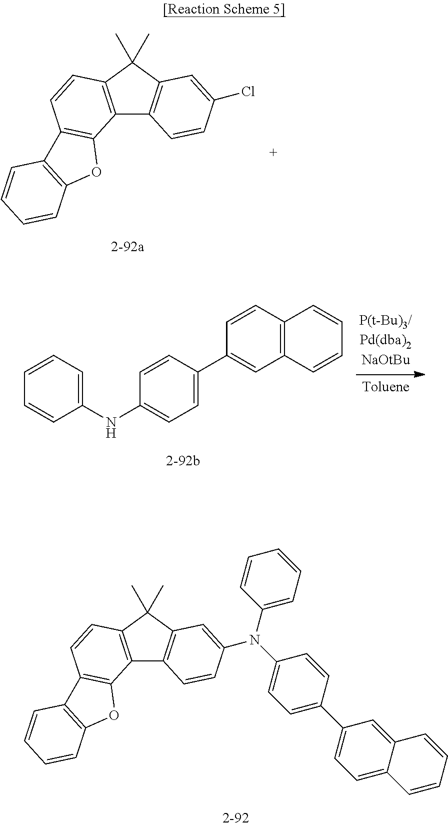 [SMILES: CC1(C)c2cc(Cl)ccc2-c2c1ccc1c2oc2ccccc21 ; c1ccc(Nc2ccc(-c3ccc4ccccc4c3)cc2)cc1 ; CC1(C)c2cc(N(c3ccccc3)c3ccc(-c4ccc5ccccc5c4)cc3)ccc2-c2c1ccc1c2oc2ccccc21]

1st step: Synthesis of Intermediate 2-92a

It was synthesized with reference to KR10-1423173 B1.

2nd step: Synthesis of Compound 2-92

5.0 g (16.93 mmol) of Intermediate 2-92a, 5.4 g (16.93 mmol) of an amine intermediate 2-92b, 2.44 g (25.39 mmol) of sodium t-butoxide, and 0.68 g (1.69 mmol) of tri-tert-butylphosphine were dissolved in 100 m1 of toluene, and 0.47 g (0.51 mmol) of Pd(dba)₂ was added thereto and then, stirred under reflux for 12 hours under a nitrogen atmosphere. After the reaction was completed, extraction was performed with toluene and distilled water, the organic layer was dried with anhydrous magnesium sulfate, filtered, and the filtrate was concentrated under reduced pressure. The product was purified by silica gel column chromatography with normal hexane/methylene chloride (volume ratio of 2:1) to obtain 8.2 g (yield 84.0%) of the target compound 2-92.

(Manufacture of Organic Light Emitting Diode)

### Example 1

A glass substrate coated with ITO (Indium tin oxide) was washed with distilled water and ultrasonic waves. After washing with the distilled water, the glass substrate was ultrasonically washed with isopropyl alcohol, acetone, or methanol, and dried and then, moved to a plasma cleaner, cleaned by using oxygen plasma for 10 minutes, and moved to a vacuum depositor. This prepared ITO transparent electrode was used as an anode, Compound A doped with 3% NDP-9 (Novaled GmbH) was vacuum-deposited on the ITO substrate to form a 100 Å-thick hole injection layer, and Compound A was deposited on the hole injection layer to a thickness of 1,300 Å to form a hole transport layer. Compound B was deposited on the hole transport layer to a thickness of 700 Å to form a hole transport auxiliary layer. On the hole transport auxiliary layer, Compound 1 obtained in Synthesis Example 1 was used as a host and 2 wt % of [Ir(piq)₂acac] was used as a dopant to form a 400 Å-thick light emitting layer by vacuum deposition. In the case of the following Examples and Comparative Examples, ratios were separately described. Subsequently, Compound C was deposited at a thickness of 50 Å on the light emitting layer to form an electron transport auxiliary layer, and Compound D and LiQ were simultaneously vacuum-deposited at a weight ratio of 1:1 to form a 300 Å-thick electron transport layer. On the electron transport layer, LiQ and Al were sequentially vacuum-deposited to be 15 Å-thick and 1,200 Å-thick, manufacturing an organic light emitting diode.

The organic light emitting device has a structure having a five-layered organic layer, specifically as follows.

ITO/Compound A (3% NDP-9 doping, 100 Å)/Compound A (1,300 Å) /Compound B (700 Å)/EML [Compound 1: [Ir(piq)₂acac]=98:2 (wt %/wt %)] (400 Å)/Compound C (50 Å)/Compound D: LiQ (300 Å)/LiQ (15 Å)/Al (1,200 Å)

Compound A: N-(biphenyl-4-yl)-9,9-dimethyl-N-(4-(9-phenyl-9H-carbazol-3-yl)phenyl)-9H-fluoren-2-amine

Compound B: N,N-di([1,1′-biphenyl]-4-yl)-7,7-dimethyl-7H-fluoreno[4,3-b]benzofuran-10-amine

Compound C: 2-(3-(3-(9,9-dimethyl-9H-fluoren-2-yl)phenyl)phenyl)-4,6-diphenyl-1,3,5-triazine

Compound D: 8-(4-(4,6-di(naphthalen-2-yl)-1,3,5-triazin-2-yl)phenyl)quinoline

### Examples 2 to 5 and Comparative Examples 1 to 5

Diodes of Examples 2 to 5 and Comparative Examples 1 to 5 were manufactured in the same manner as in Example 1, except that the host was changed as shown in Table 2.

### Example 6 to 15 and Comparative Example 6 to 11

Diodes of Examples 6 to 15 and Comparative Examples 6 to 11 were manufactured in the same manner as in Example 1, except that the host was changed as shown in Table 3, and the first host and the second host were mixed in a weight ratio of 5:5.

Evaluation

The luminous efficiency and life-span characteristics of the organic light emitting diodes according to Examples 1 to 15 and Comparative Examples 1 to 11 were evaluated. Specific measurement methods are as follows, and the results are shown in Tables 2 and 3.

(1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

(2) Measurement of Luminance Change Depending on Voltage Change

Luminance was measured by using a luminance meter (Minolta Cs-1000A), while the voltage of the organic light emitting diodes was increased from 0 V to 10 V.

(3) Measurement of Luminous Efficiency

Luminous efficiency (cd/A) at the same current density (10 mA/cm²) were calculated by using the luminance and current density from the items (1) and (2), and a voltage.

The relative values based on the luminous efficiency of Comparative Example 2 and Comparative Example 7 were calculated and shown in Tables 2 and 3.

(4) Measurement of Driving Voltage

A driving voltage of each diode was measured using a current-voltage meter (Keithley 2400) at 15 mA/cm².

The relative values based on the driving voltages of Comparative Example 7 were calculated and shown in Table 3.

TABLE 2

| | | Luminous |
|---|---|---|
| | Single host | Efficiency (%) |
| Example 1 | Compound 1 | 192 |
| Example 2 | Compound 3 | 154 |
| Example 3 | Compound 5 | 185 |
| Example 4 | Compound 6 | 138 |
| Example 5 | Compound 9 | 192 |
| Comparative Example 1 | Compound C-1 | 77 |
| Comparative Example 2 | Compound C-2 | 100 |
| Comparative Example 3 | Compound C-3 | 77 |
| Comparative Example 4 | Compound C-4 | 62 |
| Comparative Example 5 | Compound C-5 | 46 |

TABLE 3

| | Host | | Driving | Luminous |
|---|---|---|---|---|
| | First | Second | voltage | (%) |
| | host | host | (%) | Efficiency |
| Example 6 | 1 | A-84 | 79 | 179 |
| Example 7 | 3 | A-84 | 75 | 143 |
| Example 8 | 5 | A-84 | 67 | 171 |
| Example 9 | 6 | A-84 | 75 | 129 |
| Example 10 | 9 | A-84 | 58 | 179 |
| Example 11 | 1 | 2-92 | 71 | 171 |
| Example 12 | 3 | 2-92 | 67 | 132 |
| Example 13 | 5 | 2-92 | 58 | 164 |
| Example 14 | 6 | 2-92 | 67 | 121 |
| Example 15 | 9 | 2-92 | 50 | 168 |
| Comparative Example 6 | C-1 | A-84 | 83 | 71 |
| Comparative Example 7 | C-3 | A-84 | 100 | 100 |
| Comparative Example 8 | C-5 | A-84 | 75 | 50 |
| Comparative Example 9 | C-1 | 2-92 | 75 | 68 |
| Comparative Example 10 | C-3 | 2-92 | 92 | 93 |
| Comparative Example 11 | C-5 | 2-92 | 63 | 54 |

Referring to Tables 2 and 3, when the compound according to an embodiment was used as a single host or in a mixed host in combination with a second host, the luminous efficiencies or driving voltages were significantly improved, compared to those using the comparative compounds.

One or more embodiments may provide a compound for an organic optoelectronic device capable of implementing a high efficiency and long life-span organic optoelectronic device.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic optoelectronic device, the compound being represented by Chemical Formula 1: 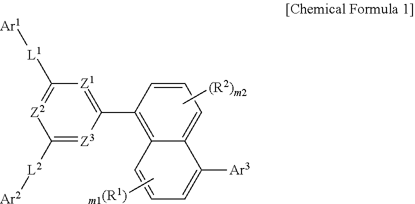
wherein, in Chemical Formula 1,
Z¹ to Z³ are each independently N or CR^{a}, provided that at least two of Z¹ to Z³ are N,
L¹ and L² are each independently a single bond, a substituted or unsubstituted C6 to C30 arylene group, or a substituted or unsubstituted C2 to C30 heterocyclic group,
Ar¹ and Ar² are each independently a substituted or unsubstituted C6 to C30 aryl group or a substituted or unsubstituted C2 to C30 heterocyclic group,
Ar³ is a substituted or unsubstituted C6 to C30 aryl group, a substituted or unsubstituted pyridyl group, a substituted or unsubstituted pyrimidinyl group, a substituted or unsubstituted triazinyl group, a substituted or unsubstituted dibenzofuranyl group, a substituted or unsubstituted dibenzothiophenyl group, a substituted or unsubstituted benzonaphthofuranyl group, or a substituted or unsubstituted benzonaphthothiophenyl group,
R^{a}, R¹, and R² are each independently hydrogen, deuterium, a substituted or unsubstituted C1 to C20 alkyl group, or a substituted or unsubstituted C6 to C20 aryl group,
m1 and m2 are each independently an integer of 1 to 3, and
when m2 is 2 or 3, each R² is separately present, or adjacent R²s are bonded with each other to form a ring.

2. The compound as claimed in claim 1, wherein:
Chemical Formula 1 is represented by Chemical Formula 1-1 or Chemical Formula 1-2: 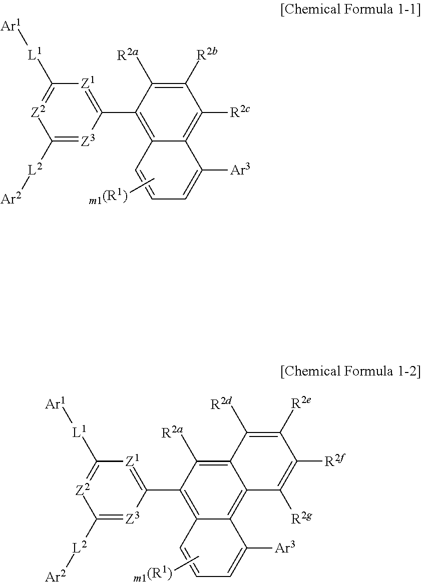
in Chemical Formula 1-1 and Chemical Formula 1-2,
Z¹ to Z³, L¹, L², Ar¹ to Ar², R¹, and m1 are defined the same as those of Chemical Formula 1, and
R^{2a}, R^{2b}, R^{2c}, R^{2d}, R^{2e}, R^{2f} and R^{2g} are each independently hydrogen, deuterium, a substituted or unsubstituted C1 to C20 alkyl group, or a substituted or unsubstituted C6 to C20 aryl group.

3. The compound as claimed in claim 1, wherein Ar¹ and Ar² are each independently a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted chrysenyl group, a substituted or unsubstituted benzophenanthrenyl group, a substituted or unsubstituted triphenylene group, a substituted or unsubstituted fluorenyl group, a substituted or unsubstituted dibenzosilolyl group, a substituted or unsubstituted dibenzofuranyl group, a substituted or unsubstituted dibenzothiophenyl group, a substituted or unsubstituted carbazolyl group, a substituted or unsubstituted benzonaphthofuranyl group, a substituted or unsubstituted benzonaphthothiophenyl group, a substituted or unsubstituted dinaphthofuranyl group, a substituted or unsubstituted dinaphthothiophenyl group, a substituted or unsubstituted fused dibenzofuranyl group, a substituted or unsubstituted fused dibenzothiophenyl group, a substituted or unsubstituted benzocarbazolyl group, a substituted or unsubstituted phenoxazinyl group, a substituted or unsubstituted thiophenoxazinyl group, a substituted or unsubstituted benzophenoxazinyl group, a substituted or unsubstituted benzothiophenoxazinyl group, a substituted or unsubstituted 10-phenyl-10H-spiro[acridine-9,9′-fluorenyl group], a substituted or unsubstituted 10H-spiro[acridine-9,9′-fluorenyl group], or a substituted or unsubstituted spiro[fluorene-9,9′-xanthenyl group].

4. The compound as claimed in claim 1, wherein AO is a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted fluorenyl group, a substituted or unsubstituted dibenzofuranyl group, a substituted or unsubstituted dibenzothiophenyl group, a substituted or unsubstituted benzonaphthofuranyl group, a substituted or unsubstituted benzonaphthothiophenyl group, a substituted or unsubstituted pyridyl group, a substituted or unsubstituted pyrimidinyl group, or a substituted or unsubstituted triazinyl group.

5. The compound as claimed in claim 1, wherein L¹ and L² are each independently a single bond, a substituted or unsubstituted phenylene group, a substituted or unsubstituted biphenylene group, a substituted or unsubstituted naphthylene group, a substituted or unsubstituted phenanthrenylene group, a substituted or unsubstituted dibenzofuranylene group, or a substituted or unsubstituted dibenzothiophenylene group.

6. The compound as claimed in claim 1, wherein:
moieties *-L¹-Ar¹ and *-L²-Ar² are each independently a moiety of Group I: 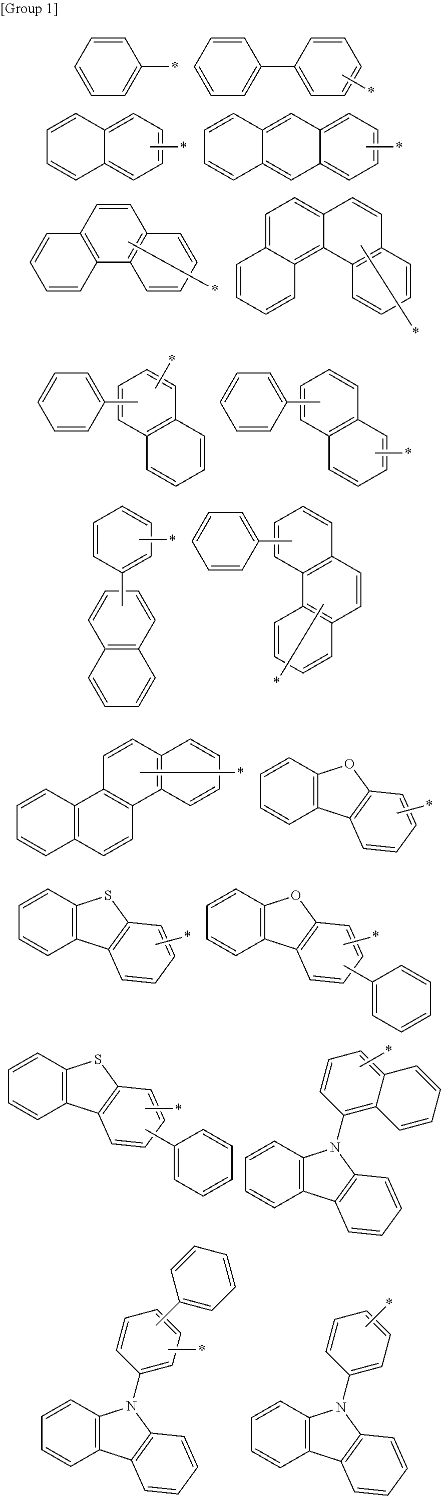 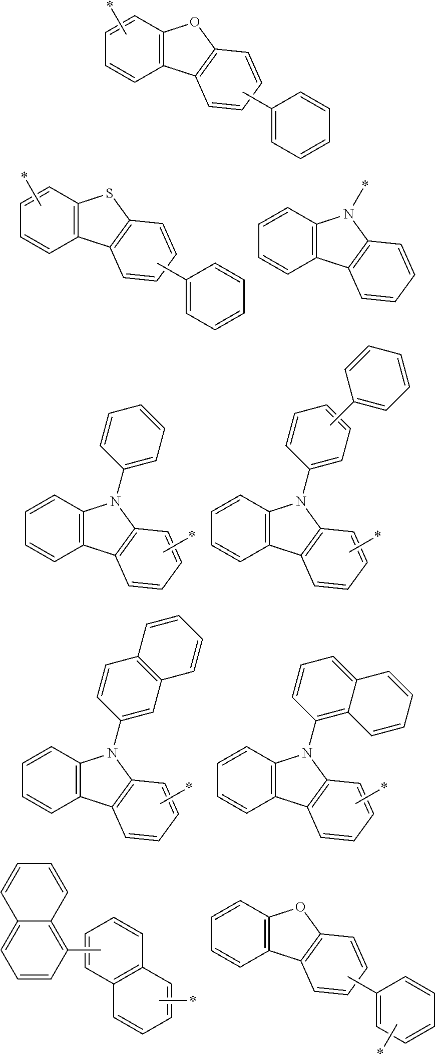 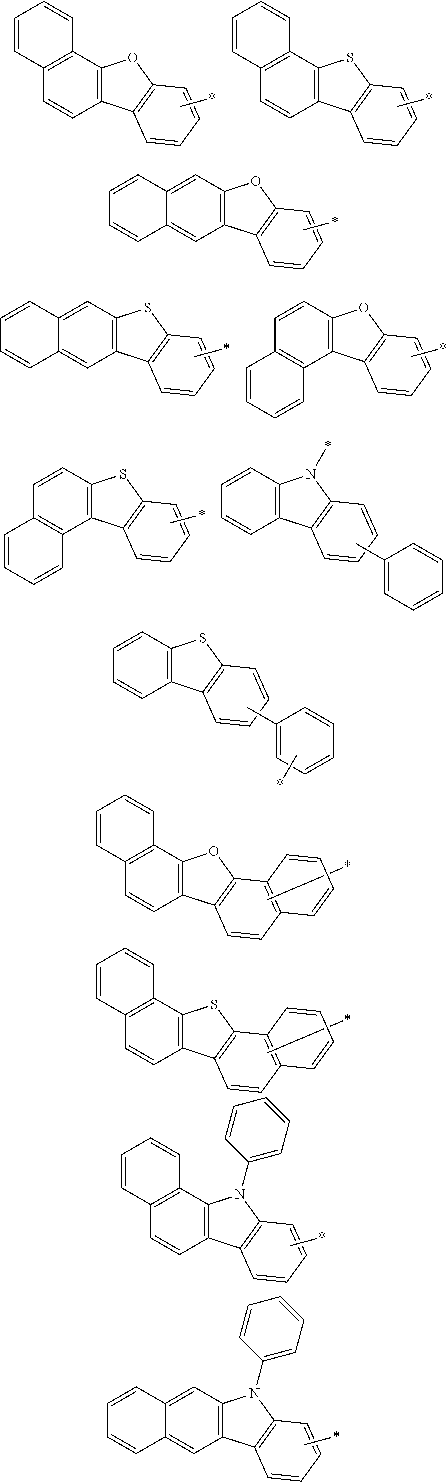 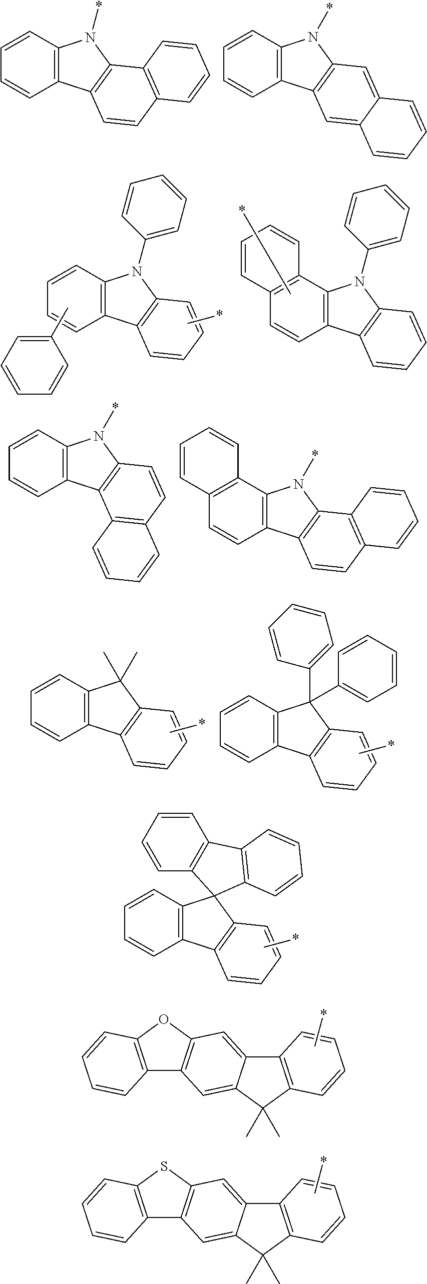 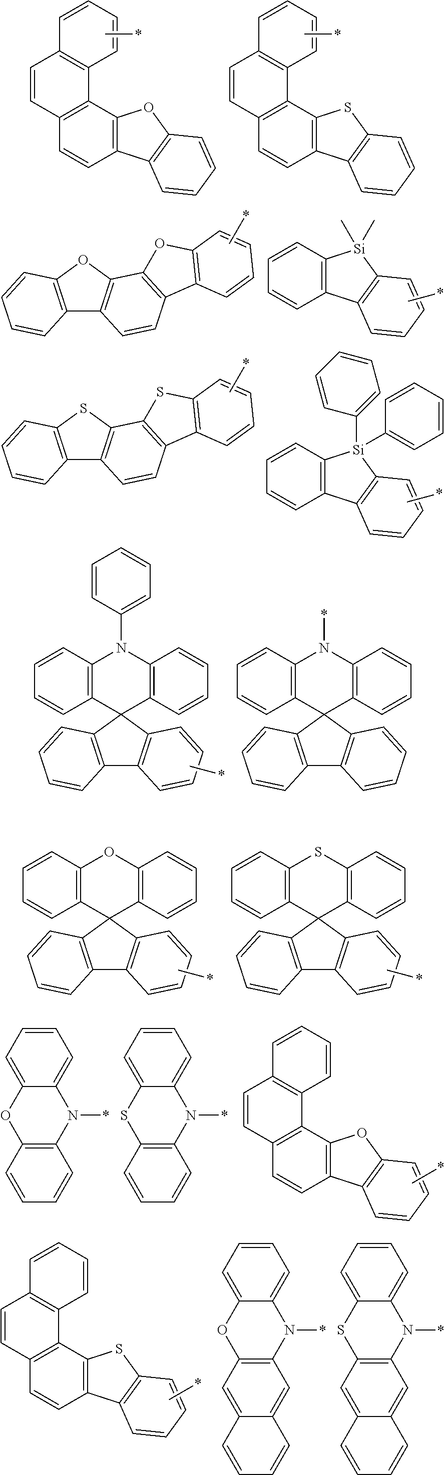 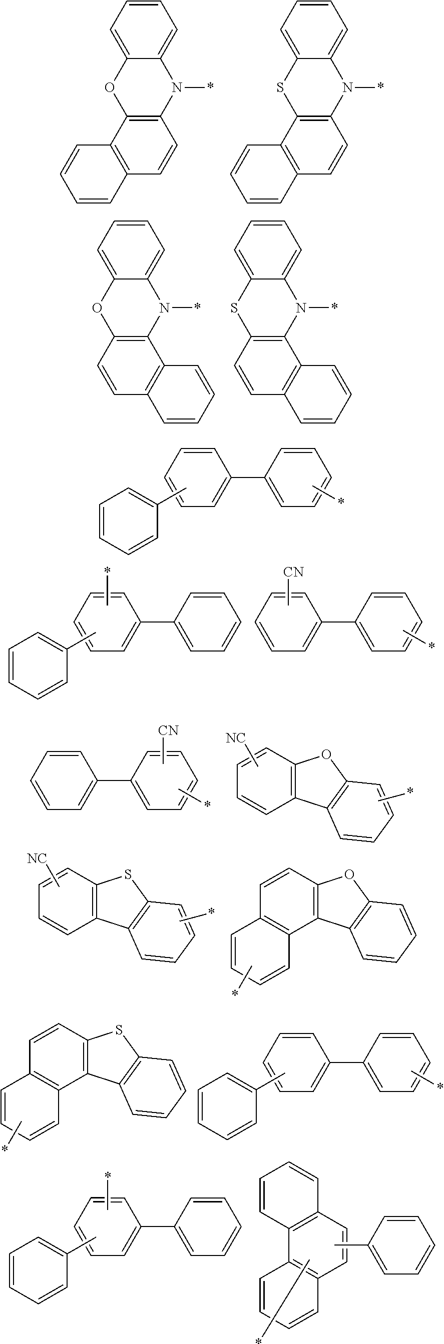 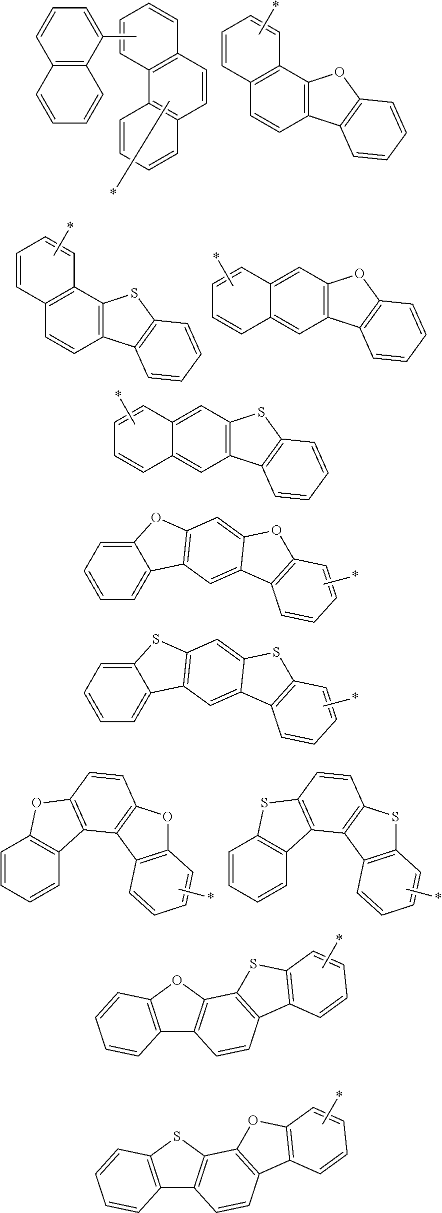
in Group I, * is a linking point.

7. The compound as claimed in claim 1, wherein the compound is a compound of Group 1:

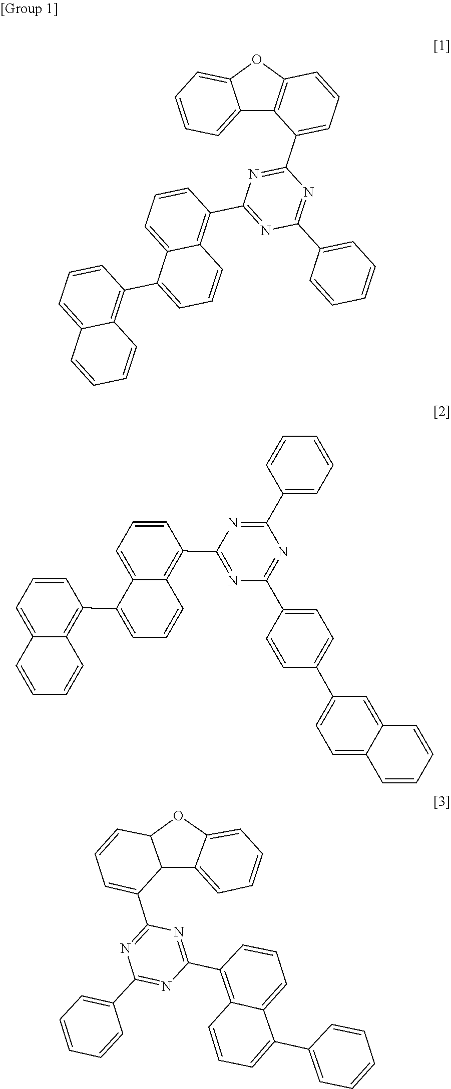 [SMILES: c1ccc(-c2nc(-c3cccc4c(-c5cccc6ccccc56)cccc34)nc(-c3cccc4oc5ccccc5c34)n2)cc1 ; c1ccc(-c2nc(-c3ccc(-c4ccc5ccccc5c4)cc3)nc(-c3cccc4c(-c5cccc6ccccc56)cccc34)n2)cc1 ; C1=CC2Oc3ccccc3C2C(c2nc(-c3ccccc3)nc(-c3cccc4c(-c5ccccc5)cccc34)n2)=C1]

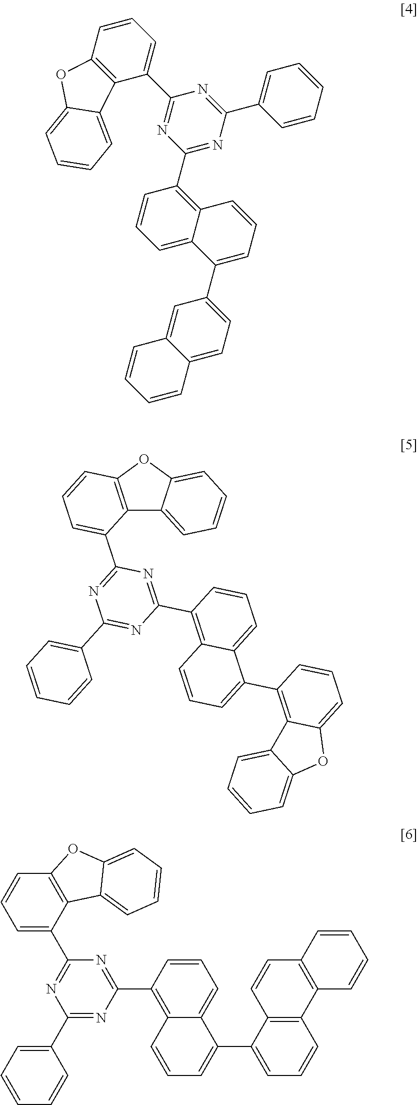 [SMILES: c1ccc(-c2nc(-c3cccc4c(-c5ccc6ccccc6c5)cccc34)nc(-c3cccc4oc5ccccc5c34)n2)cc1 ; c1ccc(-c2nc(-c3cccc4c(-c5cccc6oc7ccccc7c56)cccc34)nc(-c3cccc4oc5ccccc5c34)n2)cc1 ; c1ccc(-c2nc(-c3cccc4c(-c5cccc6c5ccc5ccccc56)cccc34)nc(-c3cccc4oc5ccccc5c34)n2)cc1]

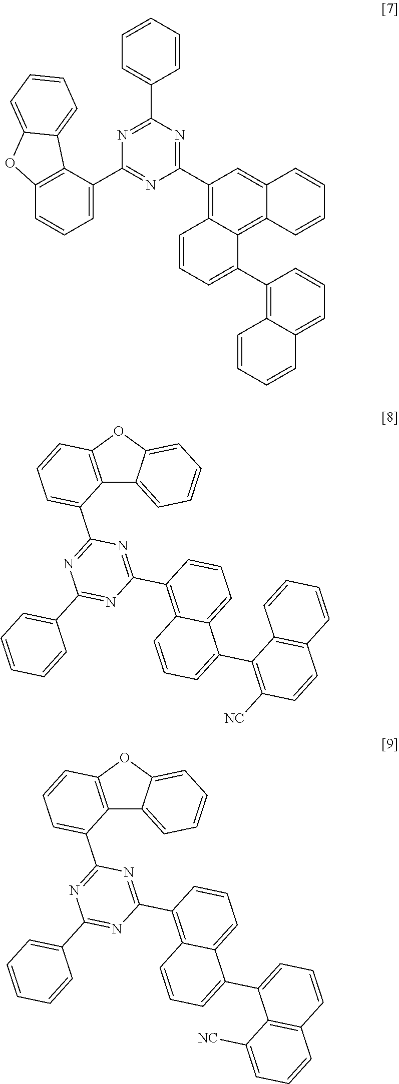 [SMILES: c1ccc(-c2nc(-c3cc4ccccc4c4c(-c5cccc6ccccc56)cccc34)nc(-c3cccc4oc5ccccc5c34)n2)cc1 ; N#Cc1ccc2ccccc2c1-c1cccc2c(-c3nc(-c4ccccc4)nc(-c4cccc5oc6ccccc6c45)n3)cccc12 ; N#Cc1cccc2cccc(-c3cccc4c(-c5nc(-c6ccccc6)nc(-c6cccc7oc8ccccc8c67)n5)cccc34)c12]

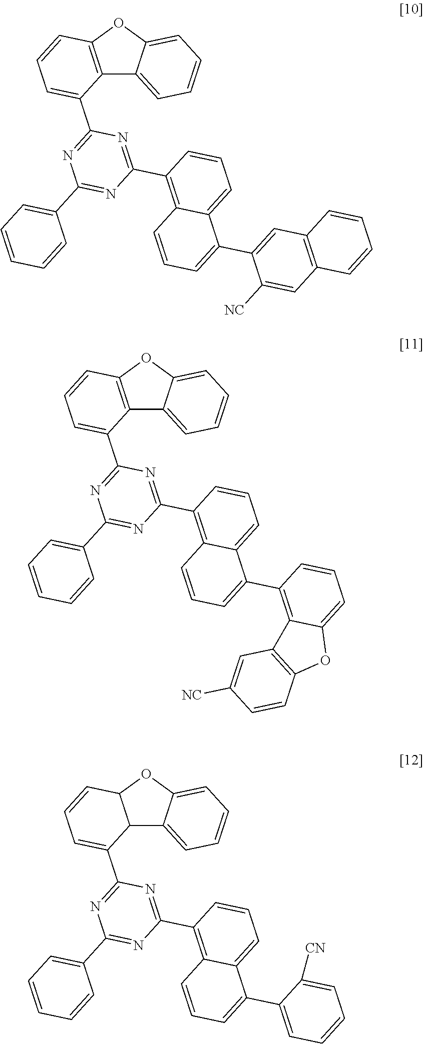 [SMILES: N#Cc1cc2ccccc2cc1-c1cccc2c(-c3nc(-c4ccccc4)nc(-c4cccc5oc6ccccc6c45)n3)cccc12 ; N#Cc1ccc2oc3cccc(-c4cccc5c(-c6nc(-c7ccccc7)nc(-c7cccc8oc9ccccc9c78)n6)cccc45)c3c2c1 ; N#Cc1ccccc1-c1cccc2c(-c3nc(C4=CC=CC5Oc6ccccc6C45)nc(-c4ccccc4)n3)cccc12]

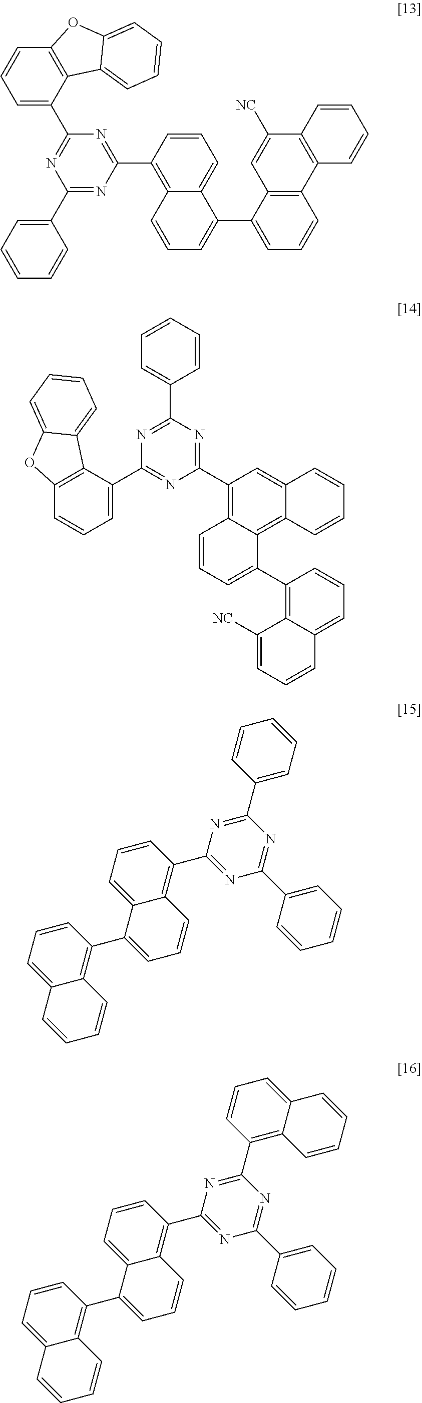

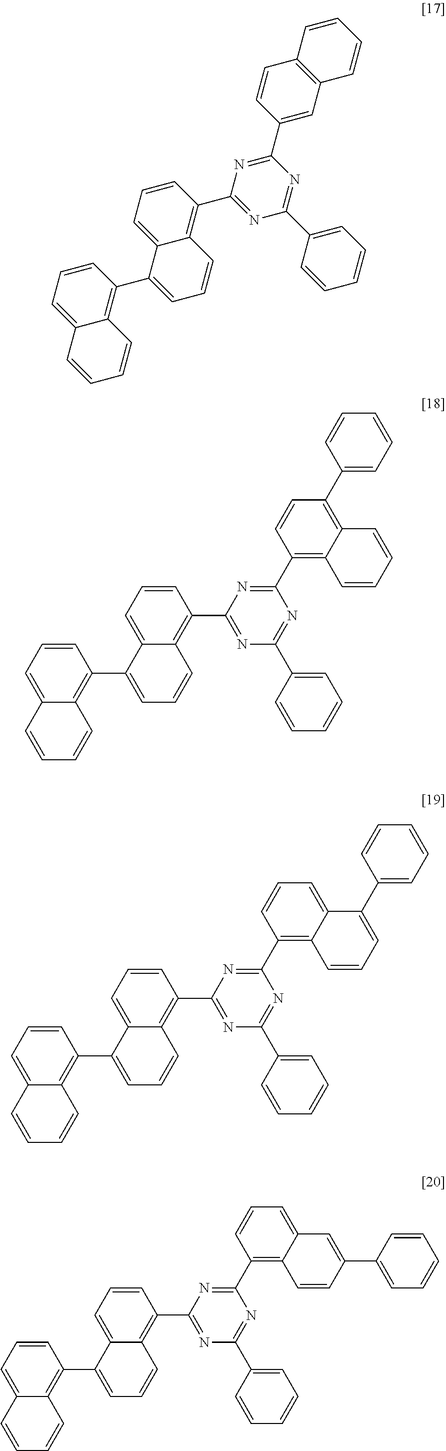 [SMILES: c1ccc(-c2nc(-c3ccc4ccccc4c3)nc(-c3cccc4c(-c5cccc6ccccc56)cccc34)n2)cc1 ; c1ccc(-c2nc(-c3ccc(-c4ccccc4)c4ccccc34)nc(-c3cccc4c(-c5cccc6ccccc56)cccc34)n2)cc1 ; c1ccc(-c2nc(-c3cccc4c(-c5ccccc5)cccc34)nc(-c3cccc4c(-c5cccc6ccccc56)cccc34)n2)cc1 ; c1ccc(-c2ccc3c(-c4nc(-c5ccccc5)nc(-c5cccc6c(-c7cccc8ccccc78)cccc56)n4)cccc3c2)cc1]

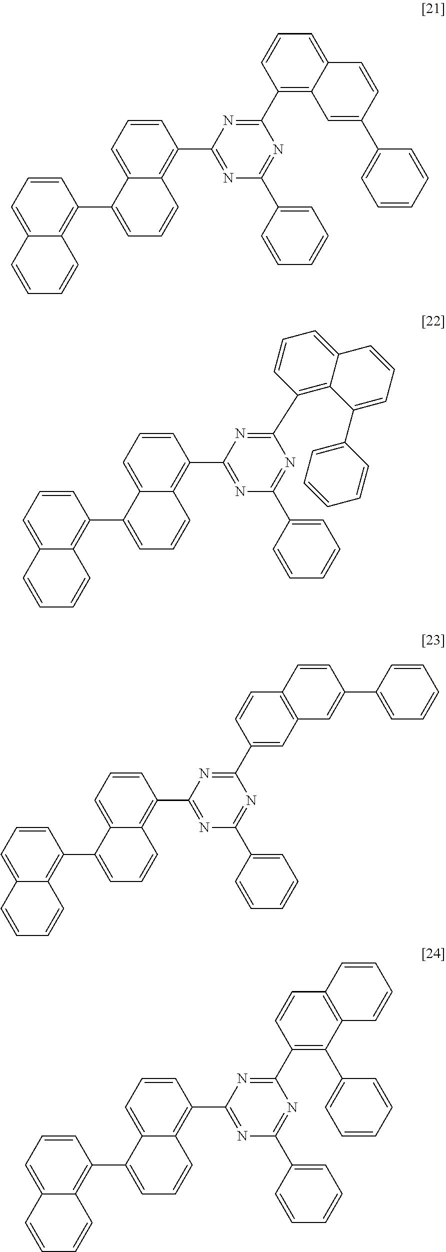 [SMILES: c1ccc(-c2ccc3cccc(-c4nc(-c5ccccc5)nc(-c5cccc6c(-c7cccc8ccccc78)cccc56)n4)c3c2)cc1 ; c1ccc(-c2nc(-c3cccc4c(-c5cccc6ccccc56)cccc34)nc(-c3cccc4cccc(-c5ccccc5)c34)n2)cc1 ; c1ccc(-c2ccc3ccc(-c4nc(-c5ccccc5)nc(-c5cccc6c(-c7cccc8ccccc78)cccc56)n4)cc3c2)cc1 ; c1ccc(-c2nc(-c3ccc4ccccc4c3-c3ccccc3)nc(-c3cccc4c(-c5cccc6ccccc56)cccc34)n2)cc1]

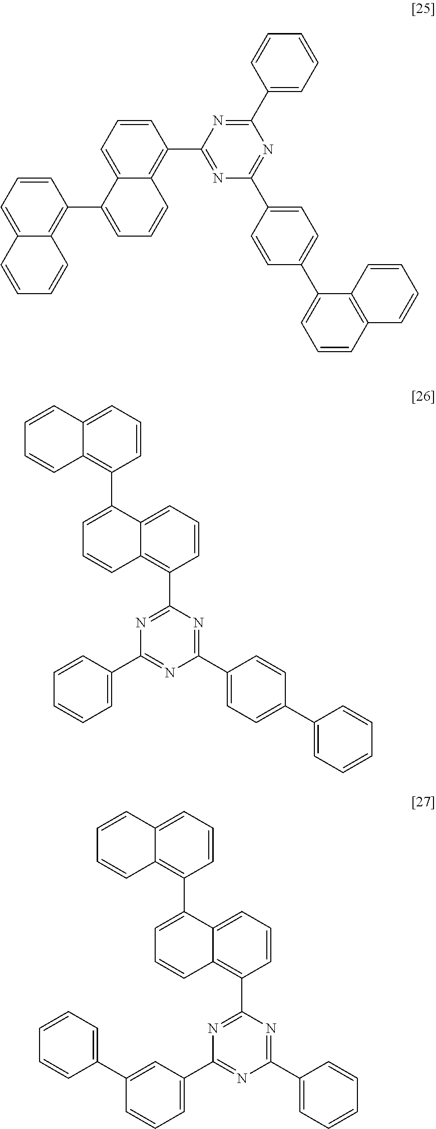 [SMILES: c1ccc(-c2nc(-c3ccc(-c4cccc5ccccc45)cc3)nc(-c3cccc4c(-c5cccc6ccccc56)cccc34)n2)cc1 ; c1ccc(-c2ccc(-c3nc(-c4ccccc4)nc(-c4cccc5c(-c6cccc7ccccc67)cccc45)n3)cc2)cc1 ; c1ccc(-c2cccc(-c3nc(-c4ccccc4)nc(-c4cccc5c(-c6cccc7ccccc67)cccc45)n3)c2)cc1]

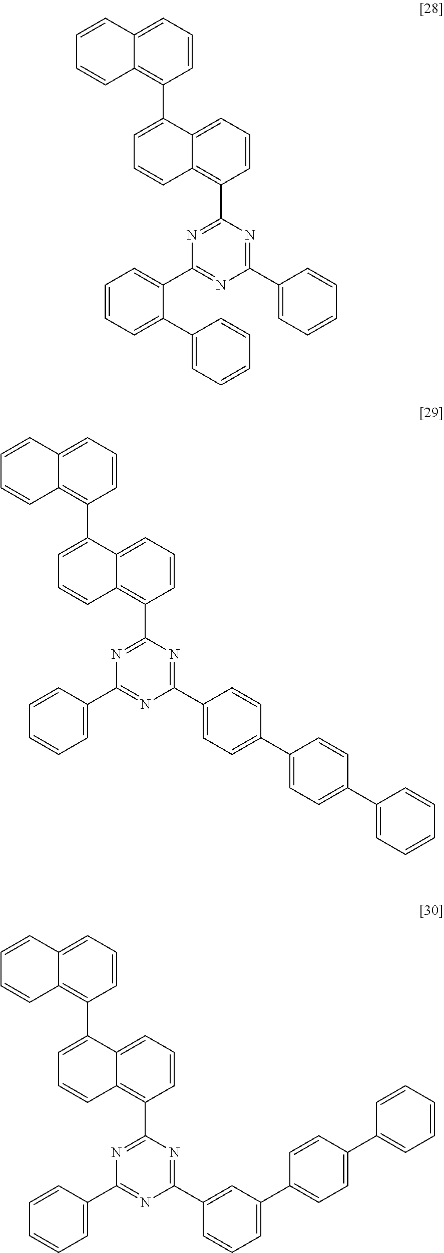 [SMILES: c1ccc(-c2nc(-c3ccccc3-c3ccccc3)nc(-c3cccc4c(-c5cccc6ccccc56)cccc34)n2)cc1 ; c1ccc(-c2ccc(-c3ccc(-c4nc(-c5ccccc5)nc(-c5cccc6c(-c7cccc8ccccc78)cccc56)n4)cc3)cc2)cc1 ; c1ccc(-c2ccc(-c3cccc(-c4nc(-c5ccccc5)nc(-c5cccc6c(-c7cccc8ccccc78)cccc56)n4)c3)cc2)cc1]

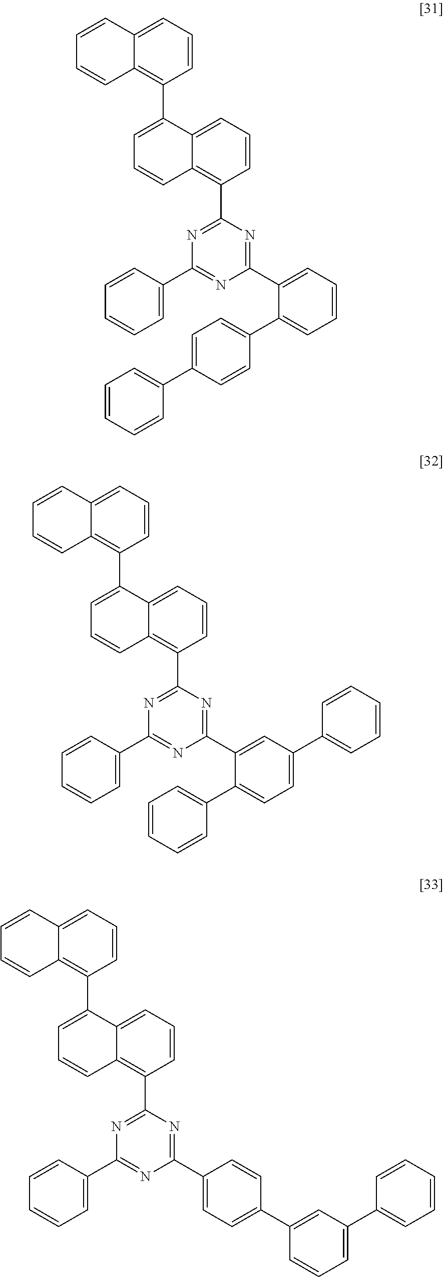 [SMILES: c1ccc(-c2ccc(-c3ccccc3-c3nc(-c4ccccc4)nc(-c4cccc5c(-c6cccc7ccccc67)cccc45)n3)cc2)cc1 ; c1ccc(-c2ccc(-c3ccccc3)c(-c3nc(-c4ccccc4)nc(-c4cccc5c(-c6cccc7ccccc67)cccc45)n3)c2)cc1 ; c1ccc(-c2cccc(-c3ccc(-c4nc(-c5ccccc5)nc(-c5cccc6c(-c7cccc8ccccc78)cccc56)n4)cc3)c2)cc1]

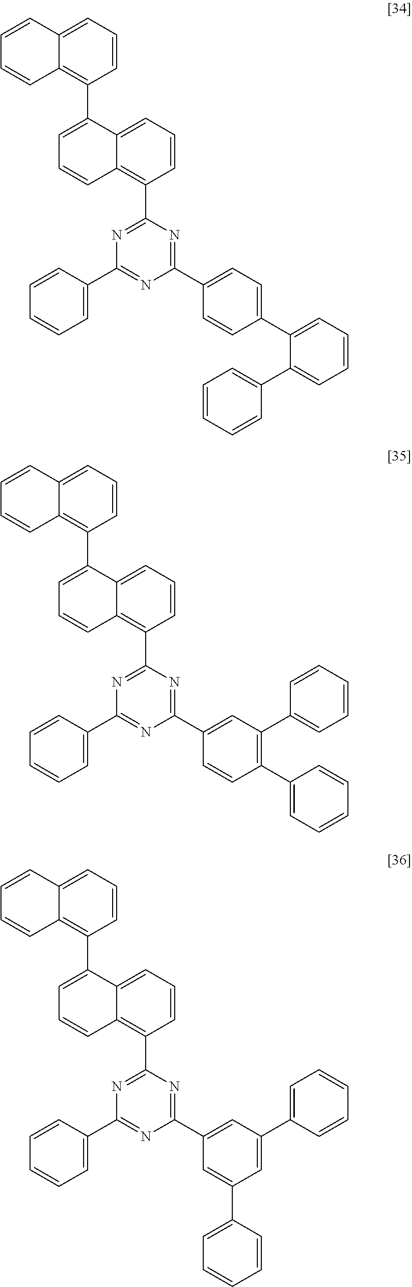 [SMILES: c1ccc(-c2nc(-c3ccc(-c4ccccc4-c4ccccc4)cc3)nc(-c3cccc4c(-c5cccc6ccccc56)cccc34)n2)cc1 ; c1ccc(-c2nc(-c3ccc(-c4ccccc4)c(-c4ccccc4)c3)nc(-c3cccc4c(-c5cccc6ccccc56)cccc34)n2)cc1 ; c1ccc(-c2cc(-c3ccccc3)cc(-c3nc(-c4ccccc4)nc(-c4cccc5c(-c6cccc7ccccc67)cccc45)n3)c2)cc1]

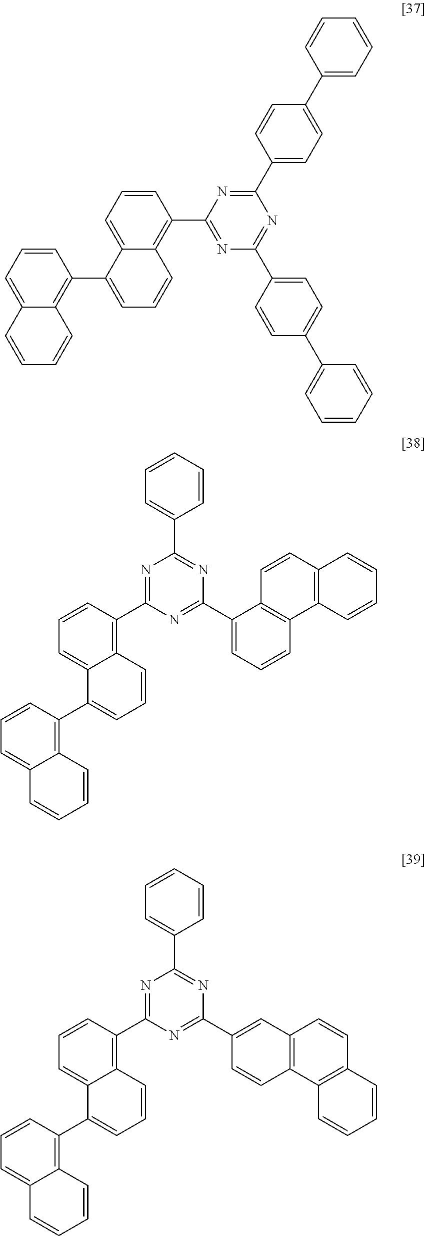 [SMILES: c1ccc(-c2ccc(-c3nc(-c4ccc(-c5ccccc5)cc4)nc(-c4cccc5c(-c6cccc7ccccc67)cccc45)n3)cc2)cc1 ; c1ccc(-c2nc(-c3cccc4c(-c5cccc6ccccc56)cccc34)nc(-c3cccc4c3ccc3ccccc34)n2)cc1 ; c1ccc(-c2nc(-c3ccc4c(ccc5ccccc54)c3)nc(-c3cccc4c(-c5cccc6ccccc56)cccc34)n2)cc1]

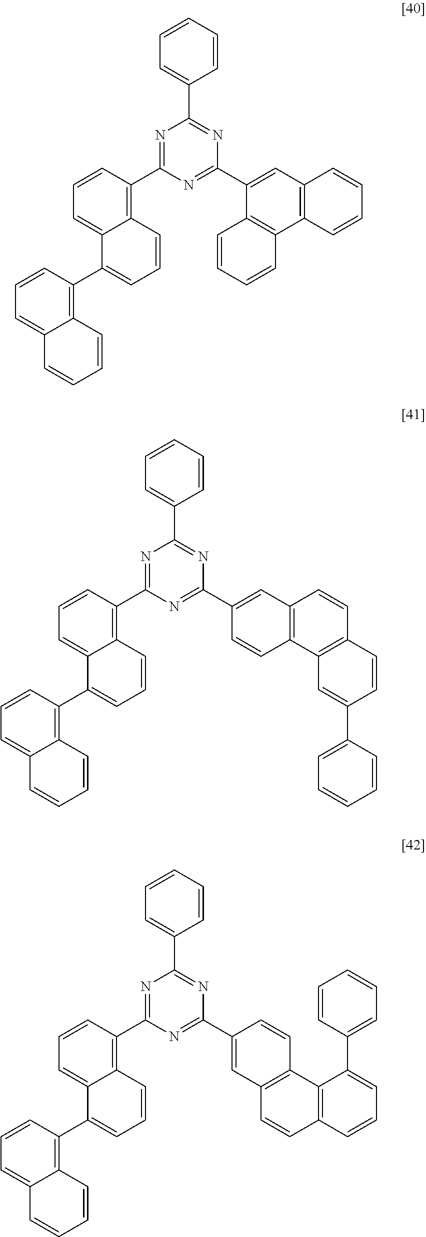 [SMILES: c1ccc(-c2nc(-c3cccc4c(-c5cccc6ccccc56)cccc34)nc(-c3cc4ccccc4c4ccccc34)n2)cc1 ; c1ccc(-c2ccc3ccc4cc(-c5nc(-c6ccccc6)nc(-c6cccc7c(-c8cccc9ccccc89)cccc67)n5)ccc4c3c2)cc1 ; c1ccc(-c2nc(-c3ccc4c(ccc5cccc(-c6ccccc6)c54)c3)nc(-c3cccc4c(-c5cccc6ccccc56)cccc34)n2)cc1]

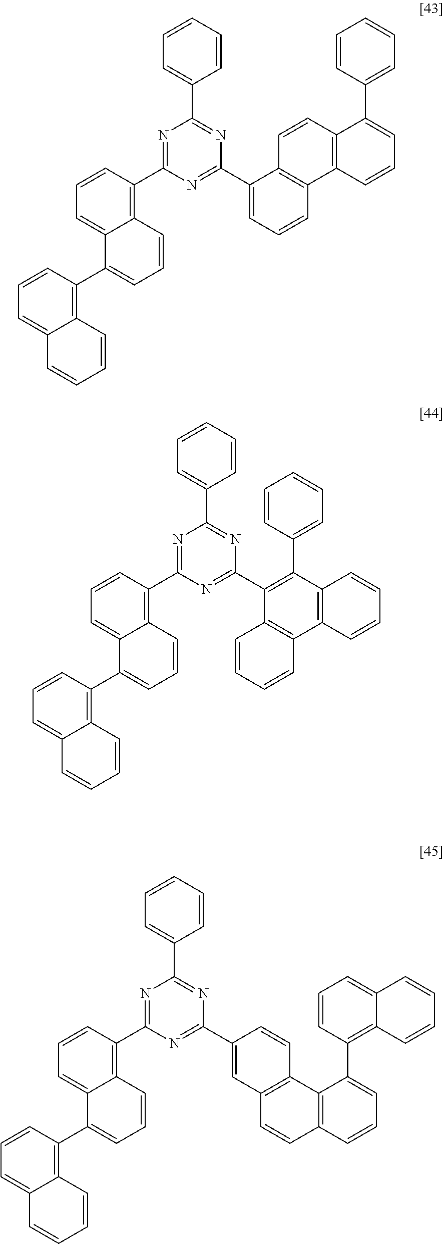 [SMILES: c1ccc(-c2nc(-c3cccc4c(-c5cccc6ccccc56)cccc34)nc(-c3cccc4c3ccc3c(-c5ccccc5)cccc34)n2)cc1 ; c1ccc(-c2nc(-c3cccc4c(-c5cccc6ccccc56)cccc34)nc(-c3c(-c4ccccc4)c4ccccc4c4ccccc34)n2)cc1 ; c1ccc(-c2nc(-c3ccc4c(ccc5cccc(-c6cccc7ccccc67)c54)c3)nc(-c3cccc4c(-c5cccc6ccccc56)cccc34)n2)cc1]

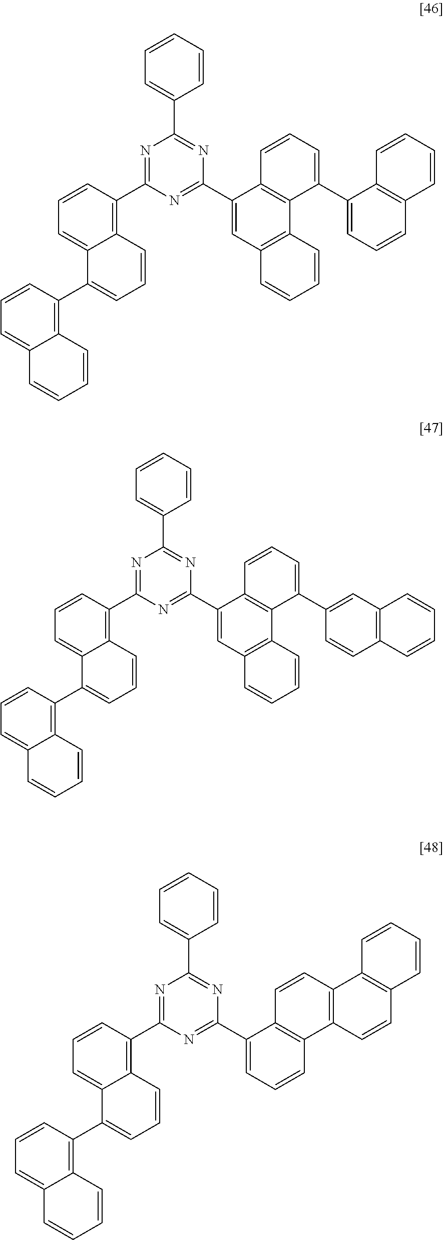 [SMILES: c1ccc(-c2nc(-c3cccc4c(-c5cccc6ccccc56)cccc34)nc(-c3cc4ccccc4c4c(-c5cccc6ccccc56)cccc34)n2)cc1 ; c1ccc(-c2nc(-c3cccc4c(-c5cccc6ccccc56)cccc34)nc(-c3cc4ccccc4c4c(-c5ccc6ccccc6c5)cccc34)n2)cc1 ; c1ccc(-c2nc(-c3cccc4c(-c5cccc6ccccc56)cccc34)nc(-c3cccc4c3ccc3c5ccccc5ccc43)n2)cc1]

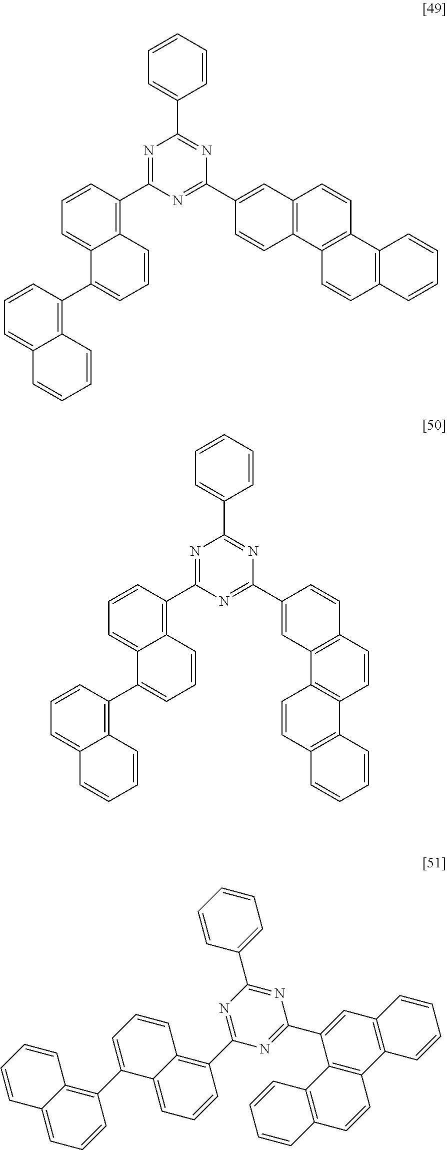 [SMILES: c1ccc(-c2nc(-c3ccc4c(ccc5c6ccccc6ccc45)c3)nc(-c3cccc4c(-c5cccc6ccccc56)cccc34)n2)cc1 ; c1ccc(-c2nc(-c3ccc4ccc5c6ccccc6ccc5c4c3)nc(-c3cccc4c(-c5cccc6ccccc56)cccc34)n2)cc1 ; c1ccc(-c2nc(-c3cccc4c(-c5cccc6ccccc56)cccc34)nc(-c3cc4ccccc4c4ccc5ccccc5c34)n2)cc1]

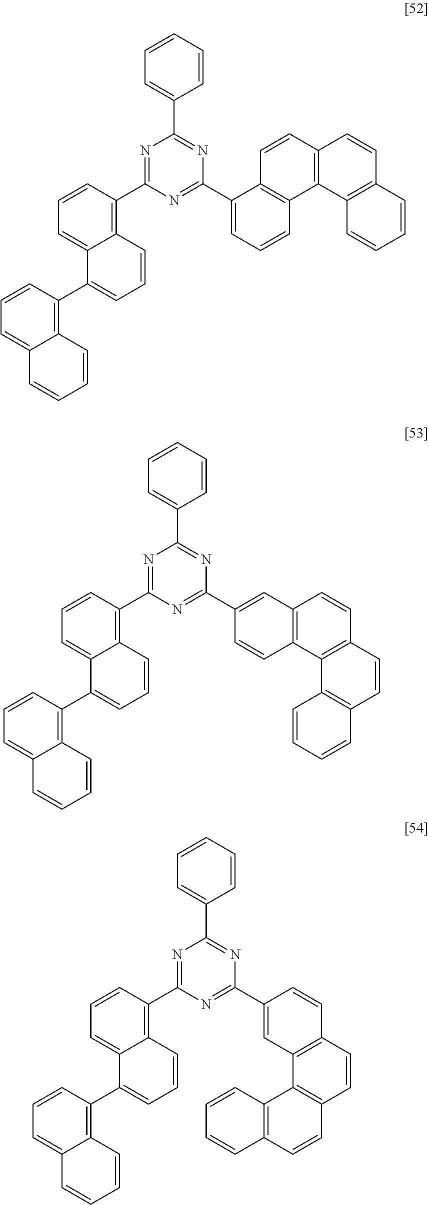 [SMILES: c1ccc(-c2nc(-c3cccc4c(-c5cccc6ccccc56)cccc34)nc(-c3cccc4c3ccc3ccc5ccccc5c34)n2)cc1 ; c1ccc(-c2nc(-c3ccc4c(ccc5ccc6ccccc6c54)c3)nc(-c3cccc4c(-c5cccc6ccccc56)cccc34)n2)cc1 ; c1ccc(-c2nc(-c3ccc4ccc5ccc6ccccc6c5c4c3)nc(-c3cccc4c(-c5cccc6ccccc56)cccc34)n2)cc1]

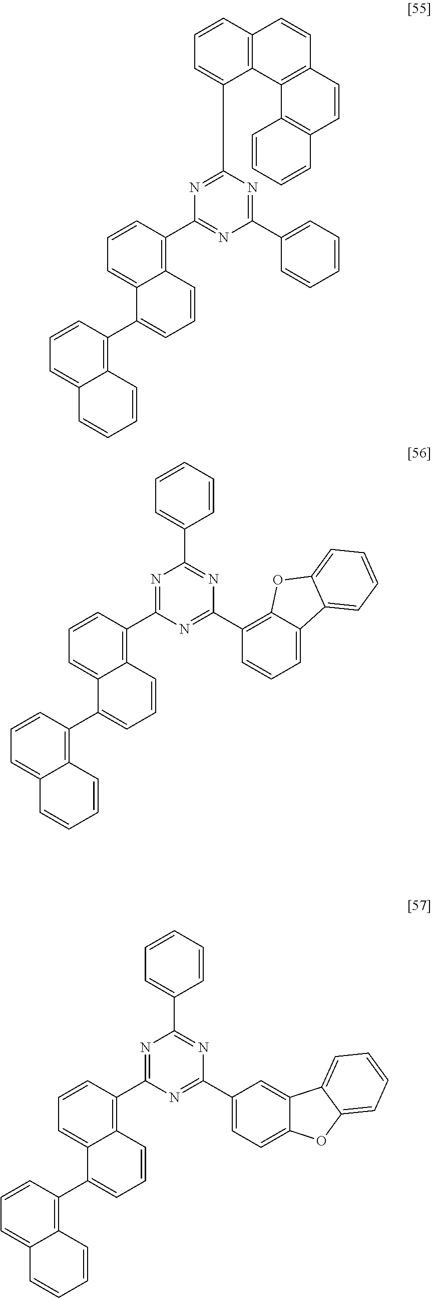 [SMILES: c1ccc(-c2nc(-c3cccc4c(-c5cccc6ccccc56)cccc34)nc(-c3cccc4ccc5ccc6ccccc6c5c34)n2)cc1 ; c1ccc(-c2nc(-c3cccc4c(-c5cccc6ccccc56)cccc34)nc(-c3cccc4c3oc3ccccc34)n2)cc1 ; c1ccc(-c2nc(-c3ccc4oc5ccccc5c4c3)nc(-c3cccc4c(-c5cccc6ccccc56)cccc34)n2)cc1]

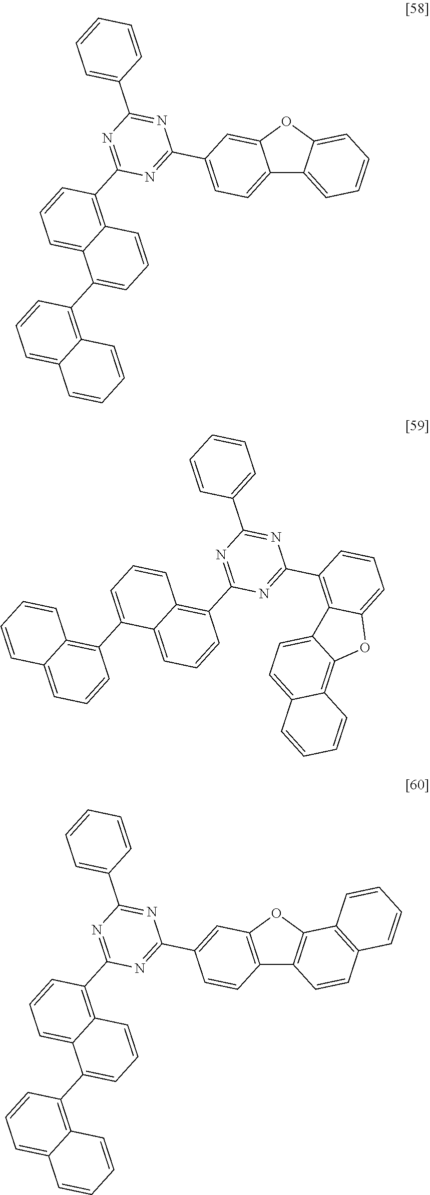 [SMILES: c1ccc(-c2nc(-c3ccc4c(c3)oc3ccccc34)nc(-c3cccc4c(-c5cccc6ccccc56)cccc34)n2)cc1 ; c1ccc(-c2nc(-c3cccc4c(-c5cccc6ccccc56)cccc34)nc(-c3cccc4oc5c6ccccc6ccc5c34)n2)cc1 ; c1ccc(-c2nc(-c3ccc4c(c3)oc3c5ccccc5ccc43)nc(-c3cccc4c(-c5cccc6ccccc56)cccc34)n2)cc1]

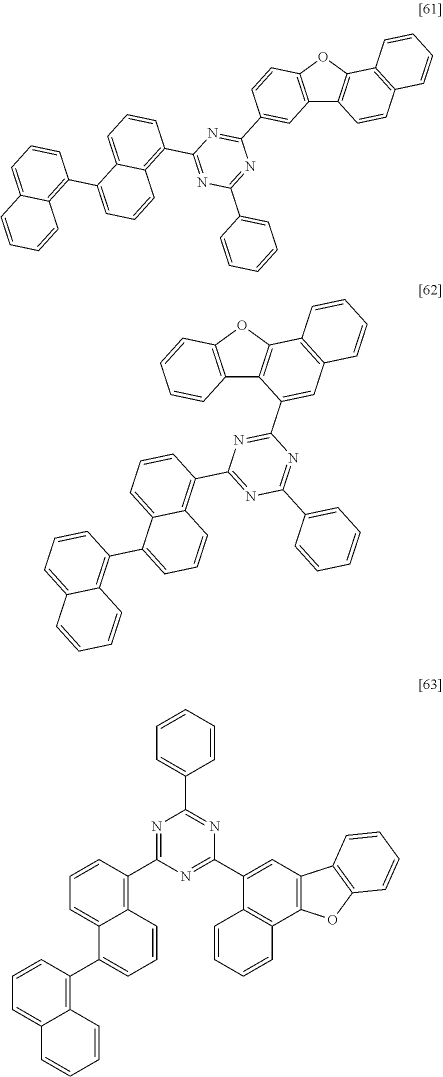 [SMILES: c1ccc(-c2nc(-c3ccc4oc5c6ccccc6ccc5c4c3)nc(-c3cccc4c(-c5cccc6ccccc56)cccc34)n2)cc1 ; c1ccc(-c2nc(-c3cccc4c(-c5cccc6ccccc56)cccc34)nc(-c3cc4ccccc4c4oc5ccccc5c34)n2)cc1 ; c1ccc(-c2nc(-c3cccc4c(-c5cccc6ccccc56)cccc34)nc(-c3cc4c5ccccc5oc4c4ccccc34)n2)cc1]

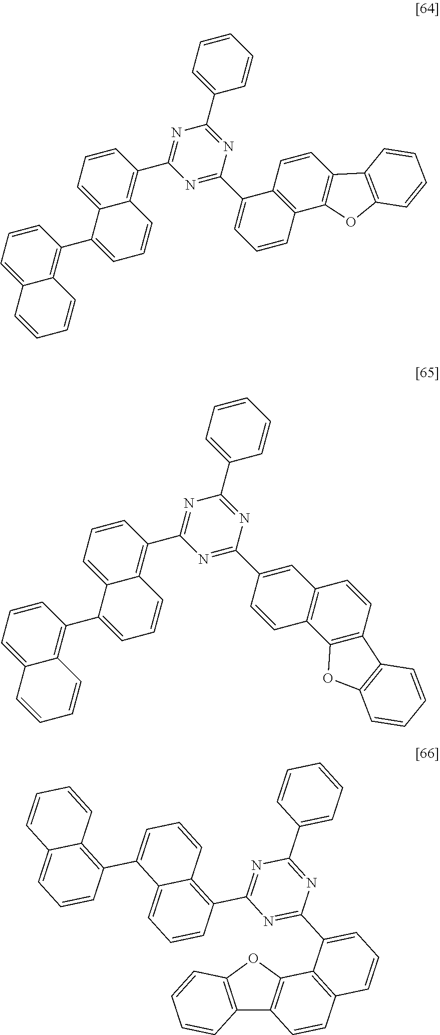 [SMILES: c1ccc(-c2nc(-c3cccc4c(-c5cccc6ccccc56)cccc34)nc(-c3cccc4c3ccc3c5ccccc5oc43)n2)cc1 ; c1ccc(-c2nc(-c3ccc4c(ccc5c6ccccc6oc45)c3)nc(-c3cccc4c(-c5cccc6ccccc56)cccc34)n2)cc1 ; c1ccc(-c2nc(-c3cccc4c(-c5cccc6ccccc56)cccc34)nc(-c3cccc4ccc5c6ccccc6oc5c34)n2)cc1]

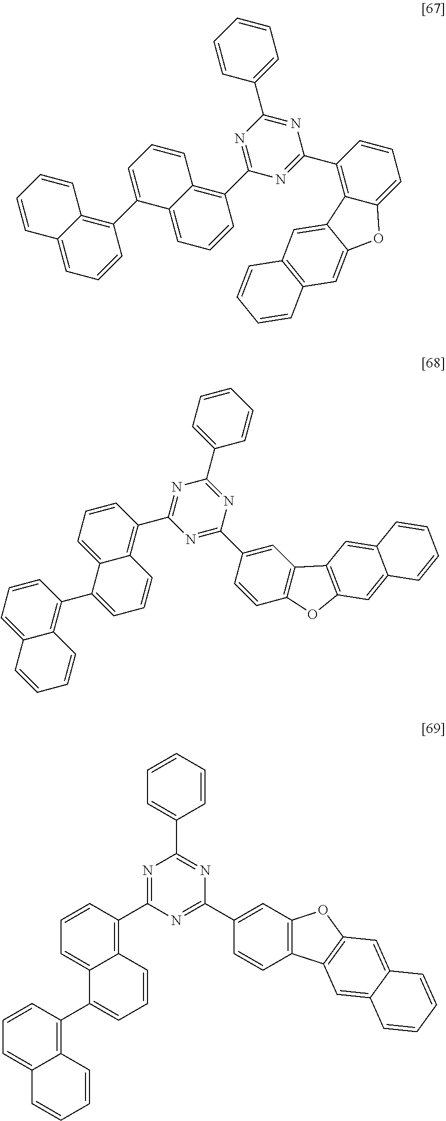 [SMILES: c1ccc(-c2nc(-c3cccc4c(-c5cccc6ccccc56)cccc34)nc(-c3cccc4oc5cc6ccccc6cc5c34)n2)cc1 ; c1ccc(-c2nc(-c3ccc4oc5cc6ccccc6cc5c4c3)nc(-c3cccc4c(-c5cccc6ccccc56)cccc34)n2)cc1 ; c1ccc(-c2nc(-c3ccc4c(c3)oc3cc5ccccc5cc34)nc(-c3cccc4c(-c5cccc6ccccc56)cccc34)n2)cc1]

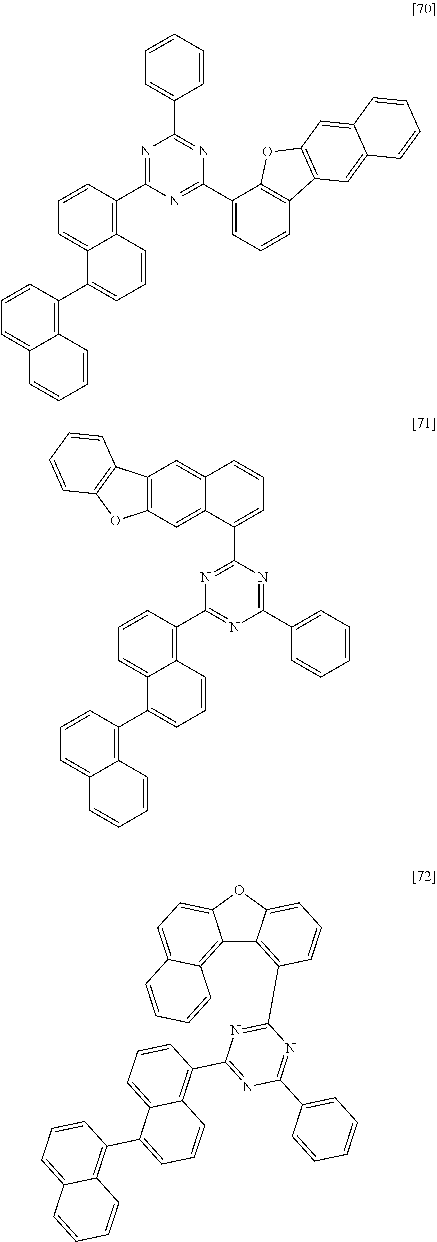

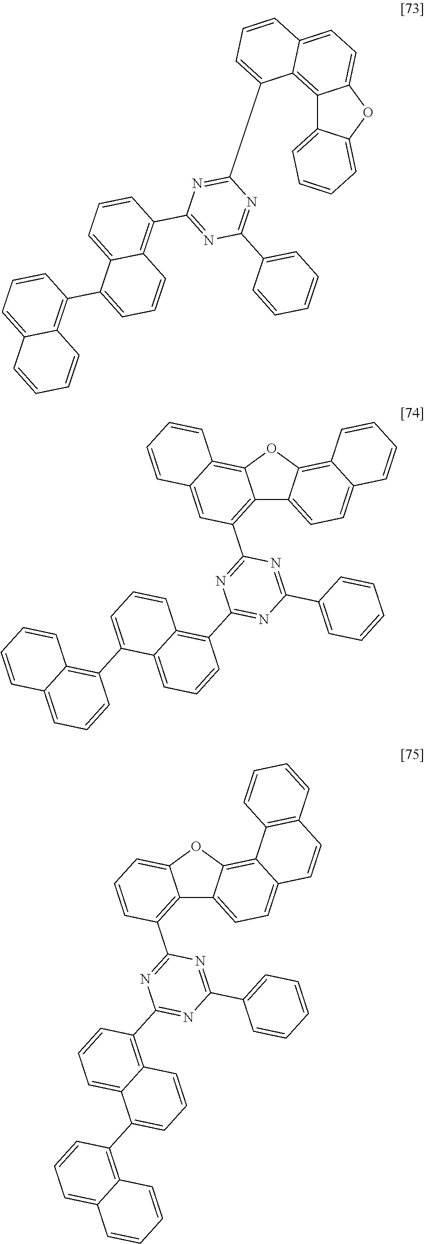 [SMILES: c1ccc(-c2nc(-c3cccc4c(-c5cccc6ccccc56)cccc34)nc(-c3cccc4ccc5oc6ccccc6c5c34)n2)cc1 ; c1ccc(-c2nc(-c3cccc4c(-c5cccc6ccccc56)cccc34)nc(-c3cc4ccccc4c4oc5c6ccccc6ccc5c34)n2)cc1 ; c1ccc(-c2nc(-c3cccc4c(-c5cccc6ccccc56)cccc34)nc(-c3cccc4oc5c(ccc6ccc7ccccc7c65)c34)n2)cc1]

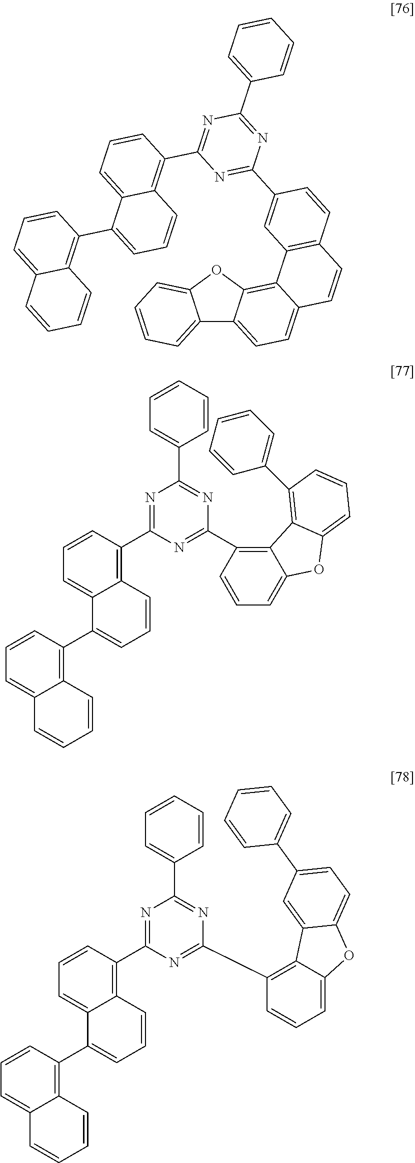

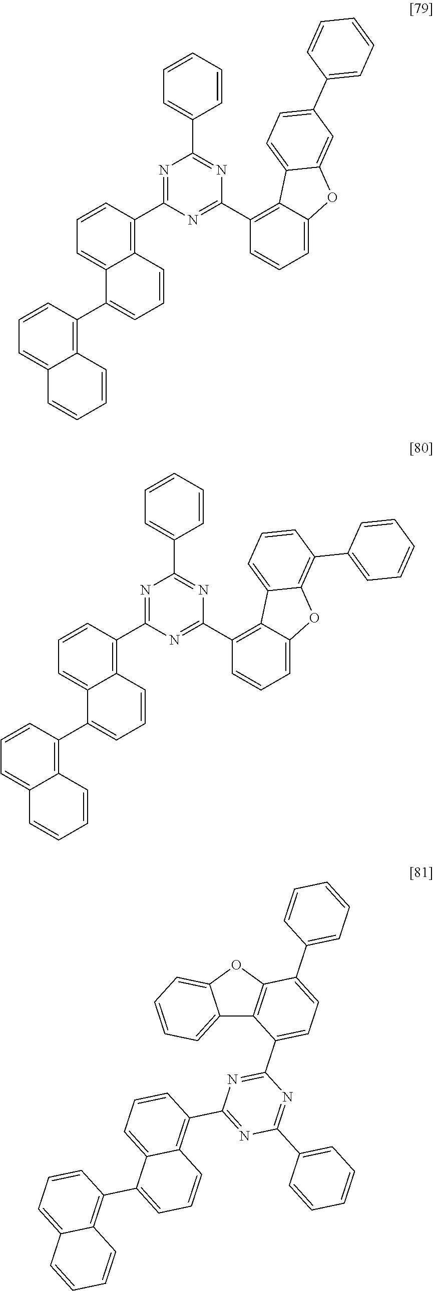 [SMILES: c1ccc(-c2ccc3c(c2)oc2cccc(-c4nc(-c5ccccc5)nc(-c5cccc6c(-c7cccc8ccccc78)cccc56)n4)c23)cc1 ; c1ccc(-c2nc(-c3cccc4c(-c5cccc6ccccc56)cccc34)nc(-c3cccc4oc5c(-c6ccccc6)cccc5c34)n2)cc1 ; c1ccc(-c2nc(-c3cccc4c(-c5cccc6ccccc56)cccc34)nc(-c3ccc(-c4ccccc4)c4oc5ccccc5c34)n2)cc1]

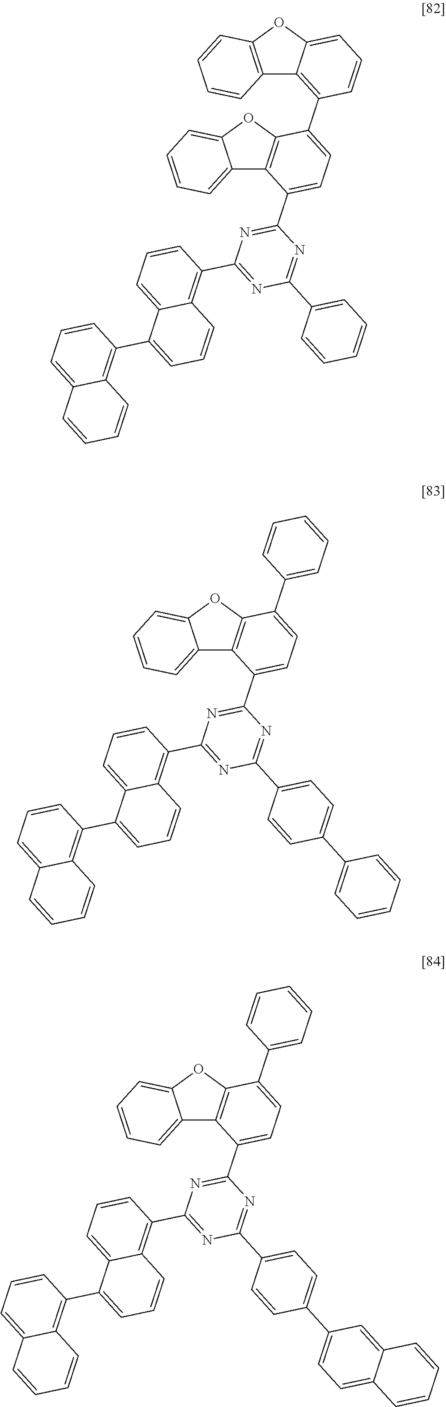 [SMILES: c1ccc(-c2nc(-c3cccc4c(-c5cccc6ccccc56)cccc34)nc(-c3ccc(-c4cccc5oc6ccccc6c45)c4oc5ccccc5c34)n2)cc1 ; c1ccc(-c2ccc(-c3nc(-c4cccc5c(-c6cccc7ccccc67)cccc45)nc(-c4ccc(-c5ccccc5)c5oc6ccccc6c45)n3)cc2)cc1 ; c1ccc(-c2ccc(-c3nc(-c4ccc(-c5ccc6ccccc6c5)cc4)nc(-c4cccc5c(-c6cccc7ccccc67)cccc45)n3)c3c2oc2ccccc23)cc1]

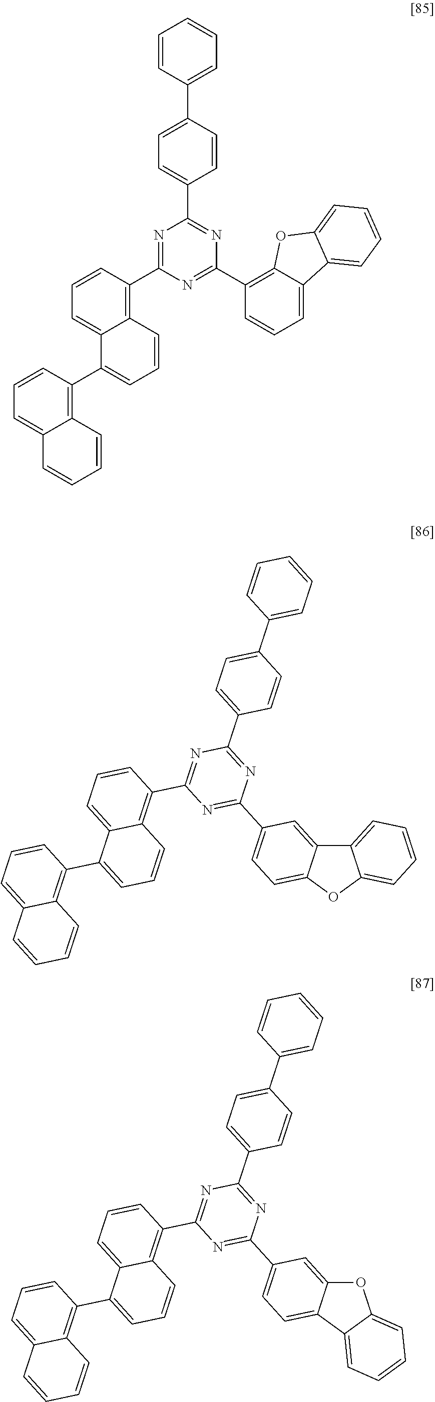 [SMILES: c1ccc(-c2ccc(-c3nc(-c4cccc5c(-c6cccc7ccccc67)cccc45)nc(-c4cccc5c4oc4ccccc45)n3)cc2)cc1 ; c1ccc(-c2ccc(-c3nc(-c4ccc5oc6ccccc6c5c4)nc(-c4cccc5c(-c6cccc7ccccc67)cccc45)n3)cc2)cc1 ; c1ccc(-c2ccc(-c3nc(-c4ccc5c(c4)oc4ccccc45)nc(-c4cccc5c(-c6cccc7ccccc67)cccc45)n3)cc2)cc1]

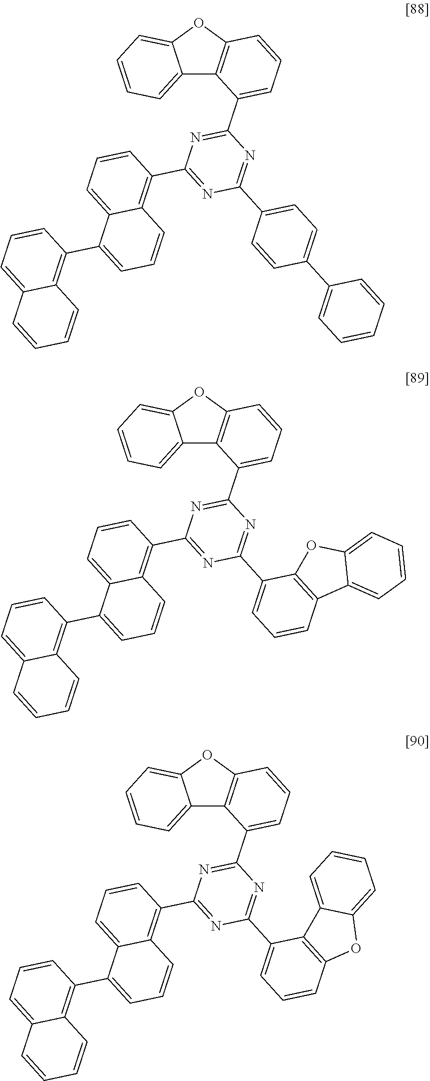 [SMILES: c1ccc(-c2ccc(-c3nc(-c4cccc5c(-c6cccc7ccccc67)cccc45)nc(-c4cccc5oc6ccccc6c45)n3)cc2)cc1 ; c1ccc2c(-c3cccc4c(-c5nc(-c6cccc7c6oc6ccccc67)nc(-c6cccc7oc8ccccc8c67)n5)cccc34)cccc2c1 ; c1ccc2c(-c3cccc4c(-c5nc(-c6cccc7oc8ccccc8c67)nc(-c6cccc7oc8ccccc8c67)n5)cccc34)cccc2c1]

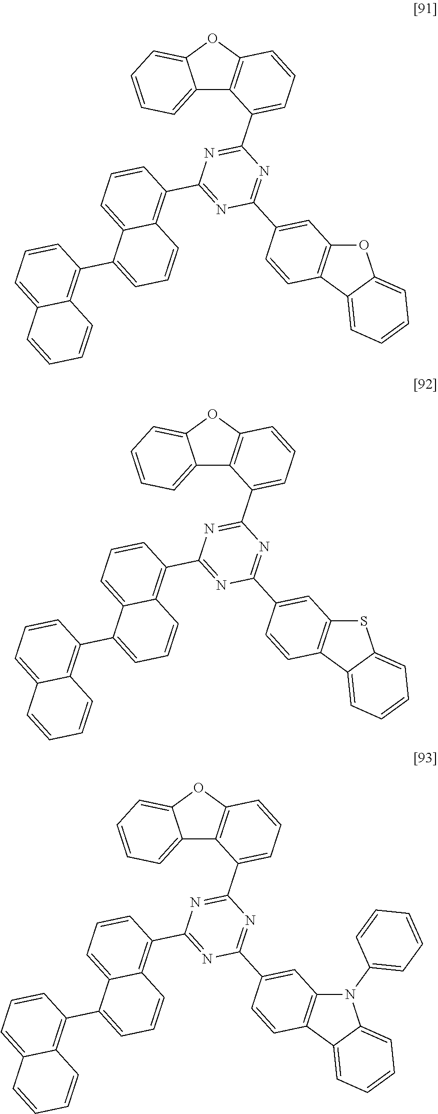 [SMILES: c1ccc2c(-c3cccc4c(-c5nc(-c6ccc7c(c6)oc6ccccc67)nc(-c6cccc7oc8ccccc8c67)n5)cccc34)cccc2c1 ; c1ccc2c(-c3cccc4c(-c5nc(-c6ccc7c(c6)sc6ccccc67)nc(-c6cccc7oc8ccccc8c67)n5)cccc34)cccc2c1 ; c1ccc(-n2c3ccccc3c3ccc(-c4nc(-c5cccc6c(-c7cccc8ccccc78)cccc56)nc(-c5cccc6oc7ccccc7c56)n4)cc32)cc1]

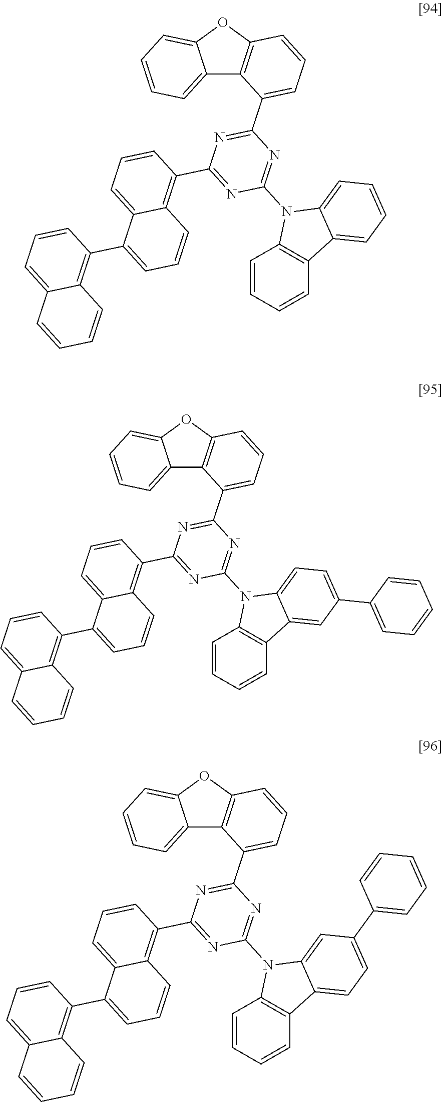 [SMILES: c1ccc2c(-c3cccc4c(-c5nc(-c6cccc7oc8ccccc8c67)nc(-n6c7ccccc7c7ccccc76)n5)cccc34)cccc2c1 ; c1ccc(-c2ccc3c(c2)c2ccccc2n3-c2nc(-c3cccc4c(-c5cccc6ccccc56)cccc34)nc(-c3cccc4oc5ccccc5c34)n2)cc1 ; c1ccc(-c2ccc3c4ccccc4n(-c4nc(-c5cccc6c(-c7cccc8ccccc78)cccc56)nc(-c5cccc6oc7ccccc7c56)n4)c3c2)cc1]

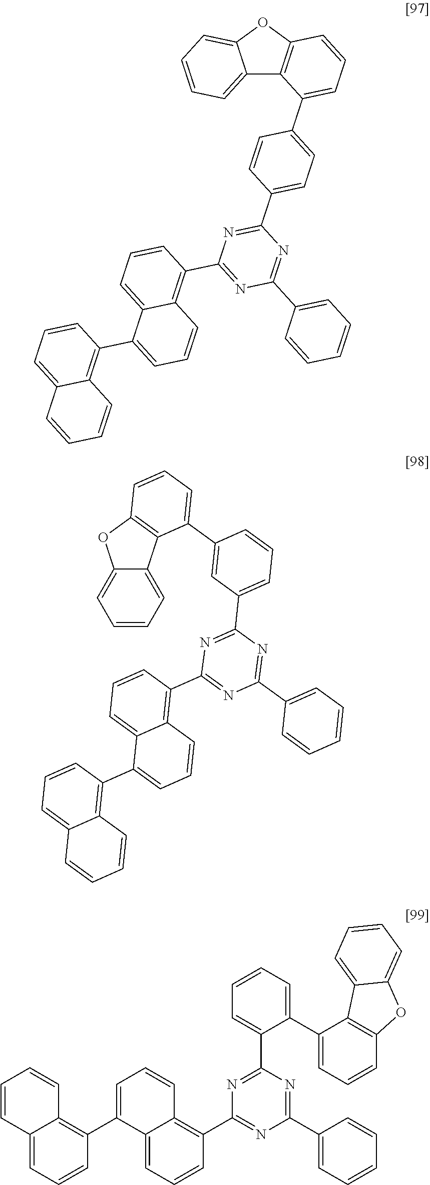 [SMILES: c1ccc(-c2nc(-c3ccc(-c4cccc5oc6ccccc6c45)cc3)nc(-c3cccc4c(-c5cccc6ccccc56)cccc34)n2)cc1 ; c1ccc(-c2nc(-c3cccc(-c4cccc5oc6ccccc6c45)c3)nc(-c3cccc4c(-c5cccc6ccccc56)cccc34)n2)cc1 ; c1ccc(-c2nc(-c3ccccc3-c3cccc4oc5ccccc5c34)nc(-c3cccc4c(-c5cccc6ccccc56)cccc34)n2)cc1]

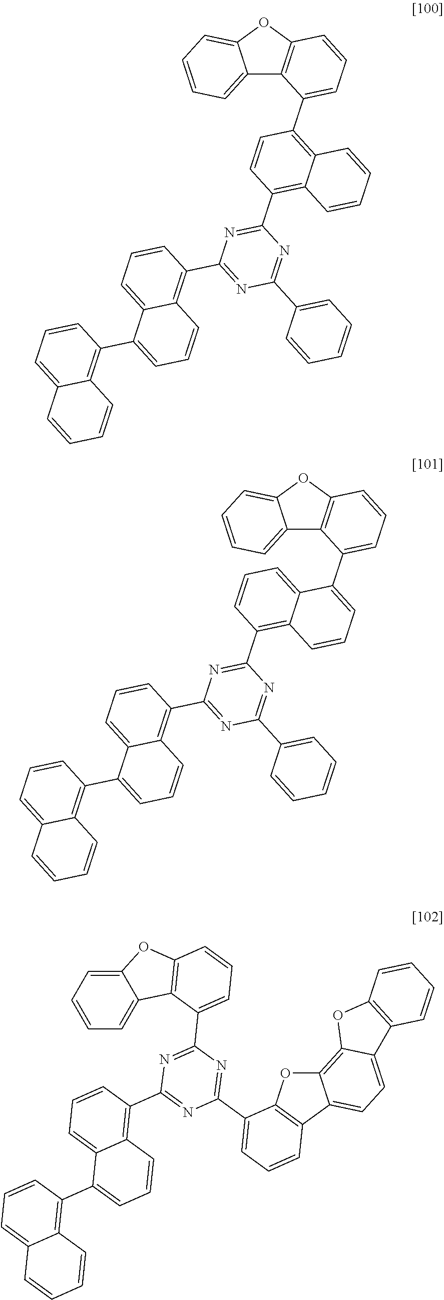 [SMILES: c1ccc(-c2nc(-c3cccc4c(-c5cccc6ccccc56)cccc34)nc(-c3ccc(-c4cccc5oc6ccccc6c45)c4ccccc34)n2)cc1 ; c1ccc(-c2nc(-c3cccc4c(-c5cccc6ccccc56)cccc34)nc(-c3cccc4c(-c5cccc6oc7ccccc7c56)cccc34)n2)cc1 ; c1ccc2c(-c3cccc4c(-c5nc(-c6cccc7c6oc6c7ccc7c8ccccc8oc76)nc(-c6cccc7oc8ccccc8c67)n5)cccc34)cccc2c1]

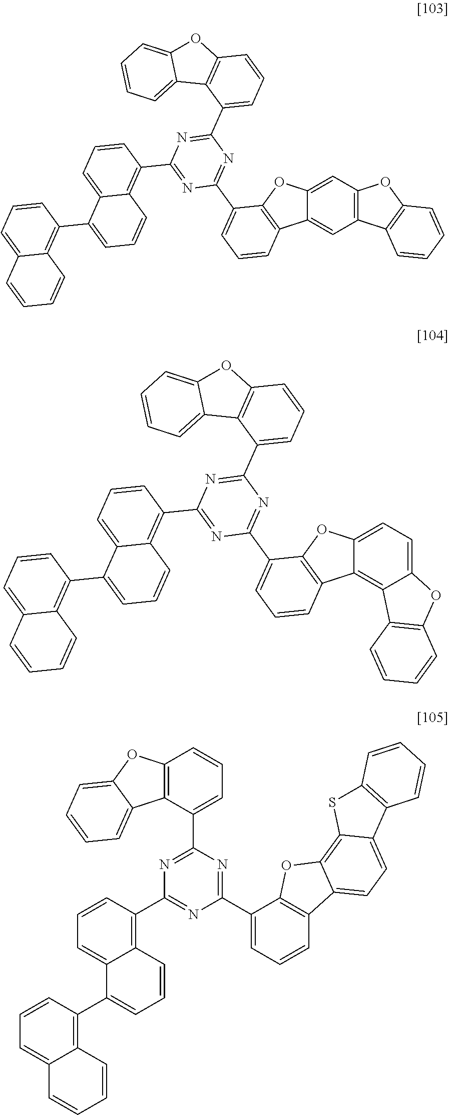 [SMILES: c1ccc2c(-c3cccc4c(-c5nc(-c6cccc7c6oc6cc8oc9ccccc9c8cc67)nc(-c6cccc7oc8ccccc8c67)n5)cccc34)cccc2c1 ; c1ccc2c(-c3cccc4c(-c5nc(-c6cccc7c6oc6ccc8oc9ccccc9c8c67)nc(-c6cccc7oc8ccccc8c67)n5)cccc34)cccc2c1 ; c1ccc2c(-c3cccc4c(-c5nc(-c6cccc7c6oc6c7ccc7c8ccccc8sc76)nc(-c6cccc7oc8ccccc8c67)n5)cccc34)cccc2c1]

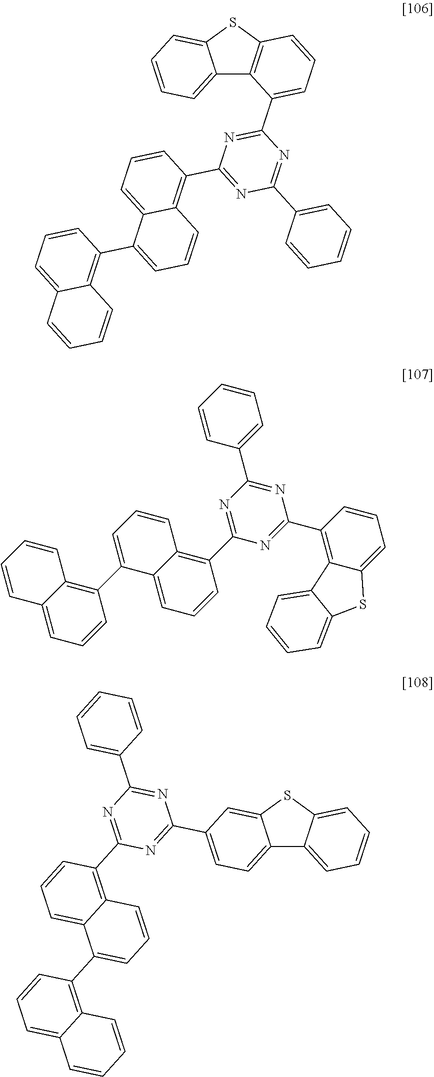 [SMILES: c1ccc(-c2nc(-c3cccc4c(-c5cccc6ccccc56)cccc34)nc(-c3cccc4sc5ccccc5c34)n2)cc1 ; c1ccc(-c2nc(-c3cccc4c(-c5cccc6ccccc56)cccc34)nc(-c3cccc4sc5ccccc5c34)n2)cc1 ; c1ccc(-c2nc(-c3ccc4c(c3)sc3ccccc34)nc(-c3cccc4c(-c5cccc6ccccc56)cccc34)n2)cc1]

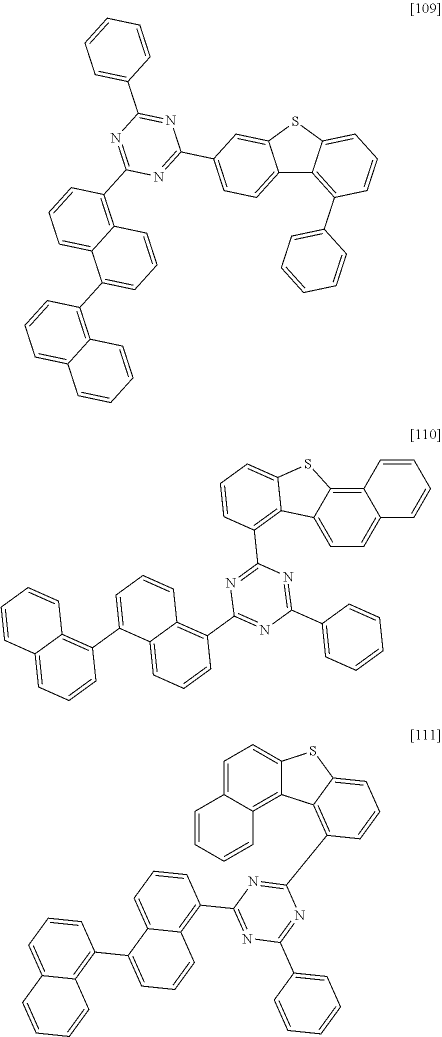 [SMILES: c1ccc(-c2nc(-c3ccc4c(c3)sc3cccc(-c5ccccc5)c34)nc(-c3cccc4c(-c5cccc6ccccc56)cccc34)n2)cc1 ; c1ccc(-c2nc(-c3cccc4c(-c5cccc6ccccc56)cccc34)nc(-c3cccc4sc5c6ccccc6ccc5c34)n2)cc1 ; c1ccc(-c2nc(-c3cccc4c(-c5cccc6ccccc56)cccc34)nc(-c3cccc4sc5ccc6ccccc6c5c34)n2)cc1]

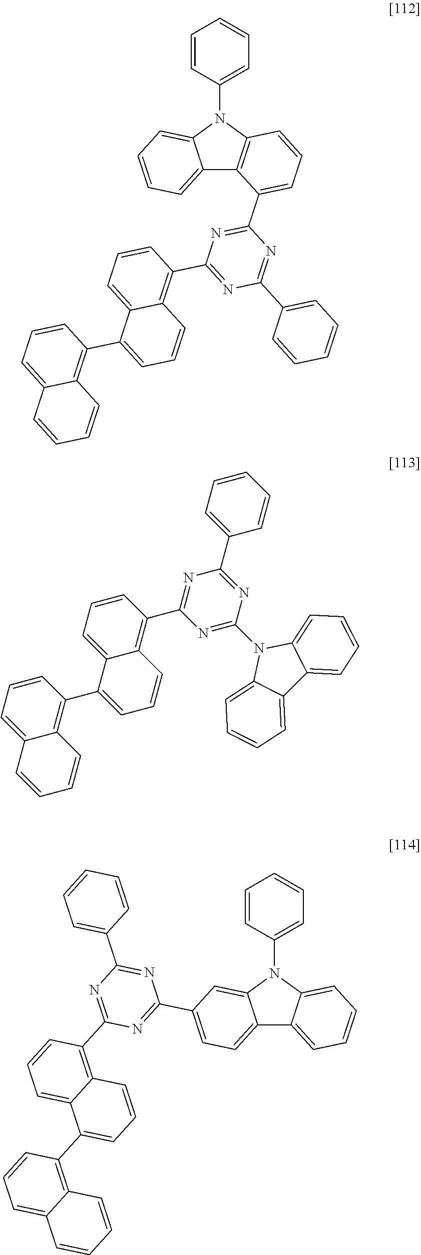 [SMILES: c1ccc(-c2nc(-c3cccc4c(-c5cccc6ccccc56)cccc34)nc(-c3cccc4c3c3ccccc3n4-c3ccccc3)n2)cc1 ; c1ccc(-c2nc(-c3cccc4c(-c5cccc6ccccc56)cccc34)nc(-n3c4ccccc4c4ccccc43)n2)cc1 ; c1ccc(-c2nc(-c3ccc4c5ccccc5n(-c5ccccc5)c4c3)nc(-c3cccc4c(-c5cccc6ccccc56)cccc34)n2)cc1]

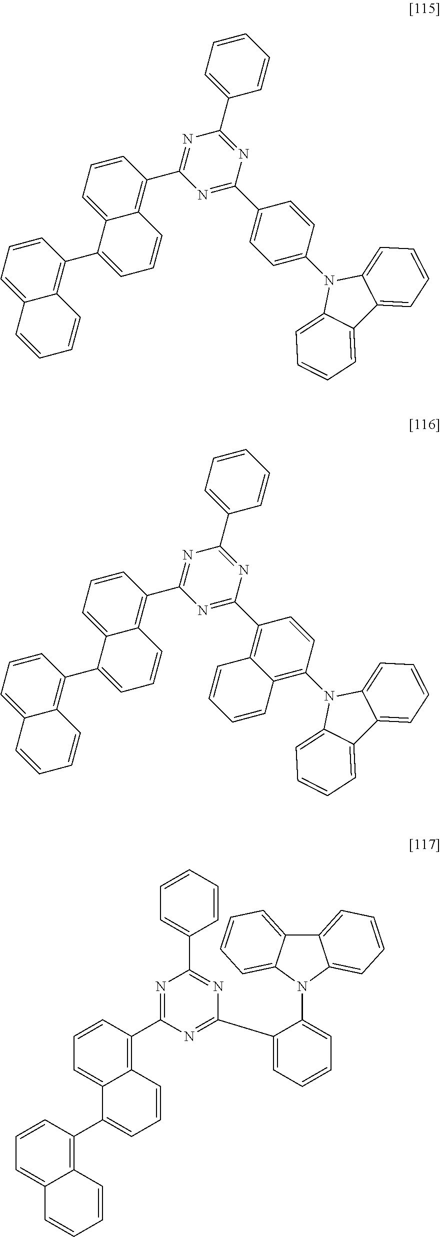 [SMILES: c1ccc(-c2nc(-c3ccc(-n4c5ccccc5c5ccccc54)cc3)nc(-c3cccc4c(-c5cccc6ccccc56)cccc34)n2)cc1 ; c1ccc(-c2nc(-c3cccc4c(-c5cccc6ccccc56)cccc34)nc(-c3ccc(-n4c5ccccc5c5ccccc54)c4ccccc34)n2)cc1 ; c1ccc(-c2nc(-c3ccccc3-n3c4ccccc4c4ccccc43)nc(-c3cccc4c(-c5cccc6ccccc56)cccc34)n2)cc1]

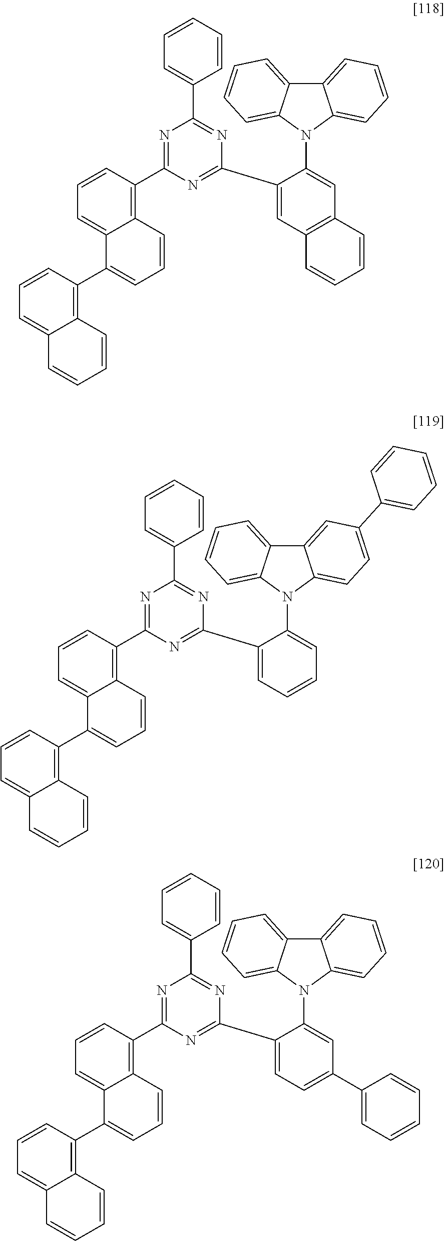 [SMILES: c1ccc(-c2nc(-c3cc4ccccc4cc3-n3c4ccccc4c4ccccc43)nc(-c3cccc4c(-c5cccc6ccccc56)cccc34)n2)cc1 ; c1ccc(-c2ccc3c(c2)c2ccccc2n3-c2ccccc2-c2nc(-c3ccccc3)nc(-c3cccc4c(-c5cccc6ccccc56)cccc34)n2)cc1 ; c1ccc(-c2ccc(-c3nc(-c4ccccc4)nc(-c4cccc5c(-c6cccc7ccccc67)cccc45)n3)c(-n3c4ccccc4c4ccccc43)c2)cc1]

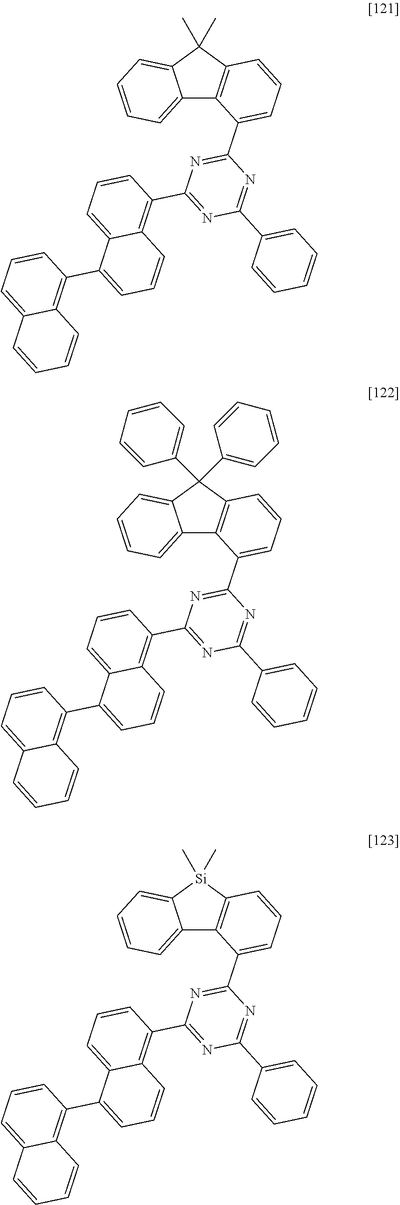 [SMILES: CC1(C)c2ccccc2-c2c(-c3nc(-c4ccccc4)nc(-c4cccc5c(-c6cccc7ccccc67)cccc45)n3)cccc21 ; c1ccc(-c2nc(-c3cccc4c3-c3ccccc3C4(c3ccccc3)c3ccccc3)nc(-c3cccc4c(-c5cccc6ccccc56)cccc34)n2)cc1 ; C[Si]1(C)c2ccccc2-c2c(-c3nc(-c4ccccc4)nc(-c4cccc5c(-c6cccc7ccccc67)cccc45)n3)cccc21]

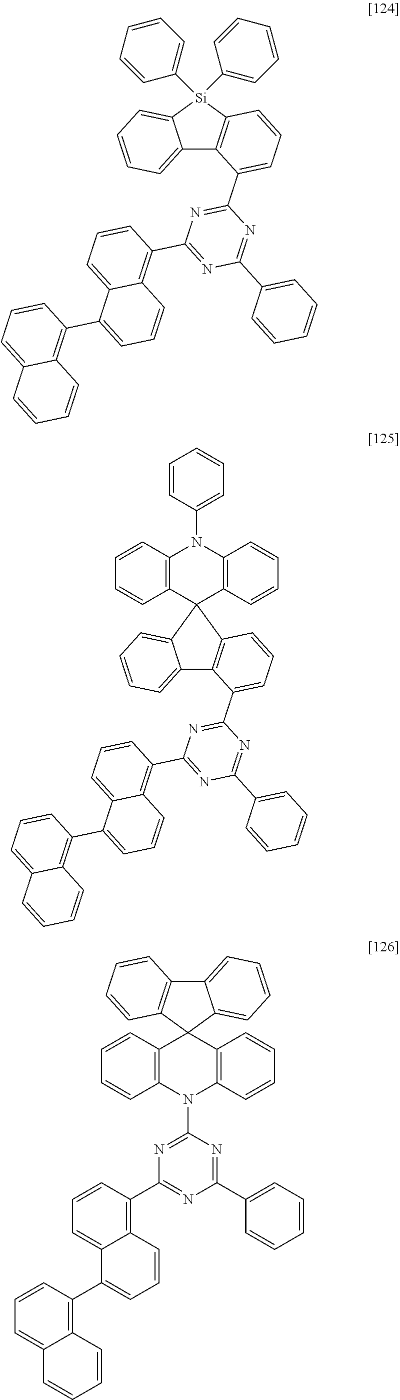 [SMILES: c1ccc(-c2nc(-c3cccc4c3-c3ccccc3[Si]4(c3ccccc3)c3ccccc3)nc(-c3cccc4c(-c5cccc6ccccc56)cccc34)n2)cc1 ; c1ccc(-c2nc(-c3cccc4c3-c3ccccc3C43c4ccccc4N(c4ccccc4)c4ccccc43)nc(-c3cccc4c(-c5cccc6ccccc56)cccc34)n2)cc1 ; c1ccc(-c2nc(-c3cccc4c(-c5cccc6ccccc56)cccc34)nc(N3c4ccccc4C4(c5ccccc5-c5ccccc54)c4ccccc43)n2)cc1]

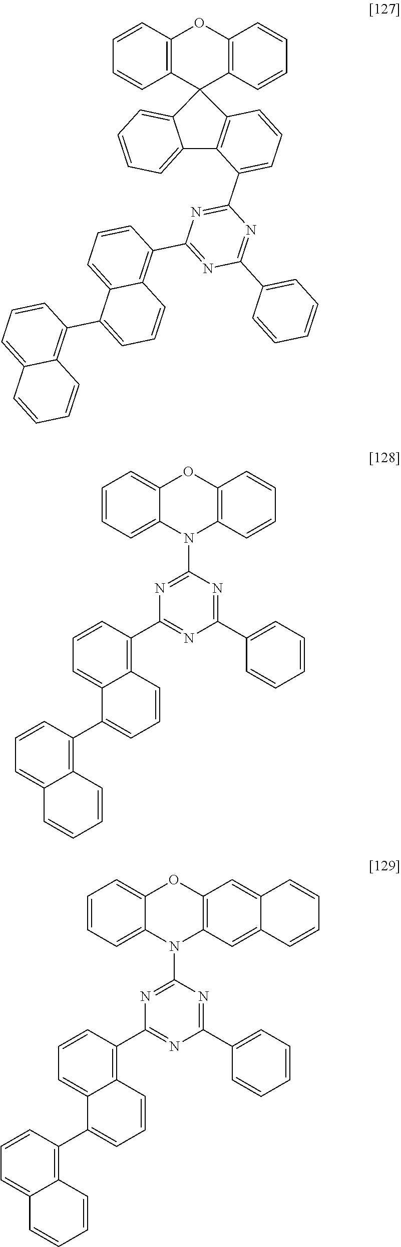 [SMILES: c1ccc(-c2nc(-c3cccc4c3-c3ccccc3C43c4ccccc4Oc4ccccc43)nc(-c3cccc4c(-c5cccc6ccccc56)cccc34)n2)cc1 ; c1ccc(-c2nc(-c3cccc4c(-c5cccc6ccccc56)cccc34)nc(N3c4ccccc4Oc4ccccc43)n2)cc1 ; c1ccc(-c2nc(-c3cccc4c(-c5cccc6ccccc56)cccc34)nc(N3c4ccccc4Oc4cc5ccccc5cc43)n2)cc1]

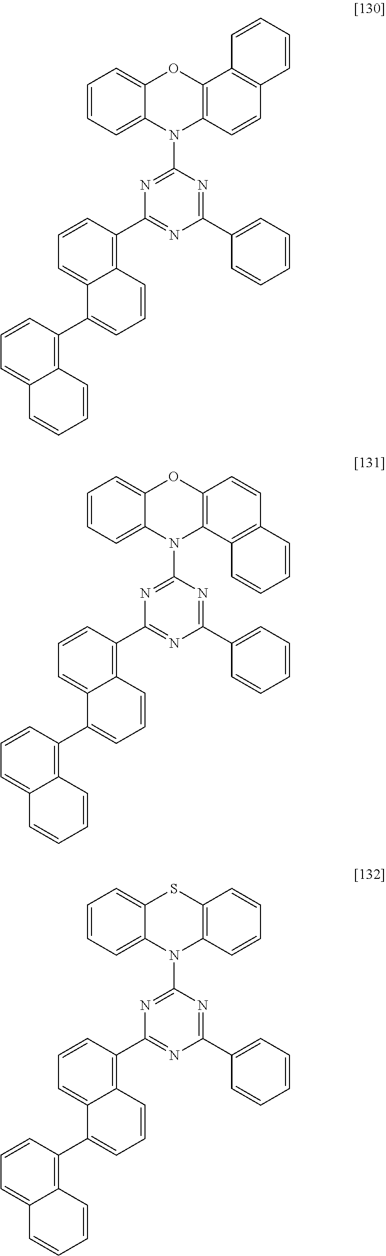 [SMILES: c1ccc(-c2nc(-c3cccc4c(-c5cccc6ccccc56)cccc34)nc(N3c4ccccc4Oc4c3ccc3ccccc43)n2)cc1 ; c1ccc(-c2nc(-c3cccc4c(-c5cccc6ccccc56)cccc34)nc(N3c4ccccc4Oc4ccc5ccccc5c43)n2)cc1 ; c1ccc(-c2nc(-c3cccc4c(-c5cccc6ccccc56)cccc34)nc(N3c4ccccc4Sc4ccccc43)n2)cc1]

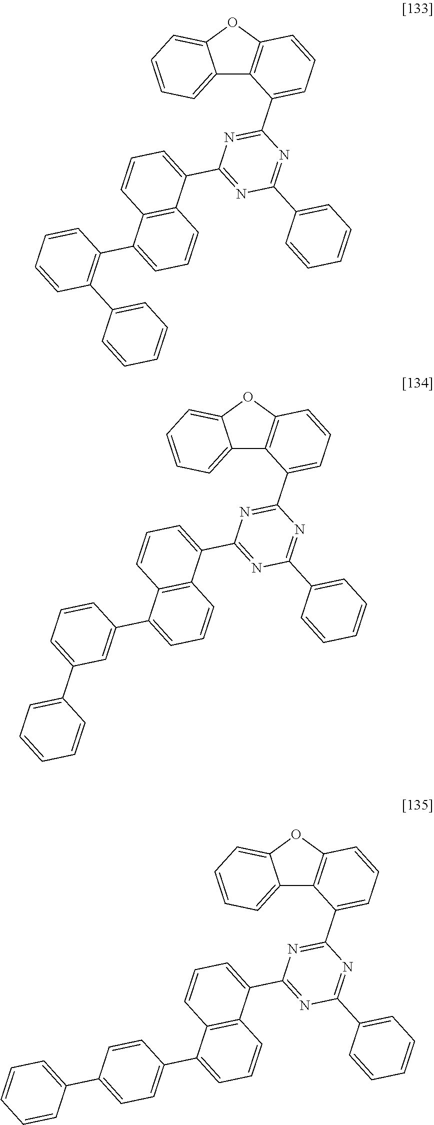 [SMILES: c1ccc(-c2nc(-c3cccc4c(-c5ccccc5-c5ccccc5)cccc34)nc(-c3cccc4oc5ccccc5c34)n2)cc1 ; c1ccc(-c2cccc(-c3cccc4c(-c5nc(-c6ccccc6)nc(-c6cccc7oc8ccccc8c67)n5)cccc34)c2)cc1 ; c1ccc(-c2ccc(-c3cccc4c(-c5nc(-c6ccccc6)nc(-c6cccc7oc8ccccc8c67)n5)cccc34)cc2)cc1]

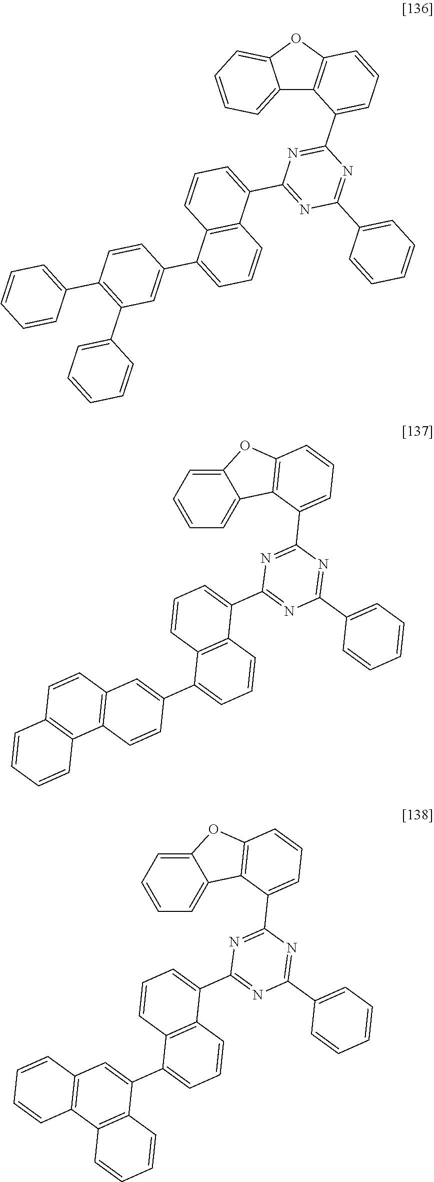 [SMILES: c1ccc(-c2nc(-c3cccc4c(-c5ccc(-c6ccccc6)c(-c6ccccc6)c5)cccc34)nc(-c3cccc4oc5ccccc5c34)n2)cc1 ; c1ccc(-c2nc(-c3cccc4c(-c5ccc6c(ccc7ccccc76)c5)cccc34)nc(-c3cccc4oc5ccccc5c34)n2)cc1 ; c1ccc(-c2nc(-c3cccc4c(-c5cc6ccccc6c6ccccc56)cccc34)nc(-c3cccc4oc5ccccc5c34)n2)cc1]

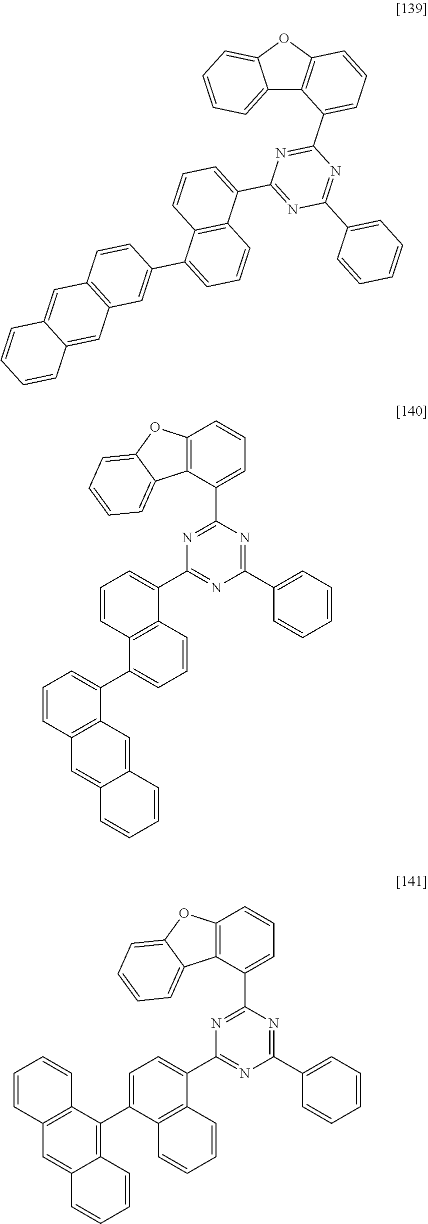 [SMILES: c1ccc(-c2nc(-c3cccc4c(-c5ccc6cc7ccccc7cc6c5)cccc34)nc(-c3cccc4oc5ccccc5c34)n2)cc1 ; c1ccc(-c2nc(-c3cccc4c(-c5cccc6cc7ccccc7cc56)cccc34)nc(-c3cccc4oc5ccccc5c34)n2)cc1 ; c1ccc(-c2nc(-c3ccc(-c4c5ccccc5cc5ccccc45)c4ccccc34)nc(-c3cccc4oc5ccccc5c34)n2)cc1]

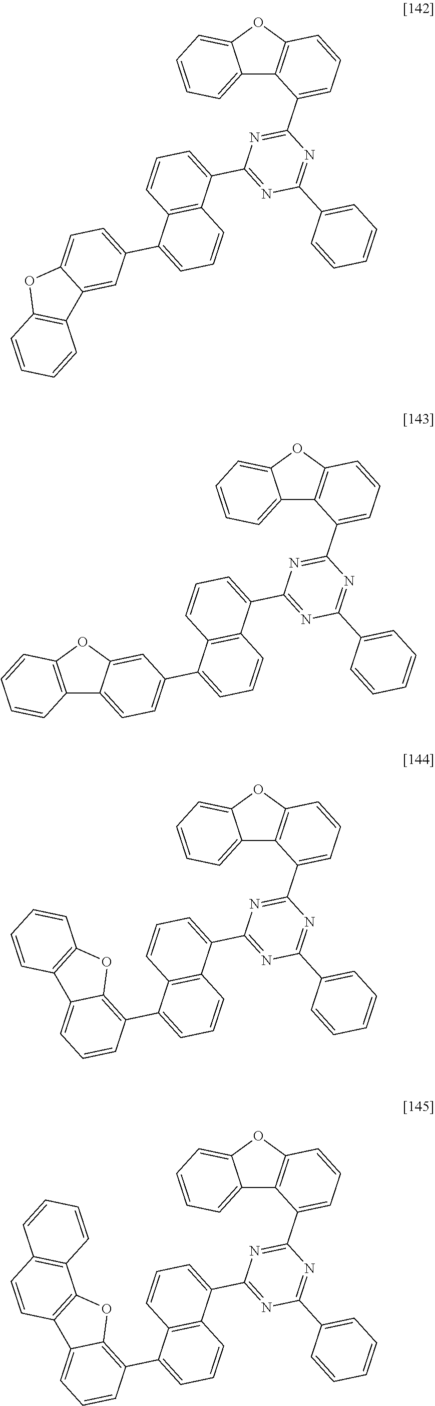 [SMILES: c1ccc(-c2nc(-c3cccc4c(-c5ccc6oc7ccccc7c6c5)cccc34)nc(-c3cccc4oc5ccccc5c34)n2)cc1 ; c1ccc(-c2nc(-c3cccc4c(-c5ccc6c(c5)oc5ccccc56)cccc34)nc(-c3cccc4oc5ccccc5c34)n2)cc1 ; c1ccc(-c2nc(-c3cccc4c(-c5cccc6c5oc5ccccc56)cccc34)nc(-c3cccc4oc5ccccc5c34)n2)cc1 ; c1ccc(-c2nc(-c3cccc4c(-c5cccc6c5oc5c7ccccc7ccc65)cccc34)nc(-c3cccc4oc5ccccc5c34)n2)cc1]

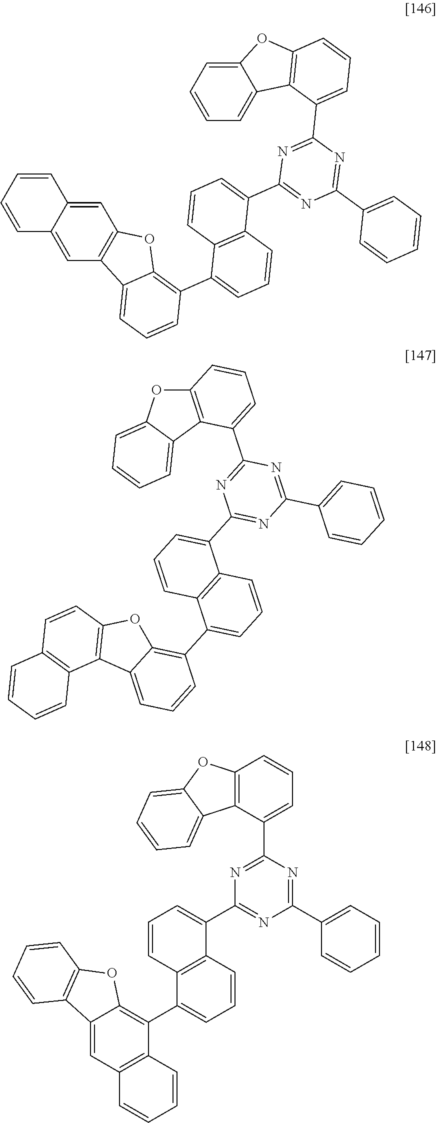 [SMILES: c1ccc(-c2nc(-c3cccc4c(-c5cccc6c5oc5cc7ccccc7cc56)cccc34)nc(-c3cccc4oc5ccccc5c34)n2)cc1 ; c1ccc(-c2nc(-c3cccc4c(-c5cccc6c5oc5ccc7ccccc7c56)cccc34)nc(-c3cccc4oc5ccccc5c34)n2)cc1 ; c1ccc(-c2nc(-c3cccc4c(-c5c6ccccc6cc6c5oc5ccccc56)cccc34)nc(-c3cccc4oc5ccccc5c34)n2)cc1]

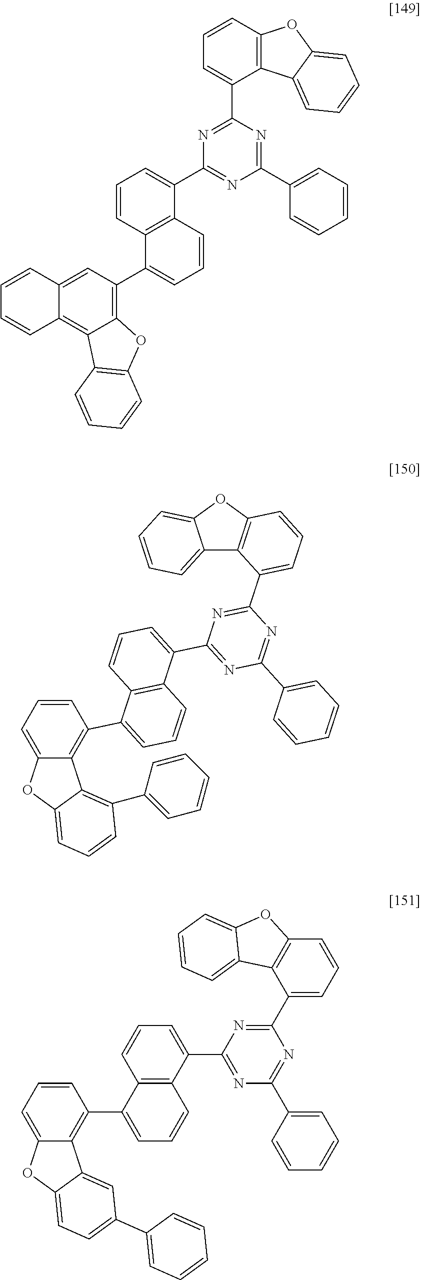 [SMILES: c1ccc(-c2nc(-c3cccc4c(-c5cc6ccccc6c6c5oc5ccccc56)cccc34)nc(-c3cccc4oc5ccccc5c34)n2)cc1 ; c1ccc(-c2nc(-c3cccc4c(-c5cccc6oc7cccc(-c8ccccc8)c7c56)cccc34)nc(-c3cccc4oc5ccccc5c34)n2)cc1 ; c1ccc(-c2ccc3oc4cccc(-c5cccc6c(-c7nc(-c8ccccc8)nc(-c8cccc9oc%10ccccc%10c89)n7)cccc56)c4c3c2)cc1]

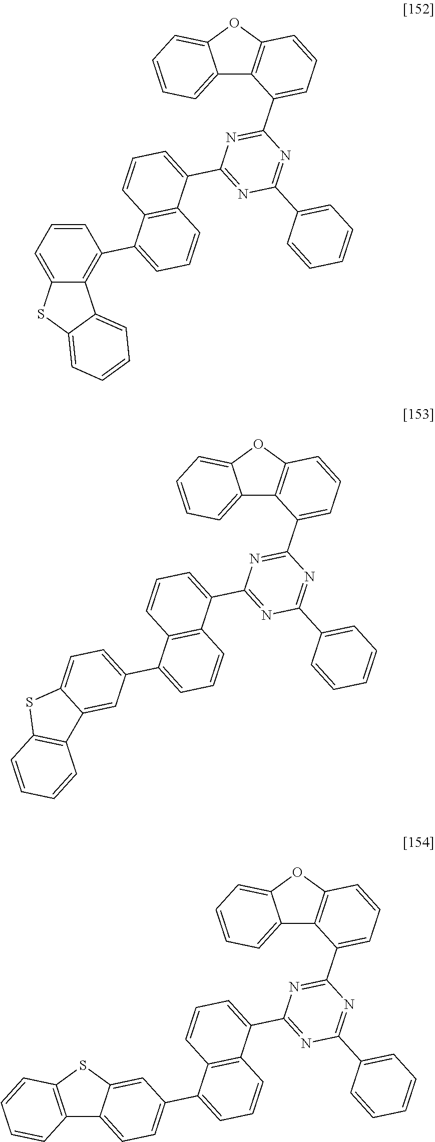 [SMILES: c1ccc(-c2nc(-c3cccc4c(-c5cccc6sc7ccccc7c56)cccc34)nc(-c3cccc4oc5ccccc5c34)n2)cc1 ; c1ccc(-c2nc(-c3cccc4c(-c5ccc6sc7ccccc7c6c5)cccc34)nc(-c3cccc4oc5ccccc5c34)n2)cc1 ; c1ccc(-c2nc(-c3cccc4c(-c5ccc6c(c5)sc5ccccc56)cccc34)nc(-c3cccc4oc5ccccc5c34)n2)cc1]

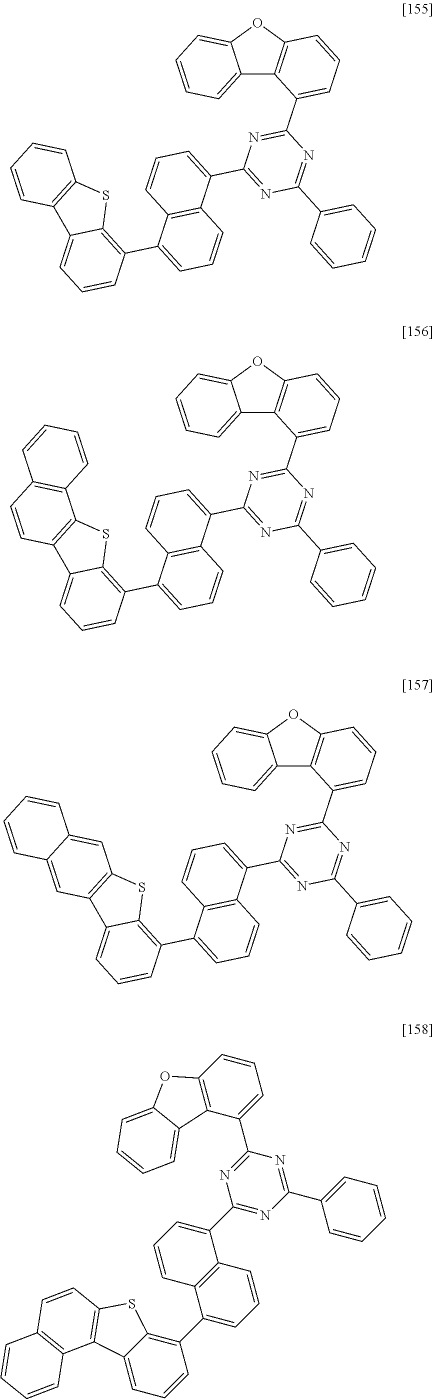 [SMILES: c1ccc(-c2nc(-c3cccc4c(-c5cccc6c5sc5ccccc56)cccc34)nc(-c3cccc4oc5ccccc5c34)n2)cc1 ; c1ccc(-c2nc(-c3cccc4c(-c5cccc6c5sc5c7ccccc7ccc65)cccc34)nc(-c3cccc4oc5ccccc5c34)n2)cc1 ; c1ccc(-c2nc(-c3cccc4c(-c5cccc6c5sc5cc7ccccc7cc56)cccc34)nc(-c3cccc4oc5ccccc5c34)n2)cc1 ; c1ccc(-c2nc(-c3cccc4c(-c5cccc6c5sc5ccc7ccccc7c56)cccc34)nc(-c3cccc4oc5ccccc5c34)n2)cc1]

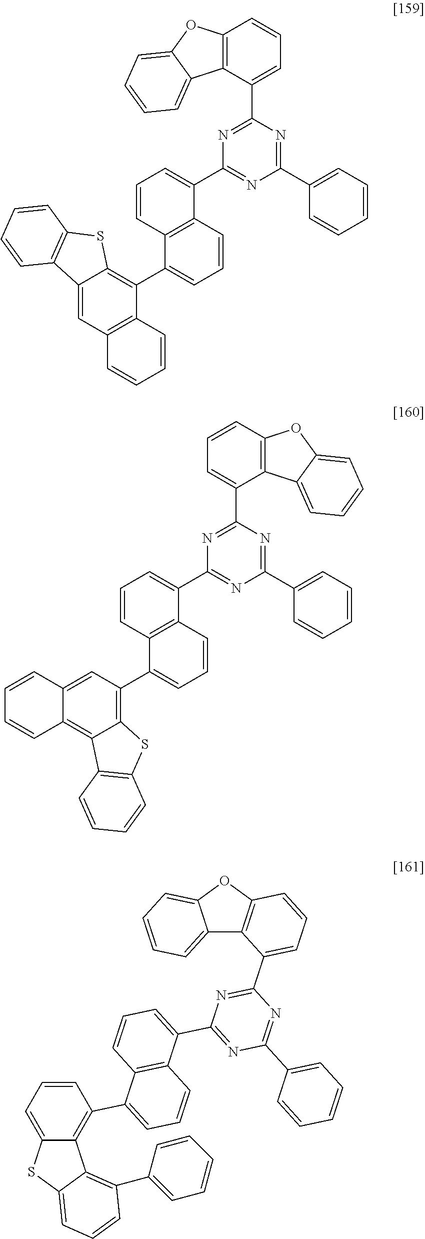 [SMILES: c1ccc(-c2nc(-c3cccc4c(-c5c6ccccc6cc6c5sc5ccccc56)cccc34)nc(-c3cccc4oc5ccccc5c34)n2)cc1 ; c1ccc(-c2nc(-c3cccc4c(-c5cc6ccccc6c6c5sc5ccccc56)cccc34)nc(-c3cccc4oc5ccccc5c34)n2)cc1 ; c1ccc(-c2nc(-c3cccc4c(-c5cccc6sc7cccc(-c8ccccc8)c7c56)cccc34)nc(-c3cccc4oc5ccccc5c34)n2)cc1]

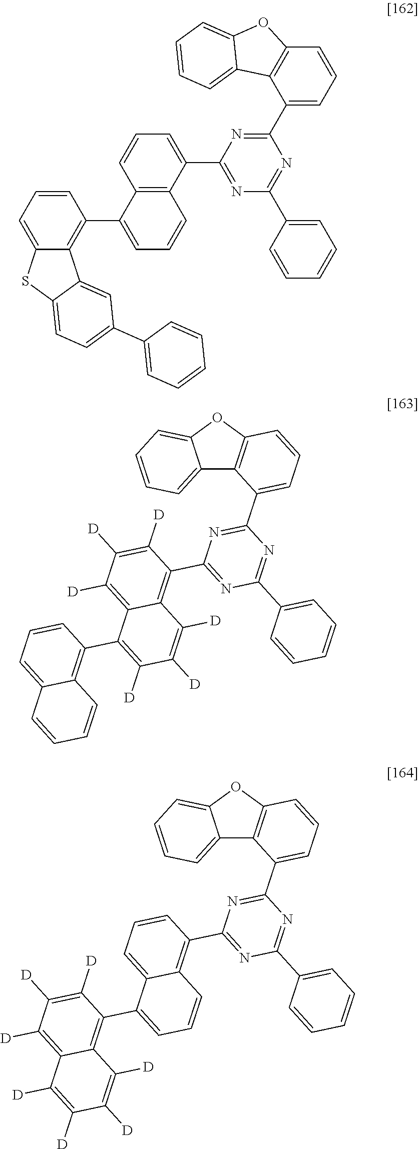

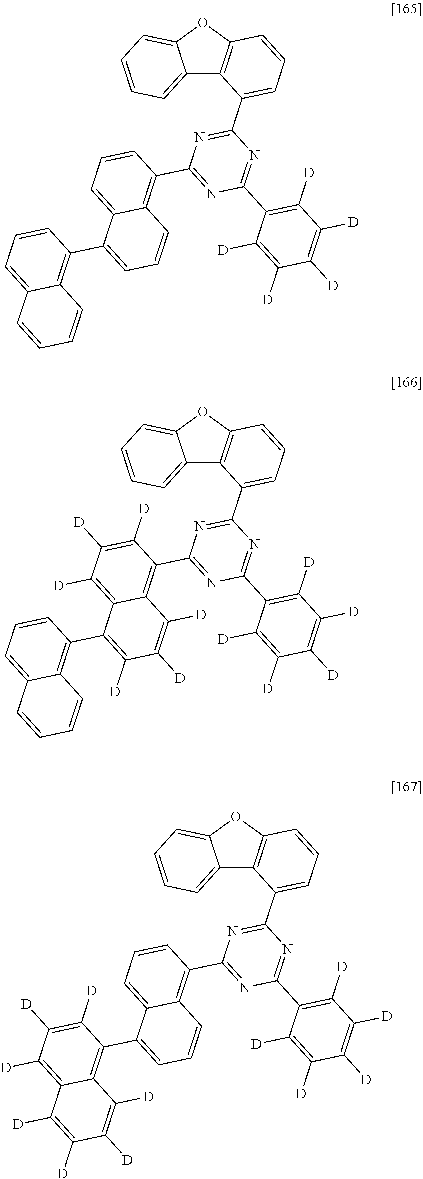 [SMILES: [2H]c1c([2H])c([2H])c(-c2nc(-c3cccc4c(-c5cccc6ccccc56)cccc34)nc(-c3cccc4oc5ccccc5c34)n2)c([2H])c1[2H] ; [2H]c1c([2H])c([2H])c(-c2nc(-c3c([2H])c([2H])c([2H])c4c(-c5cccc6ccccc56)c([2H])c([2H])c([2H])c34)nc(-c3cccc4oc5ccccc5c34)n2)c([2H])c1[2H] ; [2H]c1c([2H])c([2H])c(-c2nc(-c3cccc4c(-c5c([2H])c([2H])c([2H])c6c([2H])c([2H])c([2H])c([2H])c56)cccc34)nc(-c3cccc4oc5ccccc5c34)n2)c([2H])c1[2H]]

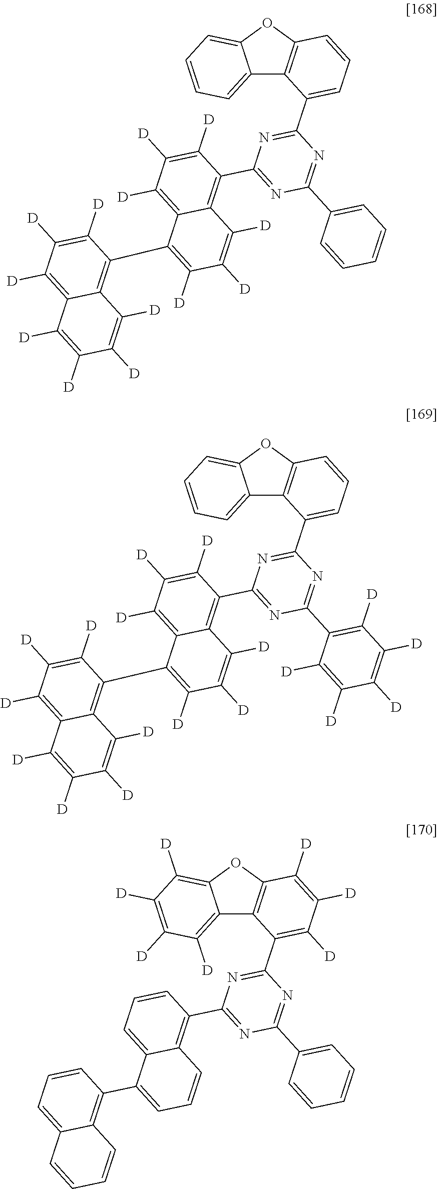 [SMILES: [2H]c1c([2H])c([2H])c2c(-c3c([2H])c([2H])c([2H])c4c(-c5nc(-c6ccccc6)nc(-c6cccc7oc8ccccc8c67)n5)c([2H])c([2H])c([2H])c34)c([2H])c([2H])c([2H])c2c1[2H] ; [2H]c1c([2H])c([2H])c(-c2nc(-c3c([2H])c([2H])c([2H])c4c(-c5c([2H])c([2H])c([2H])c6c([2H])c([2H])c([2H])c([2H])c56)c([2H])c([2H])c([2H])c34)nc(-c3cccc4oc5ccccc5c34)n2)c([2H])c1[2H] ; [2H]c1c([2H])c([2H])c2c(oc3c([2H])c([2H])c([2H])c(-c4nc(-c5ccccc5)nc(-c5cccc6c(-c7cccc8ccccc78)cccc56)n4)c32)c1[2H]]

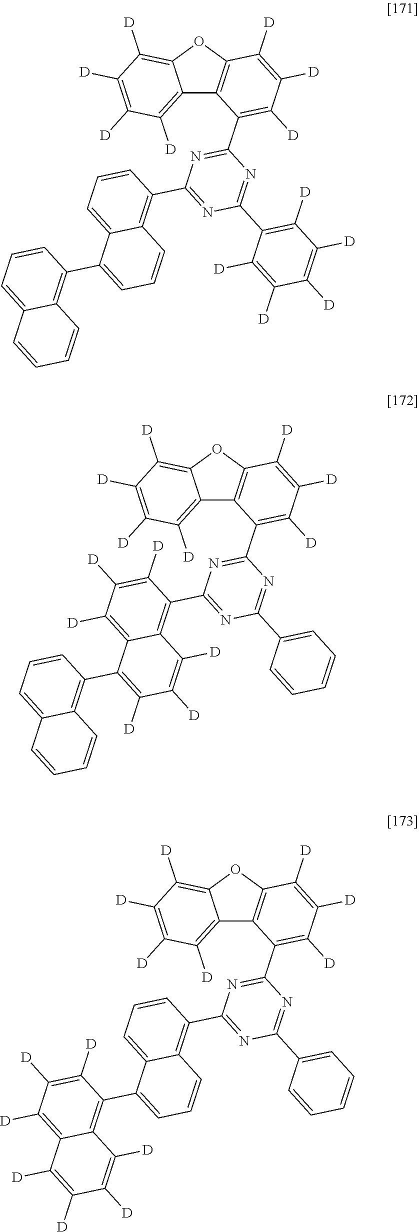 [SMILES: [2H]c1c([2H])c([2H])c(-c2nc(-c3cccc4c(-c5cccc6ccccc56)cccc34)nc(-c3c([2H])c([2H])c([2H])c4oc5c([2H])c([2H])c([2H])c([2H])c5c34)n2)c([2H])c1[2H] ; [2H]c1c([2H])c([2H])c2c(oc3c([2H])c([2H])c([2H])c(-c4nc(-c5ccccc5)nc(-c5c([2H])c([2H])c([2H])c6c(-c7cccc8ccccc78)c([2H])c([2H])c([2H])c56)n4)c32)c1[2H] ; [2H]c1c([2H])c([2H])c2c(oc3c([2H])c([2H])c([2H])c(-c4nc(-c5ccccc5)nc(-c5cccc6c(-c7c([2H])c([2H])c([2H])c8c([2H])c([2H])c([2H])c([2H])c78)cccc56)n4)c32)c1[2H]]

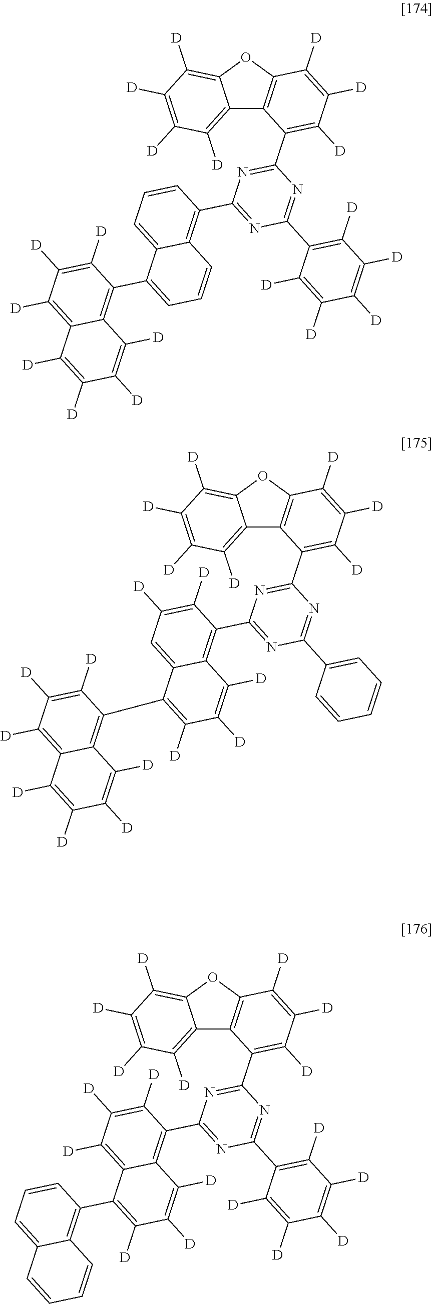

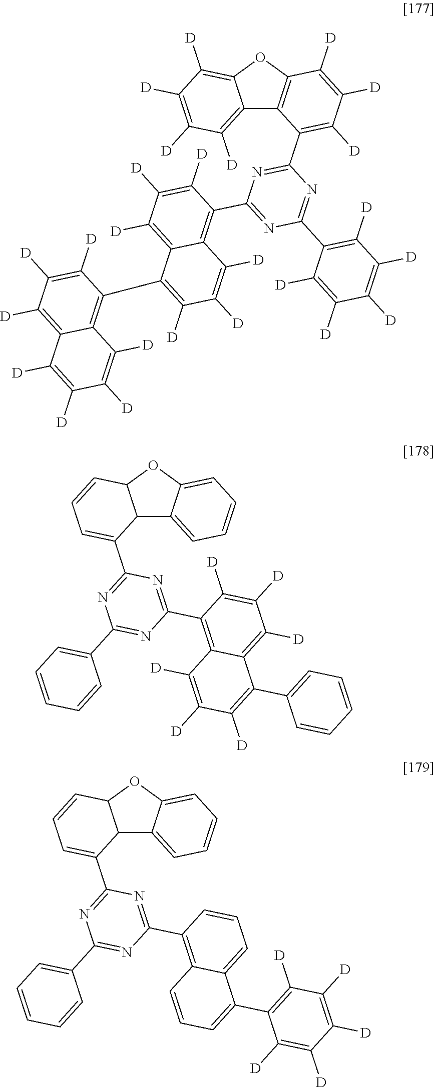 [SMILES: [2H]c1c([2H])c([2H])c(-c2nc(-c3c([2H])c([2H])c([2H])c4c(-c5c([2H])c([2H])c([2H])c6c([2H])c([2H])c([2H])c([2H])c56)c([2H])c([2H])c([2H])c34)nc(-c3c([2H])c([2H])c([2H])c4oc5c([2H])c([2H])c([2H])c([2H])c5c34)n2)c([2H])c1[2H] ; [2H]c1c([2H])c(-c2nc(C3=CC=CC4Oc5ccccc5C34)nc(-c3ccccc3)n2)c2c([2H])c([2H])c([2H])c(-c3ccccc3)c2c1[2H] ; [2H]c1c([2H])c([2H])c(-c2cccc3c(-c4nc(C5=CC=CC6Oc7ccccc7C56)nc(-c5ccccc5)n4)cccc23)c([2H])c1[2H]]

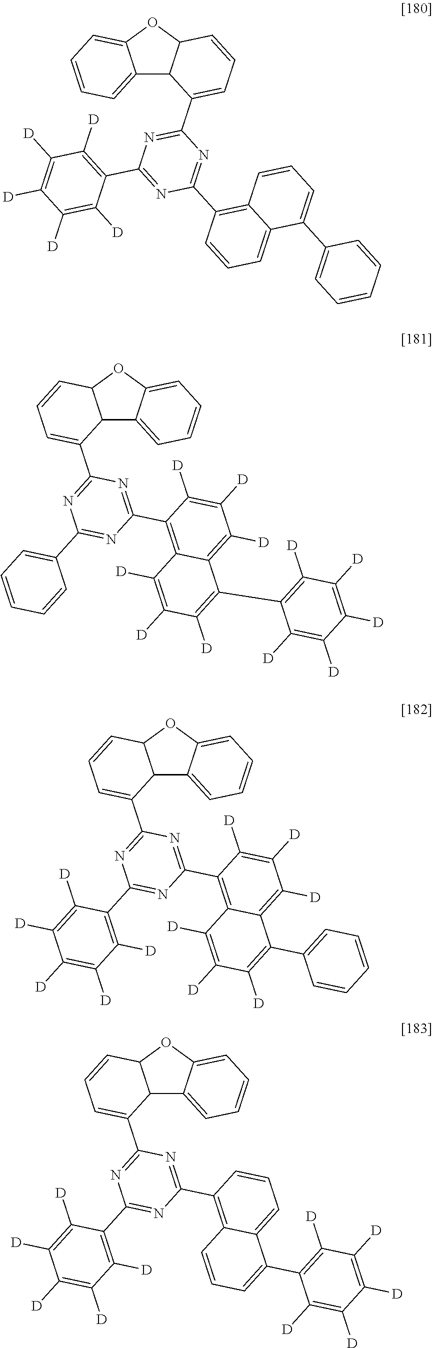 [SMILES: [2H]c1c([2H])c([2H])c(-c2nc(C3=CC=CC4Oc5ccccc5C34)nc(-c3cccc4c(-c5ccccc5)cccc34)n2)c([2H])c1[2H] ; [2H]c1c([2H])c([2H])c(-c2c([2H])c([2H])c([2H])c3c(-c4nc(C5=CC=CC6Oc7ccccc7C56)nc(-c5ccccc5)n4)c([2H])c([2H])c([2H])c23)c([2H])c1[2H] ; [2H]c1c([2H])c([2H])c(-c2nc(C3=CC=CC4Oc5ccccc5C34)nc(-c3c([2H])c([2H])c([2H])c4c(-c5ccccc5)c([2H])c([2H])c([2H])c34)n2)c([2H])c1[2H] ; [2H]c1c([2H])c([2H])c(-c2nc(C3=CC=CC4Oc5ccccc5C34)nc(-c3cccc4c(-c5c([2H])c([2H])c([2H])c([2H])c5[2H])cccc34)n2)c([2H])c1[2H]]

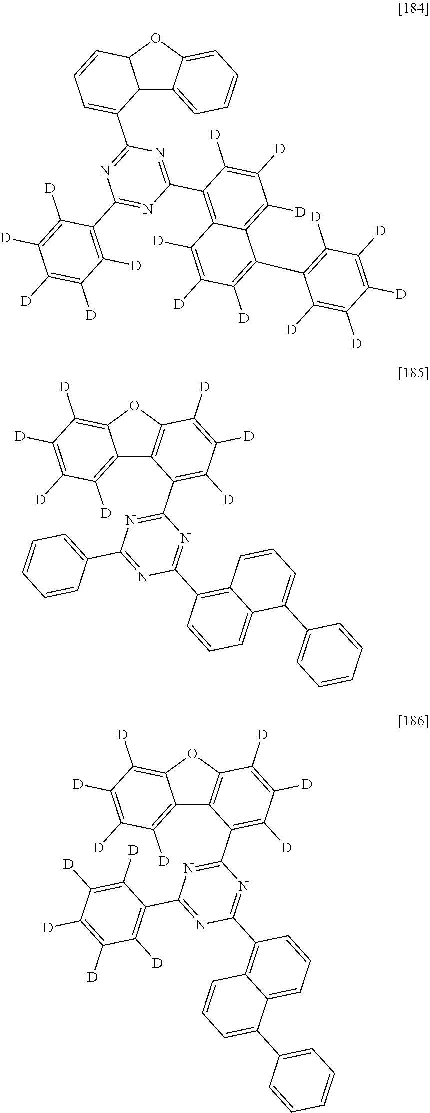 [SMILES: [2H]c1c([2H])c([2H])c(-c2nc(C3=CC=CC4Oc5ccccc5C34)nc(-c3c([2H])c([2H])c([2H])c4c(-c5c([2H])c([2H])c([2H])c([2H])c5[2H])c([2H])c([2H])c([2H])c34)n2)c([2H])c1[2H] ; [2H]c1c([2H])c([2H])c2c(oc3c([2H])c([2H])c([2H])c(-c4nc(-c5ccccc5)nc(-c5cccc6c(-c7ccccc7)cccc56)n4)c32)c1[2H] ; [2H]c1c([2H])c([2H])c(-c2nc(-c3cccc4c(-c5ccccc5)cccc34)nc(-c3c([2H])c([2H])c([2H])c4oc5c([2H])c([2H])c([2H])c([2H])c5c34)n2)c([2H])c1[2H]]

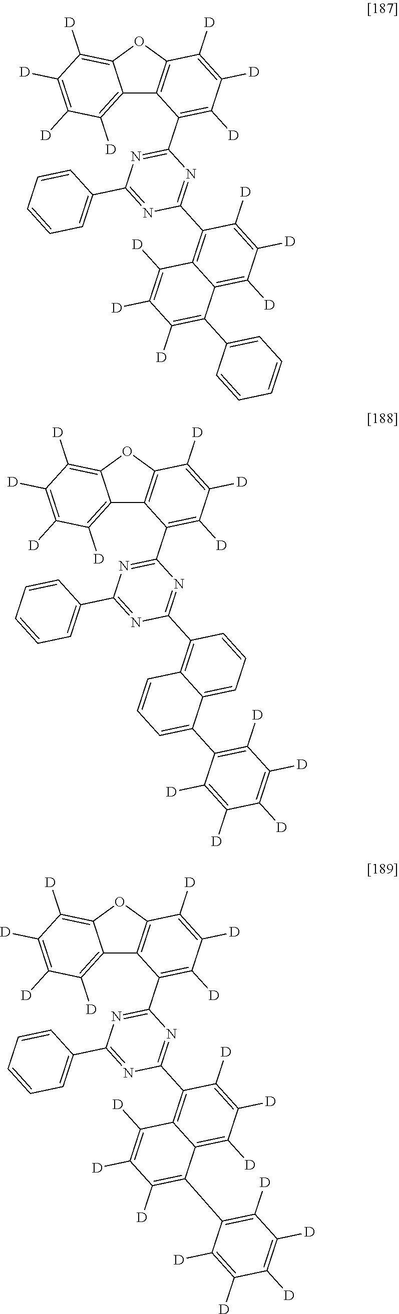 [SMILES: [2H]c1c([2H])c([2H])c2c(oc3c([2H])c([2H])c([2H])c(-c4nc(-c5ccccc5)nc(-c5c([2H])c([2H])c([2H])c6c(-c7ccccc7)c([2H])c([2H])c([2H])c56)n4)c32)c1[2H] ; [2H]c1c([2H])c([2H])c(-c2cccc3c(-c4nc(-c5ccccc5)nc(-c5c([2H])c([2H])c([2H])c6oc7c([2H])c([2H])c([2H])c([2H])c7c56)n4)cccc23)c([2H])c1[2H] ; [2H]c1c([2H])c([2H])c(-c2c([2H])c([2H])c([2H])c3c(-c4nc(-c5ccccc5)nc(-c5c([2H])c([2H])c([2H])c6oc7c([2H])c([2H])c([2H])c([2H])c7c56)n4)c([2H])c([2H])c([2H])c23)c([2H])c1[2H]]

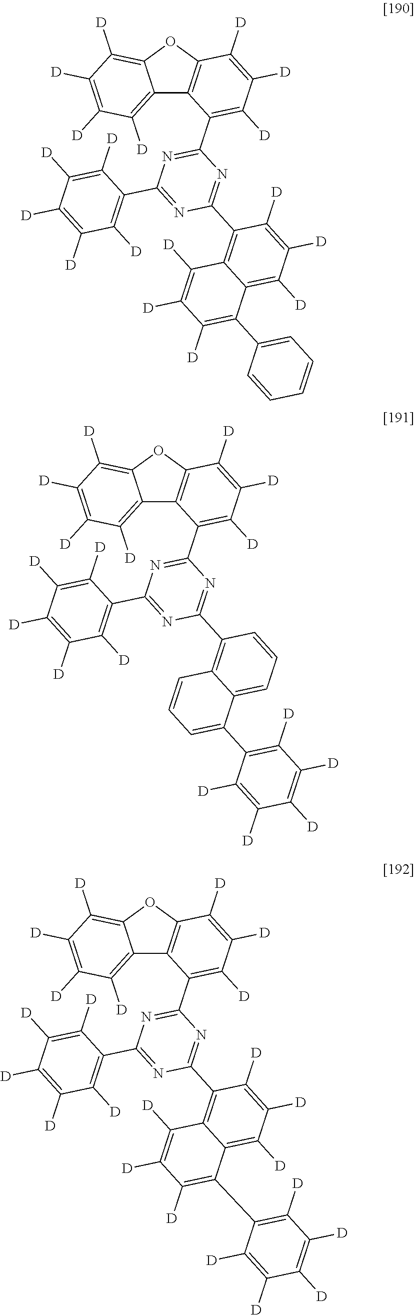

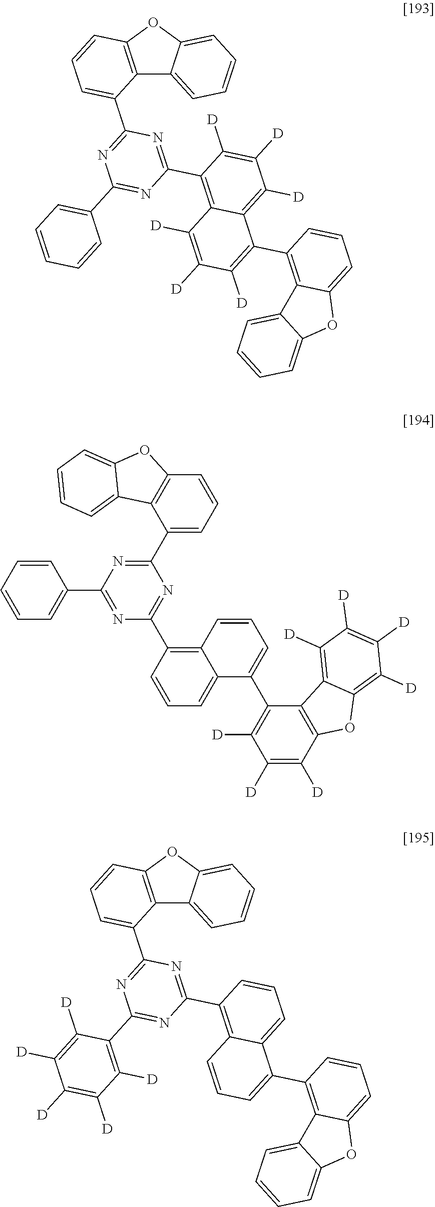 [SMILES: [2H]c1c([2H])c(-c2cccc3oc4ccccc4c23)c2c([2H])c([2H])c([2H])c(-c3nc(-c4ccccc4)nc(-c4cccc5oc6ccccc6c45)n3)c2c1[2H] ; [2H]c1c([2H])c([2H])c2c(oc3c([2H])c([2H])c([2H])c(-c4cccc5c(-c6nc(-c7ccccc7)nc(-c7cccc8oc9ccccc9c78)n6)cccc45)c32)c1[2H] ; [2H]c1c([2H])c([2H])c(-c2nc(-c3cccc4c(-c5cccc6oc7ccccc7c56)cccc34)nc(-c3cccc4oc5ccccc5c34)n2)c([2H])c1[2H]]

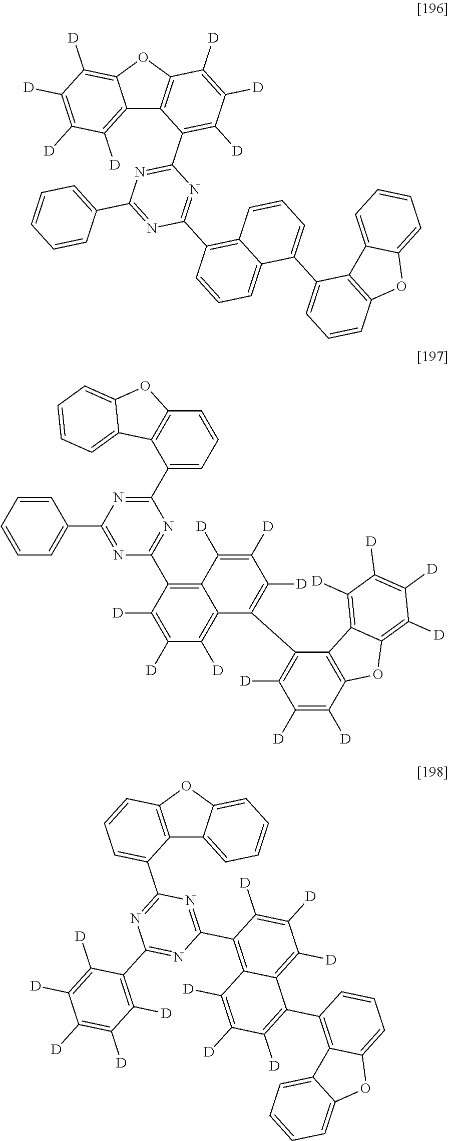 [SMILES: [2H]c1c([2H])c([2H])c2c(oc3c([2H])c([2H])c([2H])c(-c4nc(-c5ccccc5)nc(-c5cccc6c(-c7cccc8oc9ccccc9c78)cccc56)n4)c32)c1[2H] ; [2H]c1c([2H])c([2H])c2c(oc3c([2H])c([2H])c([2H])c(-c4c([2H])c([2H])c([2H])c5c(-c6nc(-c7ccccc7)nc(-c7cccc8oc9ccccc9c78)n6)c([2H])c([2H])c([2H])c45)c32)c1[2H] ; [2H]c1c([2H])c([2H])c(-c2nc(-c3c([2H])c([2H])c([2H])c4c(-c5cccc6oc7ccccc7c56)c([2H])c([2H])c([2H])c34)nc(-c3cccc4oc5ccccc5c34)n2)c([2H])c1[2H]]

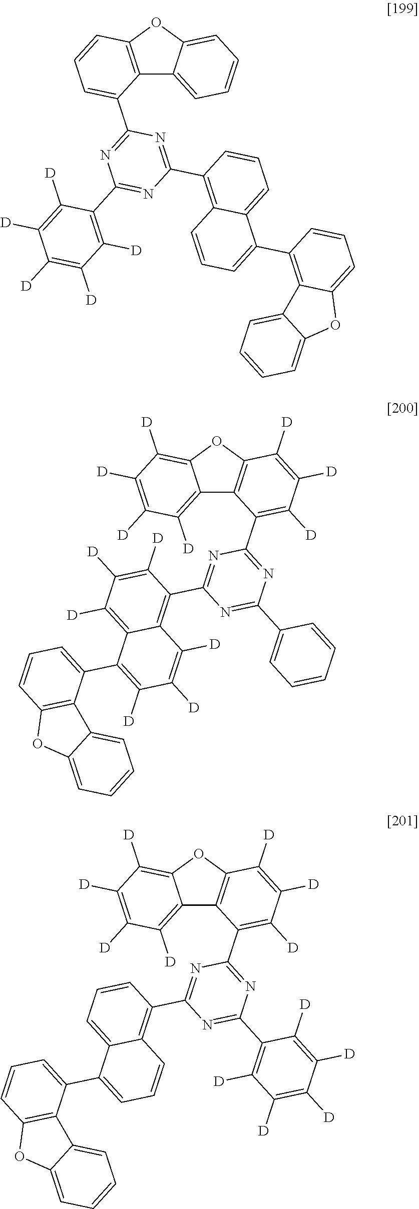

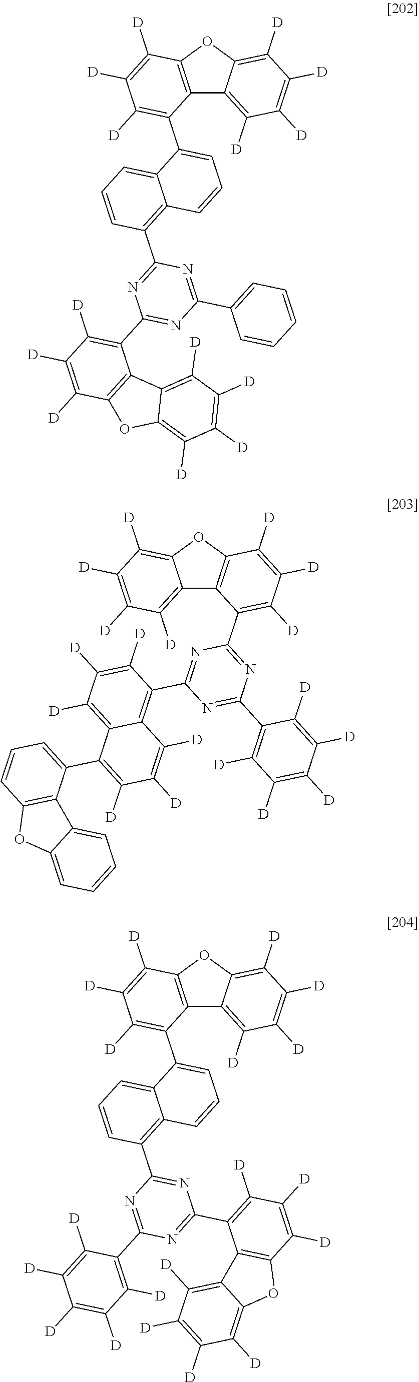

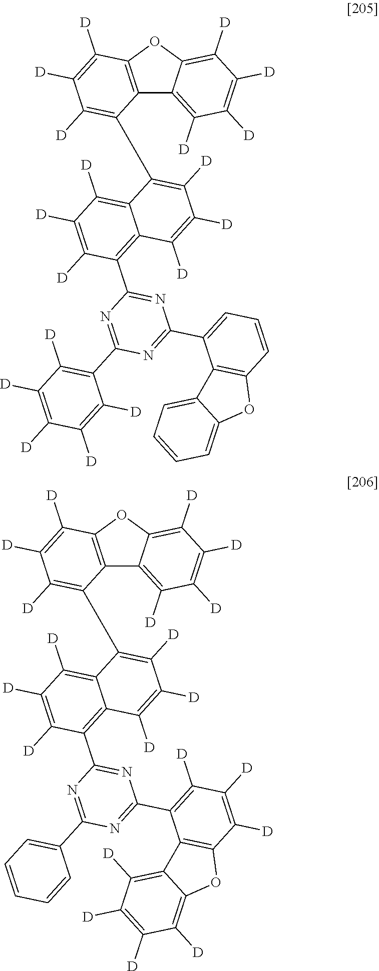

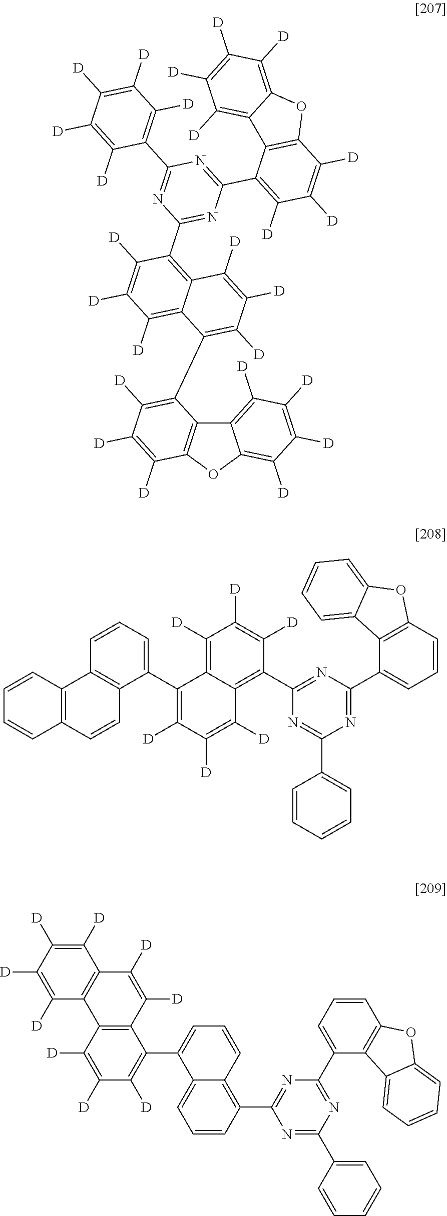 [SMILES: [2H]c1c([2H])c([2H])c(-c2nc(-c3c([2H])c([2H])c([2H])c4c(-c5c([2H])c([2H])c([2H])c6oc7c([2H])c([2H])c([2H])c([2H])c7c56)c([2H])c([2H])c([2H])c34)nc(-c3c([2H])c([2H])c([2H])c4oc5c([2H])c([2H])c([2H])c([2H])c5c34)n2)c([2H])c1[2H] ; [2H]c1c([2H])c(-c2cccc3c2ccc2ccccc23)c2c([2H])c([2H])c([2H])c(-c3nc(-c4ccccc4)nc(-c4cccc5oc6ccccc6c45)n3)c2c1[2H] ; [2H]c1c([2H])c([2H])c2c(c1[2H])c([2H])c([2H])c1c(-c3cccc4c(-c5nc(-c6ccccc6)nc(-c6cccc7oc8ccccc8c67)n5)cccc34)c([2H])c([2H])c([2H])c12]

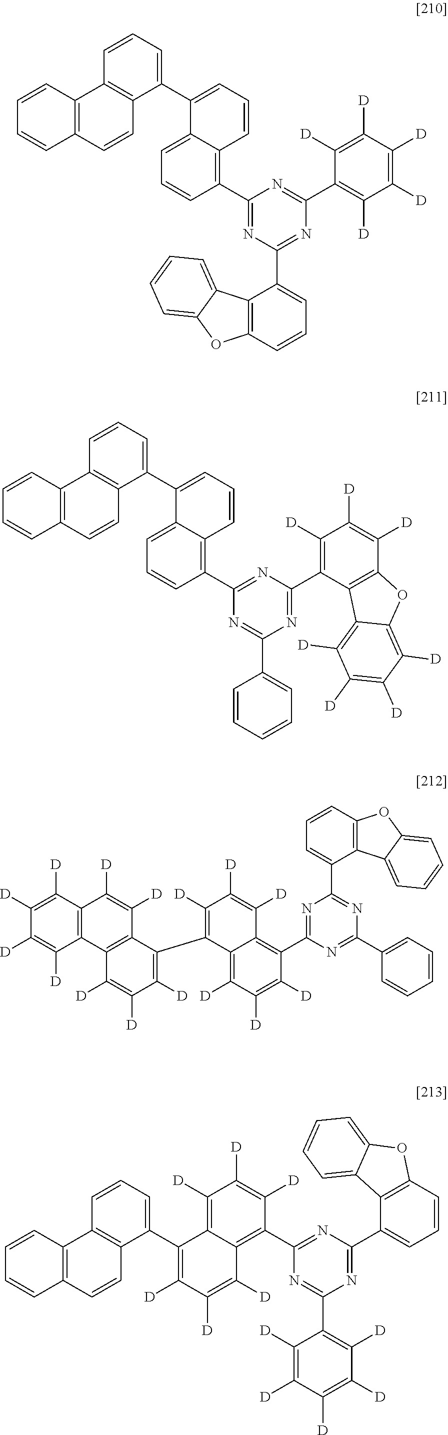

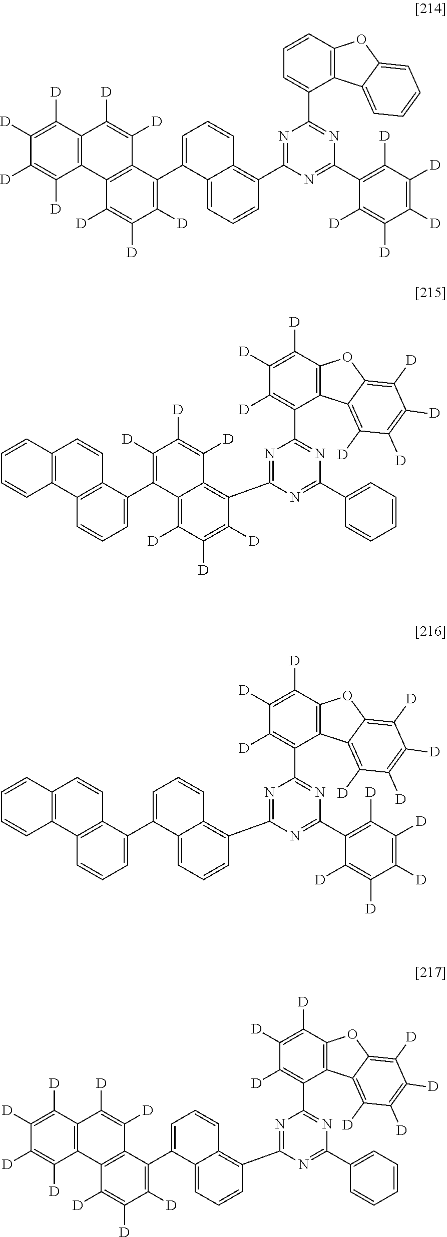

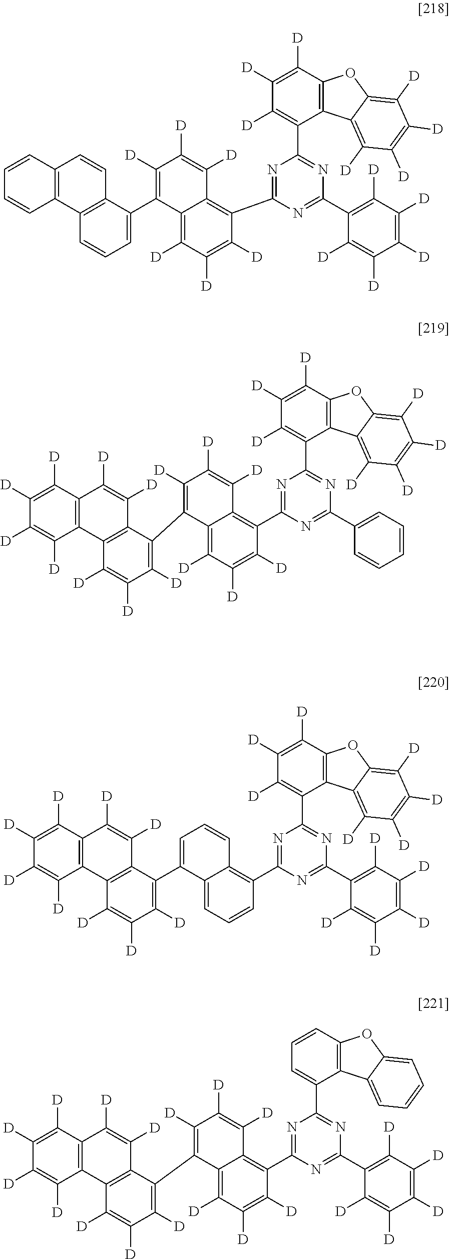

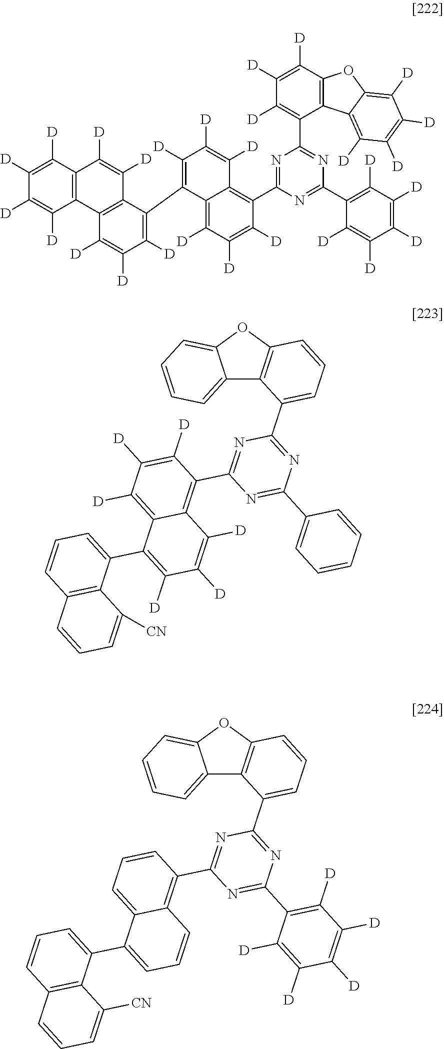 [SMILES: [2H]c1c([2H])c([2H])c(-c2nc(-c3c([2H])c([2H])c([2H])c4c(-c5c([2H])c([2H])c([2H])c6c5c([2H])c([2H])c5c([2H])c([2H])c([2H])c([2H])c56)c([2H])c([2H])c([2H])c34)nc(-c3c([2H])c([2H])c([2H])c4oc5c([2H])c([2H])c([2H])c([2H])c5c34)n2)c([2H])c1[2H] ; [2H]c1c([2H])c(-c2cccc3cccc(C#N)c23)c2c([2H])c([2H])c([2H])c(-c3nc(-c4ccccc4)nc(-c4cccc5oc6ccccc6c45)n3)c2c1[2H] ; [2H]c1c([2H])c([2H])c(-c2nc(-c3cccc4c(-c5cccc6cccc(C#N)c56)cccc34)nc(-c3cccc4oc5ccccc5c34)n2)c([2H])c1[2H]]

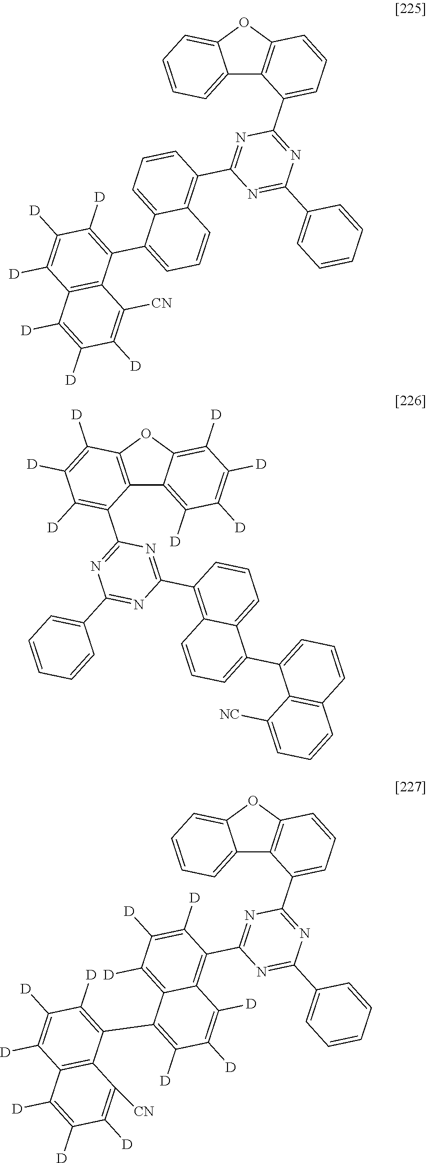

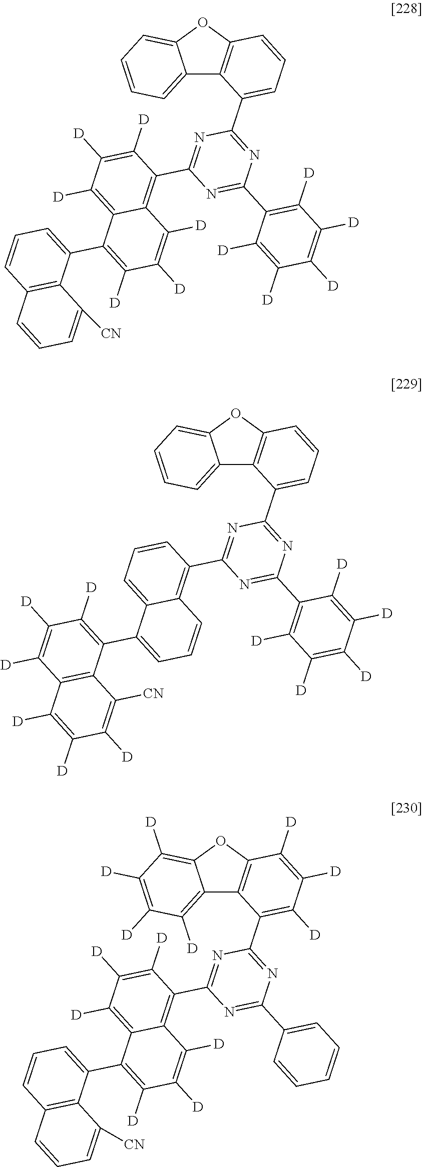 [SMILES: [2H]c1c([2H])c([2H])c(-c2nc(-c3c([2H])c([2H])c([2H])c4c(-c5cccc6cccc(C#N)c56)c([2H])c([2H])c([2H])c34)nc(-c3cccc4oc5ccccc5c34)n2)c([2H])c1[2H] ; [2H]c1c([2H])c([2H])c(-c2nc(-c3cccc4c(-c5c([2H])c([2H])c([2H])c6c([2H])c([2H])c([2H])c(C#N)c56)cccc34)nc(-c3cccc4oc5ccccc5c34)n2)c([2H])c1[2H] ; [2H]c1c([2H])c([2H])c2c(oc3c([2H])c([2H])c([2H])c(-c4nc(-c5ccccc5)nc(-c5c([2H])c([2H])c([2H])c6c(-c7cccc8cccc(C#N)c78)c([2H])c([2H])c([2H])c56)n4)c32)c1[2H]]

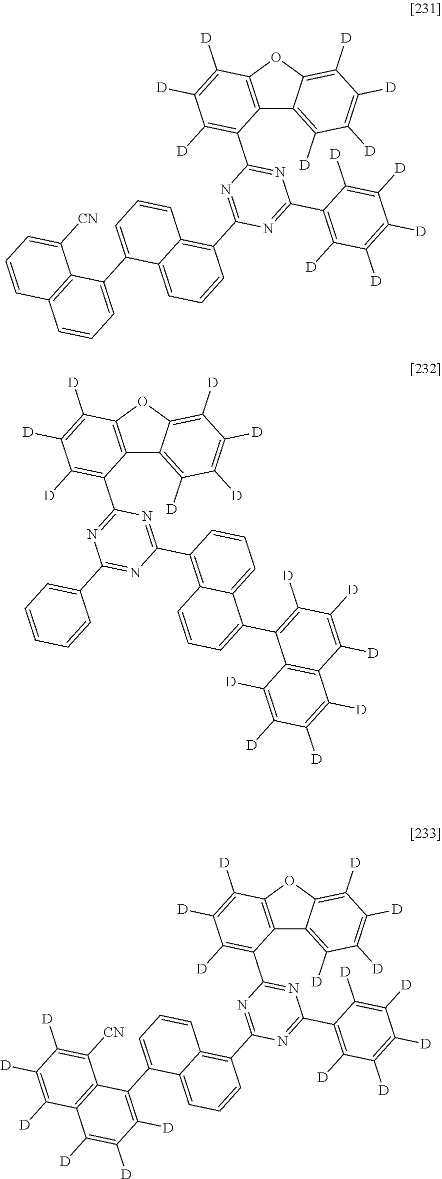 [SMILES: [2H]c1c([2H])c([2H])c(-c2nc(-c3cccc4c(-c5cccc6cccc(C#N)c56)cccc34)nc(-c3c([2H])c([2H])c([2H])c4oc5c([2H])c([2H])c([2H])c([2H])c5c34)n2)c([2H])c1[2H] ; [2H]c1c([2H])c([2H])c2c(oc3c([2H])c([2H])c([2H])c(-c4nc(-c5ccccc5)nc(-c5cccc6c(-c7c([2H])c([2H])c([2H])c8c([2H])c([2H])c([2H])c([2H])c78)cccc56)n4)c32)c1[2H] ; [2H]c1c([2H])c([2H])c(-c2nc(-c3cccc4c(-c5c([2H])c([2H])c([2H])c6c([2H])c([2H])c([2H])c(C#N)c56)cccc34)nc(-c3c([2H])c([2H])c([2H])c4oc5c([2H])c([2H])c([2H])c([2H])c5c34)n2)c([2H])c1[2H]]

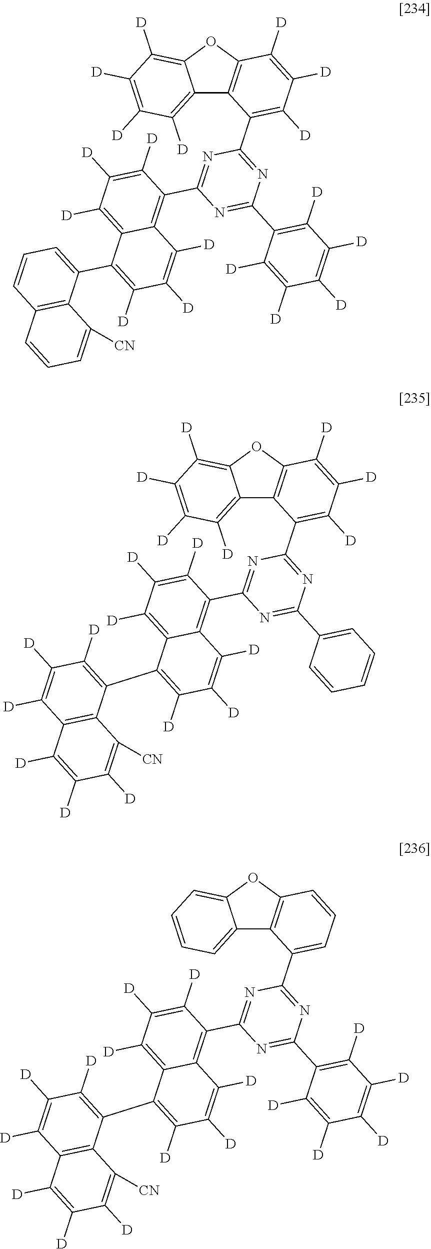

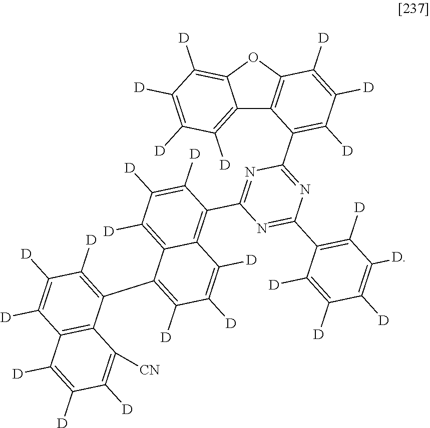

8. A composition for an organic optoelectronic device, the composition comprising:
a first compound; and
a second compound,
wherein:
the first compound is the compound as claimed in claim 1, and
the second compound is represented by Chemical Formula 2, 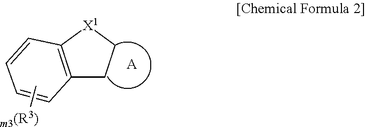
in Chemical Formula 2,
X¹ is O, S, NR^{b}, CR^{c}R^{d}, or SiR^{e}R^{f},
R^{b}, R^{c}, R^{d}, R^{e}, R^{f}, and R³ are each independently hydrogen, deuterium, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
m3 is an integer of 1 to 4, and
ring A is a ring of Group II, 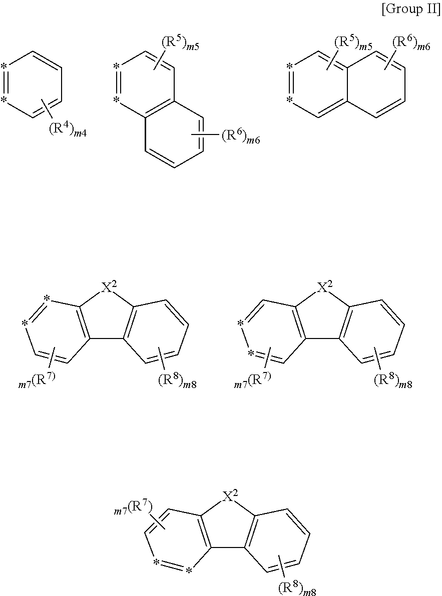
in Group II,
is a linking point,
X² is O, S, NR^{g}, CR^{h}Rⁱ, or SiR^{j}R^{k},
R^{g}, R^{h}, Rⁱ, R^{j}, R^{k}, and R⁴ to R⁸ are each independently hydrogen, deuterium, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
m4, m6, and m8 are each independently an integer of 1 to 4,
m5 and m7 are each independently 1 or 2, and
at least one of R³ to R⁸ is a group represented by Chemical Formula a, 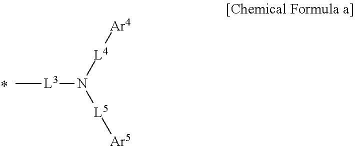
in Chemical Formula a,
L³ to L⁵ are each independently a single bond or a substituted or unsubstituted C6 to C30 arylene group,
Ar⁴ and Ar⁵ are each independently a substituted or unsubstituted amine group, a substituted or unsubstituted C6 to C30 aryl group, or a substituted or unsubstituted C2 to C30 heterocyclic group, and
is a linking point.

9. The composition as claimed in claim 8, wherein:
Chemical Formula 2 is represented by one of Chemical Formula 2-I to Chemical Formula 2-IX: 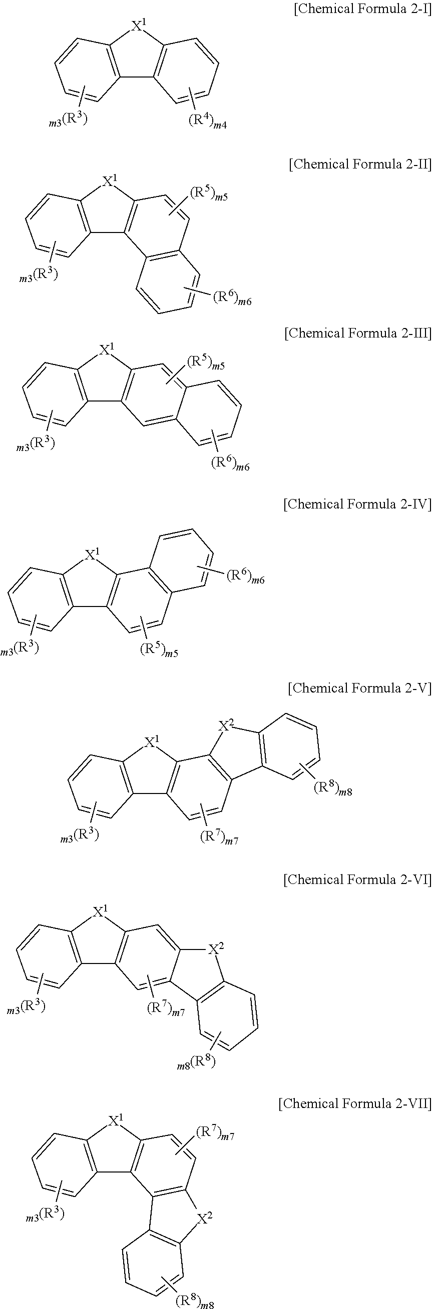 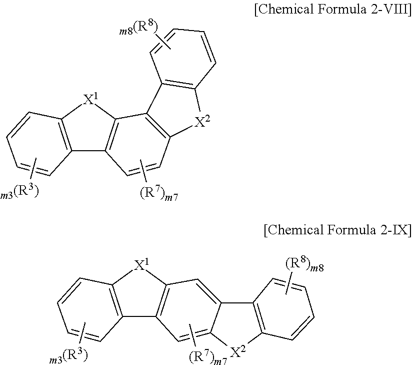
in Chemical Formula 2-I to Chemical Formula 2-IX, X¹, X², and R³ to R¹² are defined the same as those of Chemical Formula 2.

10. The composition as claimed in claim 9, wherein:
the second compound is represented by one of Chemical Formula 2-IA to Chemical Formula 2-IXA, Chemical Formula 2-IB to Chemical Formula 2-IXB, and Chemical Formula 2-IC to Chemical Formula 2-IIIC: 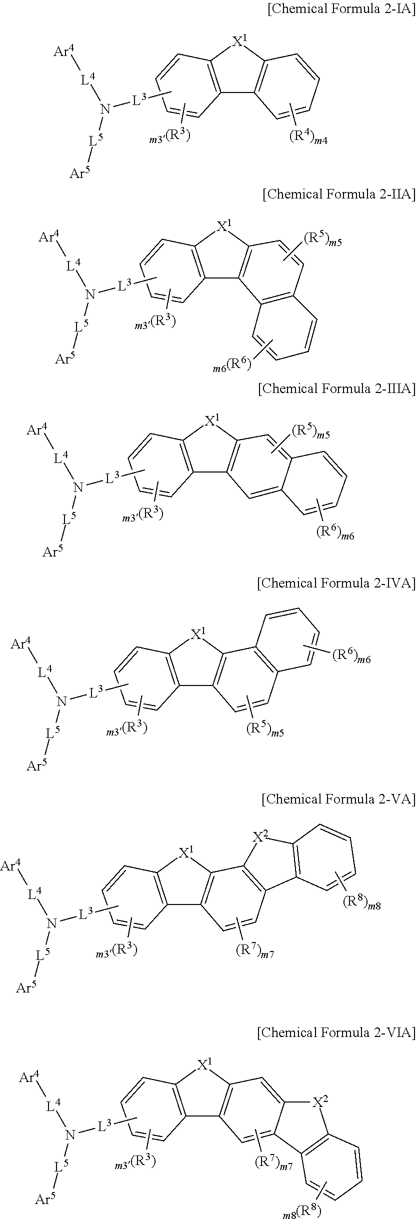 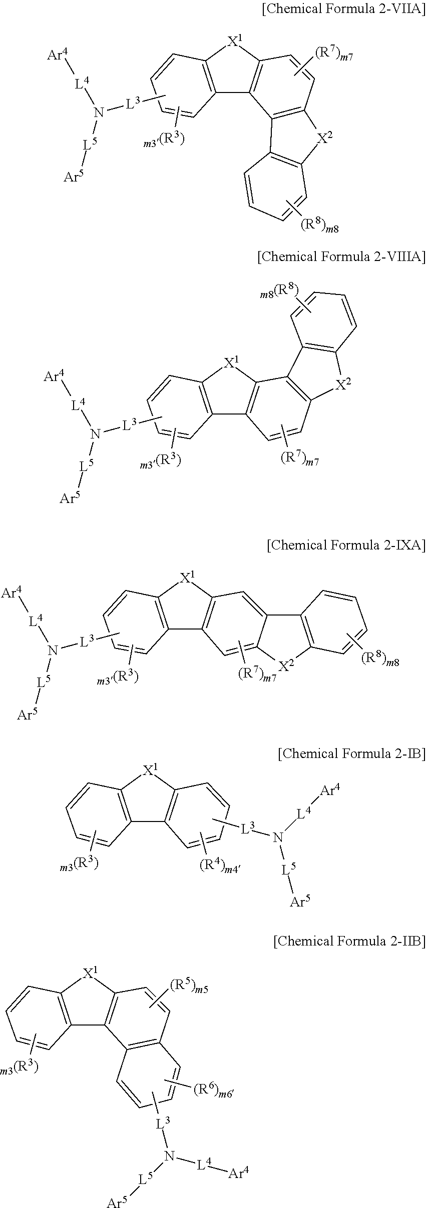 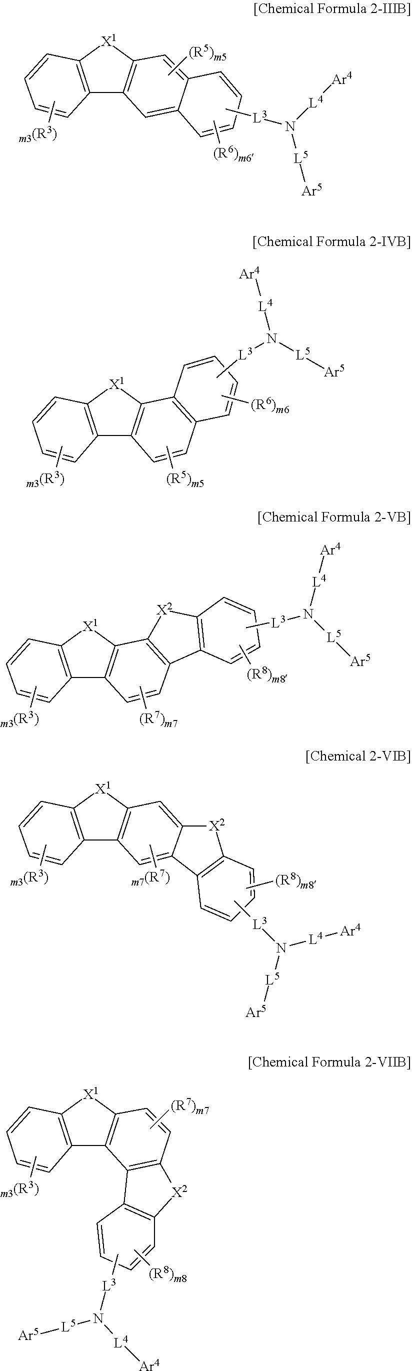 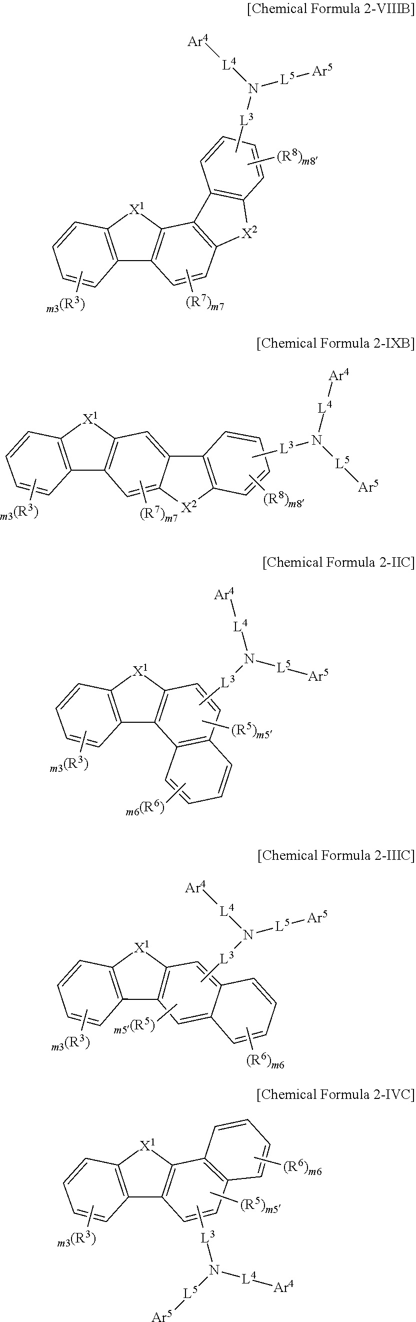
in Chemical Formula 2-IA to Chemical Formula 2-IXA, Chemical Formula 2-IB to Chemical Formula 2-IXB, and Chemical Formula 2-IIC to Chemical Formula 2-IVC,
X¹, X², L³ to L⁵, m3 to m8, Ar⁴, and Ar⁵ are defined the same as those of Chemical Formula 2,
R³ to R⁸ are each independently hydrogen, deuterium, a substituted or unsubstituted C1 to C30 alkyl group, or a substituted or unsubstituted C6 to C30 aryl group,
m3′, m4′, m6′ and m8′ are each independently an integer of 1 to 3, and
m5′ is 1.

11. The composition as claimed in claim 9, wherein:
the second compound is represented by Chemical Formula 2-IVB-2 or Chemical Formula 2-VIIIB-2: 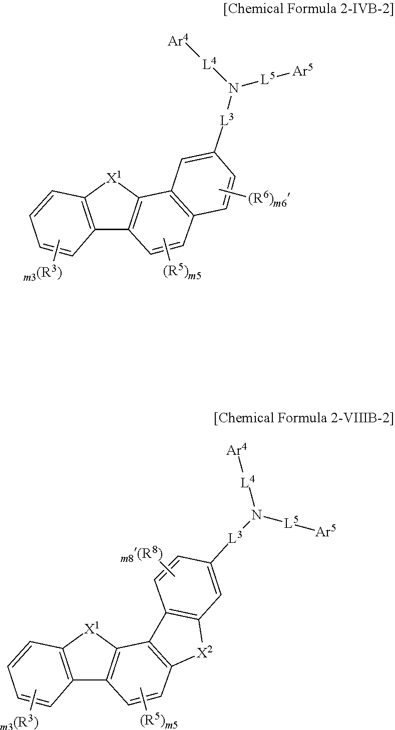
in Chemical Formula 2-IVB-2 and Chemical Formula 2-VIIIB-2,
L³ to L⁵ are each independently a single bond or a substituted or unsubstituted phenylene group,
Ar⁴ and Ar⁵ are each independently a substituted or unsubstituted phenyl group, a substituted or unsubstituted biphenyl group, or a substituted or unsubstituted naphthyl group,
X¹ is NR^{b}, O, or S,
X² is CR^{h}Rⁱ or SiR^{j}R^{k},
R^{b}, R^{h}, Rⁱ, R^{j}, and R^{k} are each independently a substituted or unsubstituted C1 to C10 alkyl group, or a substituted or unsubstituted C6 to C20 aryl group,
R³, R⁵, R⁶, R⁷, and R⁸ are each independently hydrogen, deuterium, a substituted or unsubstituted C1 to C30 alkyl group, or a substituted or unsubstituted C6 to C30 aryl group,
m3 is an integer of 1 to 4,
m5 and m7 are each independently 1 or 2, and
m6′ and m8′ are each independently an integer of 1 to 3.

12. An organic optoelectronic device, comprising:
an anode and a cathode facing each other, and
at least one organic layer between the anode and the cathode,
wherein:
the at least one organic layer includes a light emitting layer, and
the light emitting layer includes the compound as claimed in claim 1.

13. The organic optoelectronic device as claimed in claim 12, wherein the compound is a host of the light emitting layer.

14. A display device comprising the organic optoelectronic device as claimed in claim 12.

15. An organic optoelectronic device, comprising:
an anode and a cathode facing each other, and
at least one organic layer between the anode and the cathode,
wherein:
the at least one organic layer includes a light emitting layer, and
the light emitting layer includes the composition as claimed in claim 8.

16. The organic optoelectronic device as claimed in claim 15, wherein the composition is a host of the light emitting layer.

17. A display device comprising the organic optoelectronic device as claimed in claim 15.
